(19) United States
(12) Patent Application Publication
(10) Pub. No.: US 2023/0422612 A1
(43) Pub. Date: Dec. 28, 2023

(54) ORGANIC OPTOELECTRONIC DEVICE AND DISPLAY DEVICE

(71) Applicant: SAMSUNG SDI CO., LTD., Yongin-si (KR)

(72) Inventors: Ho Kuk JUNG, Suwon-si (KR); Youngmook LIM, Suwon-si (KR); Youngkyoung JO, Suwon-si (KR); Mijin LEE, Suwon-si (KR); Sung-Hyun JUNG, Suwon-si (KR); Dalho HUH, Suwon-si (KR)

(21) Appl. No.: 18/209,595

(22) Filed: Jun. 14, 2023

(30) Foreign Application Priority Data
Jun. 28, 2022 (KR) 10-2022-0079129

(51) Int. Cl. H10K 85/60; C07D 487/04; C09K 11/06; C07D 209/86; C07D 307/77

(52) U.S. Cl. CPC H10K 85/6572; C07D 487/04; C09K 11/06; H10K 85/654; C07D 209/86; C07D 307/77; H10K 85/633; H10K 50/12

(57) ABSTRACT

An organic optoelectronic device and a display device, the organic optoelectronic device includes an anode and a cathode facing each other; a light emitting layer between the anode and the cathode; a hole transport layer between the anode and the light emitting layer; and a hole transport auxiliary layer between the light emitting layer and the hole transport layer, wherein the light emitting layer includes a first compound represented by Chemical Formula 1 and a second compound represented by Chemical Formula 2 or a combination of Chemical Formula 3 and Chemical Formula 4, and the hole transport auxiliary layer includes a third compound represented by a combination of Chemical Formula 5 and Chemical Formula 6.

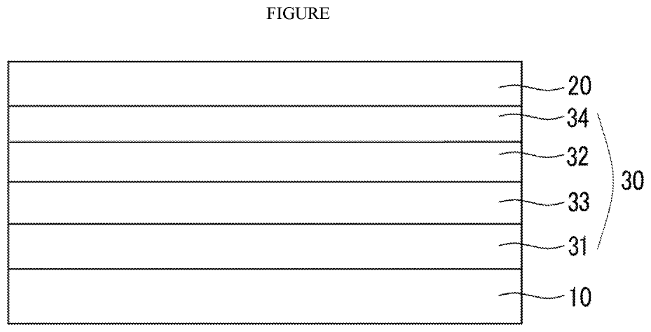

### CROSS-REFERENCE TO RELATED APPLICATION

This application claims priority to and the benefit of Korean Patent Application No. 10-2022-0079129 filed in the Korean Intellectual Property Office on Jun. 28, 2022, the entire contents of which are incorporated herein by reference.

### BACKGROUND

### 1. Field

Embodiments relate to an organic optoelectronic device and a display device.

### 2. Description of the Related Art

An organic optoelectronic device (e.g., organic optoelectronic diode) is a device that converts electrical energy into photoenergy, and vice versa.

An organic optoelectronic device may be classified as follows in accordance with its driving principles. One is a photoelectric device where excitons generated by photoenergy are separated into electrons and holes and the electrons and holes are transferred to different electrodes respectively and electrical energy is generated, and the other is a light emitting device to generate photoenergy from electrical energy by supplying a voltage or a current to electrodes.

Examples of the organic optoelectronic device include an organic photoelectric device, an organic light emitting diode, an organic solar cell, and an organic photo conductor drum.

Among them, the organic light emitting diode (OLED) has recently drawn attention due to an increase in demand for flat panel displays. The organic light emitting diode converts electrical energy into light, and the performance of organic light emitting diode may be influenced by the organic materials between electrodes.

### SUMMARY

The embodiments may be realized by providing an organic optoelectronic device including an anode and a cathode facing each other; a light emitting layer between the anode and the cathode; a hole transport layer between the anode and the light emitting layer; and a hole transport auxiliary layer between the light emitting layer and the hole transport layer, wherein the light emitting layer includes a first compound represented by Chemical Formula 1 and a second compound represented by Chemical Formula 2 or a combination of Chemical Formula 3 and Chemical Formula 4, and the hole transport auxiliary layer includes a third compound represented by a combination of Chemical Formula 5 and Chemical Formula 6,

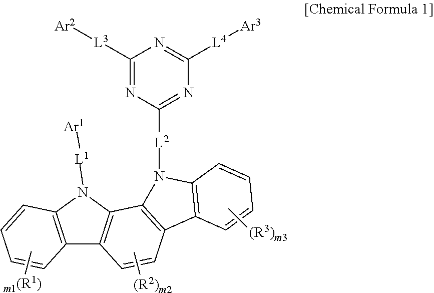

in Chemical Formula 1, Ar¹ to Ar³ are each independently a substituted or unsubstituted C6 to C30 aryl group or a substituted or unsubstituted C2 to C30 heterocyclic group, L¹ to L⁴ are each independently a single bond or a substituted or unsubstituted C6 to C20 arylene group, R¹ to R³ are each independently hydrogen, deuterium, a cyano group, a halogen, a substituted or unsubstituted C1 to C30 alkyl group, or a substituted or unsubstituted C6 to C30 aryl group, m1 and m3 are each independently an integer of 1 to 4, and m2 is 1 or 2;

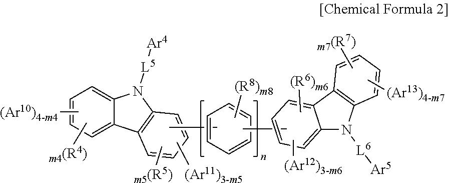

in Chemical Formula 2, Ar⁴ and Ar⁵ are each independently a substituted or unsubstituted C6 to C20 aryl group or a substituted or unsubstituted C2 to C30 heterocyclic group, Ar¹⁰ to Ar¹³ are each independently hydrogen or a substituted or unsubstituted C6 to C30 aryl group, L⁵ and L⁶ are each independently a single bond or a substituted or unsubstituted C6 to C20 arylene group, R⁴ to R⁸ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, m4, m7, and m8 are each independently an integer of 1 to 4, m5 and m6 are each independently an integer of 1 to 3, and n is an integer of 0 to 2;

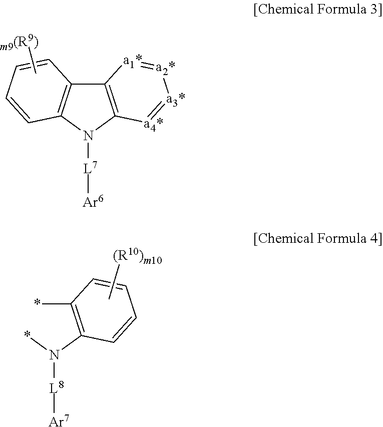

in Chemical Formula 3 and Chemical Formula 4, Ar⁶ and Ar⁷ are each independently a substituted or unsubstituted C6 to C20 aryl group or a substituted or unsubstituted C2 to C30 heterocyclic group, two adjacent ones of a1* to a4* of Chemical Formula 3 are linking carbons linked at * of Chemical Formula 4, and the remaining two of a1* to a4* of Chemical Formula 3, not linked at * of Chemical Formula 4, are C-L^{a}-R^{a}, L^{a}, L⁷, and L⁸ are each independently a single bond or a substituted or unsubstituted C6 to C20 arylene group, R^{a}, R⁹ and R¹⁰ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, and m9 and m10 are each independently an integer of 1 to 4;

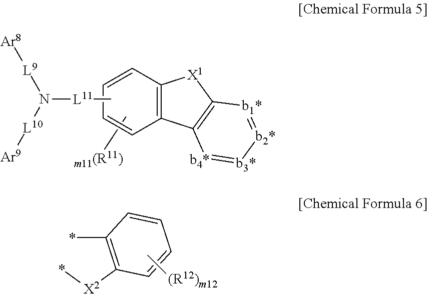

in Chemical Formula 5 and Chemical Formula 6, X¹ and X² are each independently O, S, or CR^{b}R^{c}, two adjacent ones of b1* to b4* of Chemical Formula 5 are linking carbons linked at * of Chemical Formula 6, and the remaining two of b1* to b4* of Chemical Formula 5, not linked at * of Chemical Formula 6, are C-L^{b}-R^{d}, L^{b} and L⁹ to L¹¹ are each independently a single bond, a substituted or unsubstituted C6 to C20 arylene group, or a substituted or unsubstituted C2 to C20 heterocyclic group, Ar⁸ and Ar⁹ are each independently a substituted or unsubstituted C6 to C30 aryl group or a substituted or unsubstituted C2 to C30 heterocyclic group, R^{b} and R^{c} are each independently a substituted or unsubstituted C1 to C30 alkyl group or a substituted or unsubstituted C6 to C30 aryl group, R^{d}, R¹¹ and R¹² are each independently hydrogen, deuterium, a cyano group, a halogen, a substituted or unsubstituted C1 to C30 alkyl group, a substituted or unsubstituted C6 to C30 aryl group, or a substituted or unsubstituted C2 to C30 heterocyclic group, m11 is an integer of 1 to 3, and m12 is an integer of 1 to 4.

The embodiments may be realized by providing a display device including the organic optoelectronic device according to an embodiment.

### BRIEF DESCRIPTION OF THE DRAWING

Features will become apparent to those of skill in the art by describing in detail exemplary embodiments with reference to the attached drawing in which:

the FIGURE is a cross-sectional view illustrating an organic light emitting diode according to an embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

As used herein, “unsubstituted” refers to non-replacement of a hydrogen atom by another substituent and remaining of the hydrogen atom.

As used herein, “hydrogen substitution (—H)” may include “deuterium substitution (-D)” or “tritium substitution (-T).” For example, any hydrogen in any compound described herein may be protium, deuterium, or tritium (e.g., based on natural or artificial substitution).

In one example of the present disclosure, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In addition, in specific examples of the present disclosure, “substituted” refers to replacement of at least one hydrogen of a substituent or a compound by deuterium, a C1 to C20 alkyl group, a C6 to C30 aryl group, or a cyano group. In addition, in specific examples of the present disclosure, “substituted” refers to replacement of at least one hydrogen of a substituent or a compound by deuterium, a C1 to C5 alkyl group, a C6 to C18 aryl group, or a cyano group. In addition, in specific examples of the present disclosure, “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quarterphenyl group, and the like, and a group in which two or more hydrocarbon aromatic moieties are fused directly or indirectly to provide a non-aromatic fused ring, for example, a fluorenyl group, and the like.

The aryl group may include a monocyclic, polycyclic or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to the highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to the lowest unoccupied molecular orbital (LUMO) level.

Hereinafter, an organic optoelectronic device according to an embodiment will be described.

The organic optoelectronic device may be a suitable device to convert electrical energy into photoenergy and vice versa, e.g., an organic photoelectric device, an organic light emitting diode, an organic solar cell, or an organic photo-conductor drum.

Herein, an organic light emitting diode as one example of an organic optoelectronic device is described, or it may be applied to other organic optoelectronic device in the same way.

The FIGURE is a schematic cross-sectional view of an organic optoelectronic device according to an embodiment.

Referring to the FIGURE, an organic optoelectronic device according to an embodiment may include an anode 10 and a cathode 20, and an organic layer 30 between the anode 10 and the cathode 20.

The anode 10 may be made of a conductor having a large work function to help hole injection, and may be, e.g., a metal, a metal oxide, or a conductive polymer. The anode 10 may be, e.g., a metal such as nickel, platinum, vanadium, chromium, copper, zinc, gold, or the like or an alloy thereof, a metal oxide such as zinc oxide, indium oxide, indium tin oxide (ITO), indium zinc oxide (IZO), or the like; a combination of a metal and an oxide such as ZnO and Al or SnO₂ and Sb; or a conductive polymer such as poly(3-methylthiophene), poly(3,4-(ethylene-1,2-dioxy)thiophene) (PEDOT), polypyrrole, or polyaniline. As used herein, the term “or” is not an exclusive term, e.g., “A or B” would include A, B, or A and B.

The cathode 20 may be made of a conductor having a small work function to help electron injection, and may be e.g., a metal, a metal oxide, or a conductive polymer. The cathode 20 may be, e.g., a metal such as magnesium, calcium, sodium, potassium, titanium, indium, yttrium, lithium, gadolinium, aluminum, silver, tin, lead, cesium, barium, or the like, or an alloy thereof, or a multi-layer structure material such as LiF/Al, LiO₂/Al, LiF/Ca, or BaF₂/Ca.

The organic layer 30 may include a hole transport layer 31, a light emitting layer 32, and a hole transport auxiliary layer 33 between the hole transport layer 31 and the light emitting layer 32.

The hole transport layer 31 may be a layer for facilitating hole transport from the anode 10 to the light emitting layer 32, and may include, e.g., an amine compound.

The amine compound may include, e.g., an aryl group or a heteroaryl group. In an implementation, the amine compound may be, e.g., represented by Chemical Formula a or Chemical Formula b.

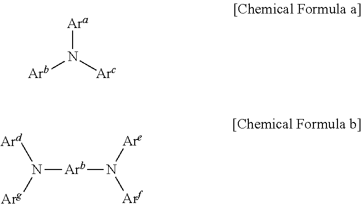

In Chemical Formulae a and b, Ar^{a} to Ar^{g} may each independently be, e.g., hydrogen, deuterium, a substituted or unsubstituted C1 to C20 alkyl group, a substituted or unsubstituted C6 to C30 aryl group, a substituted or unsubstituted C2 to C30 heteroaryl group, or a combination thereof.

In an implementation, at least one of Ar^{a} to Ar^{c} and at least one of Ar^{d} to Ar^{g} may be, e.g., a substituted or unsubstituted C6 to C30 aryl group, a substituted or unsubstituted C2 to C30 heteroaryl group, or a combination thereof.

Ar^{h} may be, e.g., a single bond, a substituted or unsubstituted C1 to C20 alkylene group, a substituted or unsubstituted C6 to C30 arylene group, a substituted or unsubstituted C2 to C30 heteroarylene group, or a combination thereof.

The light emitting layer 32 may include at least two types of hosts and dopants, e.g., the host may include a first compound having a bipolar characteristic having relatively strong electronic characteristic and a second compound having a bipolar characteristic having a relatively strong hole characteristic.

The first compound may be represented by Chemical Formula 1.

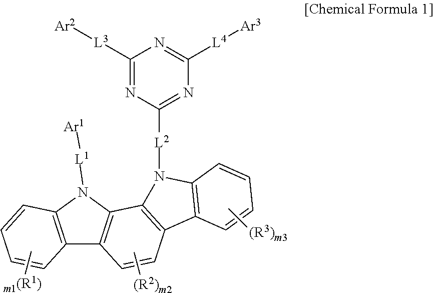

In Chemical Formula 1, Ar¹ to Ar³ may each independently be or include, e.g., a substituted or unsubstituted C6 to C30 aryl group or a substituted or unsubstituted C2 to C30 heterocyclic group.

L¹ to L⁴ may each independently be or include, e.g., a single bond or a substituted or unsubstituted C6 to C20 arylene group.

R¹ to R³ may each independently be or include, e.g., hydrogen, deuterium, a cyano group, a halogen, a substituted or unsubstituted C1 to C30 alkyl group, or a substituted or unsubstituted C6 to C30 aryl group.

m1 and m3 may each independently be, e.g., an integer of 1 to 4.

m2 may be, e.g., 1 or 2.

In Chemical Formula 1, when m1 is 2, 3, or 4, each R¹ may be the same or different from each other.

In Chemical Formula 1, when m2 is 2, each R² may be the same or different from each other.

In Chemical Formula 1, when m3 is 2, 3, or 4, each R³ may be the same or different from each other.

The first compound may have at least one substituted or unsubstituted C10 to C30 aryl group, or a substituted or unsubstituted C10 to C30 heterocyclic group as an additional substituent of triazine substituted at the N position of the indolocarbazole group and thereby the LUMO electron cloud may be expanded to exhibit smooth electron injection and transport characteristics, resulting in intramolecular hole and electron balance. In the case of an organic light emitting diode to which this is applied, a driving voltage of the diode may be lowered, and lifespan characteristics may be improved due to stability by hole and electron balance.

In an implementation, Ar¹ to Ar³ of Chemical Formula 1 may each independently be, e.g., a substituted or unsubstituted phenyl group, a substituted or unsubstituted biphenyl group, a substituted or unsubstituted terphenyl group, a substituted or unsubstituted carbazolyl group, a substituted or unsubstituted a dibenzofuranyl group, or a substituted or unsubstituted dibenzothiophenyl group.

In an implementation, at least one of Ar² and Ar³ may be, e.g., a substituted or unsubstituted biphenyl group, a substituted or unsubstituted carbazolyl group, a substituted or unsubstituted dibenzofuranyl group, or a substituted or unsubstituted dibenzothiophenyl group.

In an implementation, the first compound may be, e.g., a compound of Group 1.

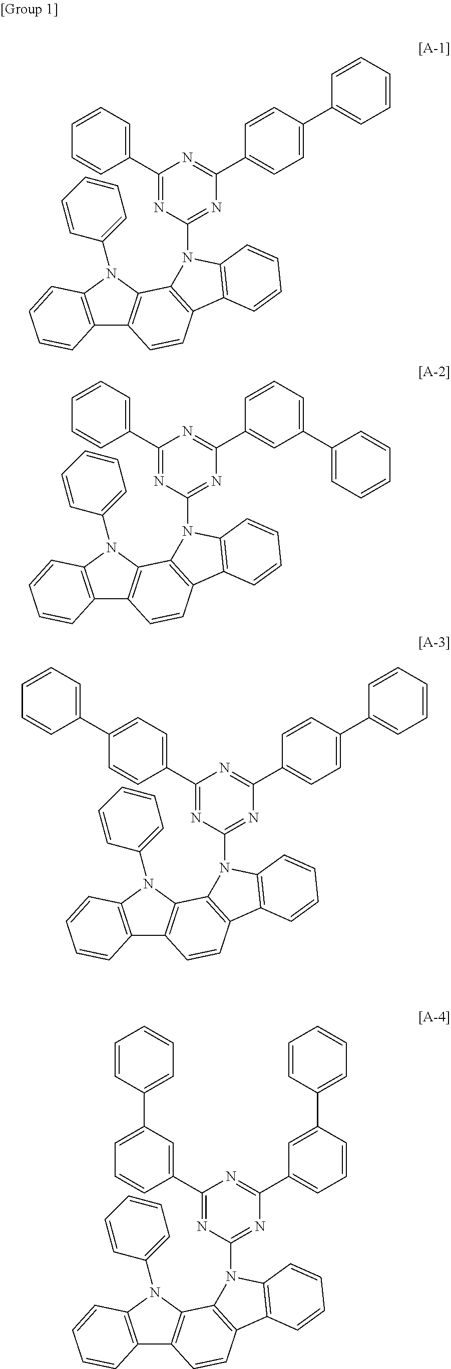

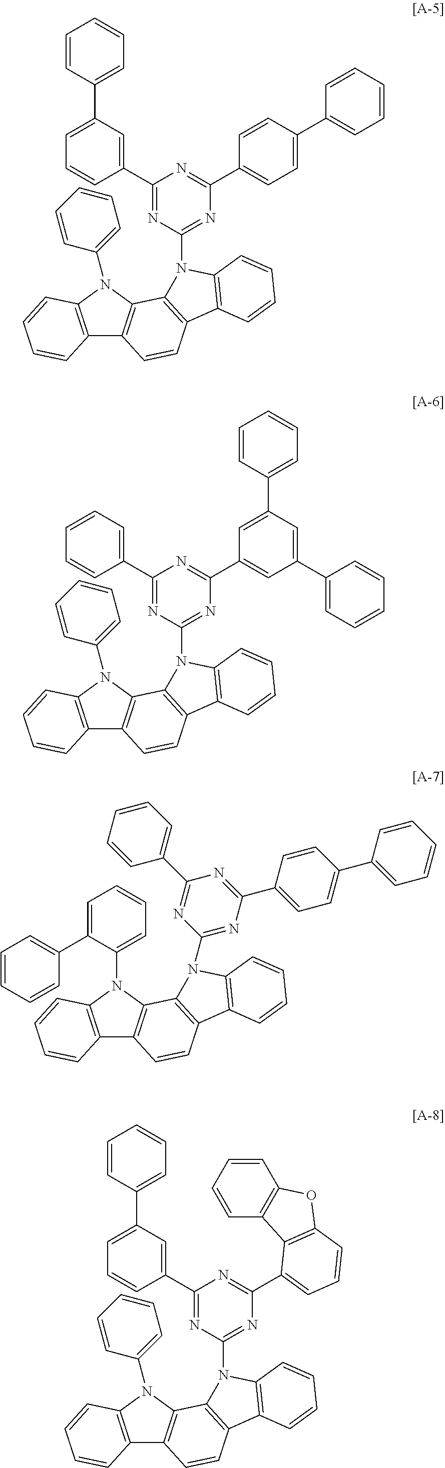 [SMILES: c1ccc(-c2ccc(-c3nc(-c4cccc(-c5ccccc5)c4)nc(-n4c5ccccc5c5ccc6c7ccccc7n(-c7ccccc7)c6c54)n3)cc2)cc1 ; c1ccc(-c2cc(-c3ccccc3)cc(-c3nc(-c4ccccc4)nc(-n4c5ccccc5c5ccc6c7ccccc7n(-c7ccccc7)c6c54)n3)c2)cc1 ; c1ccc(-c2ccc(-c3nc(-c4ccccc4)nc(-n4c5ccccc5c5ccc6c7ccccc7n(-c7ccccc7-c7ccccc7)c6c54)n3)cc2)cc1 ; c1ccc(-c2cccc(-c3nc(-c4cccc5oc6ccccc6c45)nc(-n4c5ccccc5c5ccc6c7ccccc7n(-c7ccccc7)c6c54)n3)c2)cc1]

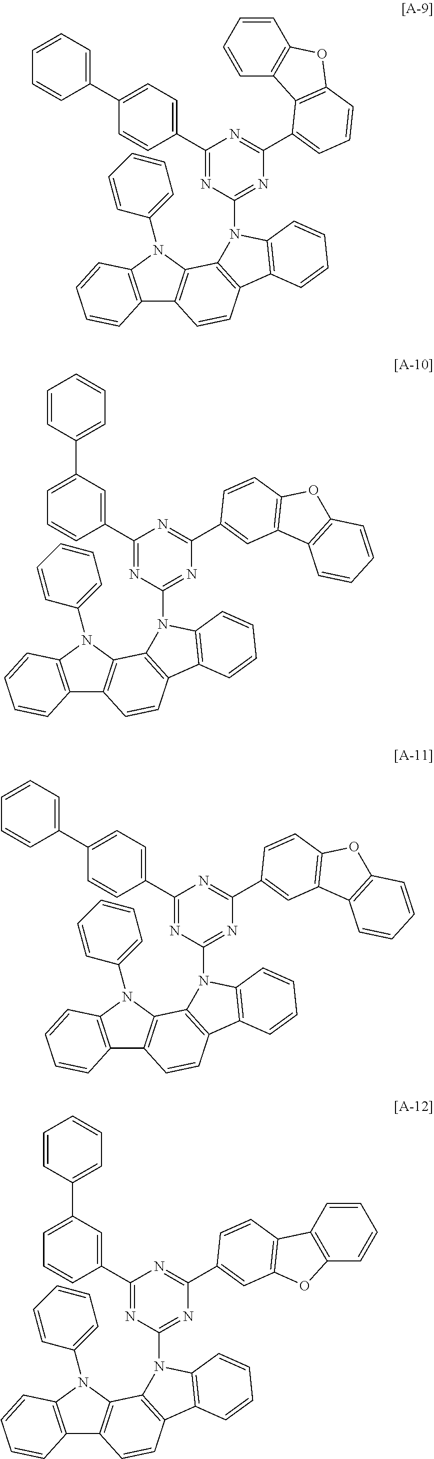

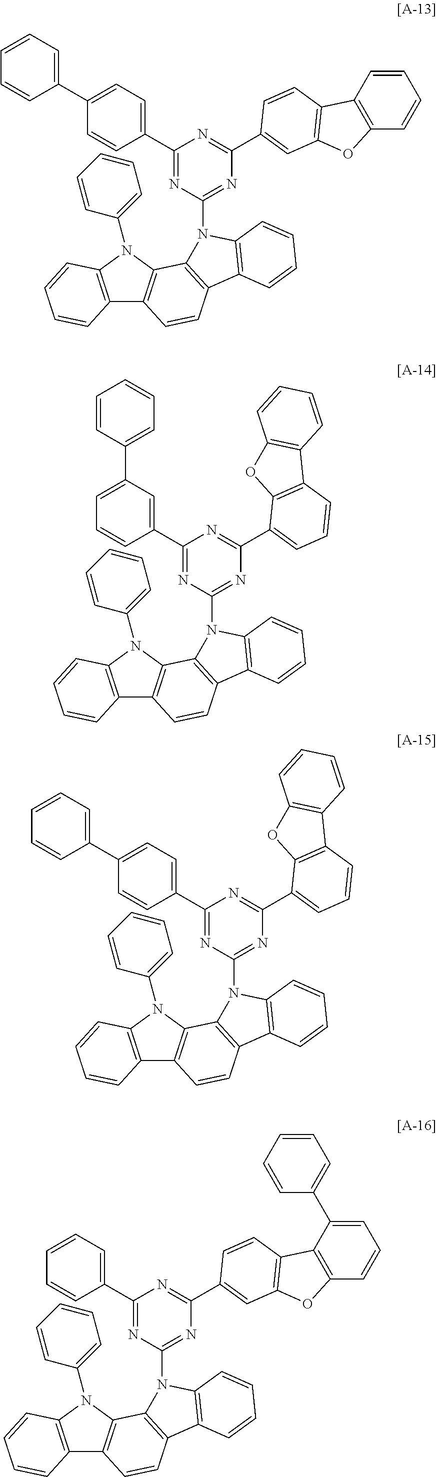 [SMILES: c1ccc(-c2ccc(-c3nc(-c4ccc5c(c4)oc4ccccc45)nc(-n4c5ccccc5c5ccc6c7ccccc7n(-c7ccccc7)c6c54)n3)cc2)cc1 ; c1ccc(-c2cccc(-c3nc(-c4cccc5c4oc4ccccc45)nc(-n4c5ccccc5c5ccc6c7ccccc7n(-c7ccccc7)c6c54)n3)c2)cc1 ; c1ccc(-c2ccc(-c3nc(-c4cccc5c4oc4ccccc45)nc(-n4c5ccccc5c5ccc6c7ccccc7n(-c7ccccc7)c6c54)n3)cc2)cc1 ; c1ccc(-c2nc(-c3ccc4c(c3)oc3cccc(-c5ccccc5)c34)nc(-n3c4ccccc4c4ccc5c6ccccc6n(-c6ccccc6)c5c43)n2)cc1]

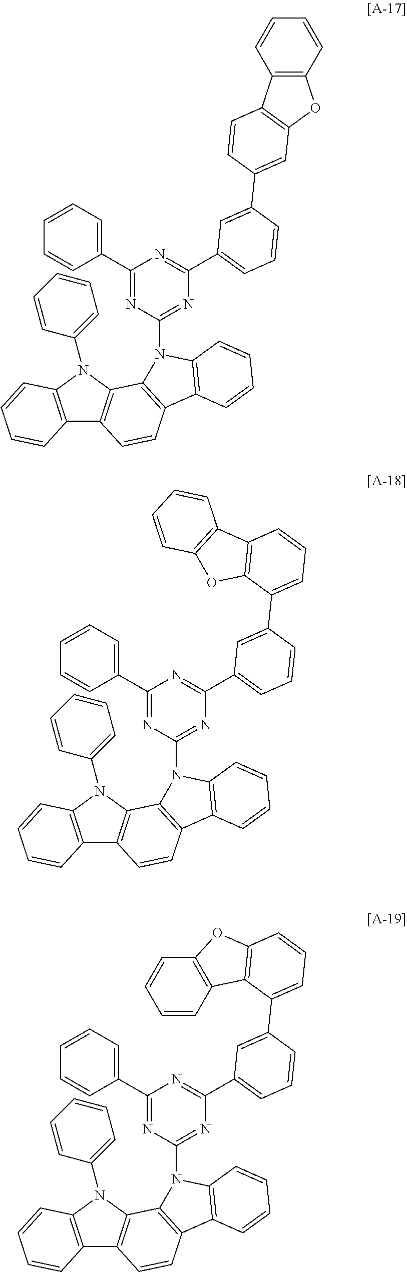 [SMILES: c1ccc(-c2nc(-c3cccc(-c4ccc5c(c4)oc4ccccc45)c3)nc(-n3c4ccccc4c4ccc5c6ccccc6n(-c6ccccc6)c5c43)n2)cc1 ; c1ccc(-c2nc(-c3cccc(-c4cccc5c4oc4ccccc45)c3)nc(-n3c4ccccc4c4ccc5c6ccccc6n(-c6ccccc6)c5c43)n2)cc1 ; c1ccc(-c2nc(-c3cccc(-c4cccc5oc6ccccc6c45)c3)nc(-n3c4ccccc4c4ccc5c6ccccc6n(-c6ccccc6)c5c43)n2)cc1]

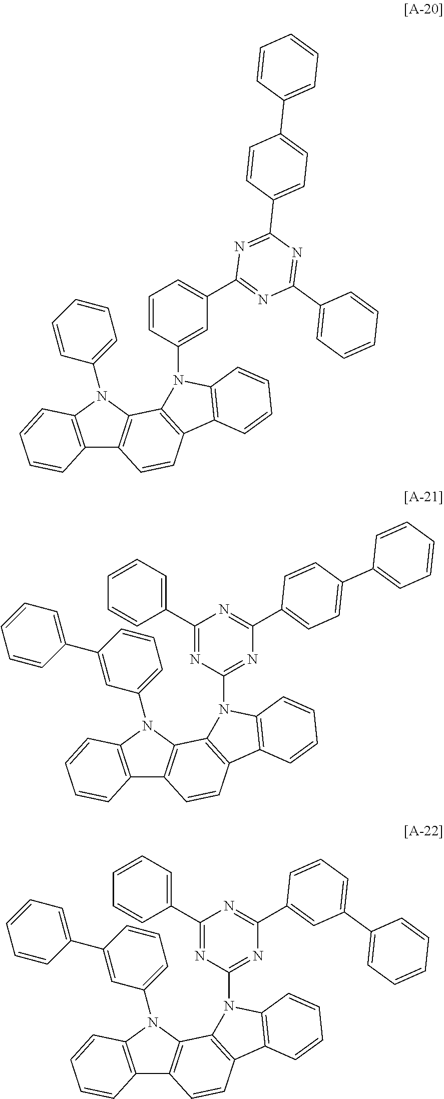 [SMILES: c1ccc(-c2ccc(-c3nc(-c4ccccc4)nc(-c4cccc(-n5c6ccccc6c6ccc7c8ccccc8n(-c8ccccc8)c7c65)c4)n3)cc2)cc1 ; c1ccc(-c2ccc(-c3nc(-c4ccccc4)nc(-n4c5ccccc5c5ccc6c7ccccc7n(-c7cccc(-c8ccccc8)c7)c6c54)n3)cc2)cc1 ; c1ccc(-c2cccc(-c3nc(-c4ccccc4)nc(-n4c5ccccc5c5ccc6c7ccccc7n(-c7cccc(-c8ccccc8)c7)c6c54)n3)c2)cc1]

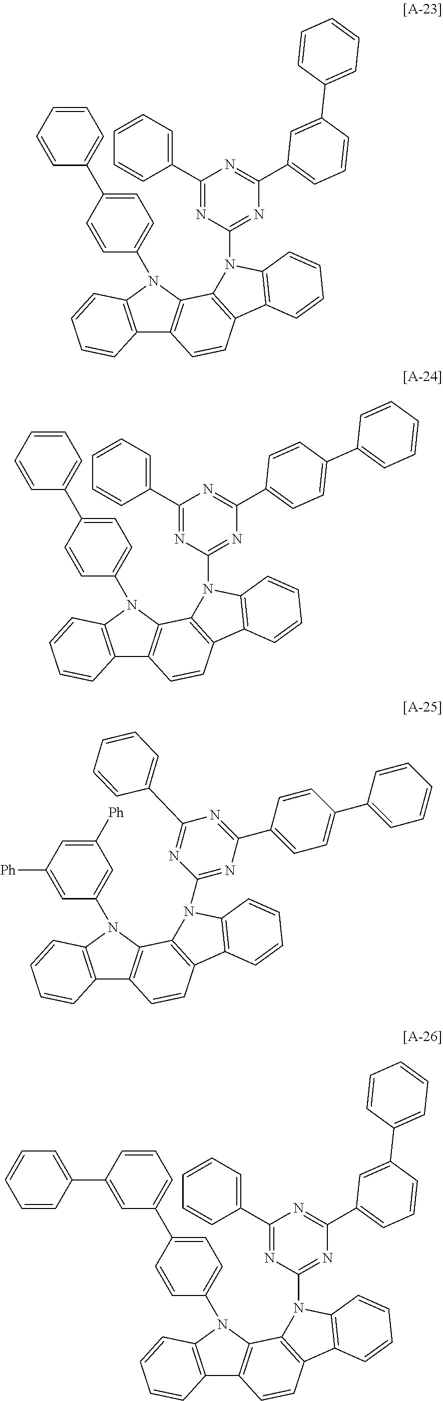 [SMILES: c1ccc(-c2ccc(-n3c4ccccc4c4ccc5c6ccccc6n(-c6nc(-c7ccccc7)nc(-c7cccc(-c8ccccc8)c7)n6)c5c43)cc2)cc1 ; c1ccc(-c2ccc(-c3nc(-c4ccccc4)nc(-n4c5ccccc5c5ccc6c7ccccc7n(-c7ccc(-c8ccccc8)cc7)c6c54)n3)cc2)cc1 ; c1ccc(-c2ccc(-c3nc(-c4ccccc4)nc(-n4c5ccccc5c5ccc6c7ccccc7n(-c7cc(-c8ccccc8)cc(-c8ccccc8)c7)c6c54)n3)cc2)cc1 ; c1ccc(-c2cccc(-c3ccc(-n4c5ccccc5c5ccc6c7ccccc7n(-c7nc(-c8ccccc8)nc(-c8cccc(-c9ccccc9)c8)n7)c6c54)cc3)c2)cc1]

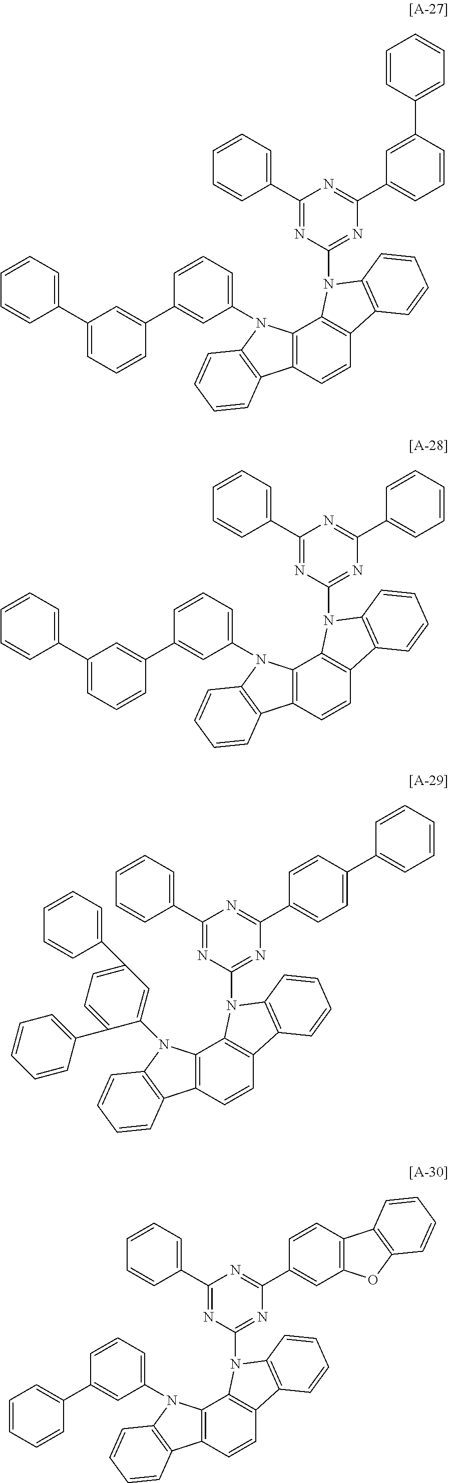 [SMILES: c1ccc(-c2cccc(-c3cccc(-n4c5ccccc5c5ccc6c7ccccc7n(-c7nc(-c8ccccc8)nc(-c8cccc(-c9ccccc9)c8)n7)c6c54)c3)c2)cc1 ; c1ccc(-c2cccc(-c3cccc(-n4c5ccccc5c5ccc6c7ccccc7n(-c7nc(-c8ccccc8)nc(-c8ccccc8)n7)c6c54)c3)c2)cc1 ; c1ccc(-c2ccc(-c3nc(-c4ccccc4)nc(-n4c5ccccc5c5ccc6c7ccccc7n(-c7cc(-c8ccccc8)ccc7-c7ccccc7)c6c54)n3)cc2)cc1 ; c1ccc(-c2cccc(-n3c4ccccc4c4ccc5c6ccccc6n(-c6nc(-c7ccccc7)nc(-c7ccc8c(c7)oc7ccccc78)n6)c5c43)c2)cc1]

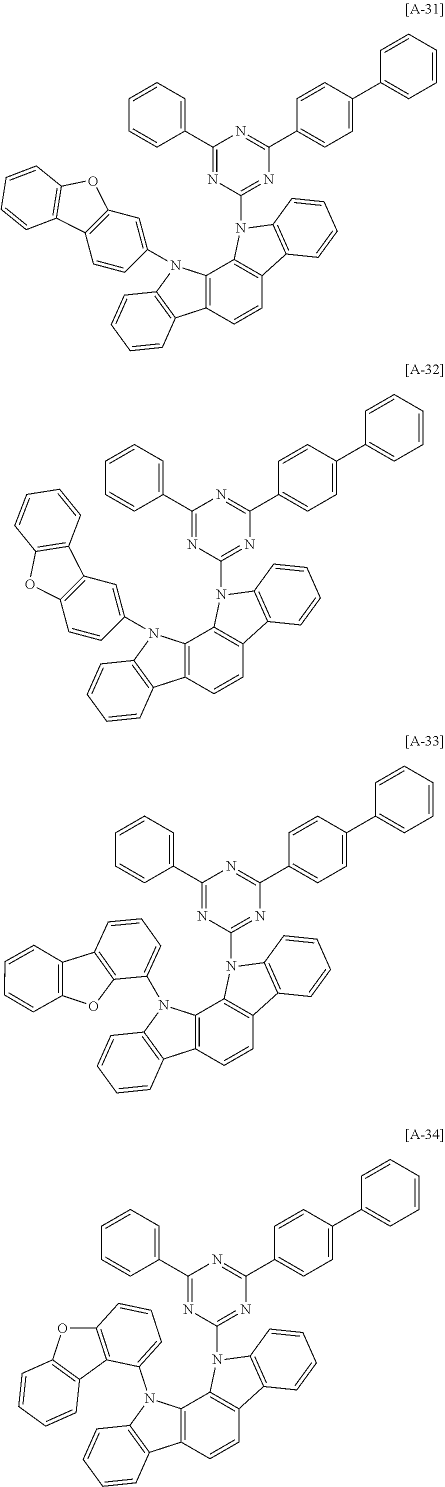 [SMILES: c1ccc(-c2ccc(-c3nc(-c4ccccc4)nc(-n4c5ccccc5c5ccc6c7ccccc7n(-c7ccc8c(c7)oc7ccccc78)c6c54)n3)cc2)cc1 ; c1ccc(-c2ccc(-c3nc(-c4ccccc4)nc(-n4c5ccccc5c5ccc6c7ccccc7n(-c7ccc8oc9ccccc9c8c7)c6c54)n3)cc2)cc1 ; c1ccc(-c2ccc(-c3nc(-c4ccccc4)nc(-n4c5ccccc5c5ccc6c7ccccc7n(-c7cccc8c7oc7ccccc78)c6c54)n3)cc2)cc1 ; c1ccc(-c2ccc(-c3nc(-c4ccccc4)nc(-n4c5ccccc5c5ccc6c7ccccc7n(-c7cccc8oc9ccccc9c78)c6c54)n3)cc2)cc1]

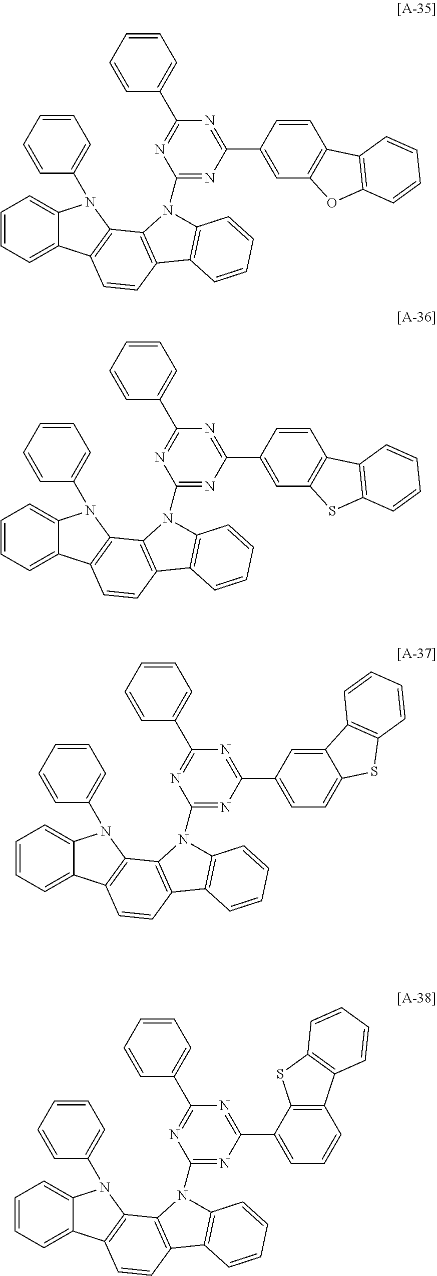 [SMILES: c1ccc(-c2nc(-c3ccc4c(c3)oc3ccccc34)nc(-n3c4ccccc4c4ccc5c6ccccc6n(-c6ccccc6)c5c43)n2)cc1 ; c1ccc(-c2nc(-c3ccc4c(c3)sc3ccccc34)nc(-n3c4ccccc4c4ccc5c6ccccc6n(-c6ccccc6)c5c43)n2)cc1 ; c1ccc(-c2nc(-c3ccc4sc5ccccc5c4c3)nc(-n3c4ccccc4c4ccc5c6ccccc6n(-c6ccccc6)c5c43)n2)cc1 ; c1ccc(-c2nc(-c3cccc4c3sc3ccccc34)nc(-n3c4ccccc4c4ccc5c6ccccc6n(-c6ccccc6)c5c43)n2)cc1]

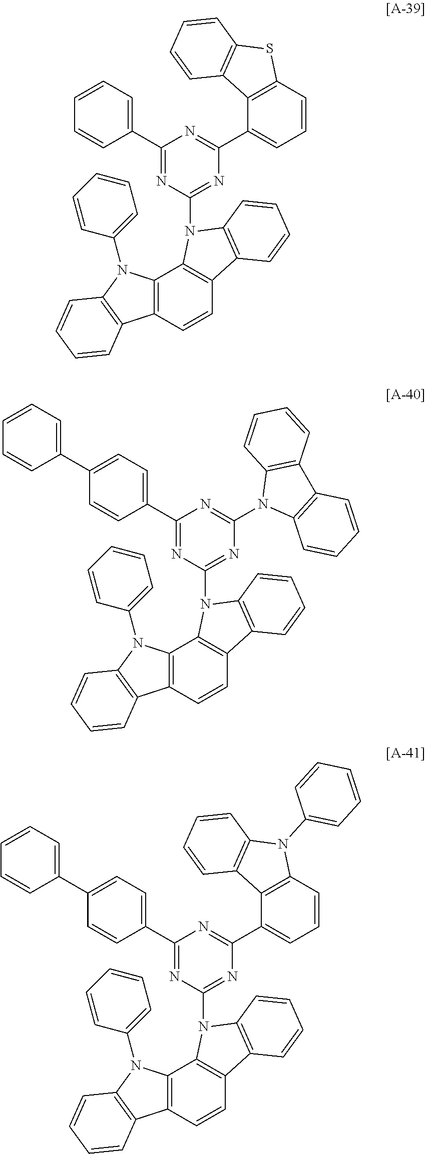 [SMILES: c1ccc(-c2nc(-c3cccc4sc5ccccc5c34)nc(-n3c4ccccc4c4ccc5c6ccccc6n(-c6ccccc6)c5c43)n2)cc1 ; c1ccc(-c2ccc(-c3nc(-n4c5ccccc5c5ccccc54)nc(-n4c5ccccc5c5ccc6c7ccccc7n(-c7ccccc7)c6c54)n3)cc2)cc1 ; c1ccc(-c2ccc(-c3nc(-c4cccc5c4c4ccccc4n5-c4ccccc4)nc(-n4c5ccccc5c5ccc6c7ccccc7n(-c7ccccc7)c6c54)n3)cc2)cc1]

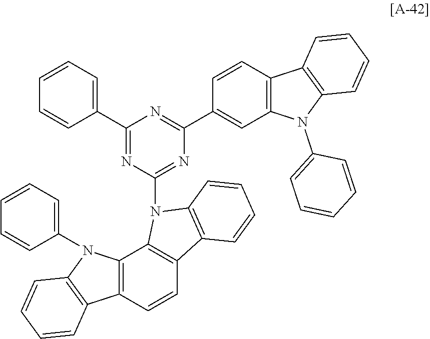 [SMILES: c1ccc(-c2nc(-c3ccc4c5ccccc5n(-c5ccccc5)c4c3)nc(-n3c4ccccc4c4ccc5c6ccccc6n(-c6ccccc6)c5c43)n2)cc1]

One type or two or more types of the first compound may be used.

The second compound may be used in the light emitting layer together with the first compound to help improve luminous efficiency and life-span characteristics by increasing charge mobility and increasing stability.

In an implementation, the second compound may be, e.g., represented by Chemical Formula 2.

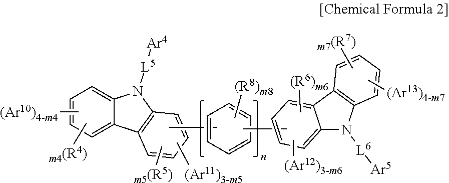

In Chemical Formula 2, Ar⁴ and Ar⁵ may each independently be or include, e.g., a substituted or unsubstituted C6 to C20 aryl group or a substituted or unsubstituted C2 to C30 heterocyclic group.

Ar¹⁰ to Ar¹³ may each independently be or include, e.g., hydrogen or a substituted or unsubstituted C6 to C30 aryl group.

L⁵ and L⁶ may each independently be or include, e.g., a single bond or a substituted or unsubstituted C6 to C20 arylene group.

R⁴ to R⁸ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

m4, m7, and m8 may each independently be, e.g., an integer of 1 to 4.

m5 and m6 may each independently be, e.g., an integer of 1 to 3.

n may be, e.g., an integer of 0 to 2.

When m4 is 2, 3, or 4, each R⁴ may be the same or different from each other.

When m5 is 2 or 3, each R⁵ may be the same or different from each other.

When m6 is 2 or 3, each R⁶ may be the same or different from each other.

When m7 is 2, 3, or 4, each R⁷ may be the same or different from each other.

When m8 is 2, 3, or 4, each R⁸ may be the same or different from each other.

In an implementation, Ar⁴ and Ar⁵ of Chemical Formula 2 may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

L⁵ and L⁶ of Chemical Formula 2 may each independently be, e.g., a single bond, a substituted or unsubstituted phenylene group, or a substituted or unsubstituted biphenylene group.

R⁴ to R⁸ of Chemical Formula 2 may each independently be, e.g., hydrogen, deuterium, or a substituted or unsubstituted C6 to C12 aryl group.

n may be 0 or 1.

In an implementation, Ar¹⁰ to Ar¹³ in Chemical Formula 1 may each independently be, e.g., hydrogen or a substituted or unsubstituted C6 to C12 aryl group.

In an implementation, Ar¹⁰ to Ar¹³ in Chemical Formula 1 may each independently be, e.g., hydrogen or a substituted or unsubstituted phenyl group.

For example, “substitution” in Chemical Formula 2 means that at least one hydrogen is replaced by deuterium, a C1 to C4 alkyl group, a C6 to C18 aryl group, or a C2 to C30 heteroaryl group.

In an implementation, Chemical Formula 2 may be represented by one of Chemical Formula 2-1 to Chemical Formula 2-15.

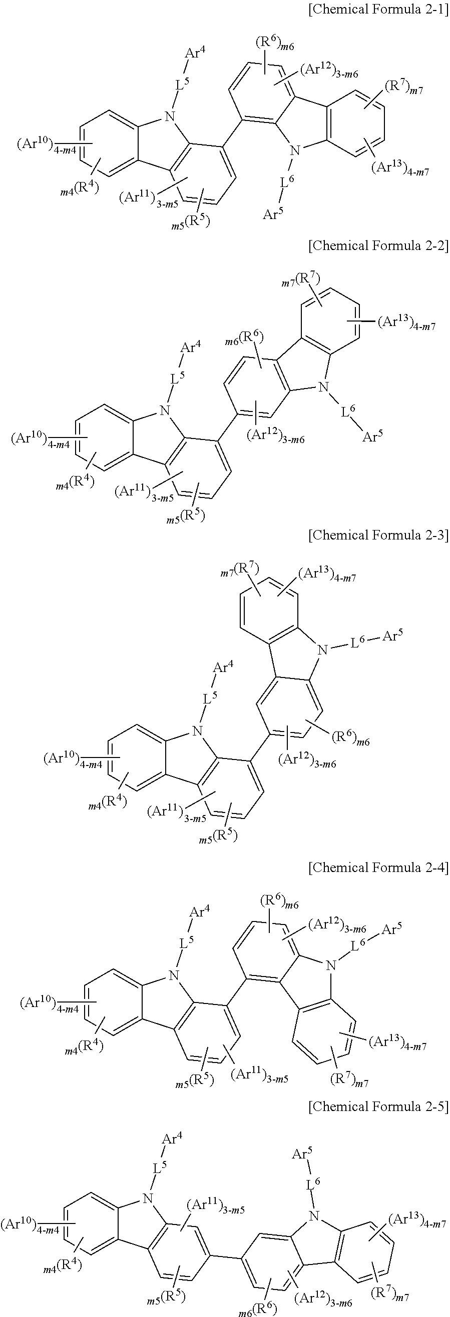

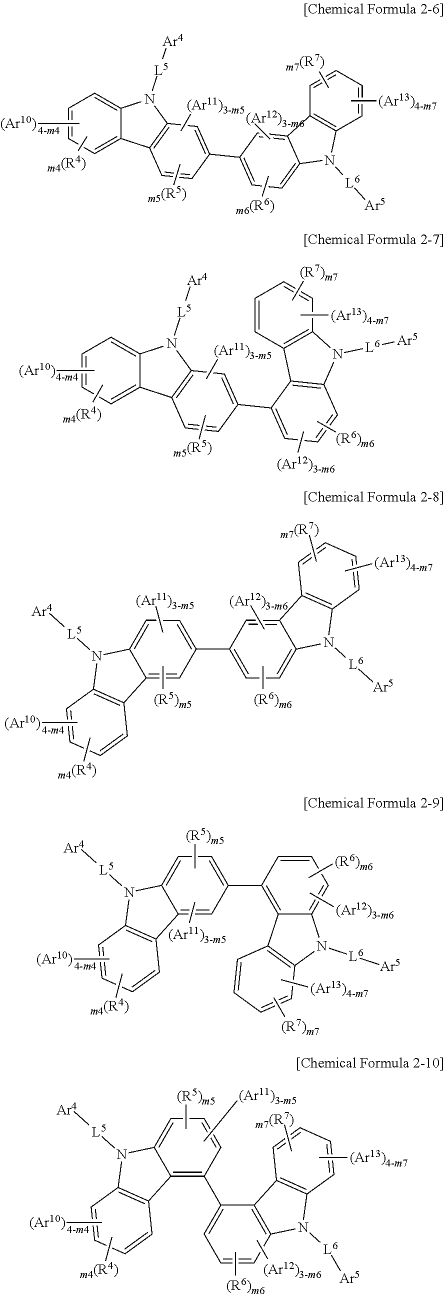

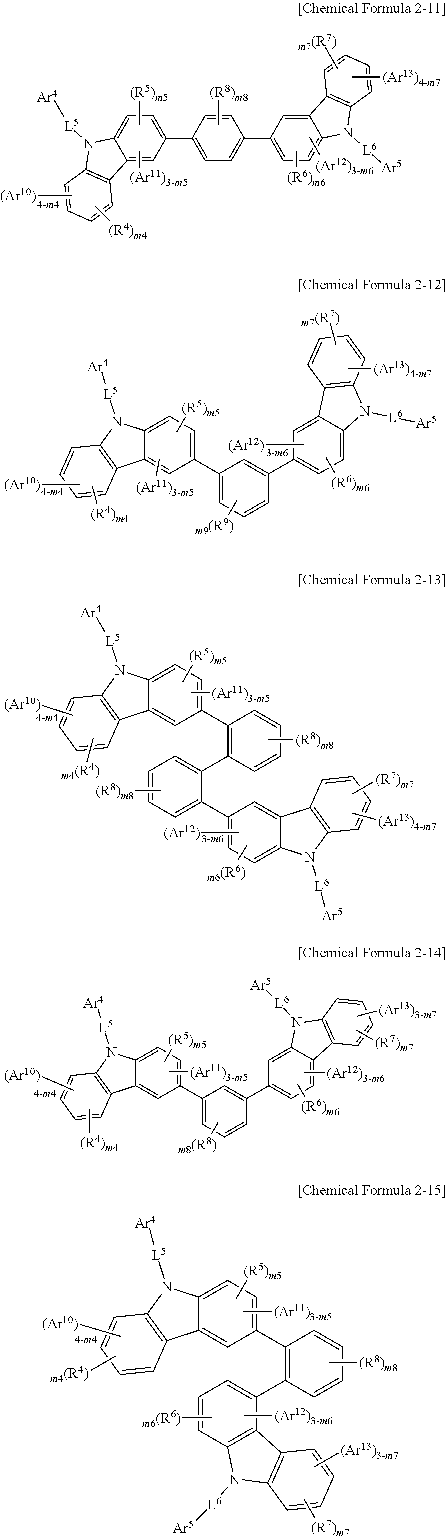

In Chemical Formula 2-1 to Chemical Formula 2-15, R⁴ to R⁸ may each independently be, e.g., hydrogen, deuterium, or a substituted or unsubstituted C6 to C12 aryl group, Ar¹⁰ to Ar¹³ may each independently be, e.g., hydrogen or a substituted or unsubstituted a C6 to C12 aryl group, and moieties *-L⁵-Ar⁴ and *-L⁶-Ar⁵ may each independently be, e.g., a moiety of Group II.

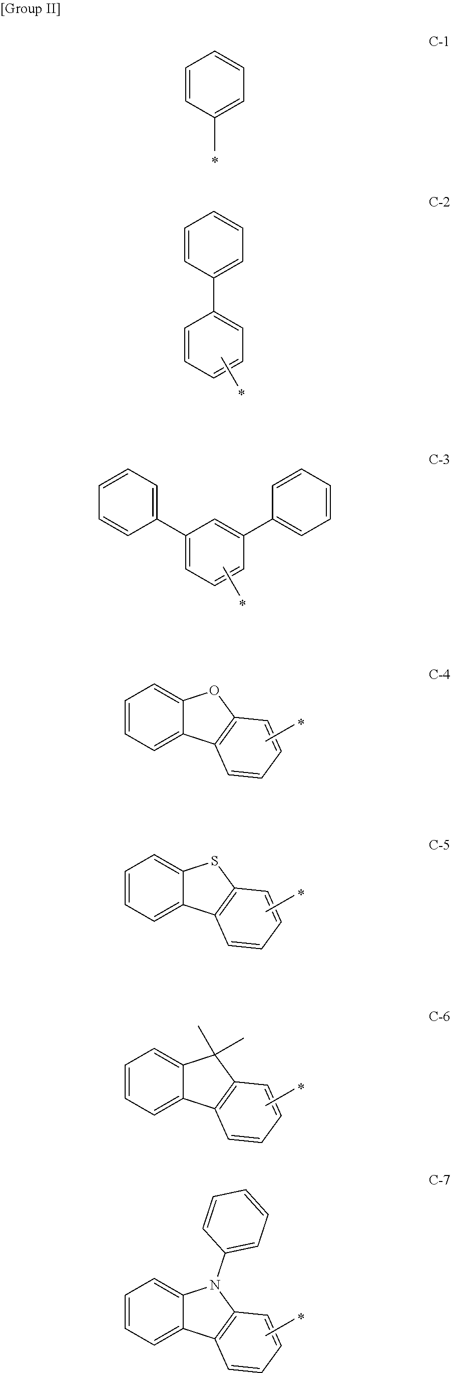

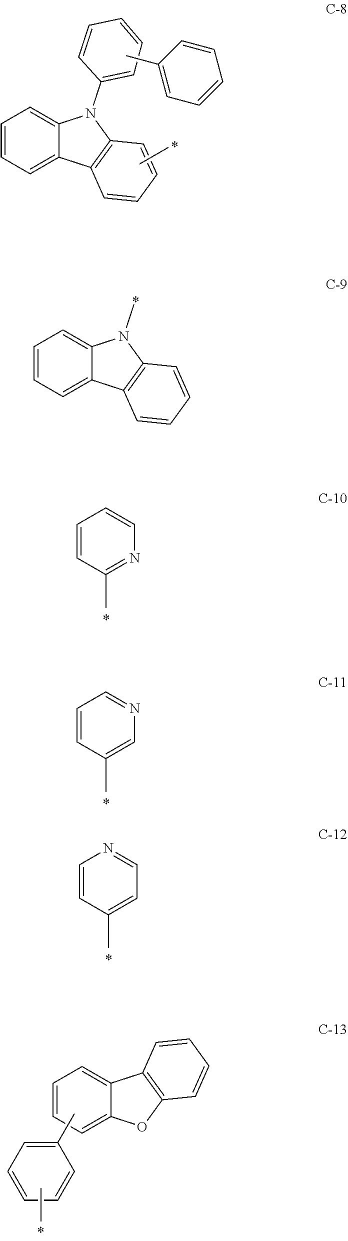

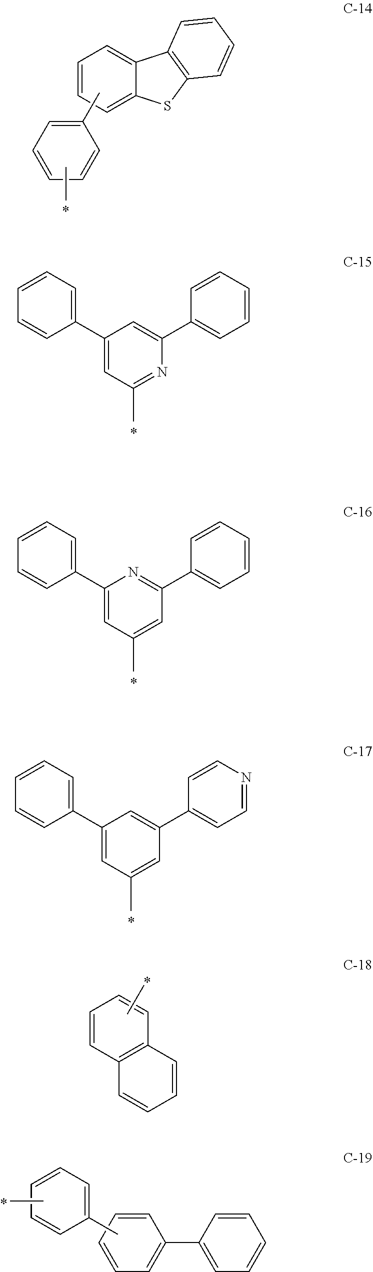

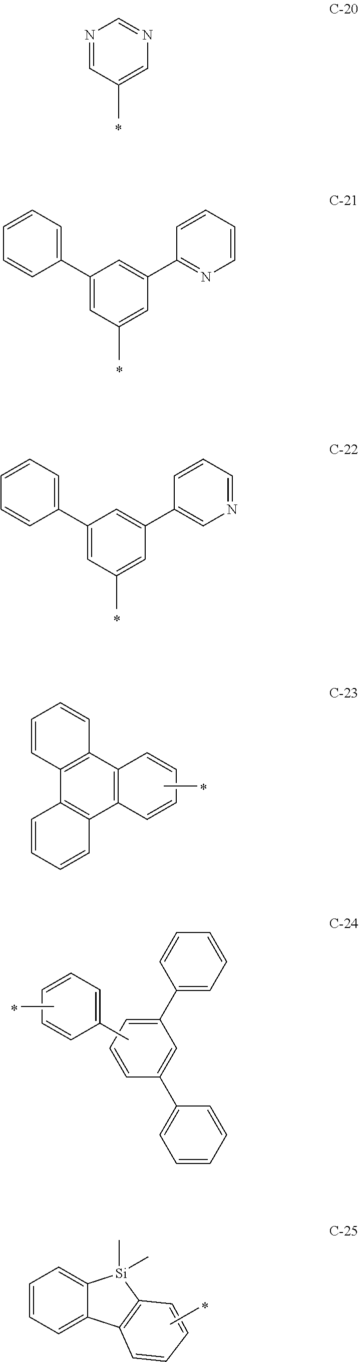

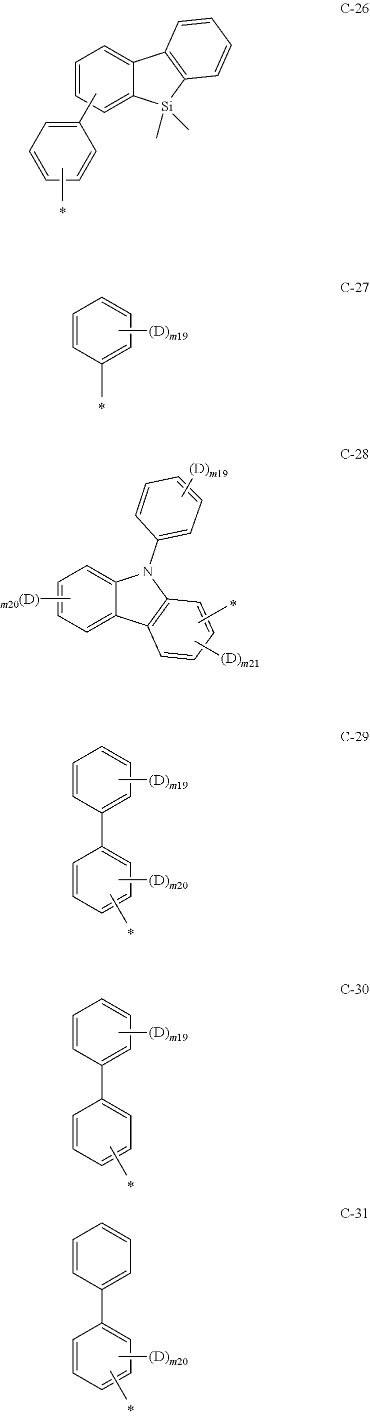

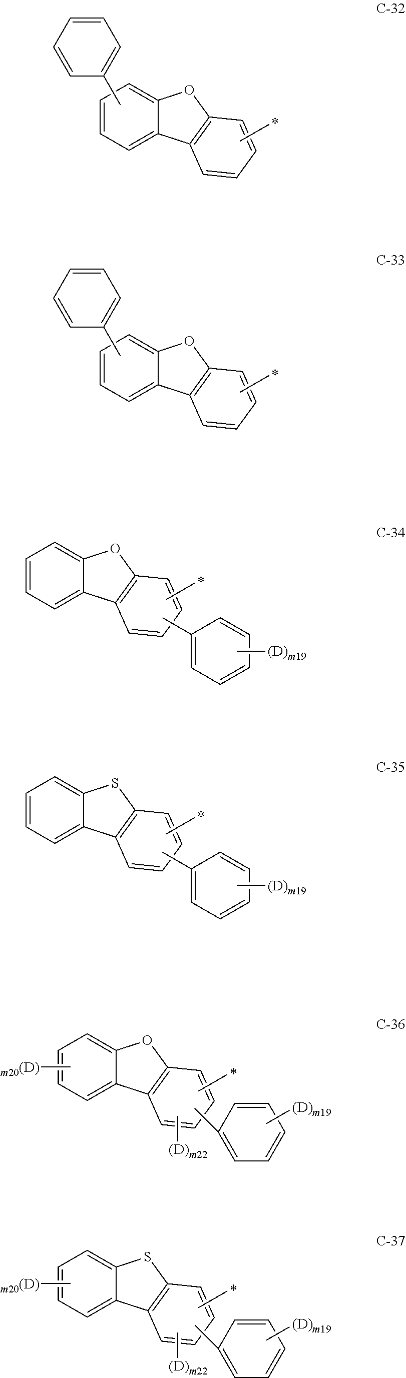

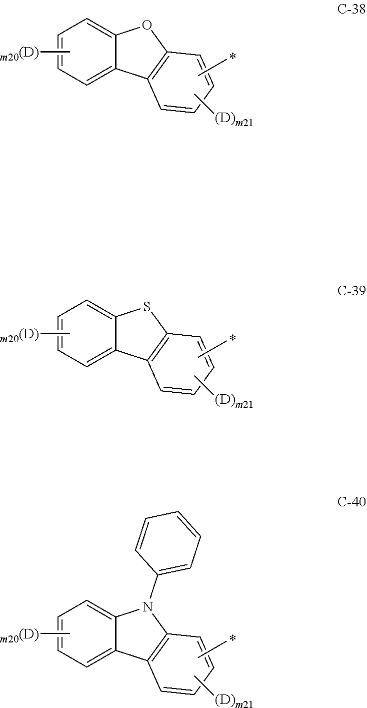

In Group II, D is deuterium.

m19 may be, e.g., an integer of 1 to 5.

m20 may be, e.g., an integer of 1 to 4.

m21 may be, e.g., an integer of 1 to 3.

m22 may be, e.g., 1 or 2.

* is a linking point.

In an implementation, Chemical Formula 2 may be represented by Chemical Formula 2-8.

In an implementation, moieties *-L⁵-Ar⁴ and *-L⁶-Ar⁵ of Chemical Formula 2-8 may each independently be, e.g., C-1, C-2, C-3, C-4, C-7, C-8, or C-9 of Group II.

In an implementation, the second compound may be represented by, e.g., a combination of Chemical Formula 3 and Chemical Formula 4.

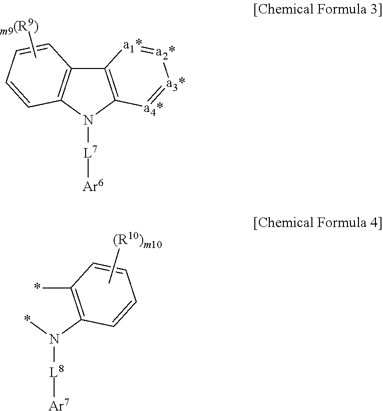

In Chemical Formula 3 and Chemical Formula 4, Ar⁶ and Ar⁷ may each independently be or include, e.g., a substituted or unsubstituted C6 to C20 aryl group or a substituted or unsubstituted C2 to C30 heterocyclic group.

Two adjacent ones of a1* to a4* of Chemical Formula 3 may be linking carbons linked at * of Chemical Formula 4, and the remaining two of a1* to a4* of Chemical Formula 3, not linked at * of Chemical Formula 4, may be, e.g., C-L^{a}-R^{a}. As used herein, the term “linking carbon” refers to a shared carbon at which fused rings are linked.

L^{a}, L⁷, and L⁸ may each independently be or include, e.g., a single bond or a substituted or unsubstituted C6 to C20 arylene group.

R^{a}, R⁹, and R¹⁰ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

m9 and m10 may each independently be, e.g., an integer of 1 to 4.

When m9 is 2, 3, or 4, each R⁹ may be the same or different from each other.

When m10 is 2, 3, or 4, each R¹⁰ may be the same or different from each other.

In an implementation, the second compound represented by the combination of Chemical Formula 3 and Chemical Formula 4 may be represented by, e.g., Chemical Formula 3A, Chemical Formula 3B, Chemical Formula 3C, Chemical Formula 3D, or Chemical Formula 3E.

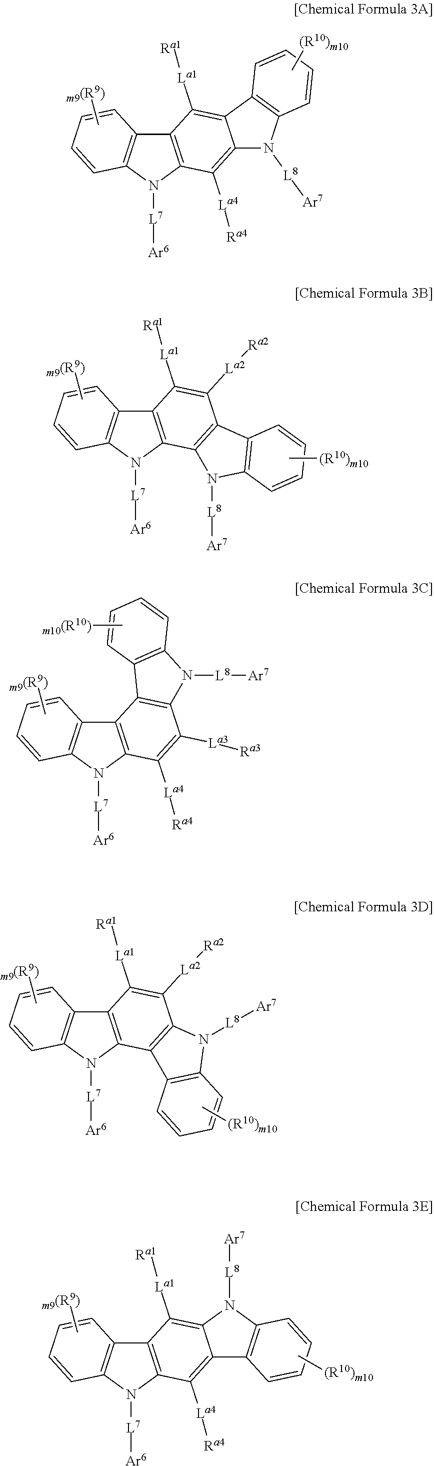

In Chemical Formula 3A to Chemical Formula 3E, Ar⁶, Ar⁷, L⁷, L⁸, R⁹, and R¹⁰ may be defined the same as those described above.

L^{a1} to L^{a4} may be defined the same as L⁷ and L⁸ described above.

R^{a1} to R^{a4} may be defined the same as R⁹ and R¹⁰ described above.

In an implementation, Ar⁶ and Ar⁷ of Chemical Formulas 3 and 4 may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

Rai to R^{a4}, R⁹, and R¹⁰ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, Ar⁶ and Ar⁷ of Chemical Formulas 3 and 4 may each independently be, e.g., a moiety of Group IT.

In an implementation, R^{a1} to R^{a4}, R⁹, and R¹⁰ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, R^{a1} to R^{a4}, R⁹, and R¹⁰ may each independently be, e.g., hydrogen, deuterium, a cyano group, or a substituted or unsubstituted phenyl group.

In an implementation, R^{a1} to R^{a4}, R⁹, and R¹⁰ may each independently be, e.g., hydrogen, deuterium, or a substituted or unsubstituted phenyl group.

In an implementation, the second compound may be represented by Chemical Formula 2-8, and in Chemical Formula 2-8, Ar⁴ and Ar⁵ may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L⁵ and L⁶ may each independently be, e.g., a single bond, or a substituted or unsubstituted C6 to C20 arylene group, and R⁴ to R⁸ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, the second compound may be represented by Chemical Formula 3C, and in Chemical Formula 3C, L^{a3} and L^{a4} may each be, e.g., a single bond, L⁷ and L⁸ may each independently be, e.g., a single bond or a substituted or unsubstituted C6 to C12 arylene group, R⁹, R¹⁰, R^{a3}, and R^{a4} may each independently be, e.g., hydrogen, deuterium, or a substituted or unsubstituted phenyl group, and Ar⁶ and Ar⁷ may each independently be, e.g., a substituted or unsubstituted phenyl group, a substituted or unsubstituted biphenyl group, or a substituted or unsubstituted terphenyl group.

In an implementation, the second compound may be, e.g., a compound of Group 2.

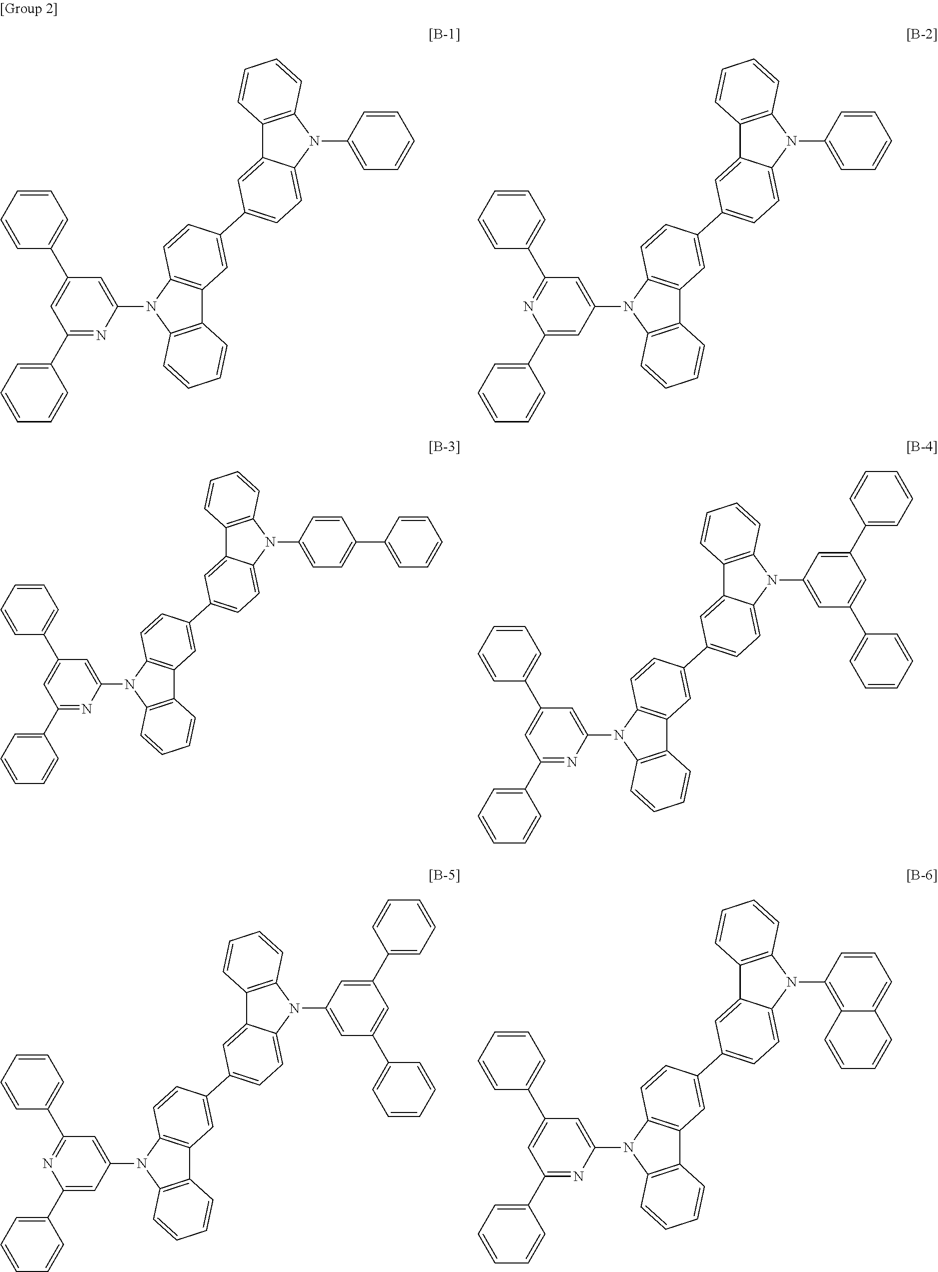

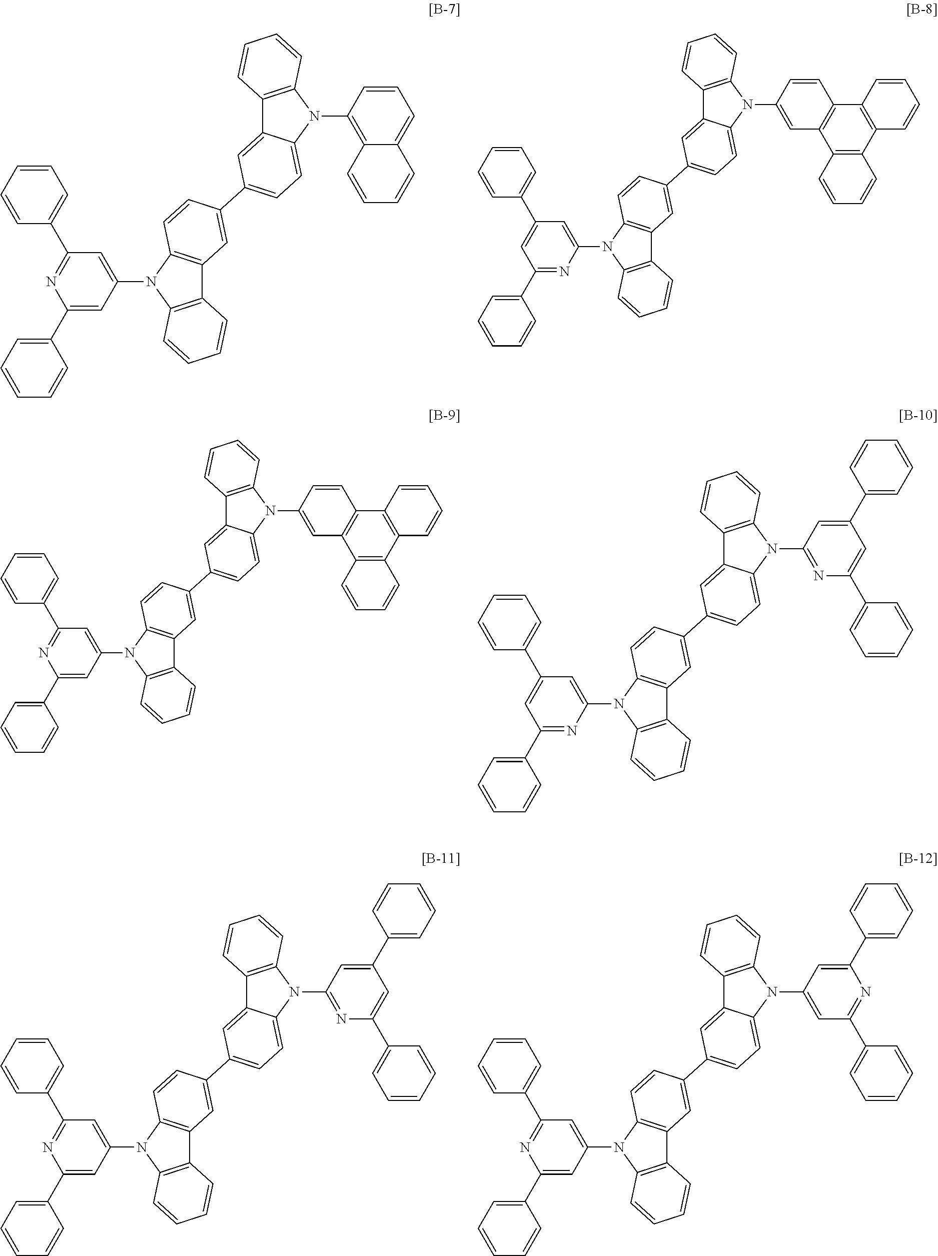

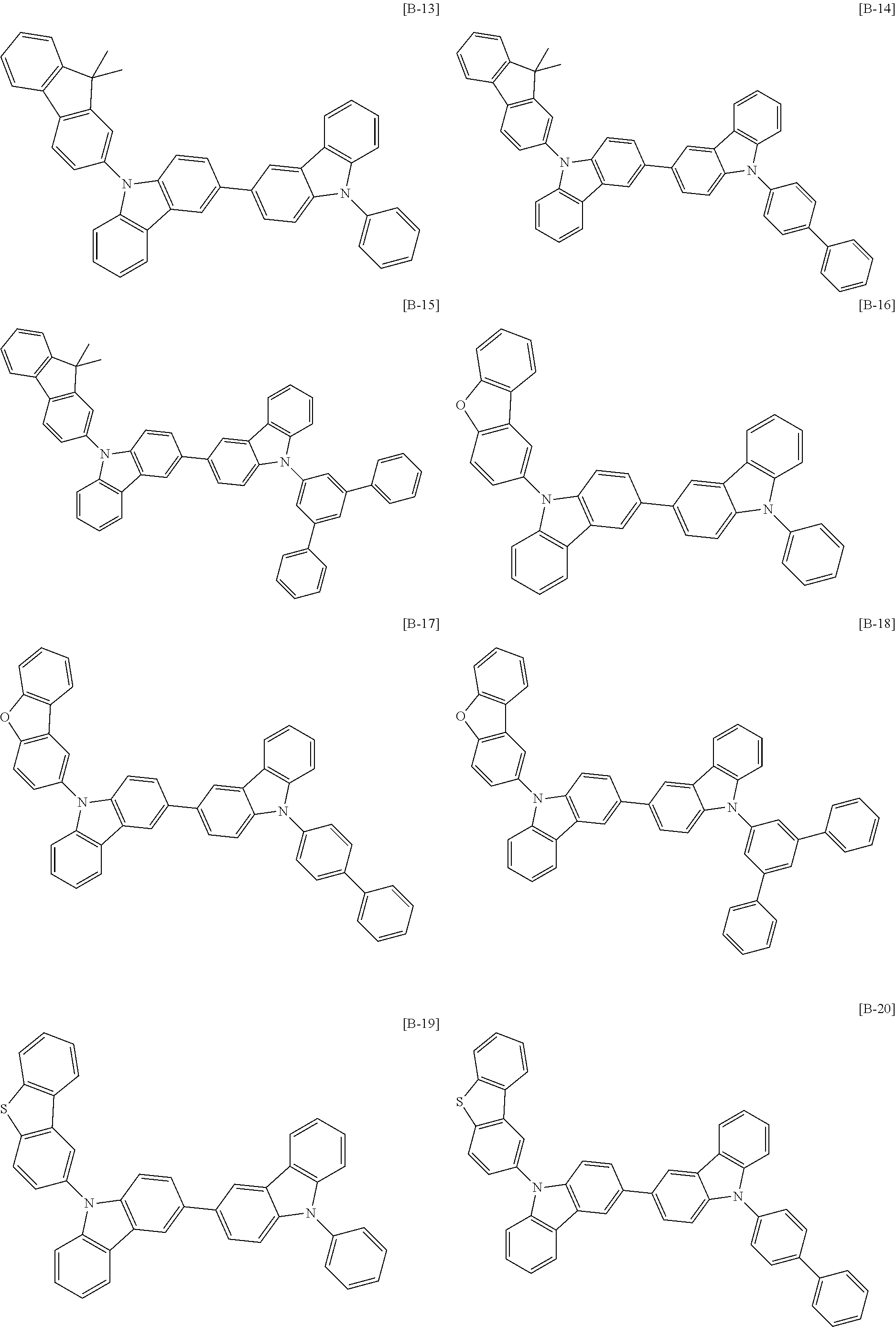

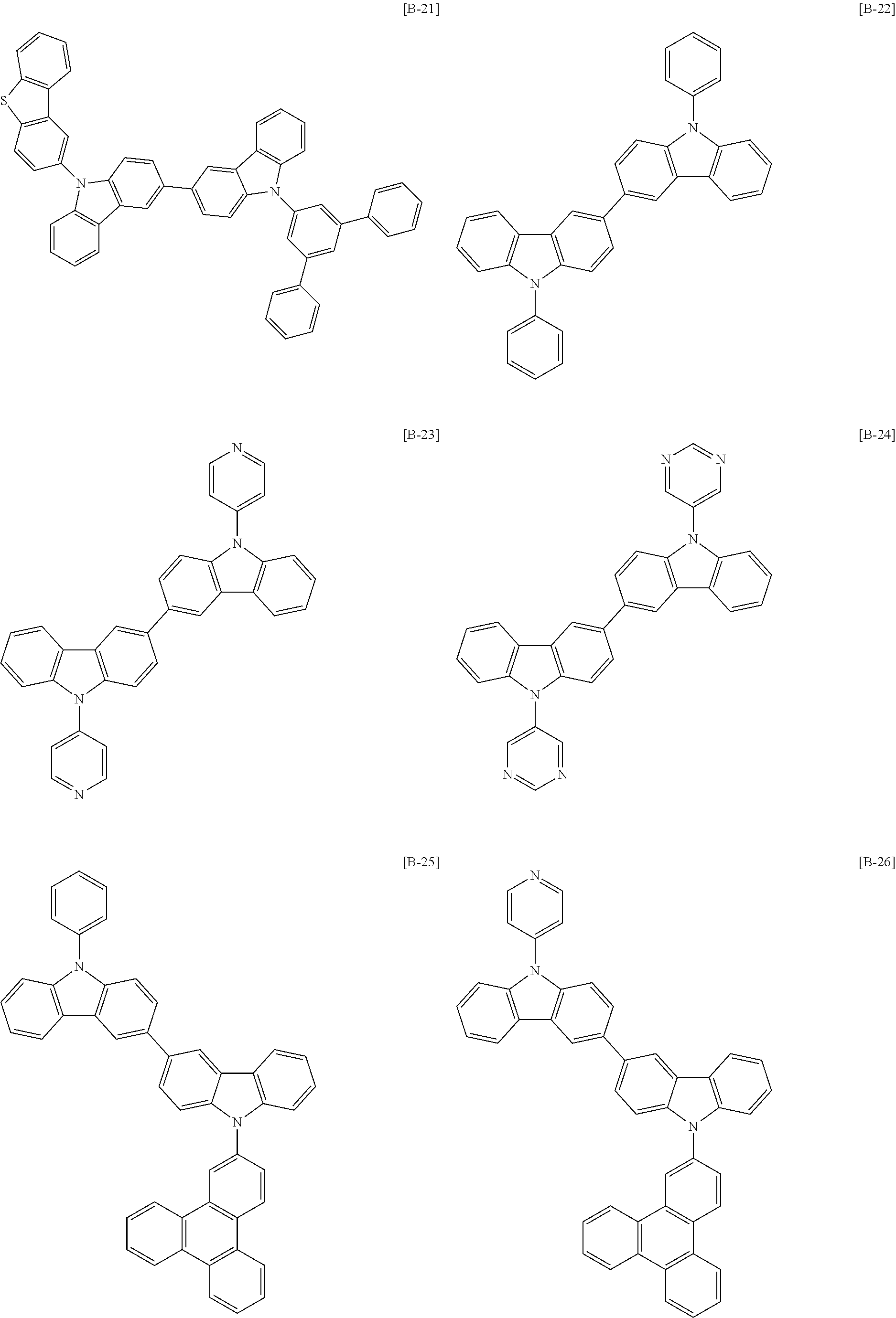 [SMILES: c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1 ; c1ccc(-n2c3ccccc3c3cc(-c4ccc5c(c4)c4ccccc4n5-c4ccc5c6ccccc6c6ccccc6c5c4)ccc32)cc1 ; c1ccc2c(c1)c1ccccc1c1cc(-n3c4ccccc4c4cc(-c5ccc6c(c5)c5ccccc5n6-c5ccncc5)ccc43)ccc21]

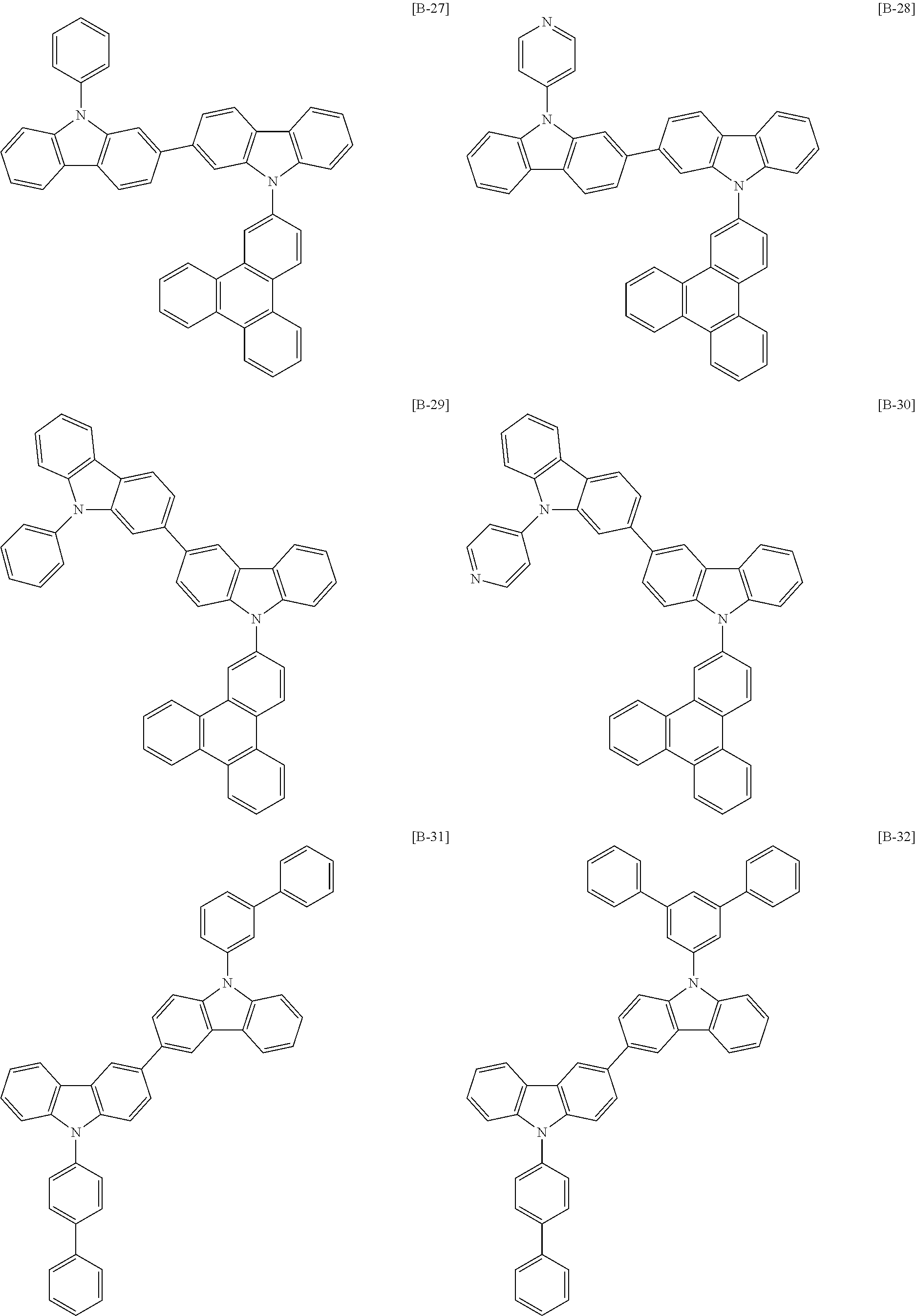

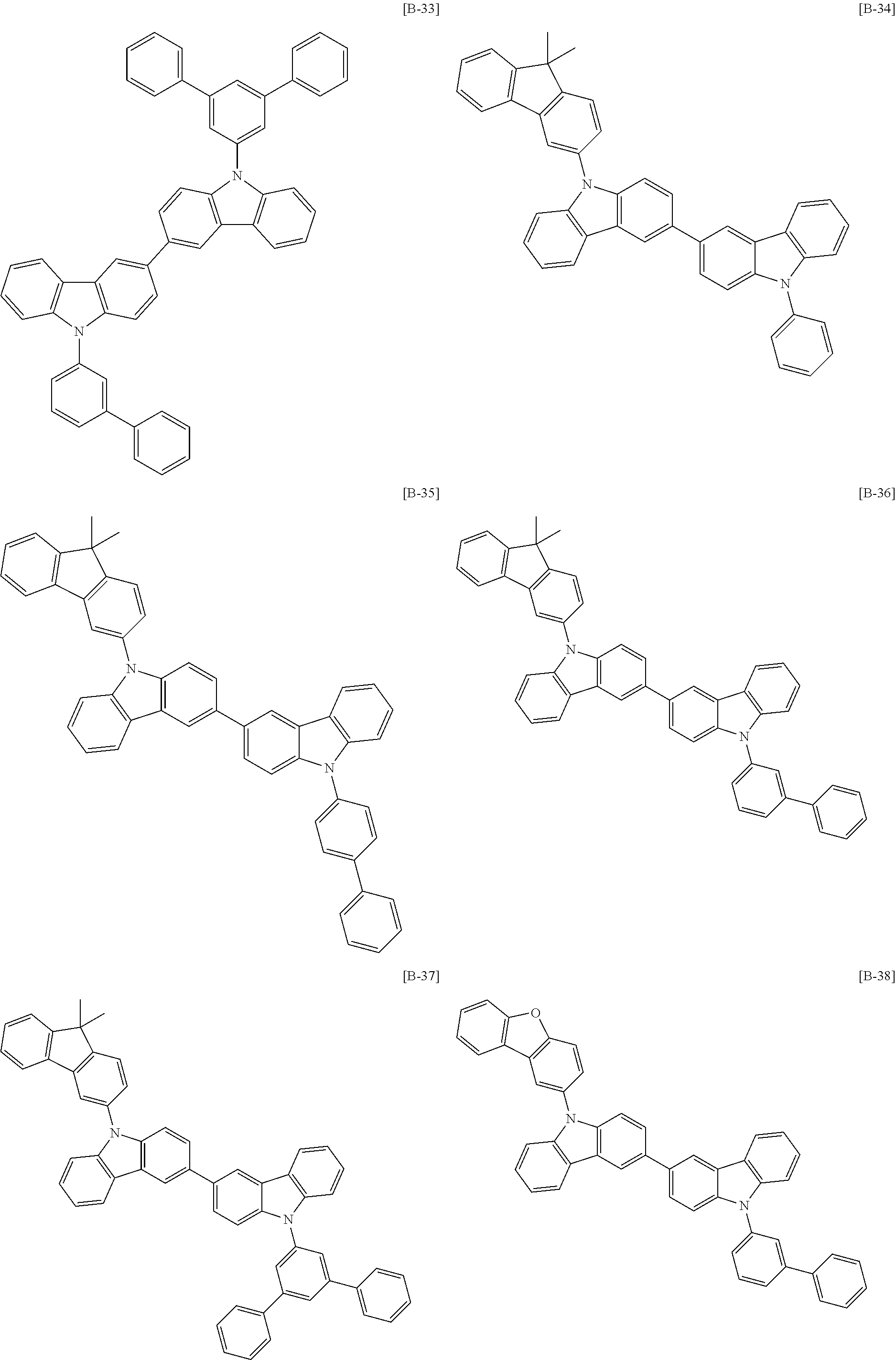

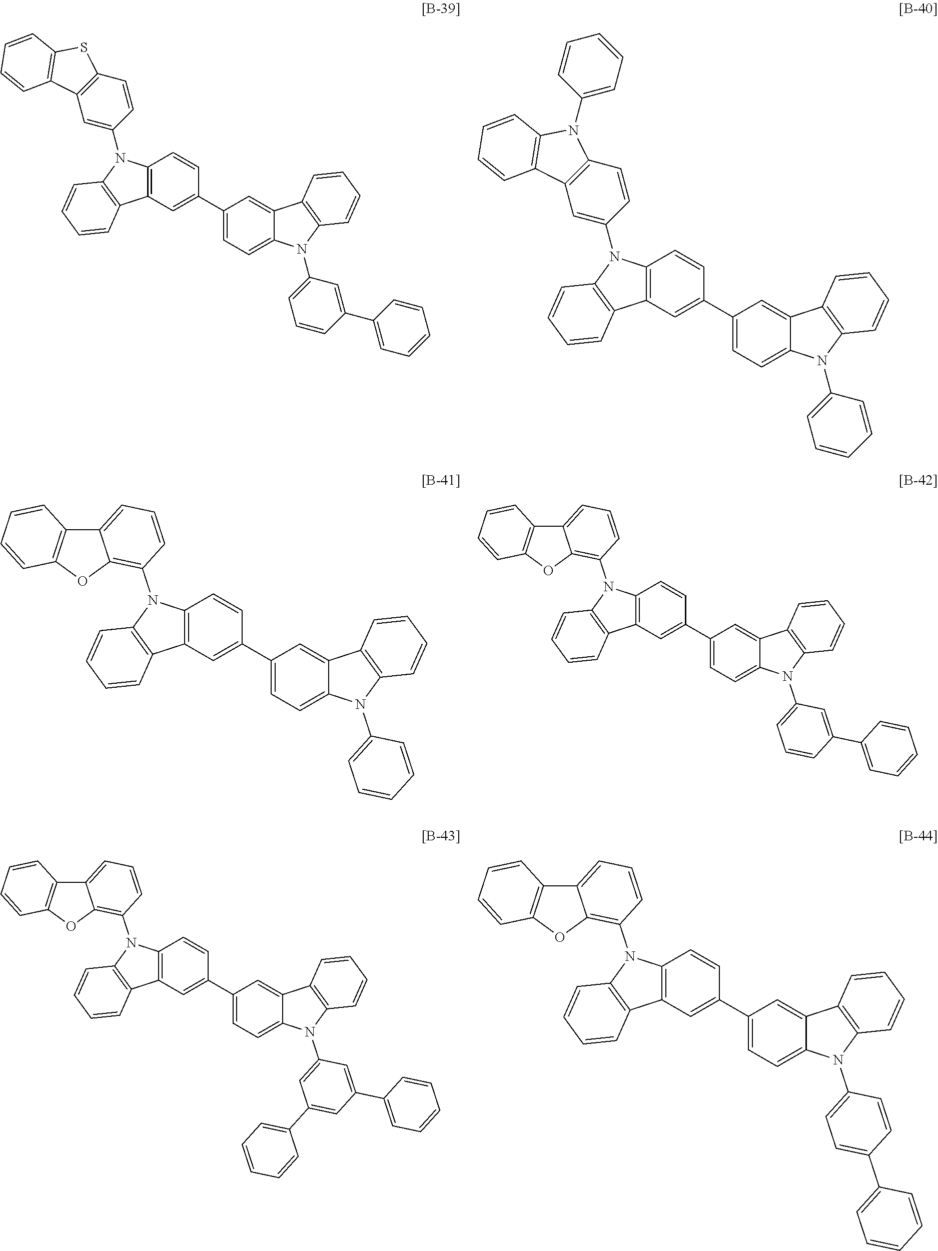

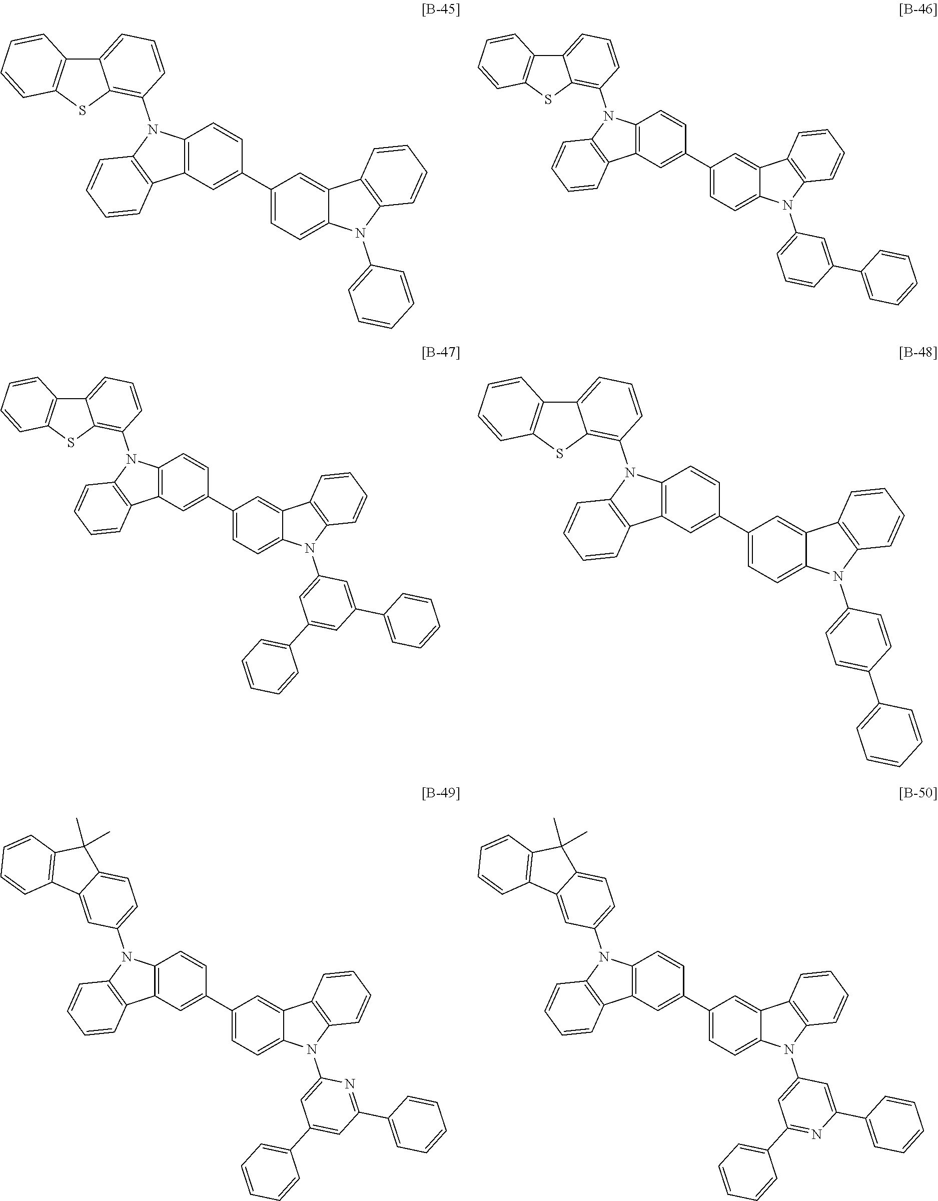

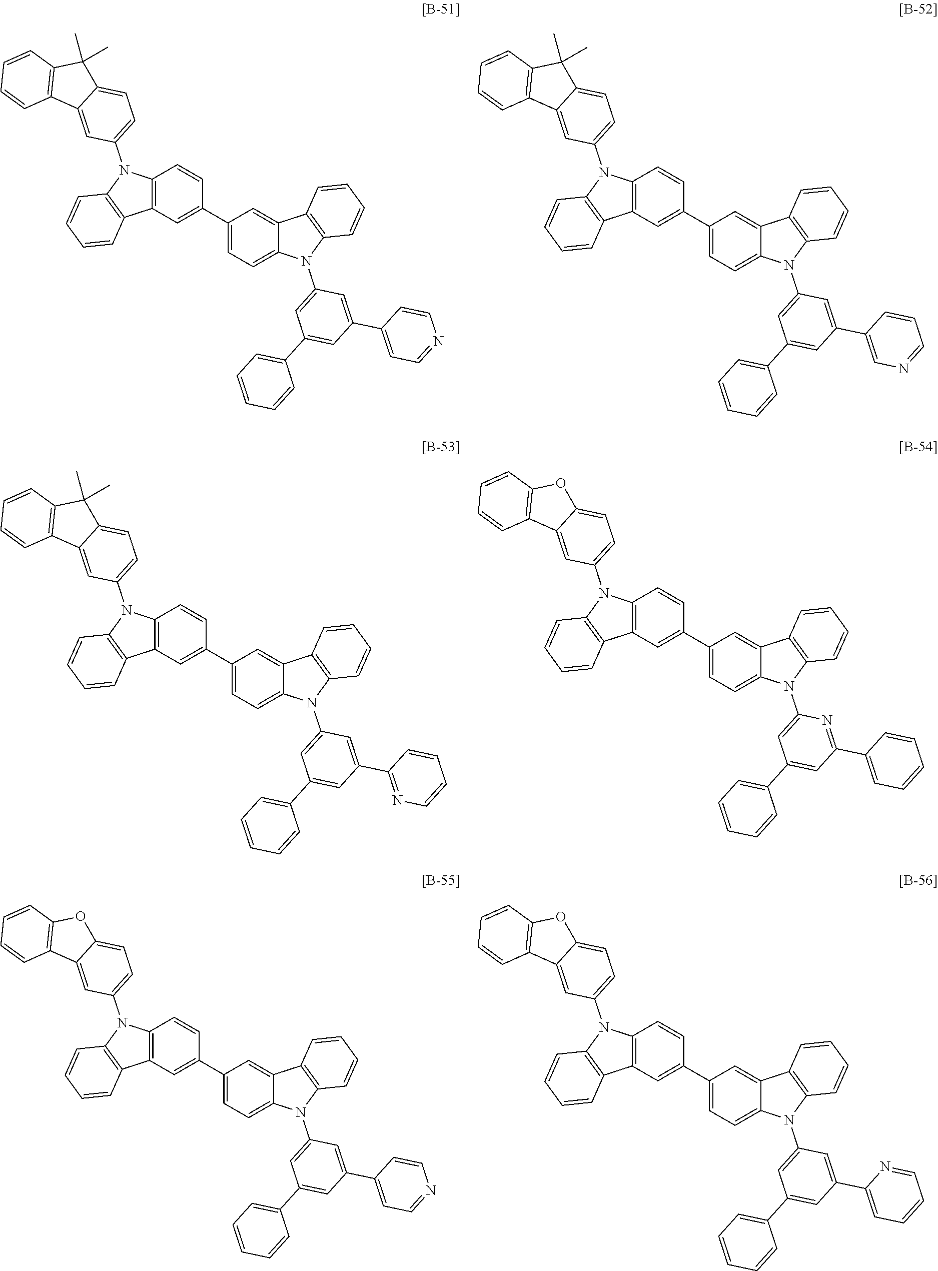

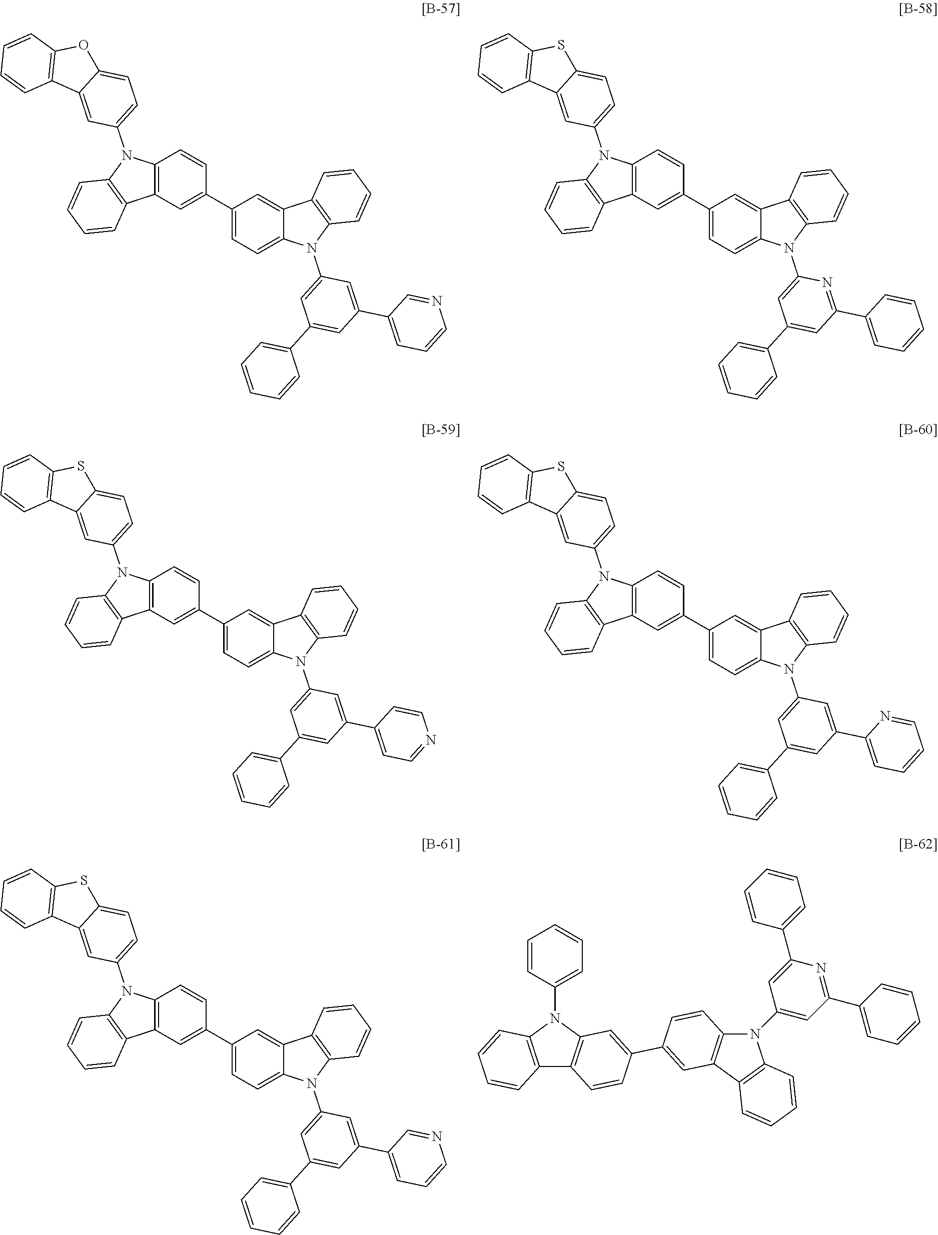

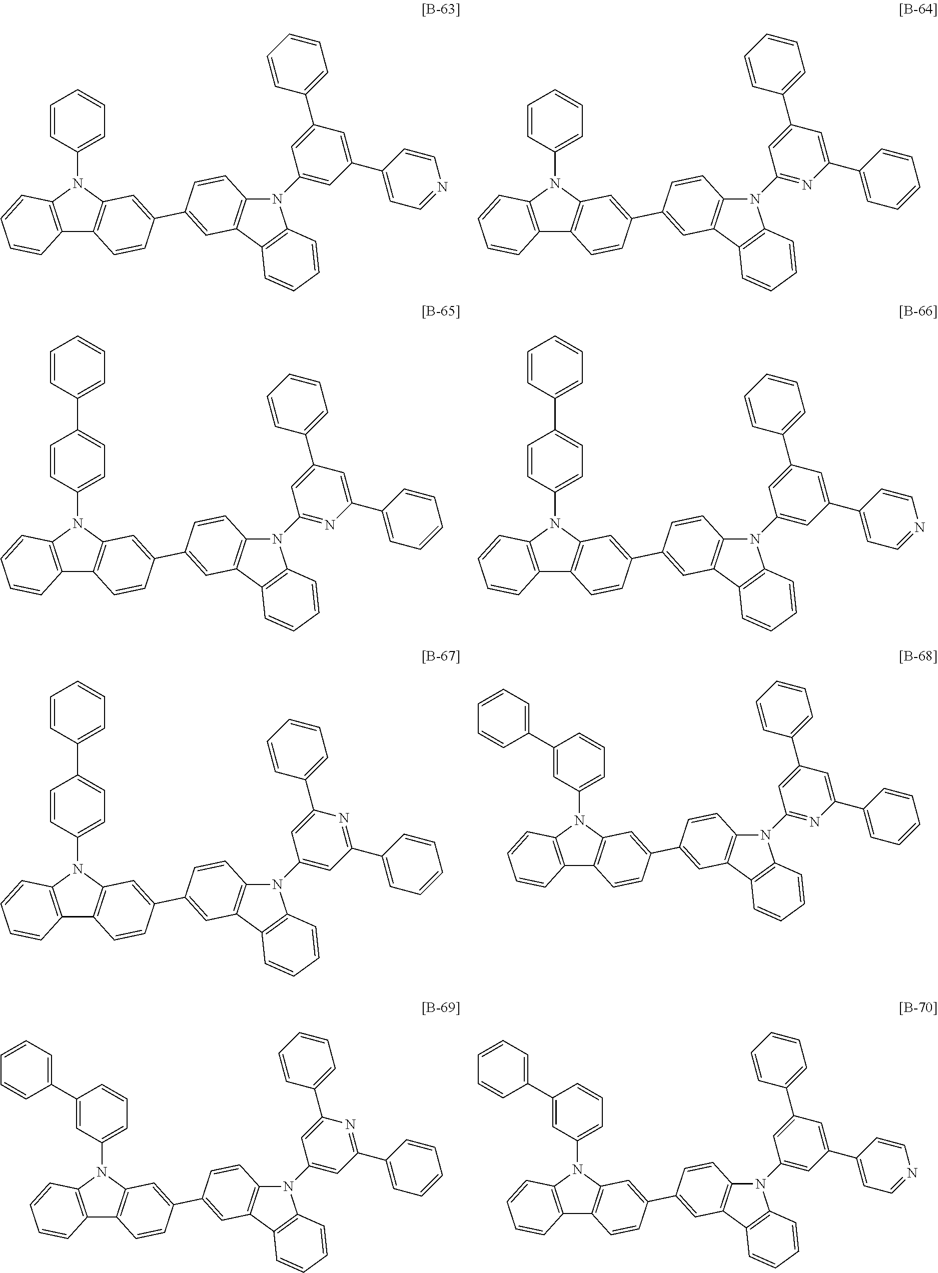

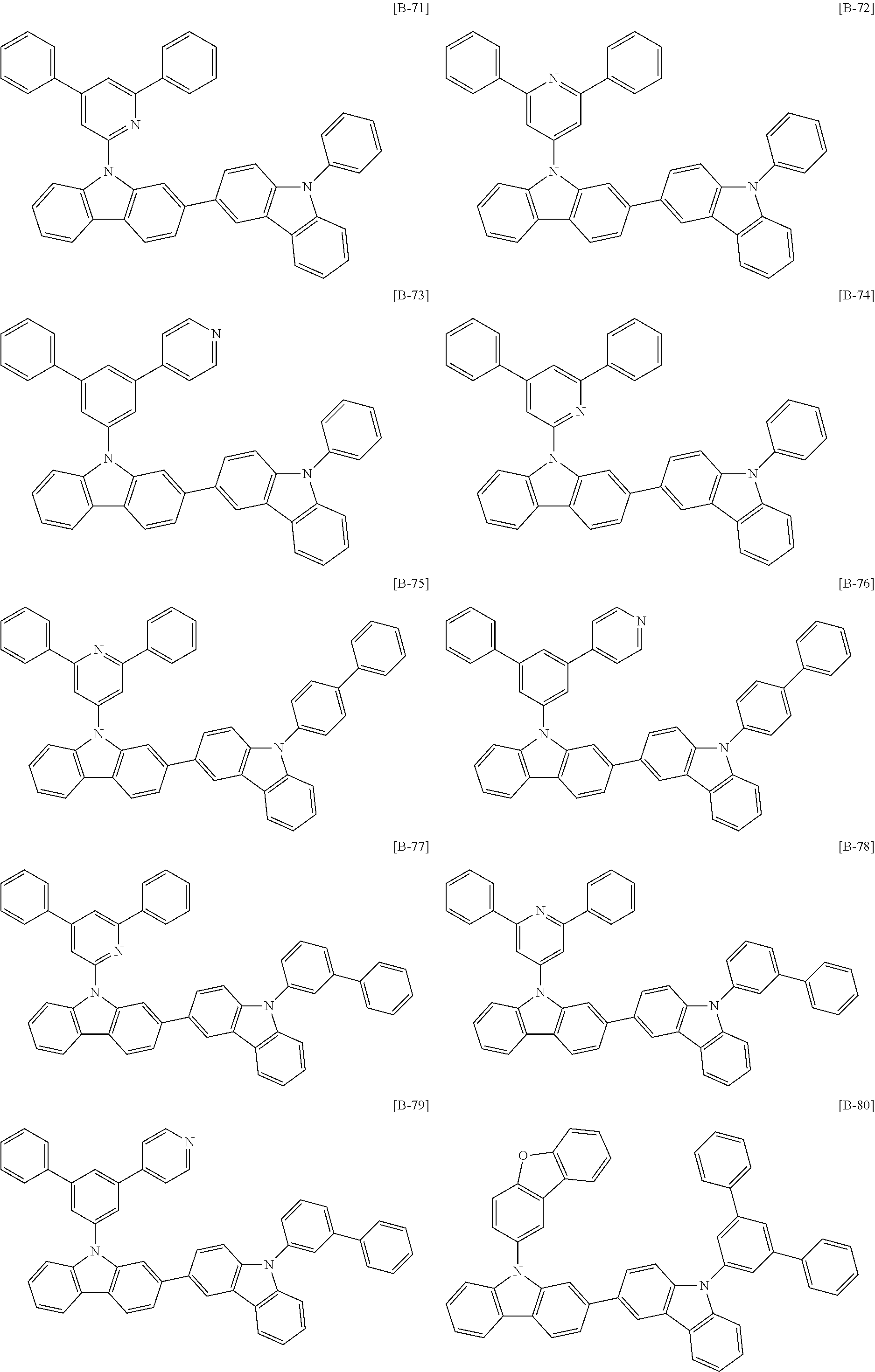

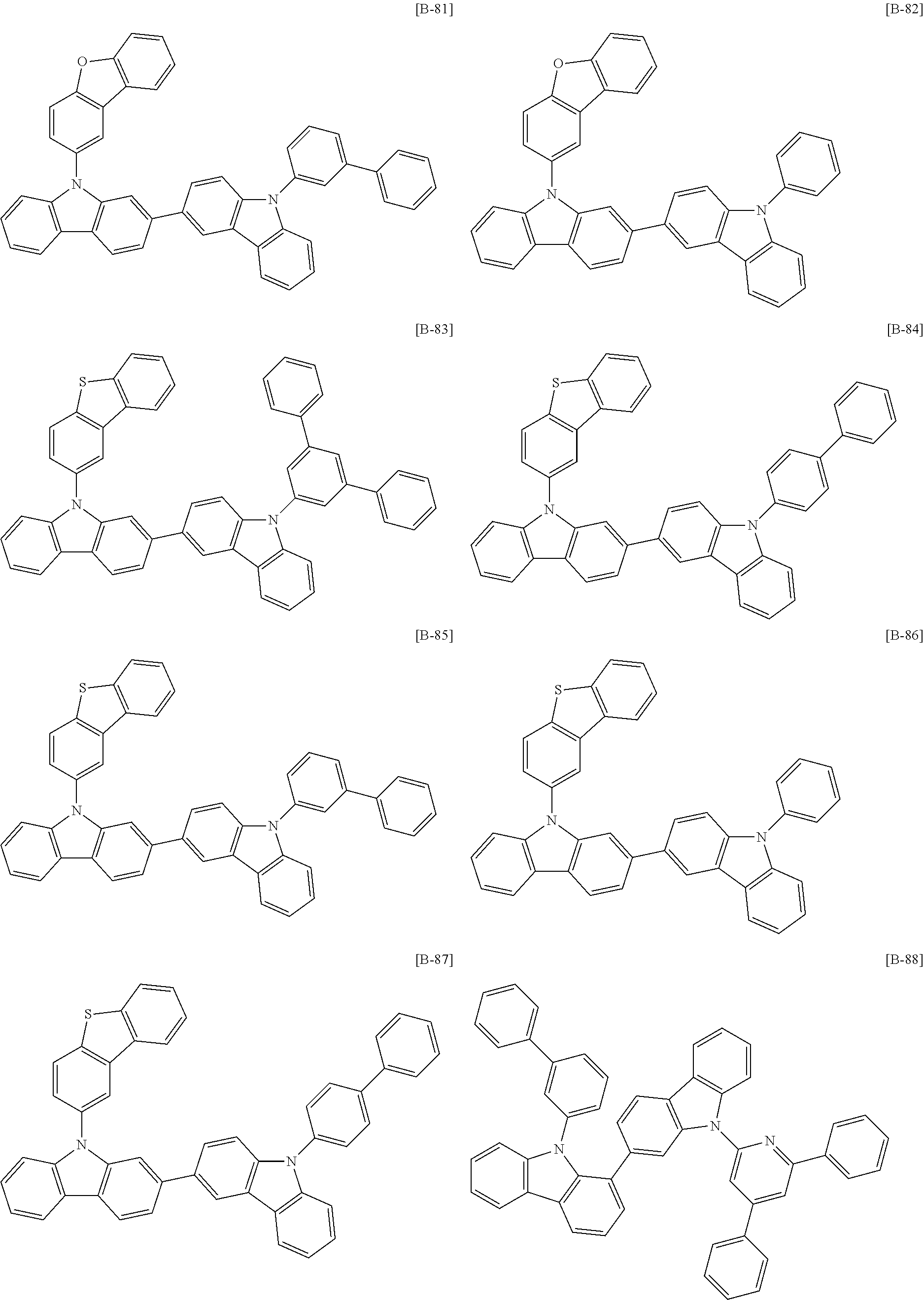

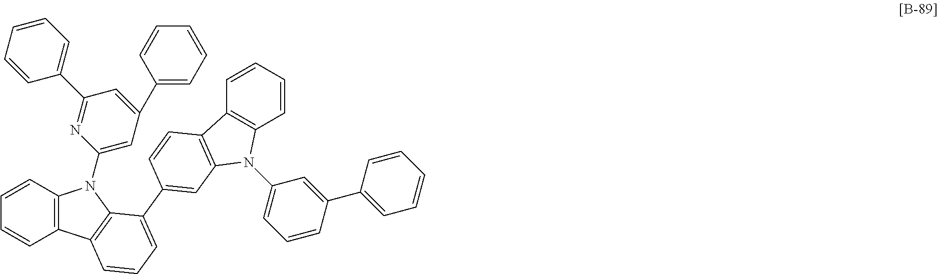 [SMILES: c1ccc(-c2cccc(-n3c4ccccc4c4ccc(-c5cccc6c7ccccc7n(-c7cc(-c8ccccc8)cc(-c8ccccc8)n7)c56)cc43)c2)cc1]

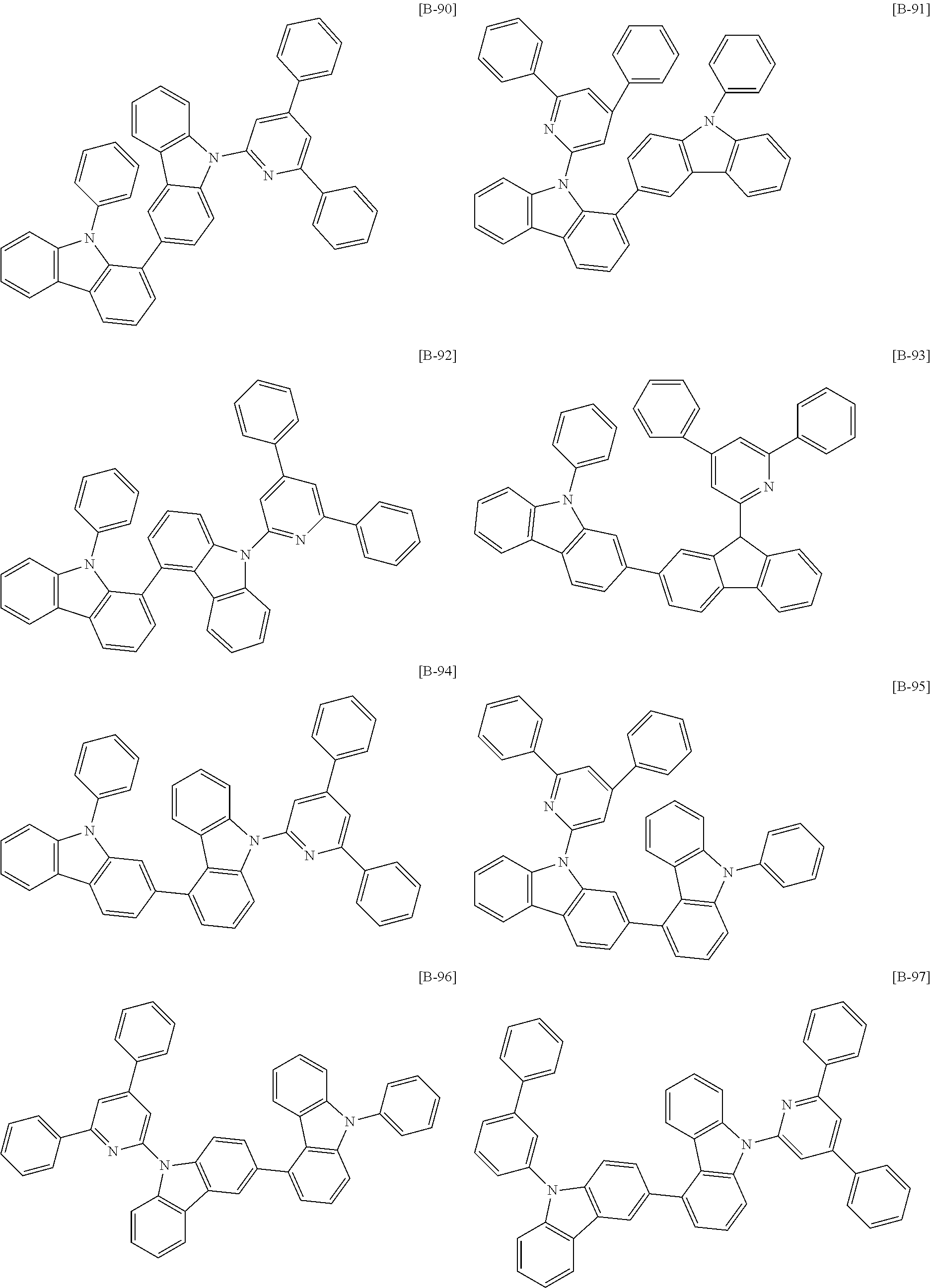

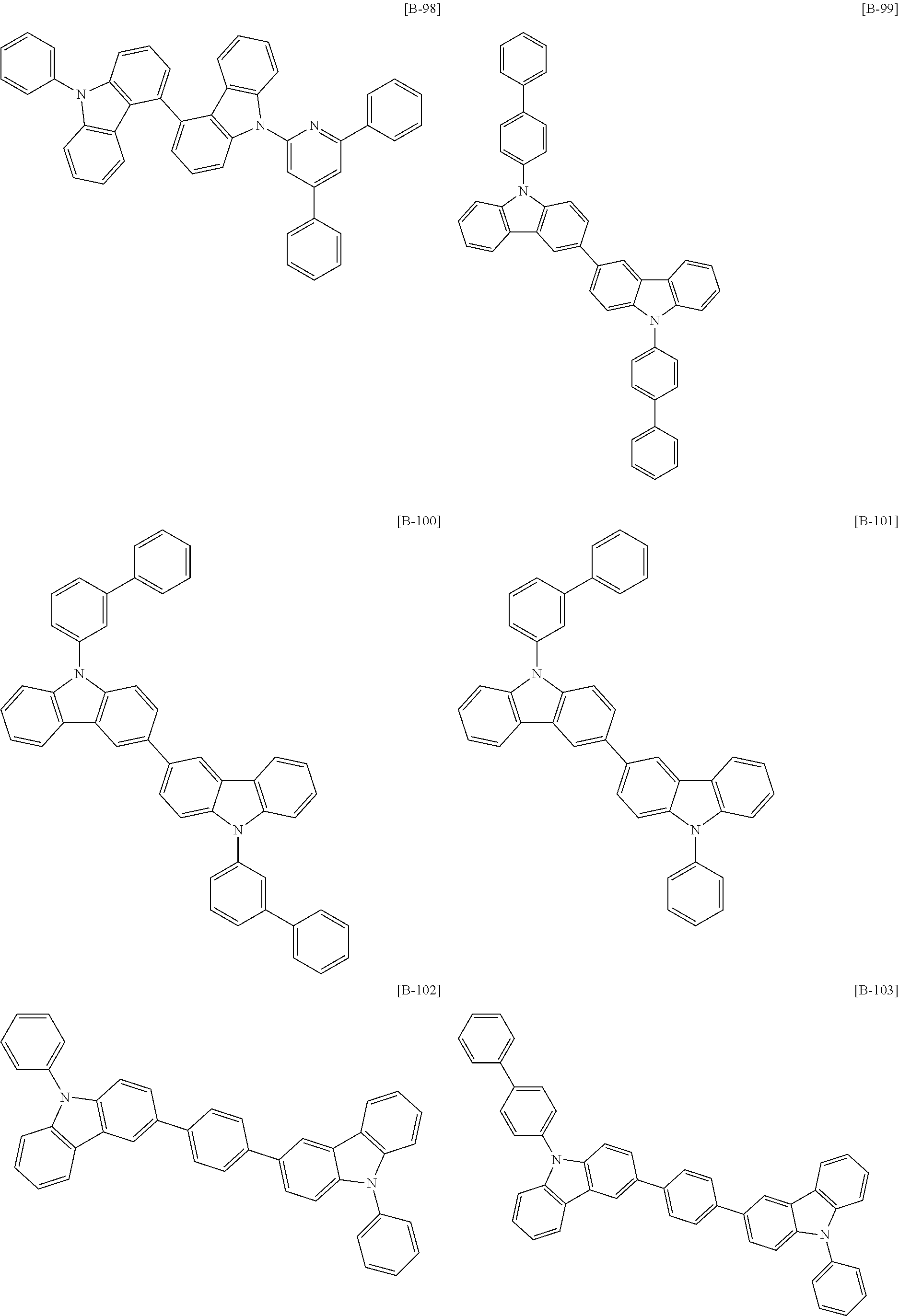

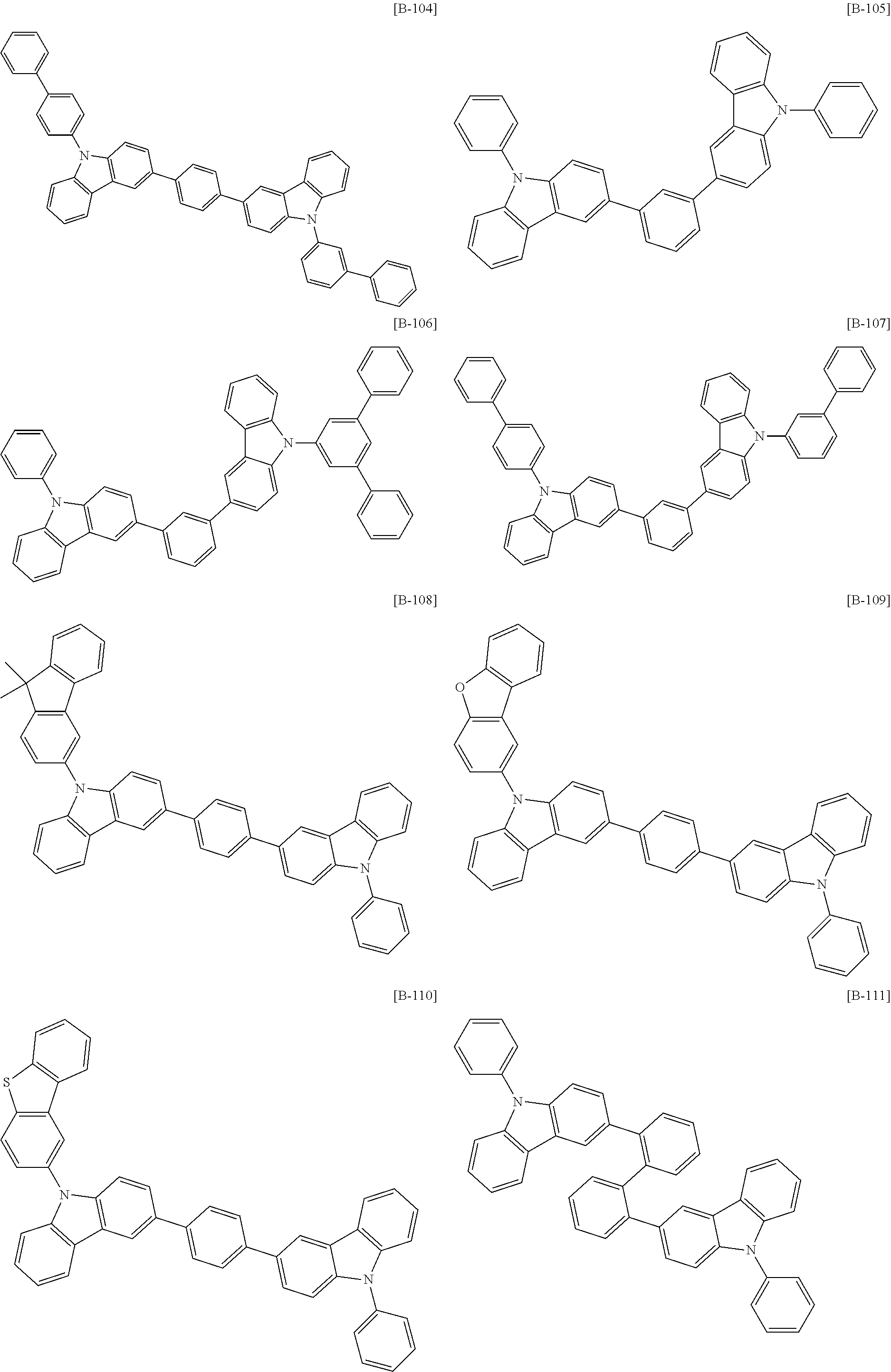

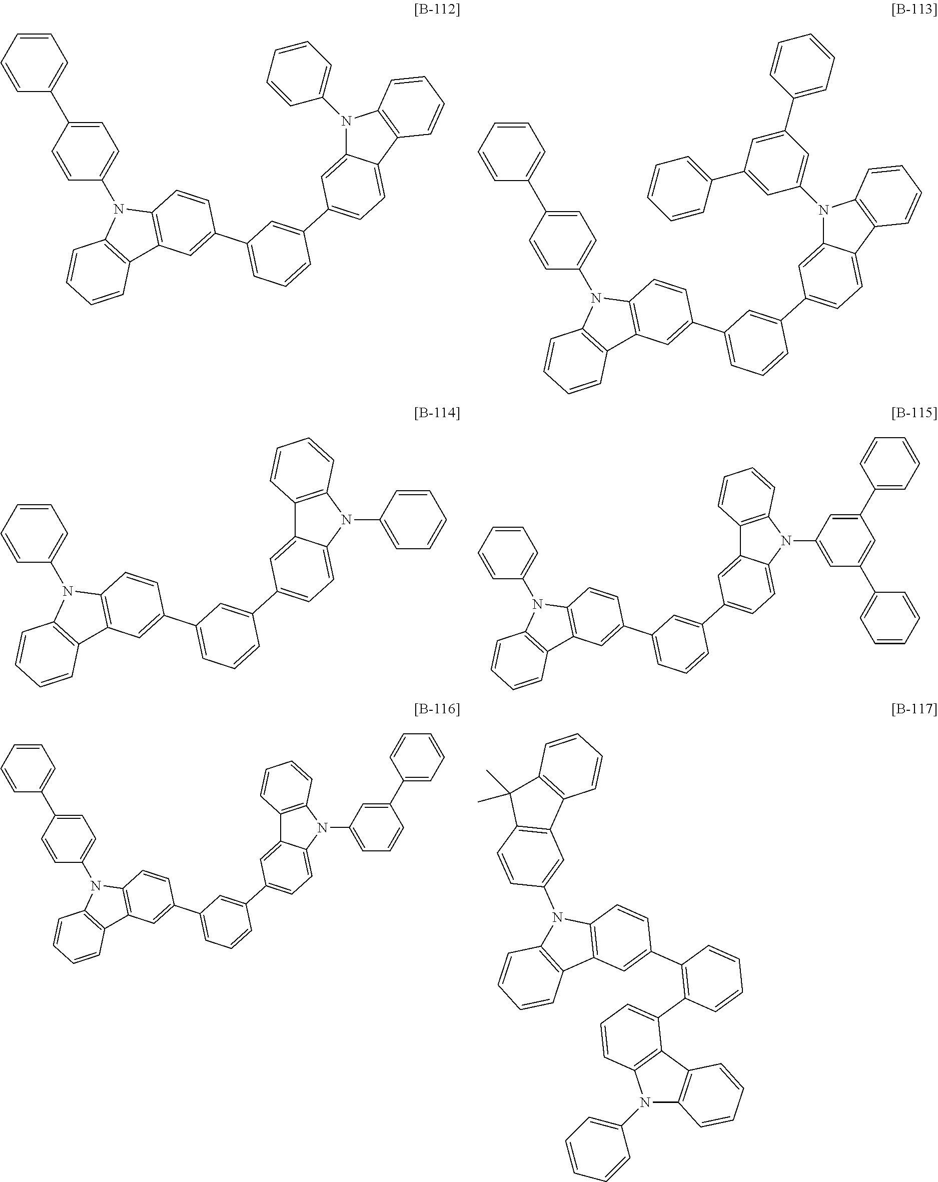

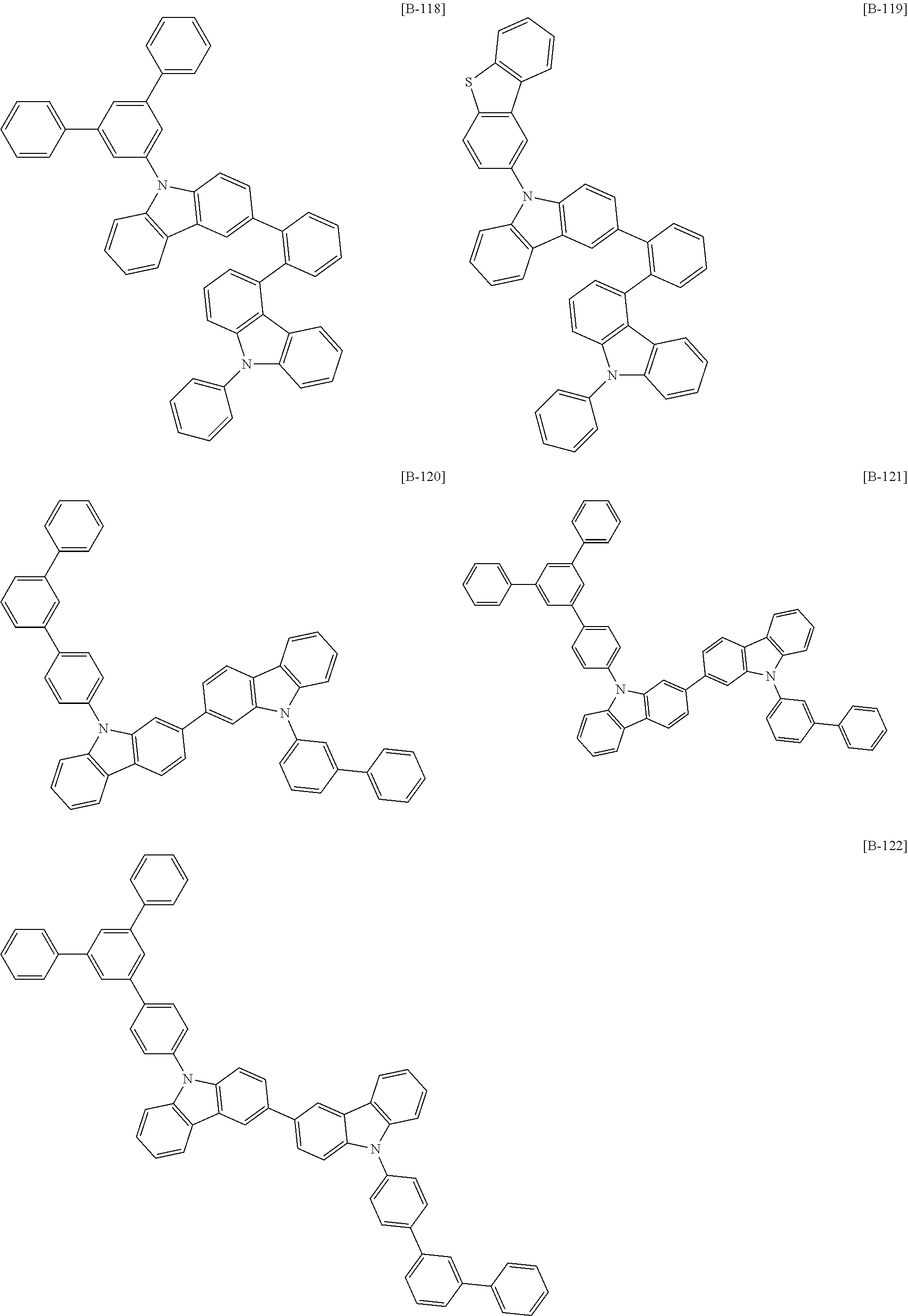

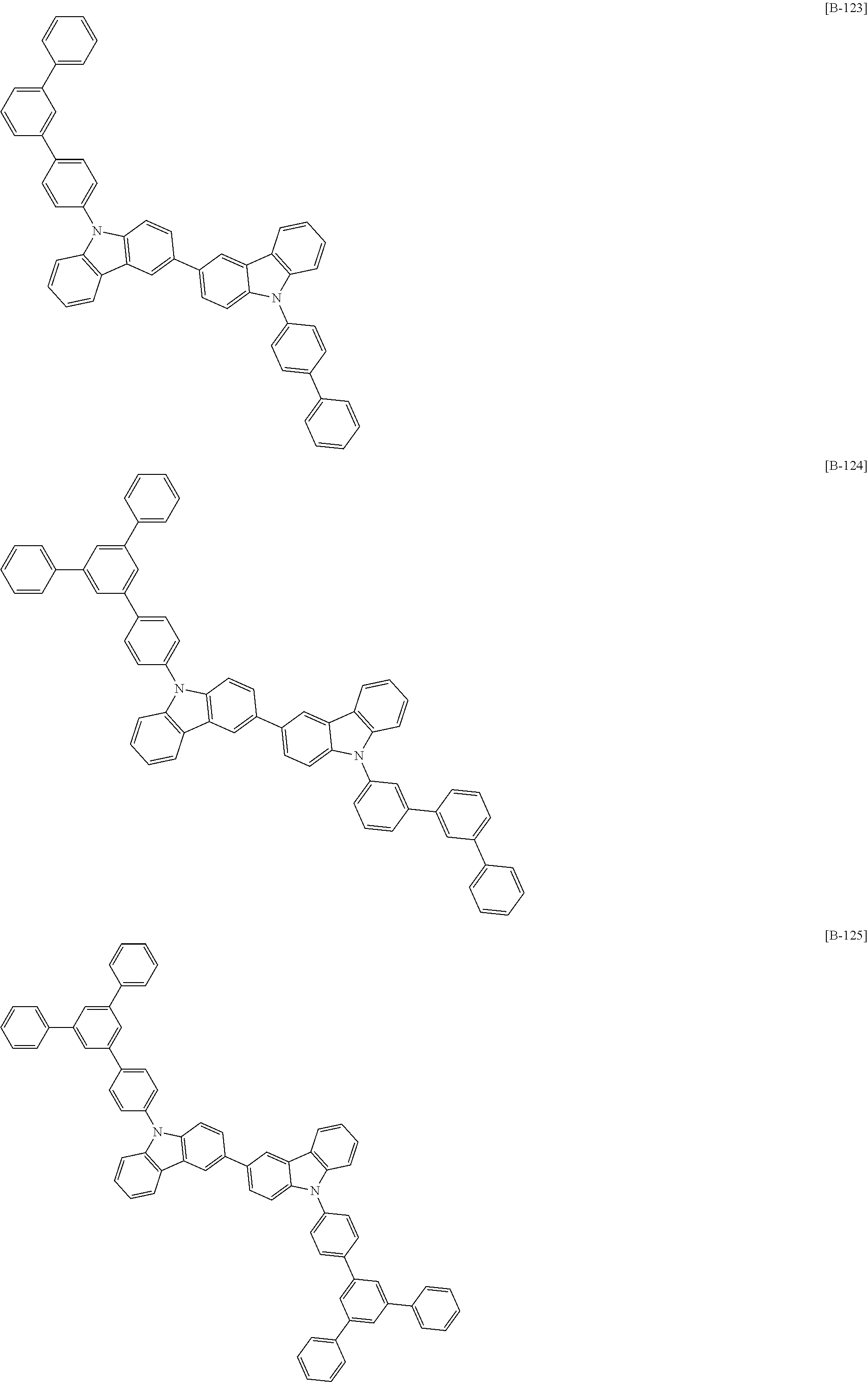 [SMILES: c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1 ; c1ccc(-c2cccc(-c3cccc(-n4c5ccccc5c5cc(-c6ccc7c(c6)c6ccccc6n7-c6ccc(-c7cc(-c8ccccc8)cc(-c8ccccc8)c7)cc6)ccc54)c3)c2)cc1 ; c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1]

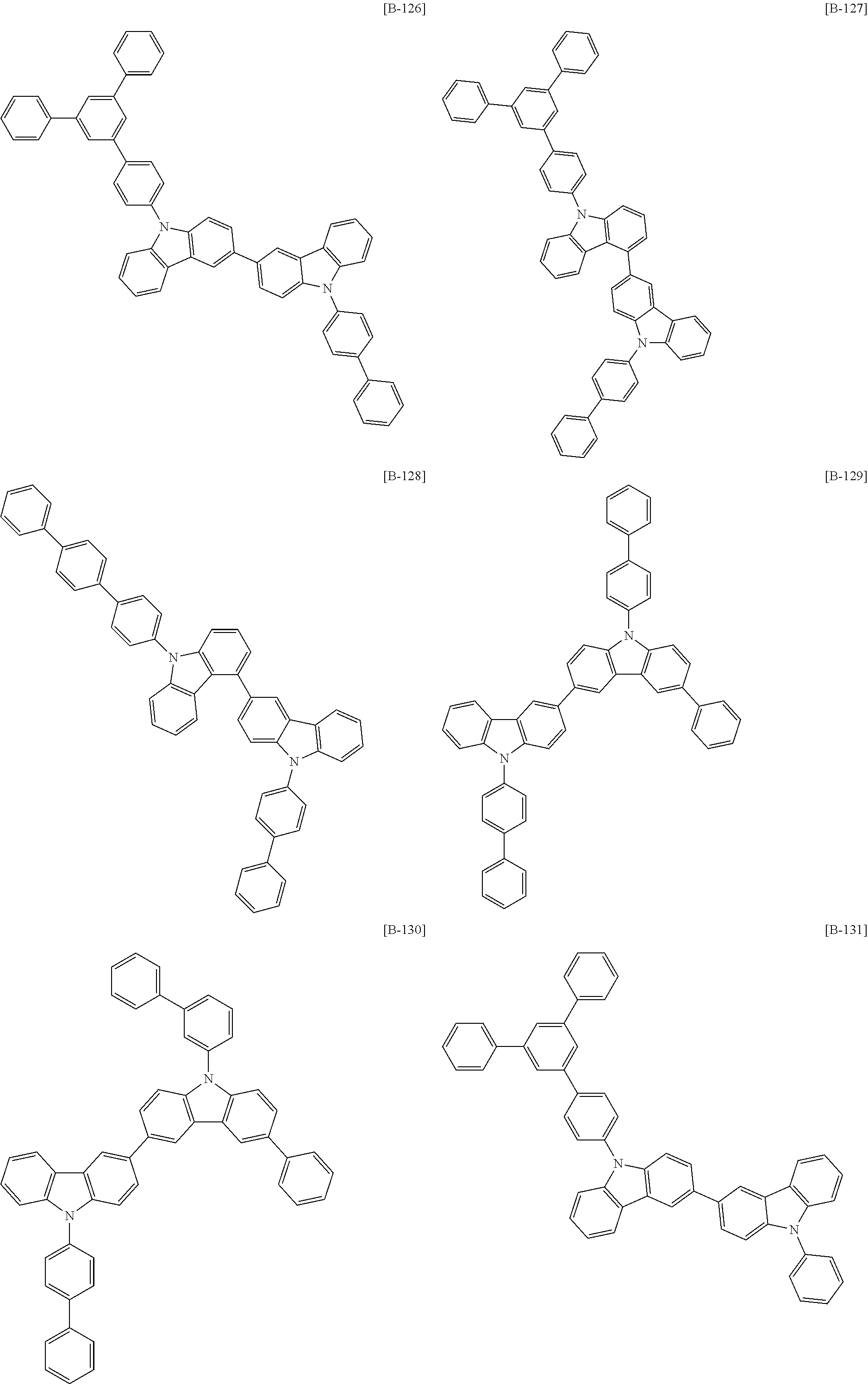

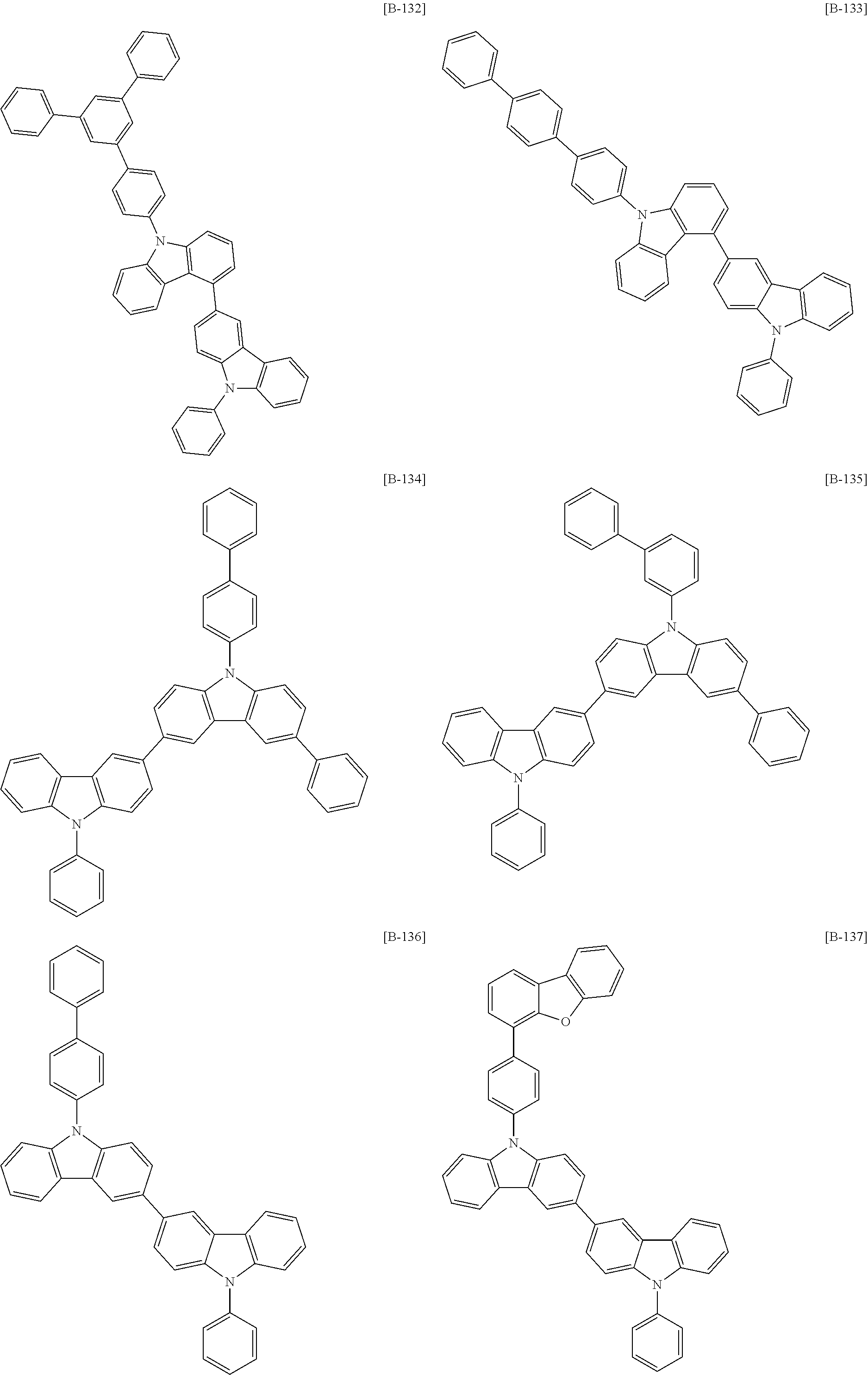

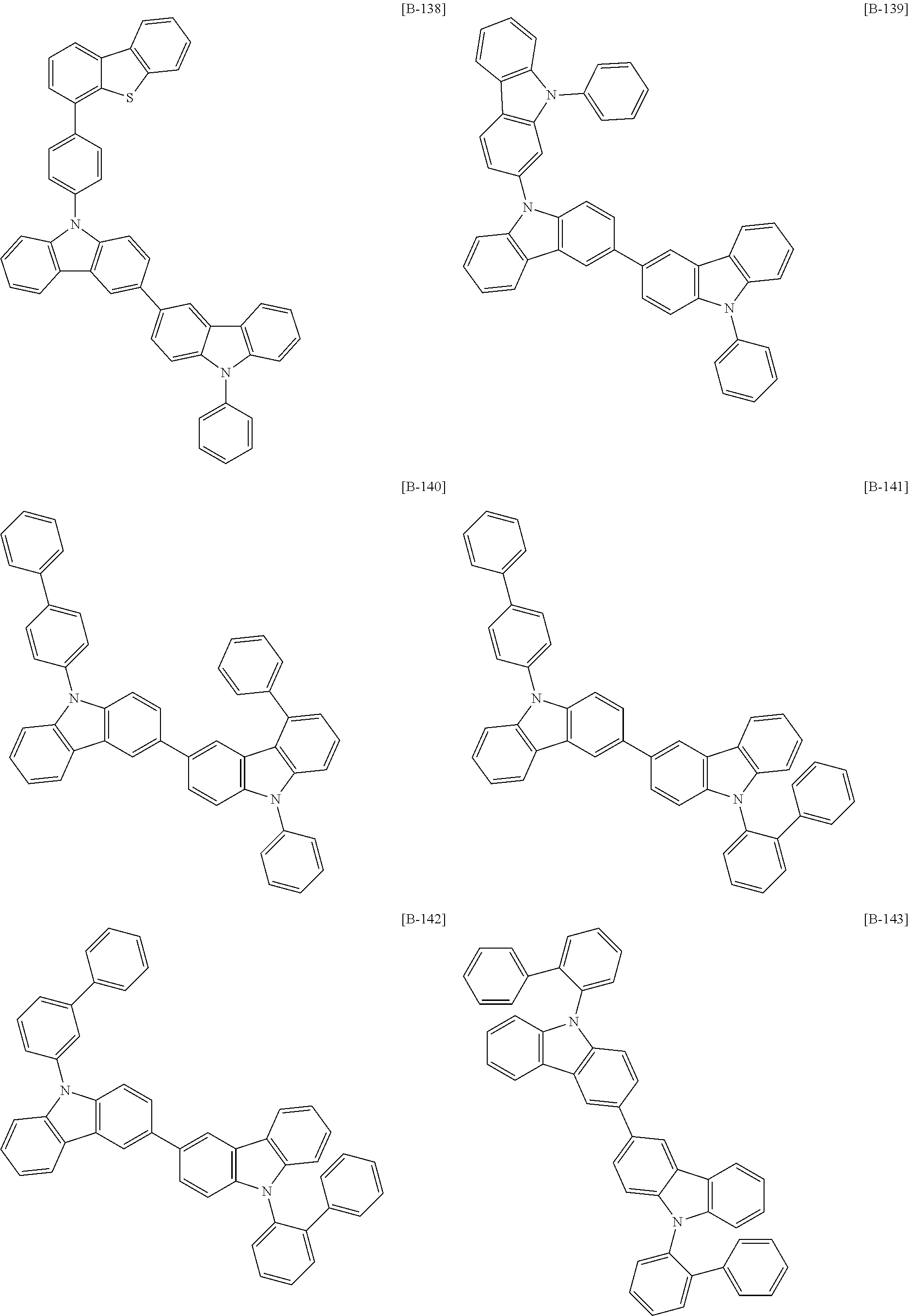

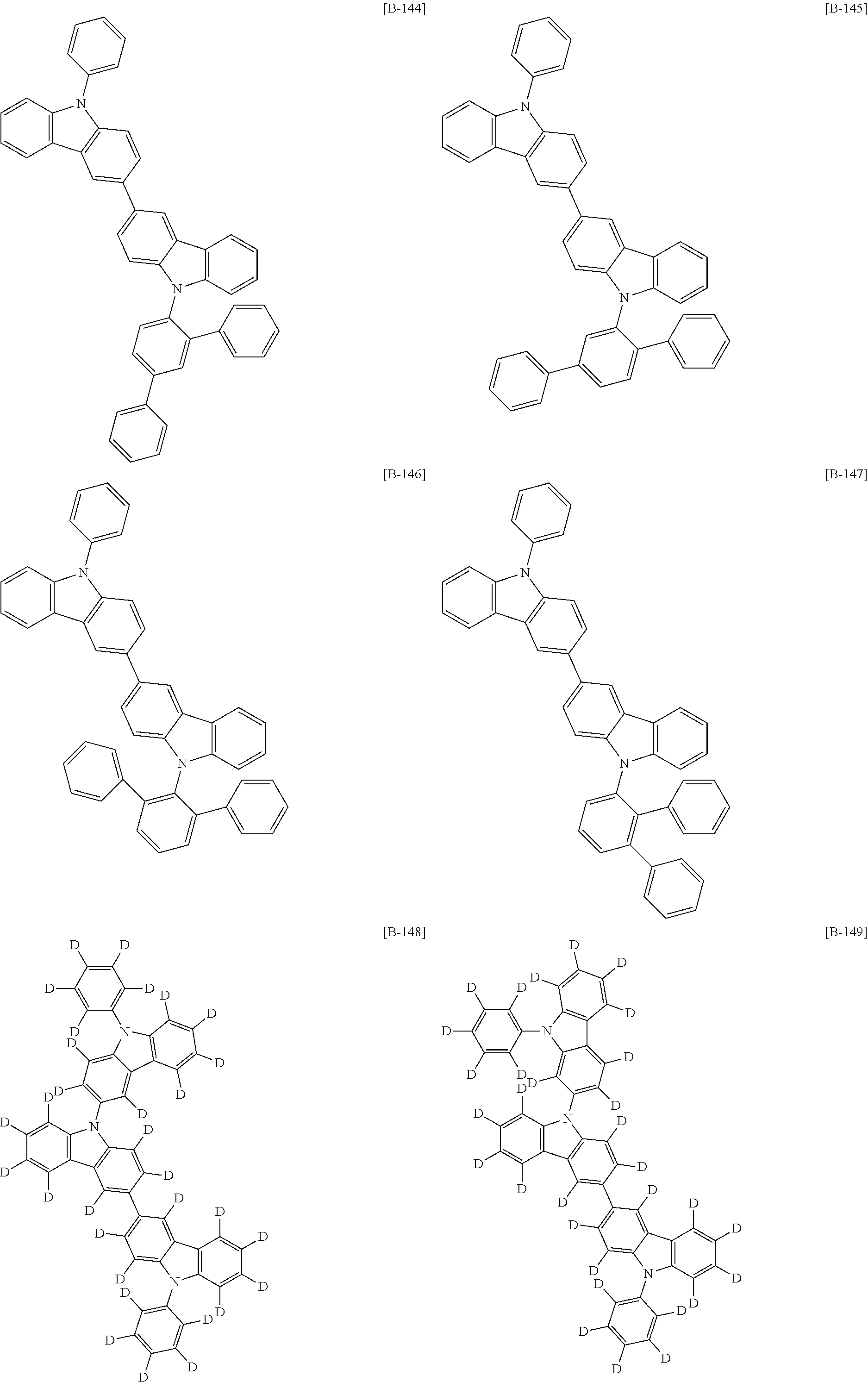

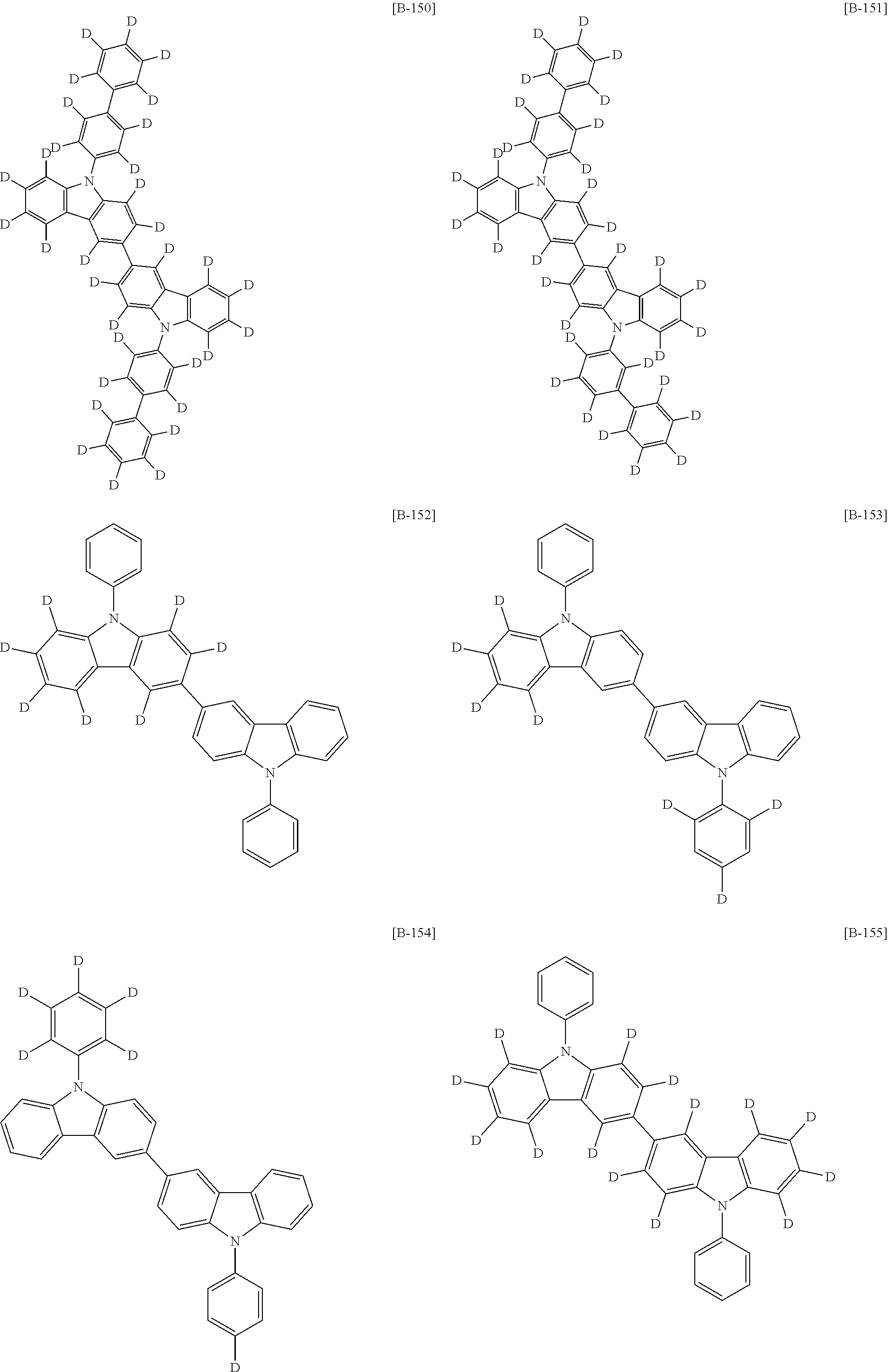

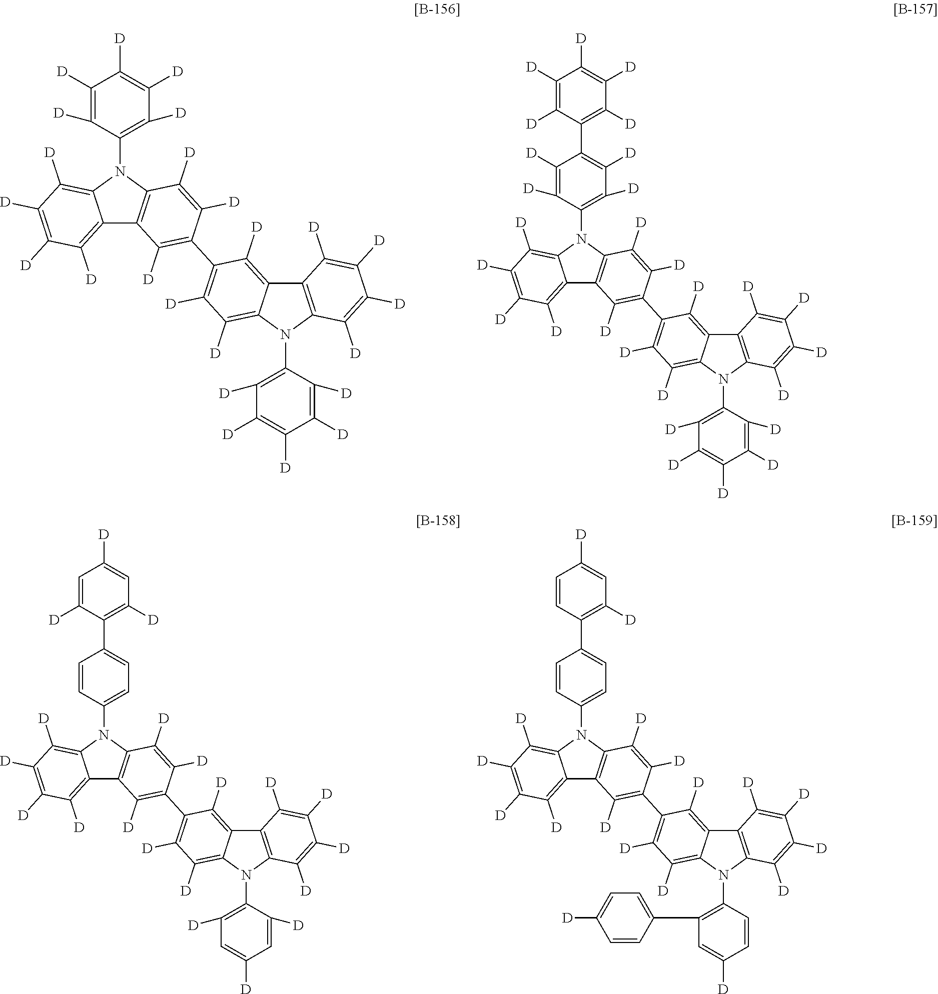

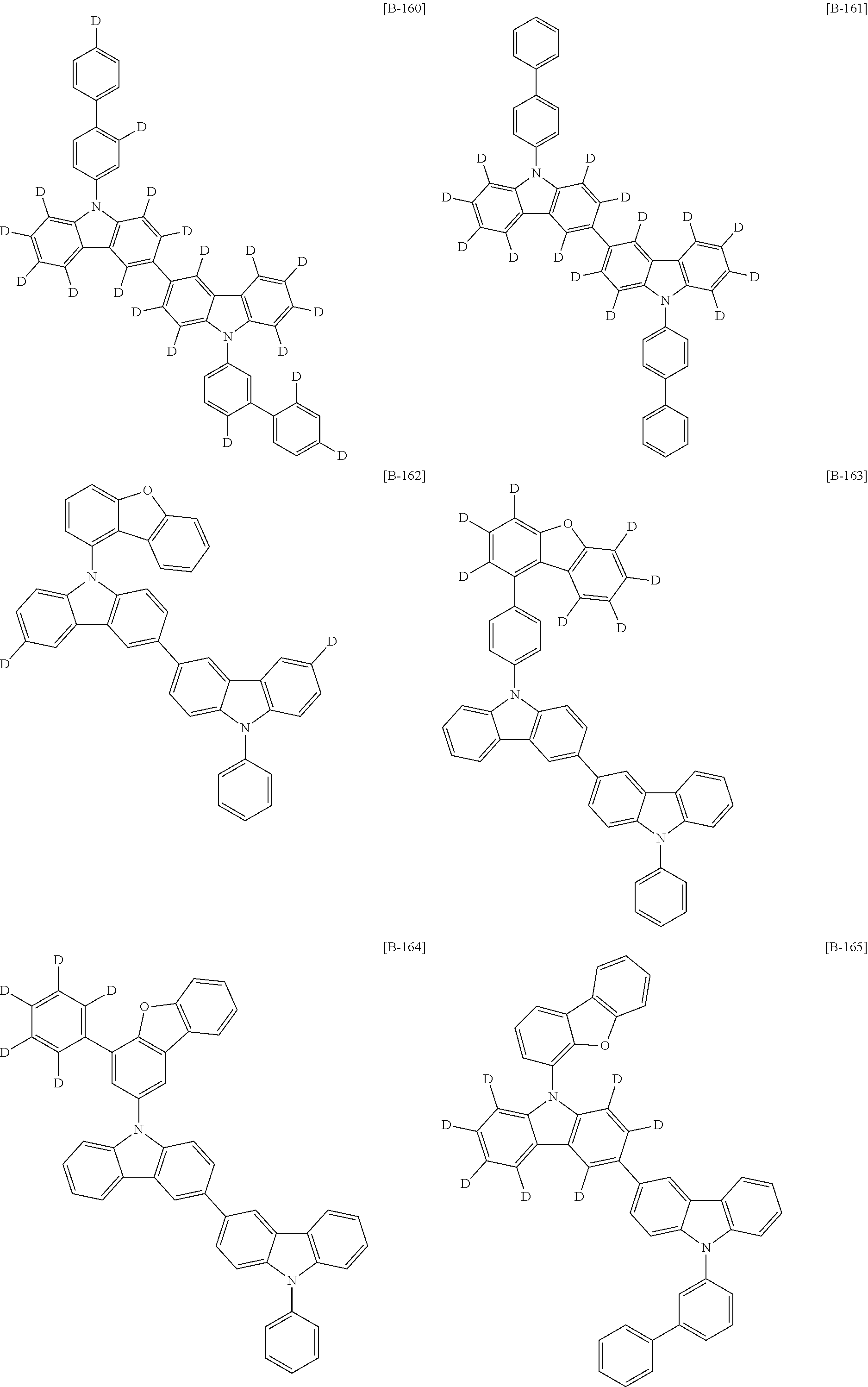

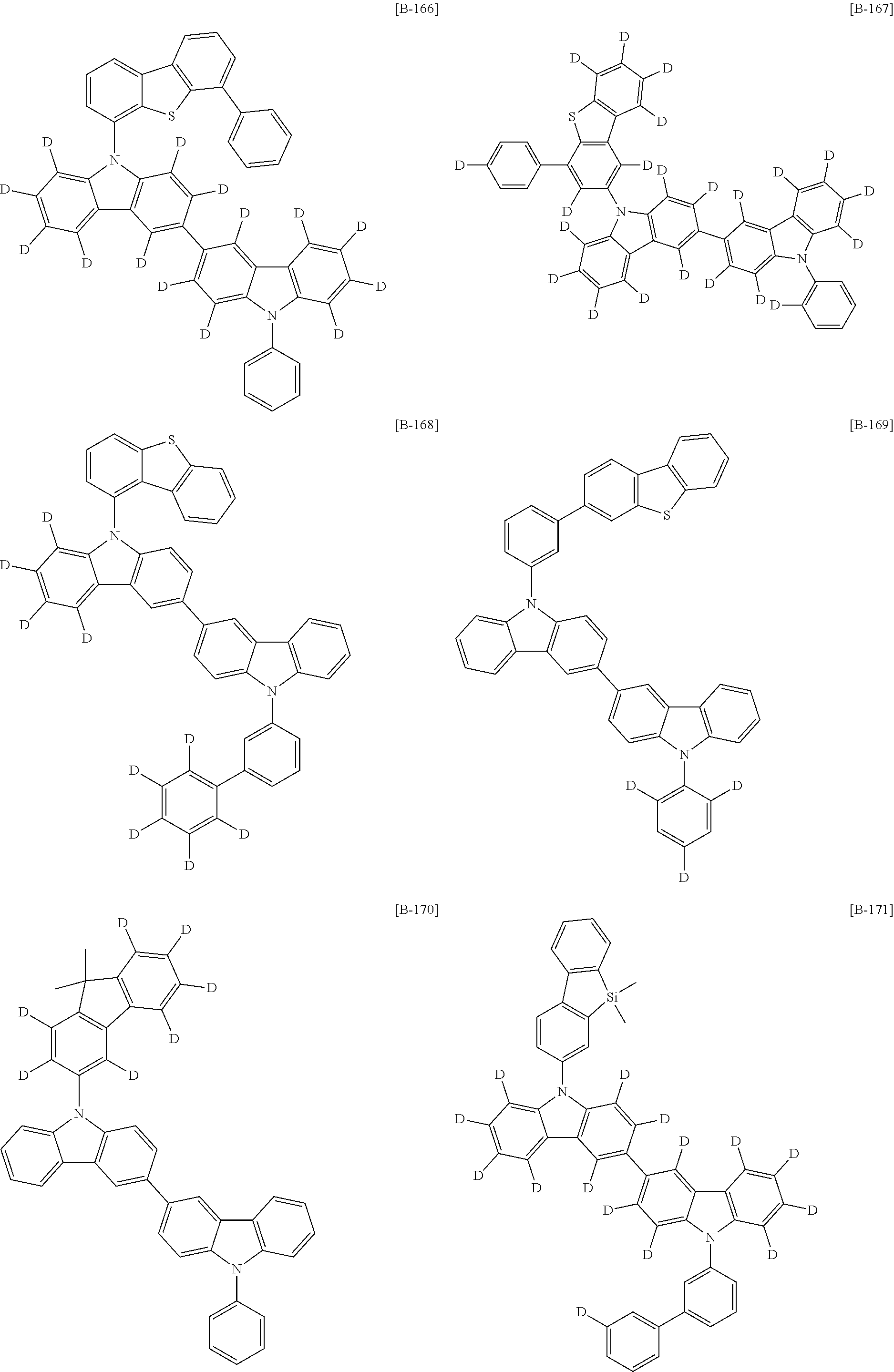

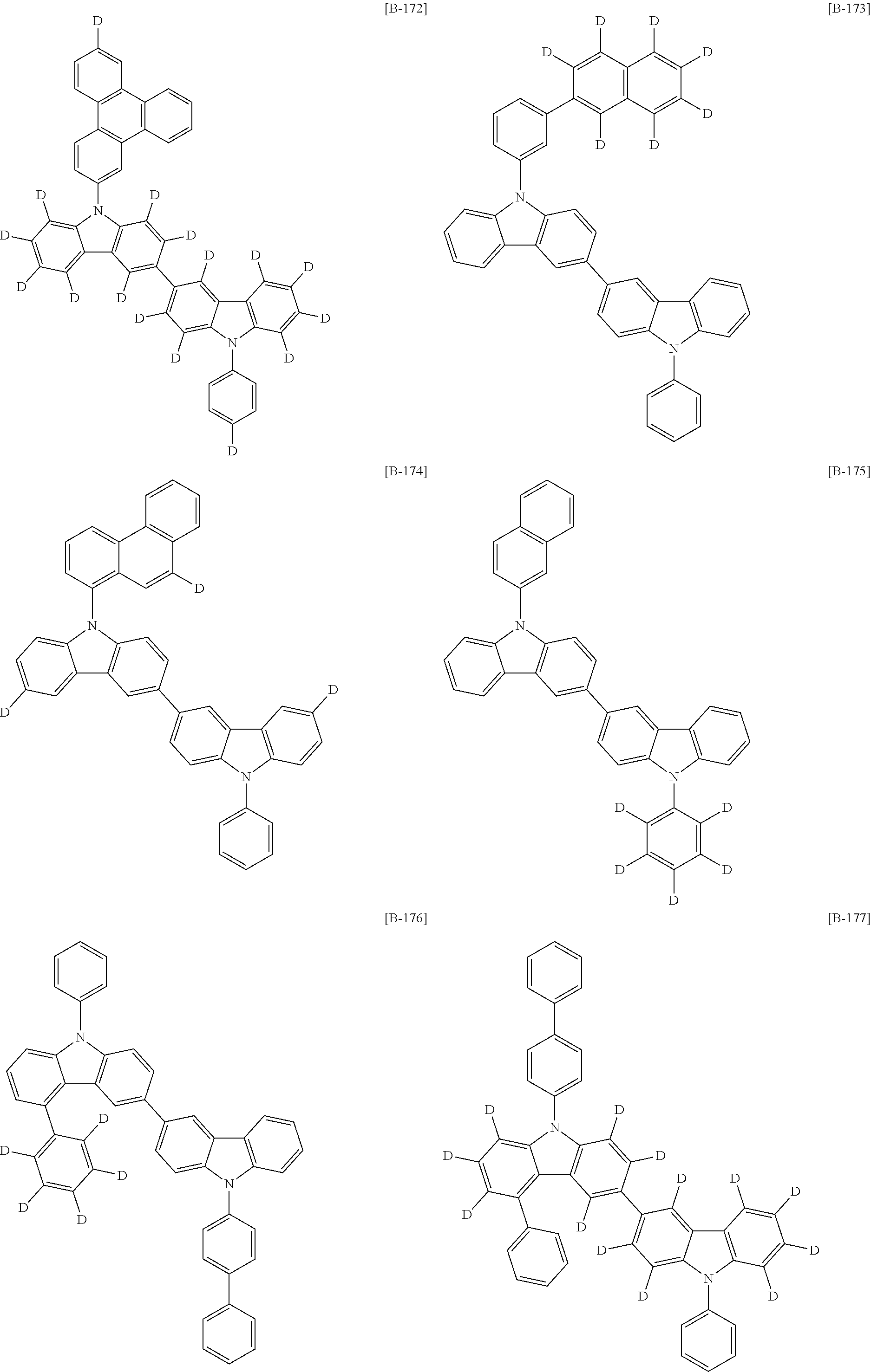

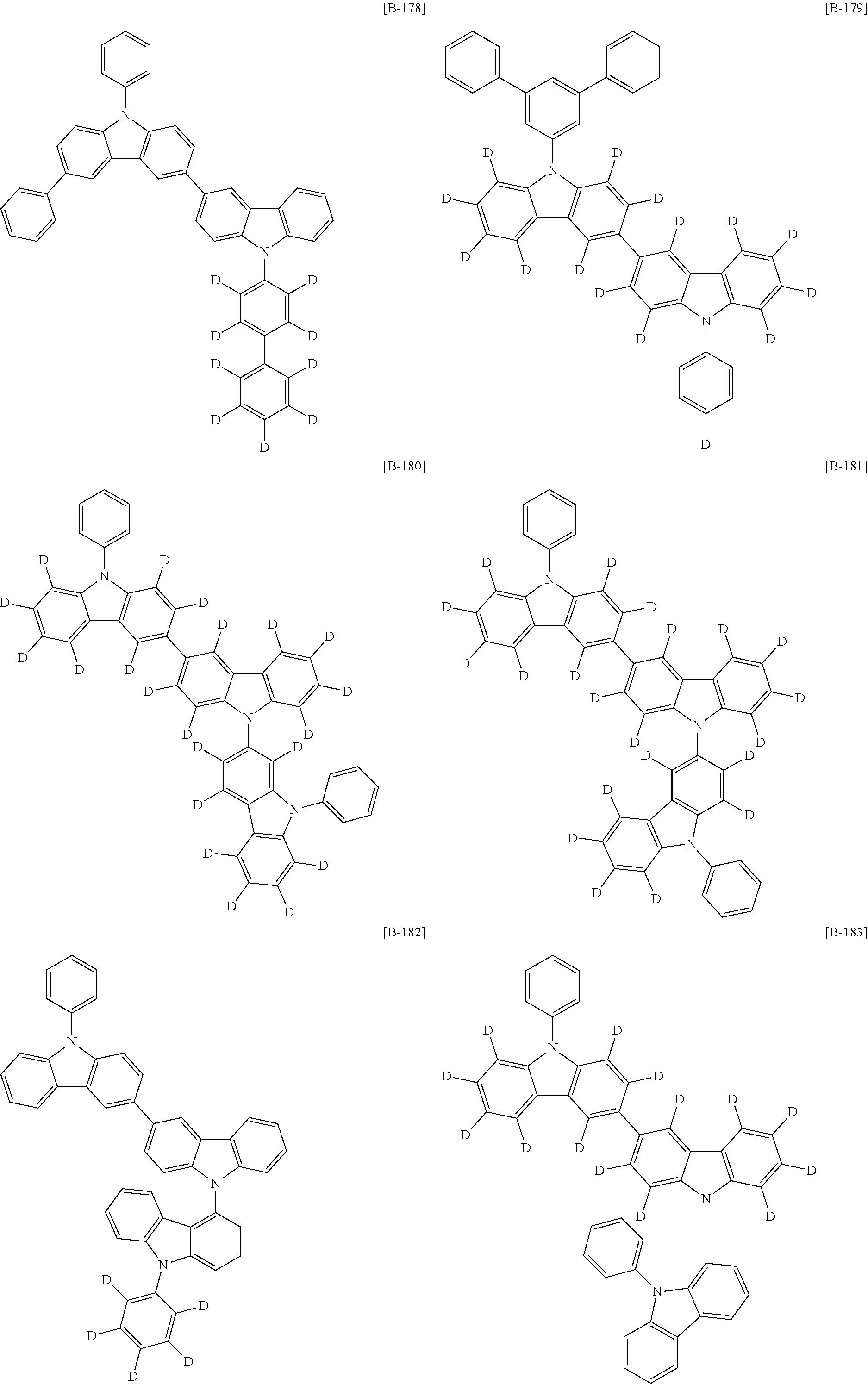

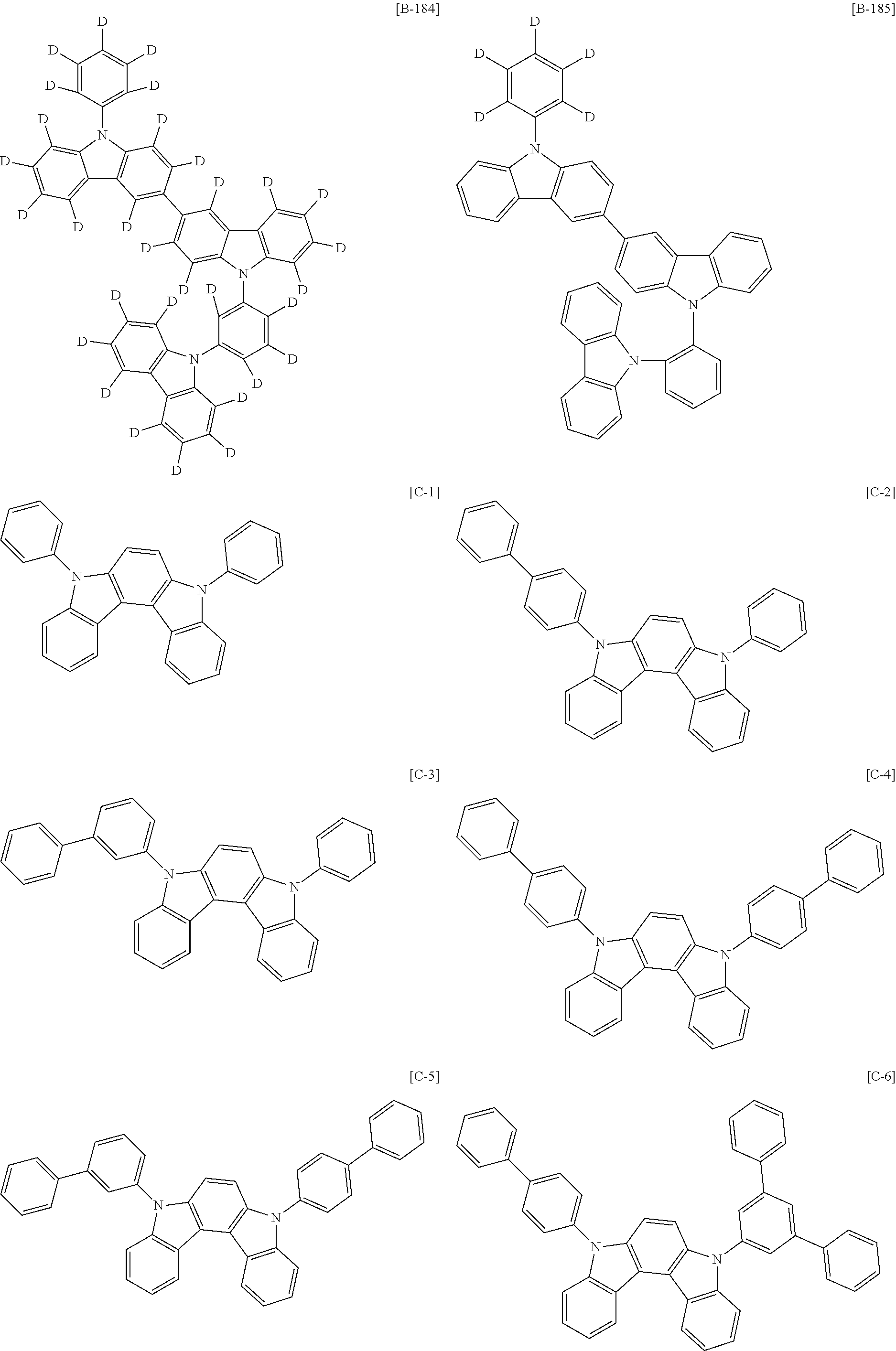

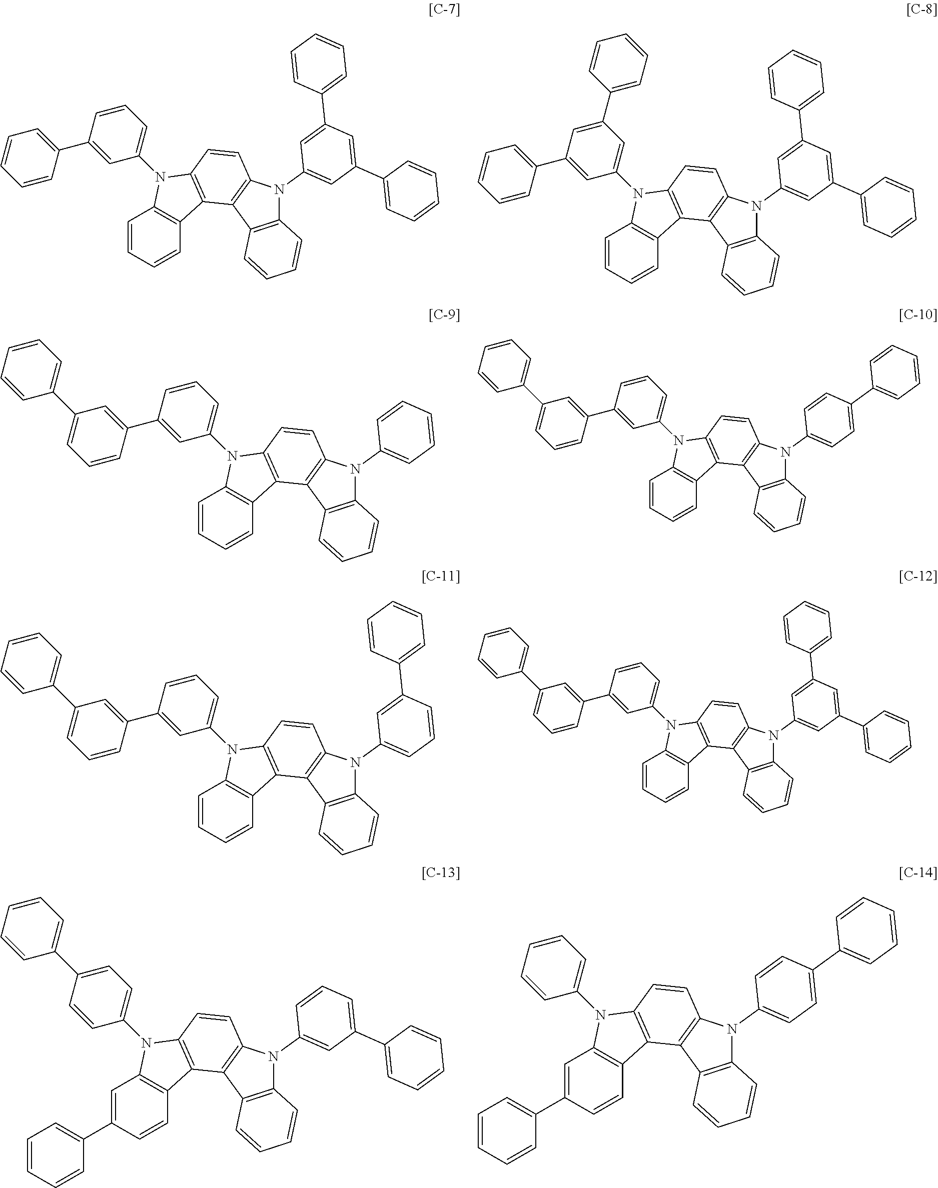

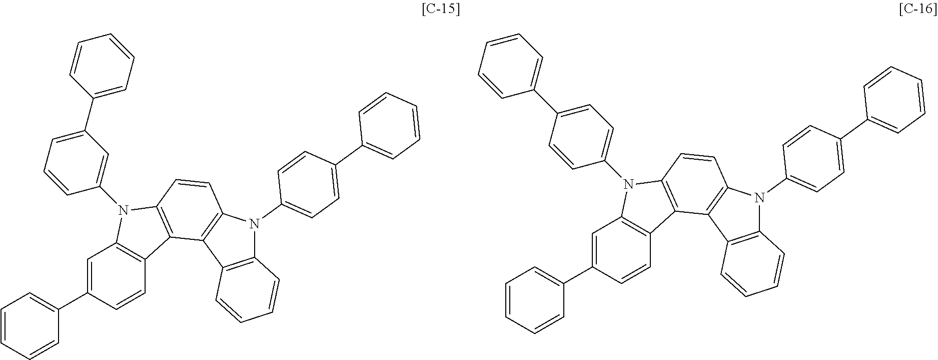 [SMILES: c1ccc(-c2ccc(-n3c4ccccc4c4c5c6ccc(-c7ccccc7)cc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccc(-c7ccccc7)cc6)c5ccc43)cc2)cc1]

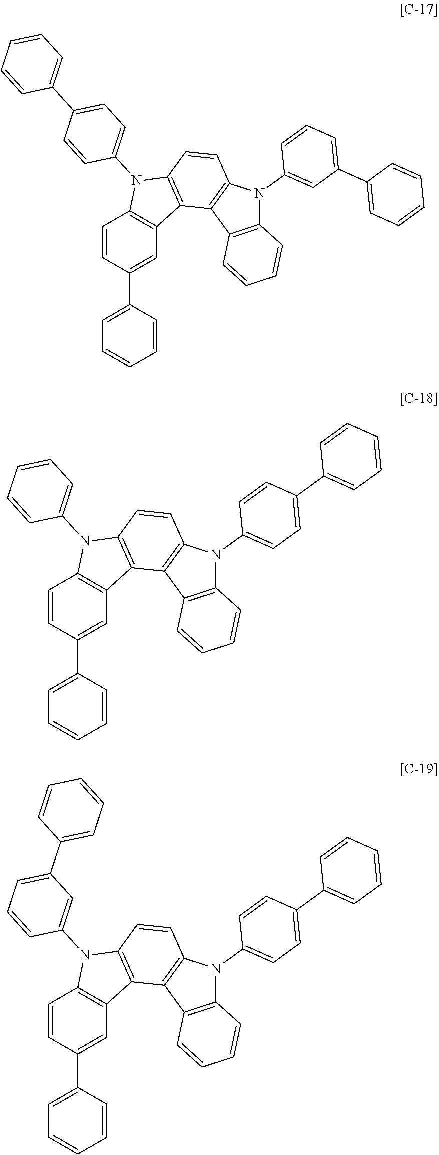

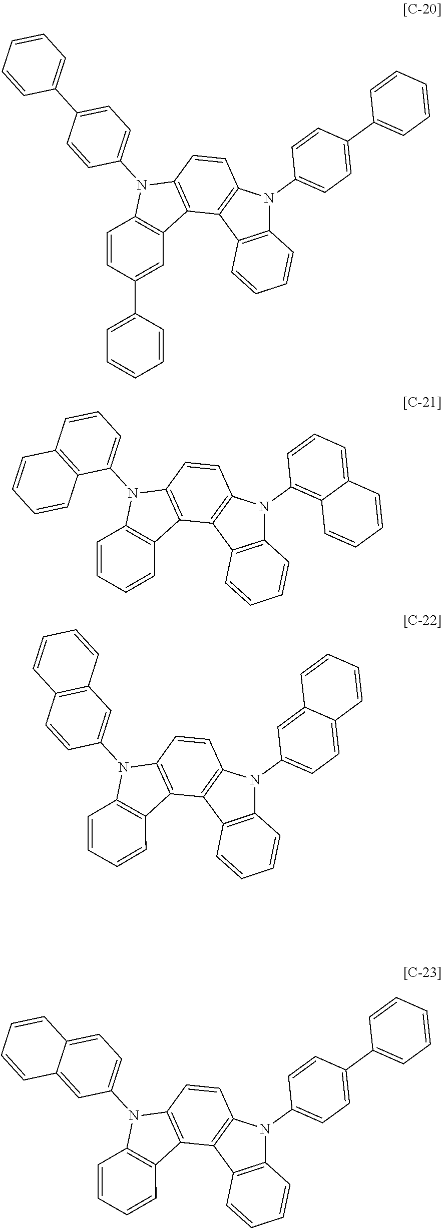 [SMILES: c1ccc(-c2ccc(-n3c4ccccc4c4c5c6cc(-c7ccccc7)ccc6n(-c6ccc(-c7ccccc7)cc6)c5ccc43)cc2)cc1 ; c1ccc2c(-n3c4ccccc4c4c5c6ccccc6n(-c6cccc7ccccc67)c5ccc43)cccc2c1 ; c1ccc2cc(-n3c4ccccc4c4c5c6ccccc6n(-c6ccc7ccccc7c6)c5ccc43)ccc2c1 ; c1ccc(-c2ccc(-n3c4ccccc4c4c5c6ccccc6n(-c6ccc7ccccc7c6)c5ccc43)cc2)cc1]

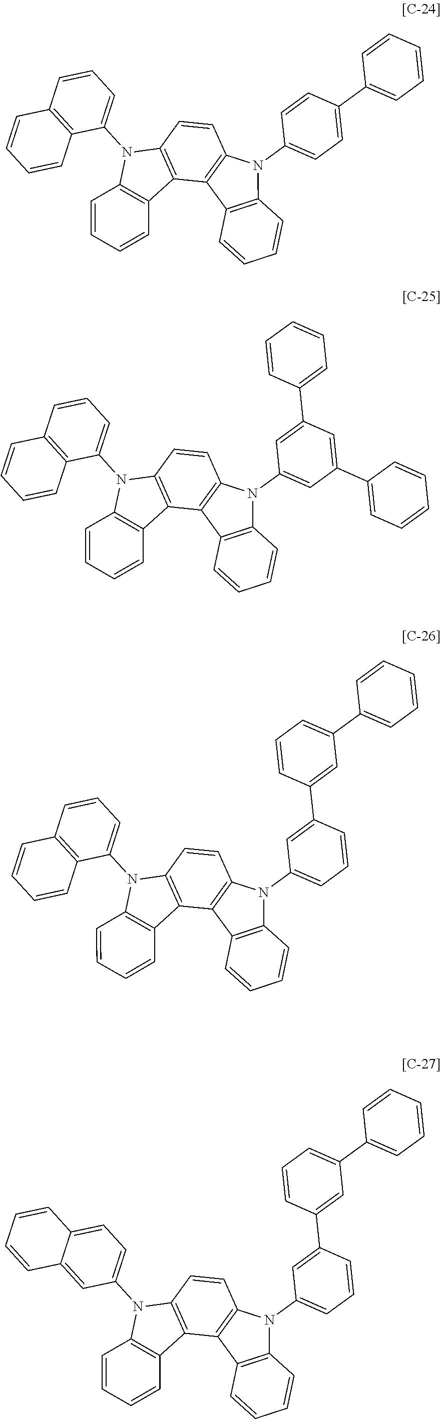 [SMILES: c1ccc(-c2ccc(-n3c4ccccc4c4c5c6ccccc6n(-c6cccc7ccccc67)c5ccc43)cc2)cc1 ; c1ccc(-c2cc(-c3ccccc3)cc(-n3c4ccccc4c4c5c6ccccc6n(-c6cccc7ccccc67)c5ccc43)c2)cc1 ; c1ccc(-c2cccc(-c3cccc(-n4c5ccccc5c5c6c7ccccc7n(-c7cccc8ccccc78)c6ccc54)c3)c2)cc1 ; c1ccc(-c2cccc(-c3cccc(-n4c5ccccc5c5c6c7ccccc7n(-c7ccc8ccccc8c7)c6ccc54)c3)c2)cc1]

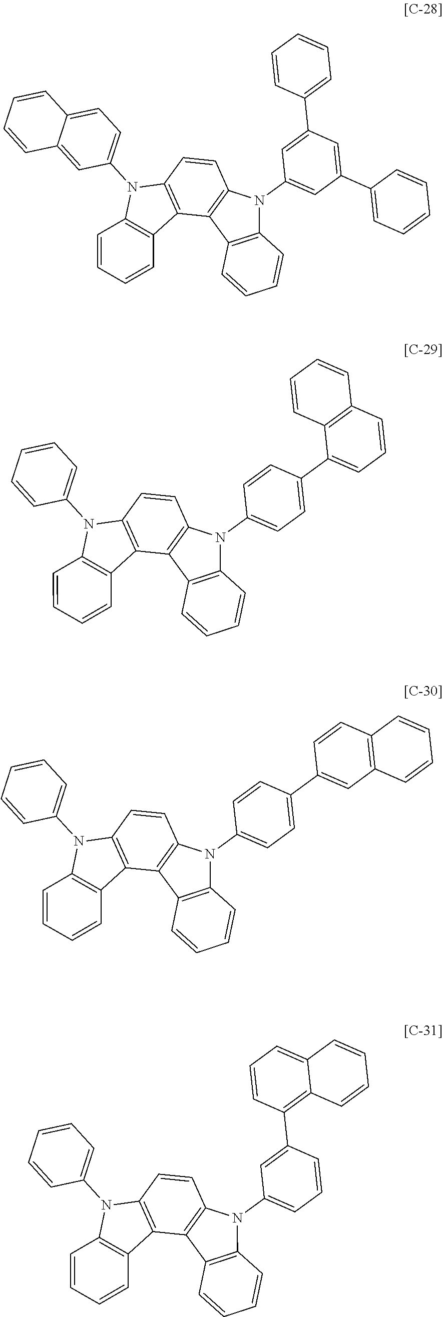 [SMILES: c1ccc(-c2cc(-c3ccccc3)cc(-n3c4ccccc4c4c5c6ccccc6n(-c6ccc7ccccc7c6)c5ccc43)c2)cc1 ; c1ccc(-n2c3ccccc3c3c4c5ccccc5n(-c5ccc(-c6cccc7ccccc67)cc5)c4ccc32)cc1 ; c1ccc(-n2c3ccccc3c3c4c5ccccc5n(-c5ccc(-c6ccc7ccccc7c6)cc5)c4ccc32)cc1 ; c1ccc(-n2c3ccccc3c3c4c5ccccc5n(-c5cccc(-c6cccc7ccccc67)c5)c4ccc32)cc1]

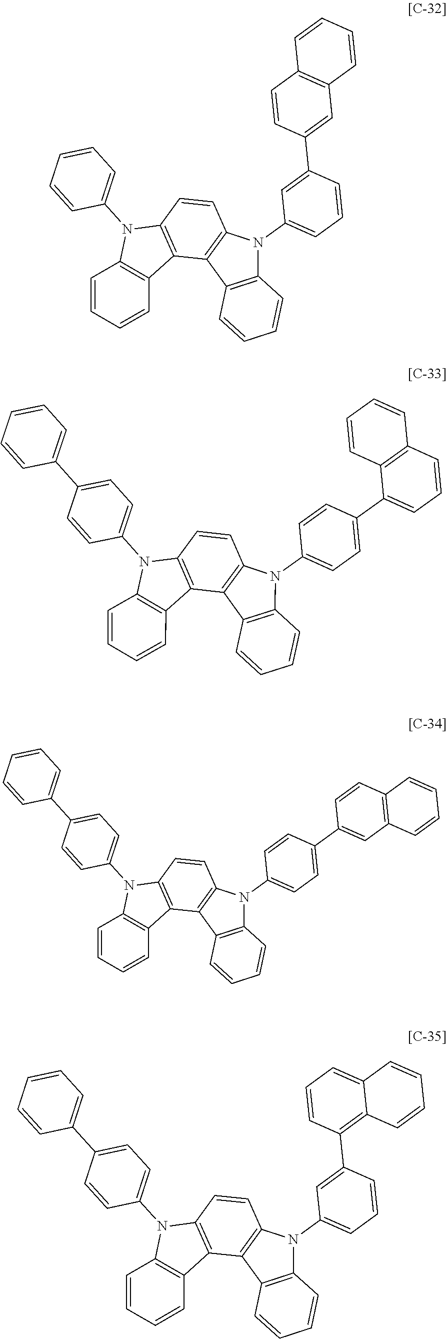 [SMILES: c1ccc(-n2c3ccccc3c3c4c5ccccc5n(-c5cccc(-c6ccc7ccccc7c6)c5)c4ccc32)cc1 ; c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1 ; c1ccc(-c2ccc(-n3c4ccccc4c4c5c6ccccc6n(-c6cccc(-c7cccc8ccccc78)c6)c5ccc43)cc2)cc1]

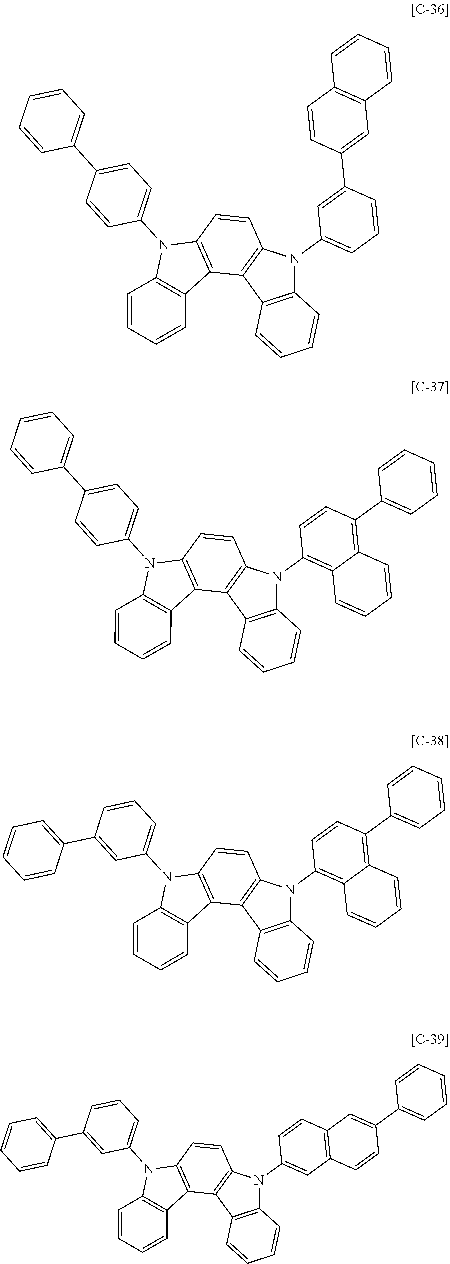 [SMILES: c1ccc(-c2ccc(-n3c4ccccc4c4c5c6ccccc6n(-c6cccc(-c7ccc8ccccc8c7)c6)c5ccc43)cc2)cc1 ; c1ccc(-c2ccc(-n3c4ccccc4c4c5c6ccccc6n(-c6ccc(-c7ccccc7)c7ccccc67)c5ccc43)cc2)cc1 ; c1ccc(-c2cccc(-n3c4ccccc4c4c5c6ccccc6n(-c6ccc(-c7ccccc7)c7ccccc67)c5ccc43)c2)cc1 ; c1ccc(-c2cccc(-n3c4ccccc4c4c5c6ccccc6n(-c6ccc7cc(-c8ccccc8)ccc7c6)c5ccc43)c2)cc1]

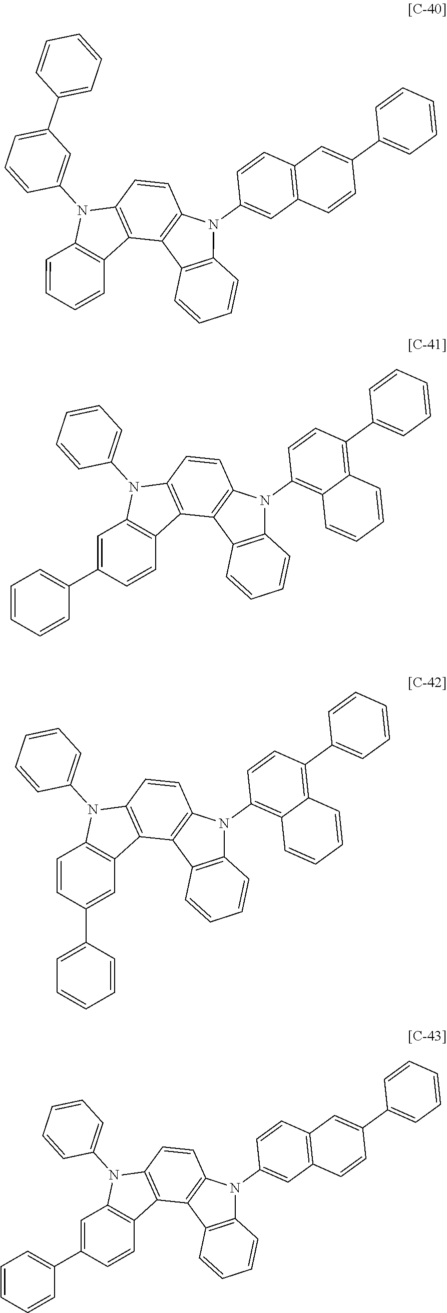 [SMILES: c1ccc(-c2cccc(-n3c4ccccc4c4c5c6ccccc6n(-c6ccc7cc(-c8ccccc8)ccc7c6)c5ccc43)c2)cc1 ; c1ccc(-c2ccc3c4c5c6ccccc6n(-c6ccc(-c7ccccc7)c7ccccc67)c5ccc4n(-c4ccccc4)c3c2)cc1 ; c1ccc(-c2ccc3c(c2)c2c4c5ccccc5n(-c5ccc(-c6ccccc6)c6ccccc56)c4ccc2n3-c2ccccc2)cc1 ; c1ccc(-c2ccc3cc(-n4c5ccccc5c5c6c7ccc(-c8ccccc8)cc7n(-c7ccccc7)c6ccc54)ccc3c2)cc1]

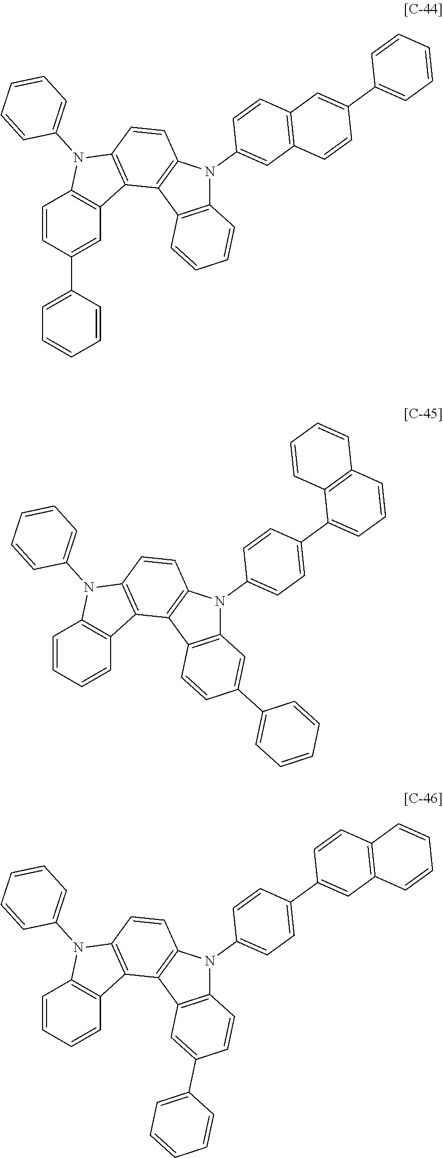 [SMILES: c1ccc(-c2ccc3cc(-n4c5ccccc5c5c6c7cc(-c8ccccc8)ccc7n(-c7ccccc7)c6ccc54)ccc3c2)cc1 ; c1ccc(-c2ccc3c4c5c6ccccc6n(-c6ccccc6)c5ccc4n(-c4ccc(-c5cccc6ccccc56)cc4)c3c2)cc1 ; c1ccc(-c2ccc3c(c2)c2c4c5ccccc5n(-c5ccccc5)c4ccc2n3-c2ccc(-c3ccc4ccccc4c3)cc2)cc1]

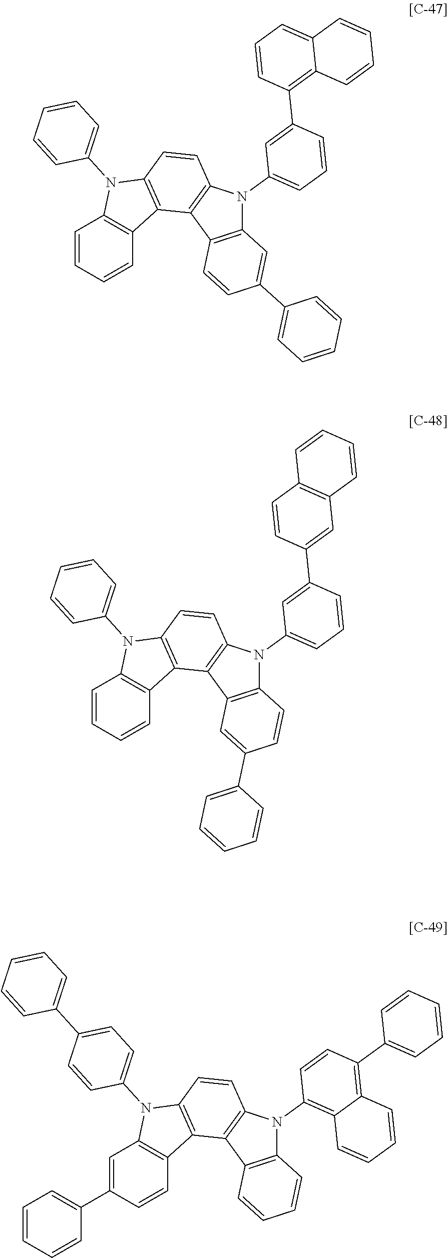 [SMILES: c1ccc(-c2ccc3c4c5c6ccccc6n(-c6ccccc6)c5ccc4n(-c4cccc(-c5cccc6ccccc56)c4)c3c2)cc1 ; c1ccc(-c2ccc3c(c2)c2c4c5ccccc5n(-c5ccccc5)c4ccc2n3-c2cccc(-c3ccc4ccccc4c3)c2)cc1 ; c1ccc(-c2ccc(-n3c4cc(-c5ccccc5)ccc4c4c5c6ccccc6n(-c6ccc(-c7ccccc7)c7ccccc67)c5ccc43)cc2)cc1]

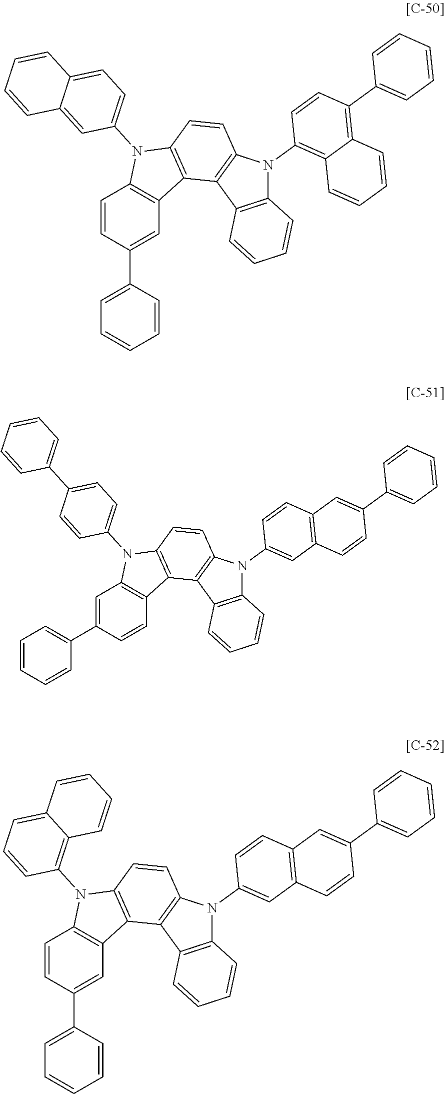 [SMILES: c1ccc(-c2ccc3c(c2)c2c4c5ccccc5n(-c5ccc(-c6ccccc6)c6ccccc56)c4ccc2n3-c2ccc3ccccc3c2)cc1 ; c1ccc(-c2ccc(-n3c4cc(-c5ccccc5)ccc4c4c5c6ccccc6n(-c6ccc7cc(-c8ccccc8)ccc7c6)c5ccc43)cc2)cc1 ; c1ccc(-c2ccc3cc(-n4c5ccccc5c5c6c7cc(-c8ccccc8)ccc7n(-c7cccc8ccccc78)c6ccc54)ccc3c2)cc1]

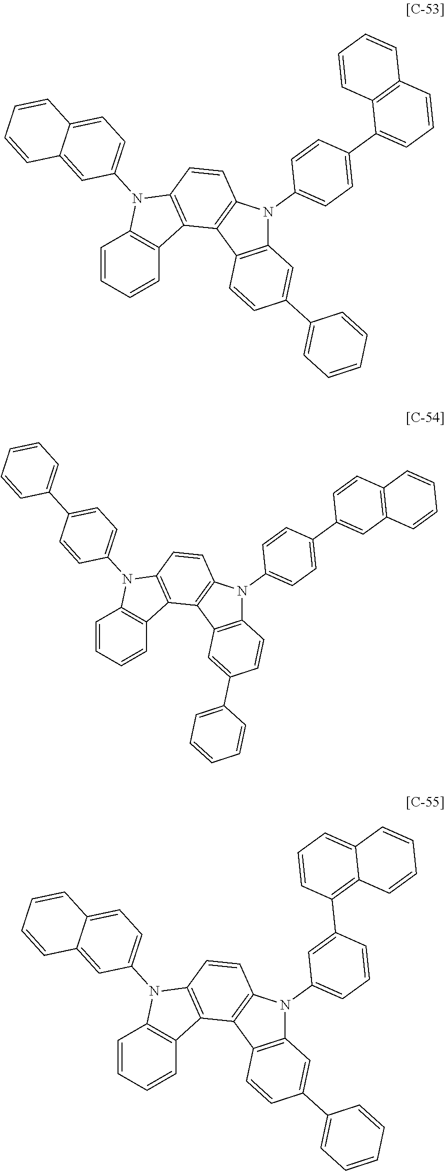 [SMILES: c1ccc(-c2ccc3c4c5c6ccccc6n(-c6ccc7ccccc7c6)c5ccc4n(-c4ccc(-c5cccc6ccccc56)cc4)c3c2)cc1 ; c1ccc(-c2ccc(-n3c4ccccc4c4c5c6cc(-c7ccccc7)ccc6n(-c6ccc(-c7ccc8ccccc8c7)cc6)c5ccc43)cc2)cc1 ; c1ccc(-c2ccc3c4c5c6ccccc6n(-c6ccc7ccccc7c6)c5ccc4n(-c4cccc(-c5cccc6ccccc56)c4)c3c2)cc1]

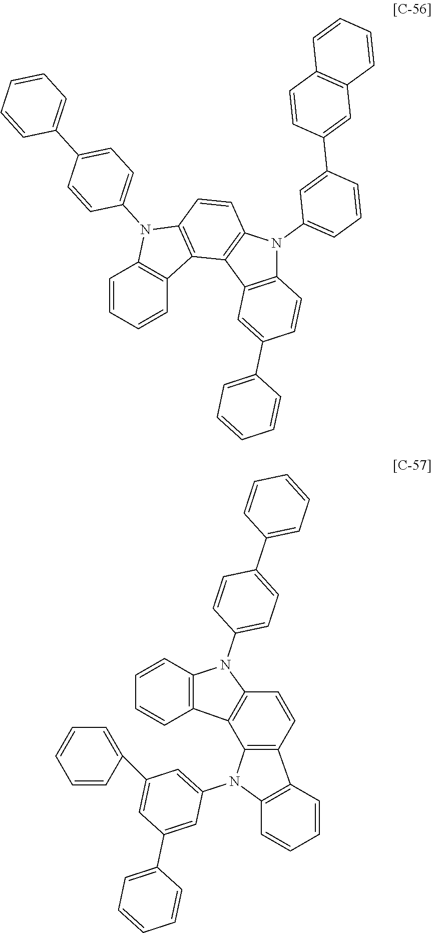

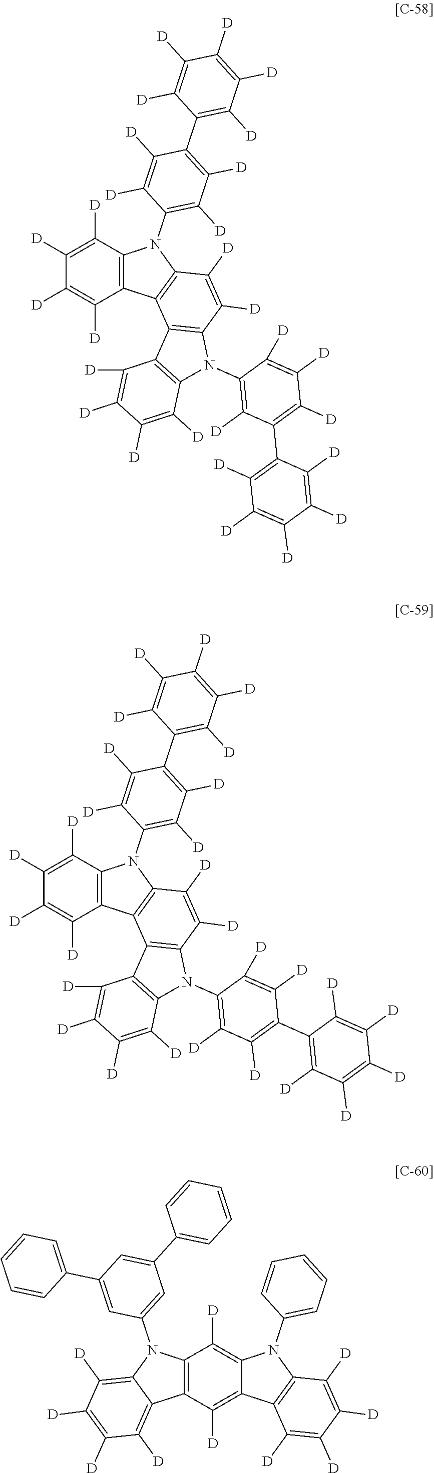

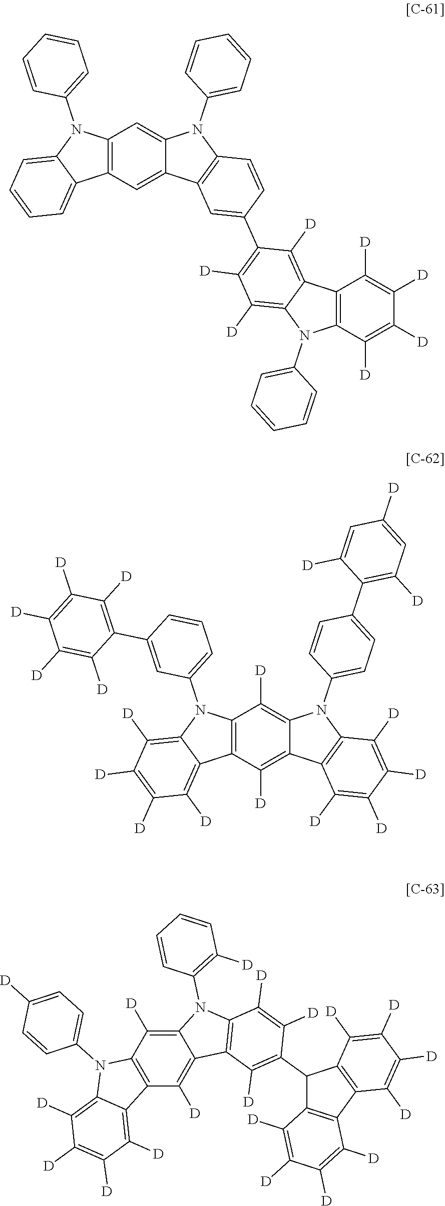 [SMILES: [2H]c1c([2H])c([2H])c2c(c1[2H])c1c([2H])c(-c3ccc4c(c3)c3cc5c6ccccc6n(-c6ccccc6)c5cc3n4-c3ccccc3)c([2H])c([2H])c1n2-c1ccccc1 ; [2H]c1cc([2H])c(-c2ccc(-n3c4c([2H])c([2H])c([2H])c([2H])c4c4c([2H])c5c6c([2H])c([2H])c([2H])c([2H])c6n(-c6cccc(-c7c([2H])c([2H])c([2H])c([2H])c7[2H])c6)c5c([2H])c43)cc2)c([2H])c1 ; [2H]c1ccc(-n2c3c([2H])c([2H])c([2H])c([2H])c3c3c([2H])c4c5c([2H])c(C6c7c([2H])c([2H])c([2H])c([2H])c7-c7c([2H])c([2H])c([2H])c([2H])c76)c([2H])c([2H])c5n(-c5ccccc5[2H])c4c([2H])c32)cc1]

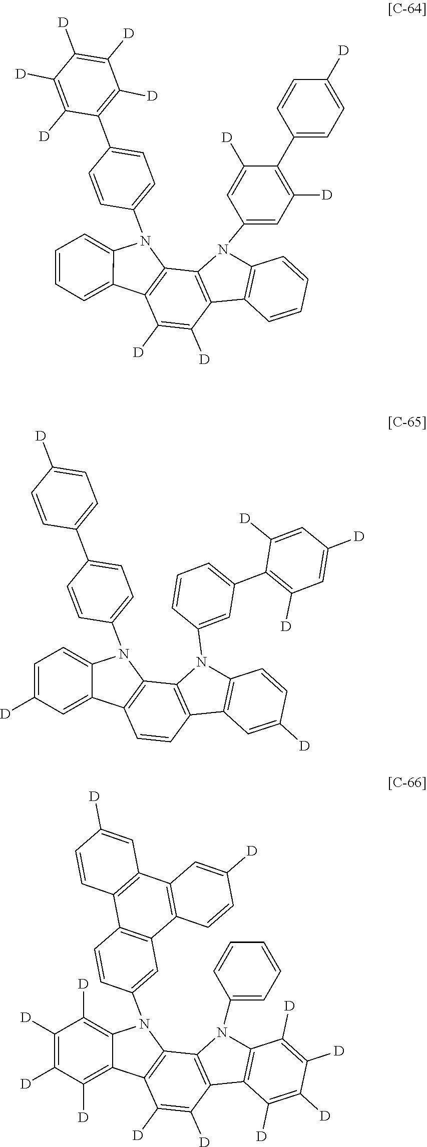 [SMILES: [2H]c1ccc(-c2c([2H])cc(-n3c4ccccc4c4c([2H])c([2H])c5c6ccccc6n(-c6ccc(-c7c([2H])c([2H])c([2H])c([2H])c7[2H])cc6)c5c43)cc2[2H])cc1 ; [2H]c1ccc(-c2ccc(-n3c4ccc([2H])cc4c4ccc5c6cc([2H])ccc6n(-c6cccc(-c7c([2H])cc([2H])cc7[2H])c6)c5c43)cc2)cc1 ; [2H]c1ccc2c3ccc(-n4c5c([2H])c([2H])c([2H])c([2H])c5c5c([2H])c([2H])c6c7c([2H])c([2H])c([2H])c([2H])c7n(-c7ccccc7)c6c54)cc3c3ccc([2H])cc3c2c1]

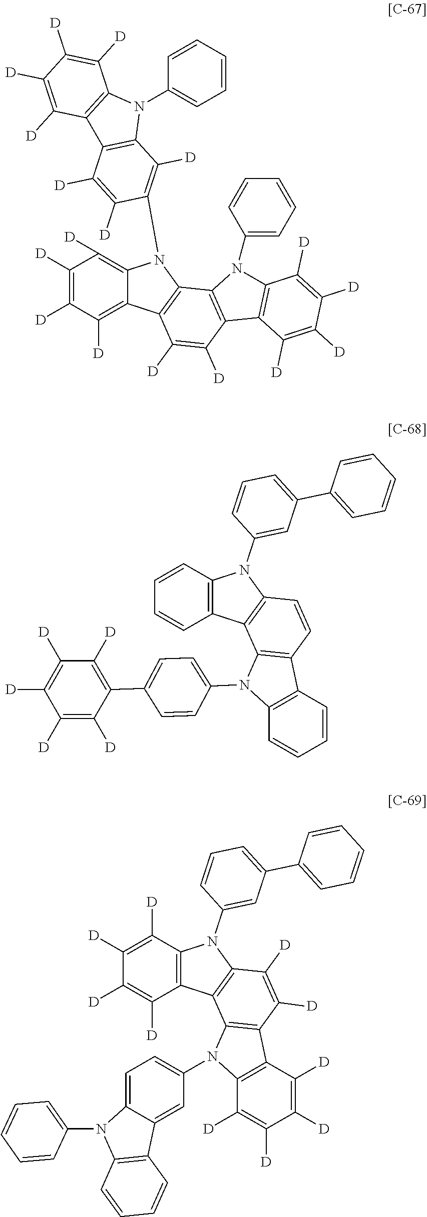 [SMILES: [2H]c1c([2H])c([2H])c2c(c1[2H])c1c([2H])c([2H])c(-n3c4c([2H])c([2H])c([2H])c([2H])c4c4c([2H])c([2H])c5c6c([2H])c([2H])c([2H])c([2H])c6n(-c6ccccc6)c5c43)c([2H])c1n2-c1ccccc1 ; [2H]c1c([2H])c([2H])c(-c2ccc(-n3c4ccccc4c4ccc5c(c6ccccc6n5-c5cccc(-c6ccccc6)c5)c43)cc2)c([2H])c1[2H] ; [2H]c1c([2H])c([2H])c2c(c1[2H])c1c(c([2H])c([2H])c3c4c([2H])c([2H])c([2H])c([2H])c4n(-c4ccc5c(c4)c4ccccc4n5-c4ccccc4)c31)n2-c1cccc(-c2ccccc2)c1]

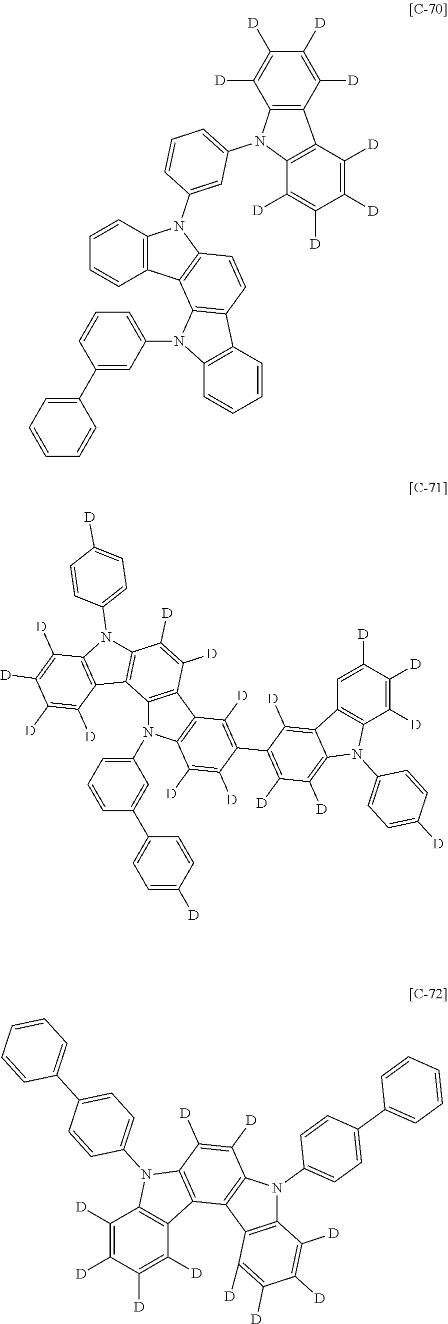 [SMILES: [2H]c1c([2H])c([2H])c2c(c1[2H])c1c([2H])c([2H])c([2H])c([2H])c1n2-c1cccc(-n2c3ccccc3c3c2ccc2c4ccccc4n(-c4cccc(-c5ccccc5)c4)c23)c1 ; [2H]c1ccc(-c2cccc(-n3c4c([2H])c([2H])c(-c5c([2H])c([2H])c6c(c5[2H])c5cc([2H])c([2H])c([2H])c5n6-c5ccc([2H])cc5)c([2H])c4c4c([2H])c([2H])c5c(c6c([2H])c([2H])c([2H])c([2H])c6n5-c5ccc([2H])cc5)c43)c2)cc1 ; [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1]

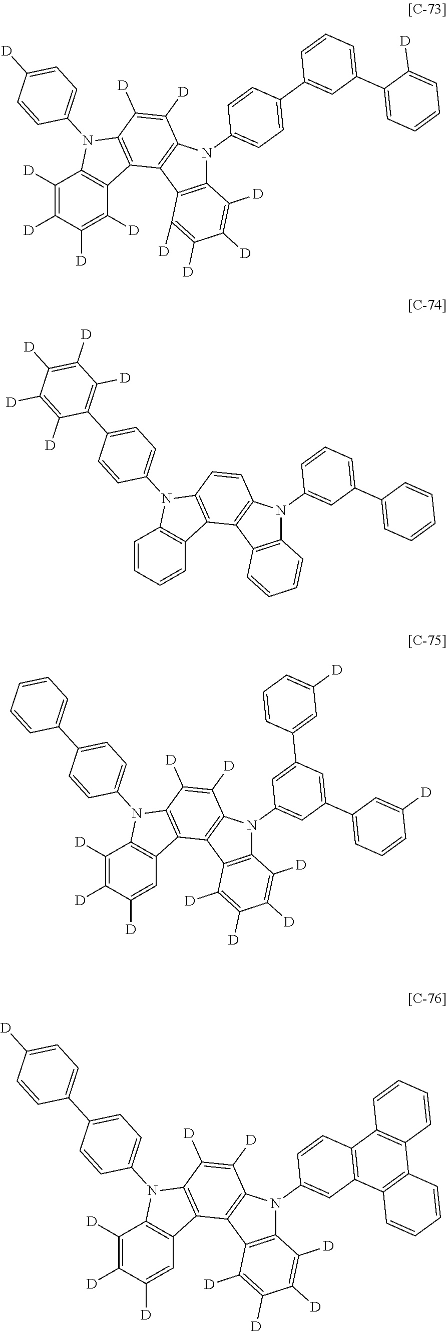

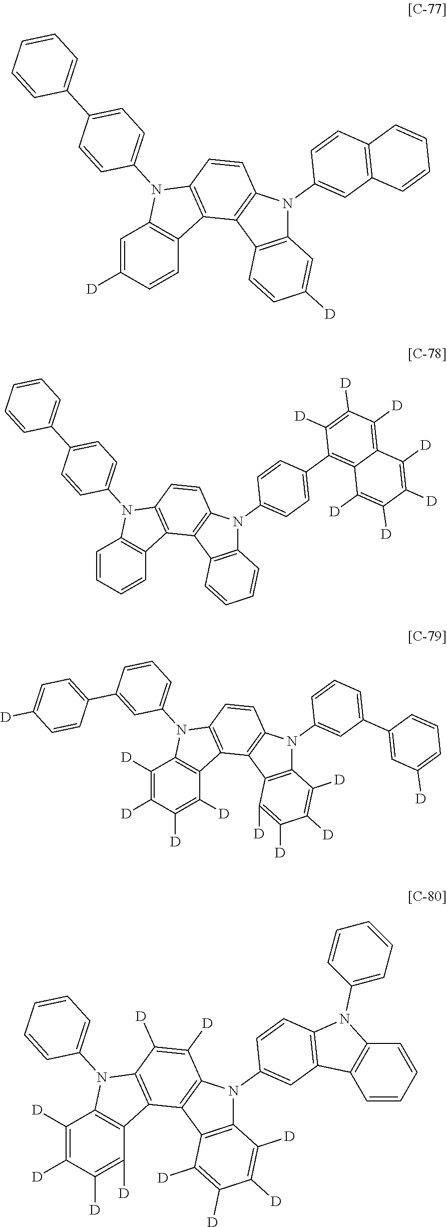 [SMILES: [2H]c1ccc2c3c4c5ccc([2H])cc5n(-c5ccc6ccccc6c5)c4ccc3n(-c3ccc(-c4ccccc4)cc3)c2c1 ; [2H]c1c([2H])c([2H])c2c(-c3ccc(-n4c5ccccc5c5c6c7ccccc7n(-c7ccc(-c8ccccc8)cc7)c6ccc54)cc3)c([2H])c([2H])c([2H])c2c1[2H] ; [2H]c1ccc(-c2cccc(-n3c4ccc5c(c6c([2H])c([2H])c([2H])c([2H])c6n5-c5cccc(-c6cccc([2H])c6)c5)c4c4c([2H])c([2H])c([2H])c([2H])c43)c2)cc1 ; [2H]c1c([2H])c([2H])c2c(c1[2H])c1c3c4c([2H])c([2H])c([2H])c([2H])c4n(-c4ccc5c(c4)c4ccccc4n5-c4ccccc4)c3c([2H])c([2H])c1n2-c1ccccc1]

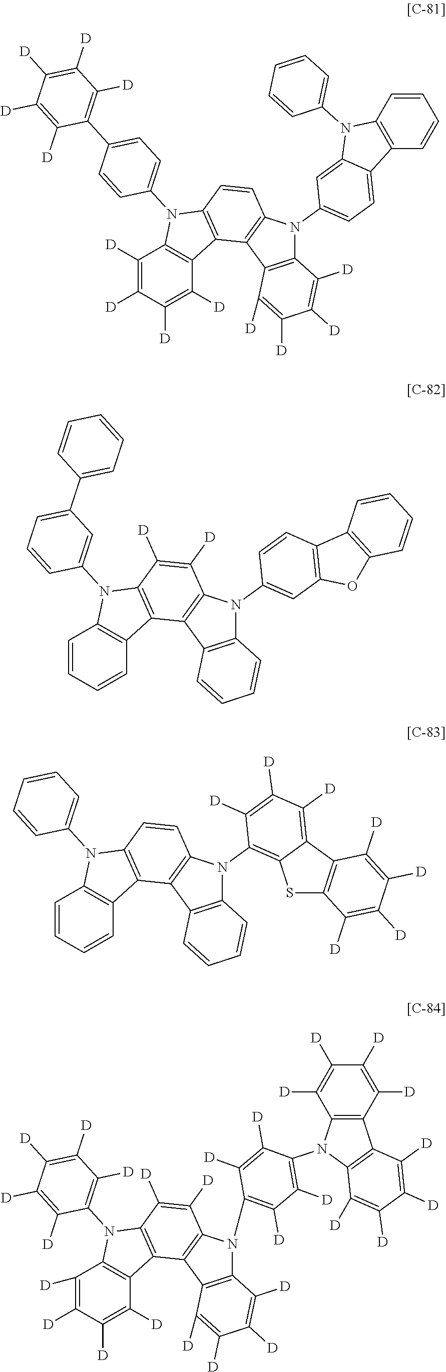

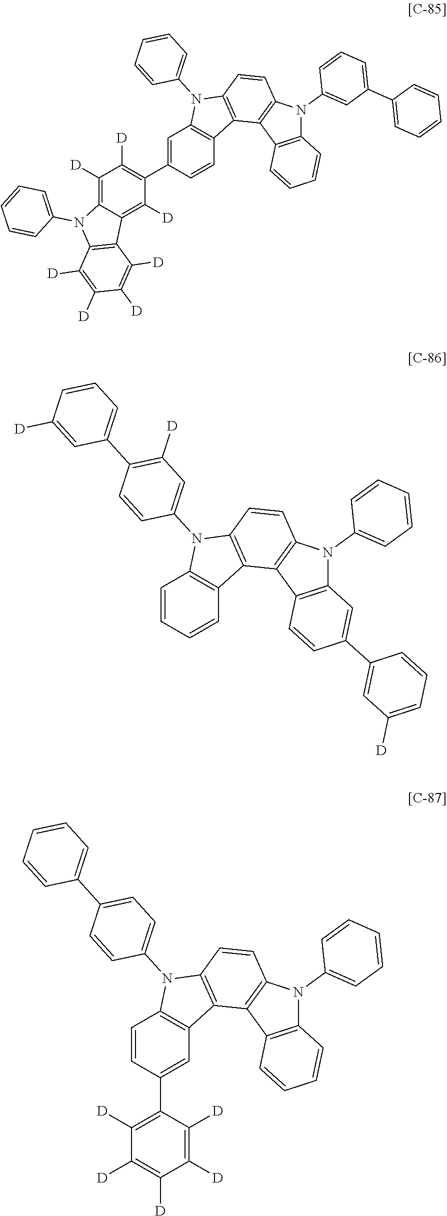 [SMILES: [2H]c1c([2H])c([2H])c2c(c1[2H])c1c([2H])c(-c3ccc4c5c6c7ccccc7n(-c7cccc(-c8ccccc8)c7)c6ccc5n(-c5ccccc5)c4c3)c([2H])c([2H])c1n2-c1ccccc1 ; [2H]c1cccc(-c2ccc3c4c5c6ccccc6n(-c6ccc(-c7cccc([2H])c7)c([2H])c6)c5ccc4n(-c4ccccc4)c3c2)c1 ; [2H]c1c([2H])c([2H])c(-c2ccc3c(c2)c2c4c5ccccc5n(-c5ccccc5)c4ccc2n3-c2ccc(-c3ccccc3)cc2)c([2H])c1[2H]]

One type or two or more types of the second compound may be used.

In the light emitting layer 32, the first compound and the second compound may be included (e.g., mixed) as a host, e.g., in a weight ratio of about 1:99 to about 99:1. Within the above range, bipolar characteristics may be implemented by matching an appropriate weight ratio using electron transport capability of the first compound and the hole transport capability of the second compound, to improve efficiency and life-span. In an implementation, they may be included in a weight ratio of about 10:90 to about 90:10, about 20:80 to about 80:20, e.g. about 20:80 to about 70:30, about 20:80 to about 60:40, or about 20:80 to about 50:50. In an implementation, they may be included in a weight ratio of about 20:80, about 30:70, or about 40:60.

The light emitting layer 32 may further include one or more compounds in addition to the aforementioned first and second compounds described above as a host.

The light emitting layer 32 may further include a dopant.

The dopant may be, e.g., a phosphorescent dopant, for example a red, green, or blue phosphorescent dopant, e.g., a red or green phosphorescent dopant.

The dopant is a material mixed with the compound for the organic optoelectronic device in a small amount to cause light emission and may be generally a material such as a metal complex that emits light by multiple excitation into a triplet or more. The dopant may be, e.g., an inorganic, organic, or organic-inorganic compound, and one or more types thereof may be used.

Examples of the dopant may include a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, e.g., a compound represented by Chemical Formula Z.

L¹²MX³ [Chemical Formula Z]

In Chemical Formula Z, M may be, e.g., a metal, and L¹² and X³ may each independently be, e.g., ligands forming a complex compound with M.

The M may be, e.g., Ir, Pt, Os, Ti, Zr, Hf, Eu, Tb, Tm, Fe, Co, Ni, Ru, Rh, Pd, or a combination thereof, and L¹² and X³ may be, e.g., a bidentate ligand.

Examples of the ligands represented by L¹² and X³ may be selected from the chemical formulae of Group A.

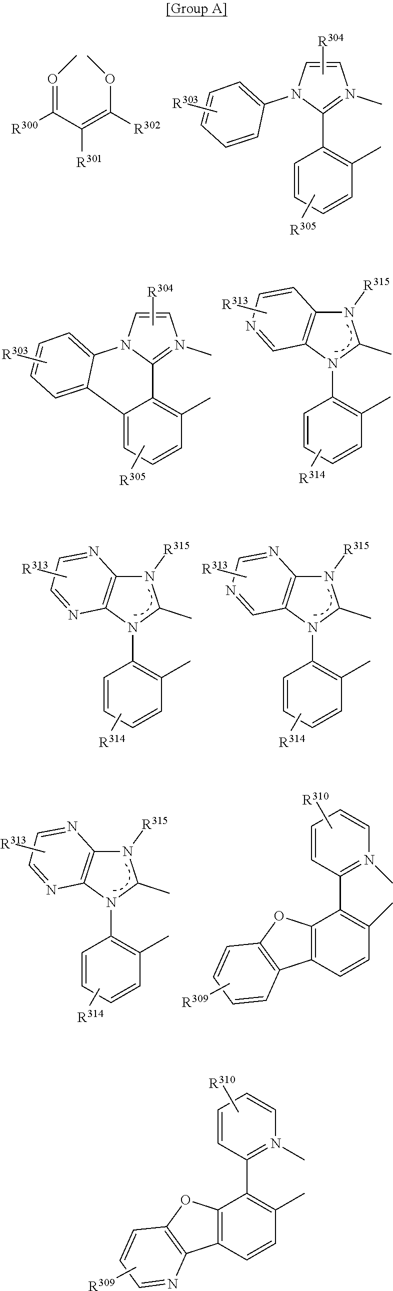

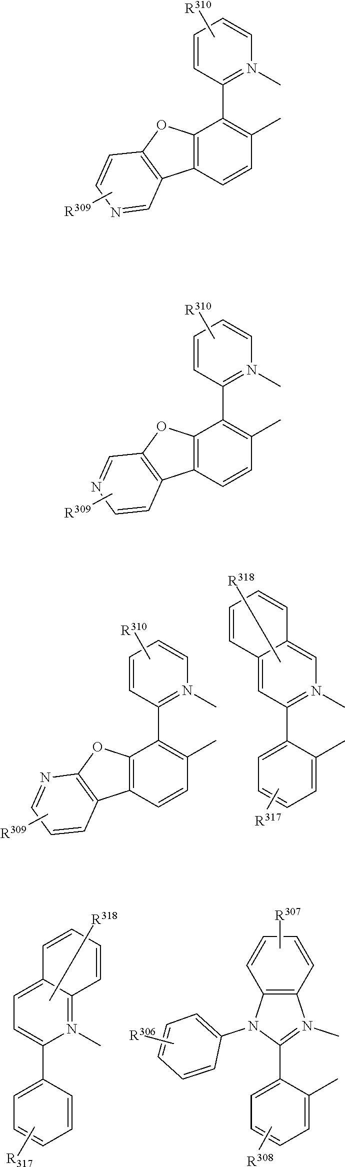

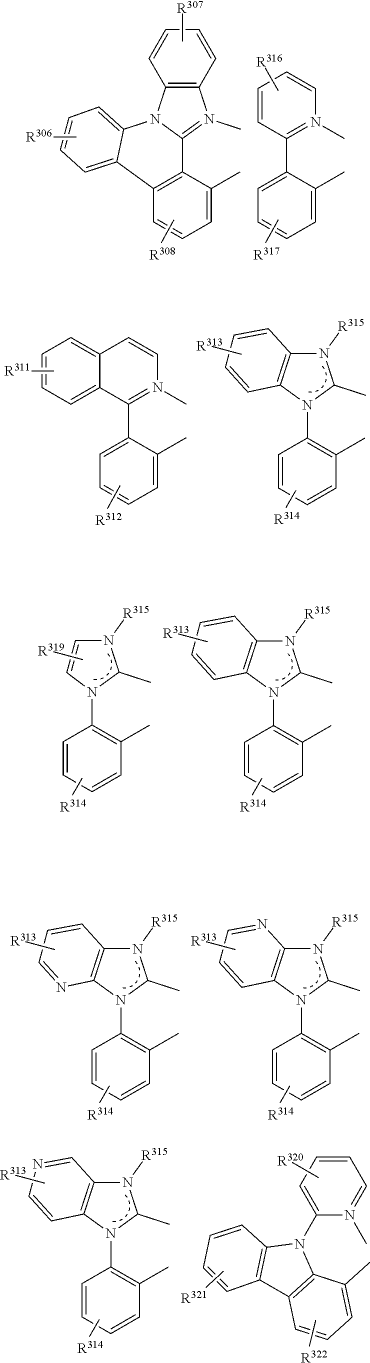

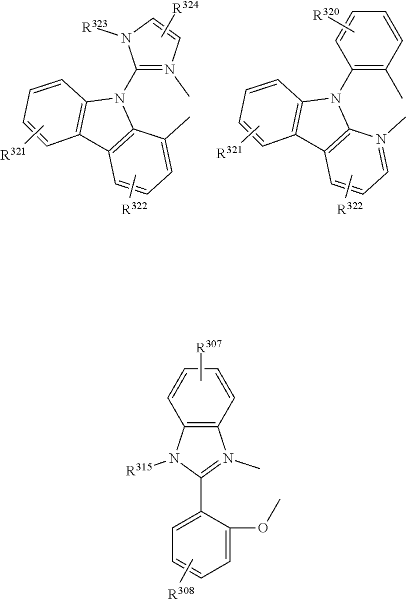

In Group A, R³⁰⁰ to R³⁰² may each independently be, e.g., hydrogen, deuterium, a C1 to C30 alkyl group that is substituted or unsubstituted with a halogen, a C6 to C30 aryl group that is substituted or unsubstituted with a C1 to C30 alkyl, or a halogen.

R³⁰ to R³²⁴ may each independently be, e.g., hydrogen, deuterium, halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SFs, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

In an implementation, it may include a dopant represented by Chemical Formula V.

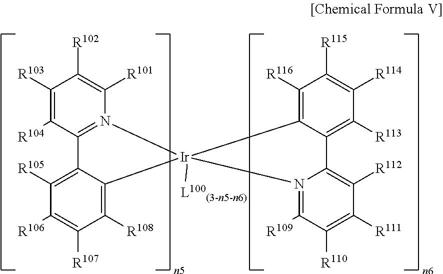

In Chemical Formula V, R¹⁰¹ to R¹¹⁶ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a substituted or unsubstituted C1 to C6 alkyl group.

In an implementation, at least one of R¹⁰¹ to R¹¹⁶ may be, e.g., a functional group represented by Chemical Formula V-1,

L¹⁰⁰ may be, e.g., a bidentate ligand of a monovalent anion, and is a ligand that coordinates to iridium through a lone pair of electrons of carbon or heteroatom.

n5 and n6 may each independently be, e.g., an integer of 0 to 3, and n5+n6 may be, e.g., an integer of 1 to 3.

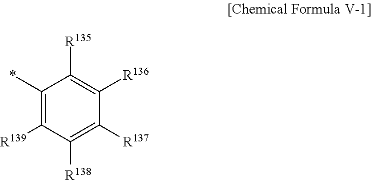

In Chemical Formula V-1, R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a substituted or unsubstituted C1 to C6 alkyl group.

* means a portion linked to a carbon atom.

In an implementation, a dopant represented by Chemical Formula Z-1 may be included.

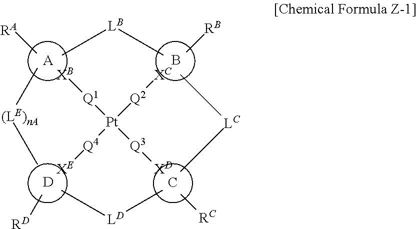

In Chemical Formula Z-1, rings A, B, C, and D each independently represent a 5-membered or 6-membered carbocyclic or heterocyclic ring.

R^{A}, R^{B}, R^{C}, and R^{D} may each independently represent, e.g., mono-, di-, tri-, or tetra-substitution, or unsubstitution.

L^{B}, L^{C}, and L^{D} may each independently be, e.g., a direct bond, BR, NR, PR, 0, S, Se, C═O, S═O, SO₂, CRR′, SiRR′, GeRR′, or a combination thereof.

In an implementation, when nA is 1, L^{E} may be a direct bond, BR, NR, PR, 0, S, Se, C═O, S═O, SO₂, CRR′, SiRR′, GeRR′, or a combination thereof, and when nA is 0, L^{E} does not exist.

R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof, any adjacent R^{A}, R^{B}, R^{C}, R^{D}, R, and R′ may be optionally linked to each other to provide a ring; X^{B}, X^{C}, X^{D}, and X^{E} may each independently be, e.g., carbon or nitrogen; and Q¹, Q², Q³, and Q⁴ may each independently be, e.g., oxygen or a direct bond.

The dopant according to an embodiment may be a platinum complex, and may be, e.g., represented by Chemical Formula VI.

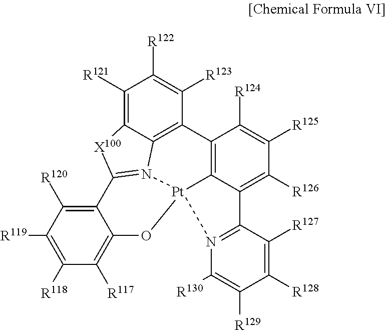

In Chemical Formula VI, X¹⁰⁰ may be, e.g., O, S, or NR¹³¹.

R¹¹⁷ to R¹³¹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a substituted or unsubstituted C1 to C6 alkyl group.

In an implementation, at least one of R¹⁷ to R¹³¹ may be, e.g., —SiR¹³²R¹³³R¹³⁴ or a tert-butyl group.

R¹³² to R¹³⁴ may each independently be, e.g., a substituted or unsubstituted C1 to C6 alkyl group.

The hole transport auxiliary layer 33 may include, e.g., a third compound having a relatively strong bipolar characteristic.

As described above, the light emitting layer 32 may include a first compound having a bipolar characteristic in which an electron characteristic is relatively strong and a second compound having a relatively strong hole characteristic together and thus the mobility of electrons and holes may be increased compared with the case of being used alone to significantly improve luminous efficiency.

In a device in which a material having biased electronic or hole characteristics is introduced into the light emitting layer, a recombination of carriers could occur at the interface between the light emitting layer and the electron or charge transport layer, and formation of excitons could occur relatively frequently. As a result, due to the interaction between molecular excitons in the light emitting layer and charges at the interface of the hole transport layer, a roll-off phenomenon in which efficiency is rapidly reduced could occur, and light emission life-span characteristics could also be rapidly reduced.

In view of the above, the first and second compounds may be introduced into the light emitting layer at the same time so that the light emitting region is not biased toward either the electron transport layer or the hole transport layer and in addition, a hole transport auxiliary layer including the third compound having a relatively strong bipolar characteristic may be included between the hole transport layer and the light emitting layer, and thereby, the device may be capable of preventing charge accumulation at the interface between the hole transport layer and the light emitting layer and balancing carriers in the light emitting layer. Accordingly, it is possible to improve the roll-off characteristics of the organic optoelectronic device and also significantly improve the life-span characteristics.

The third compound may be a compound represented by, e.g., a combination of Chemical Formula 5 and Chemical Formula 6.

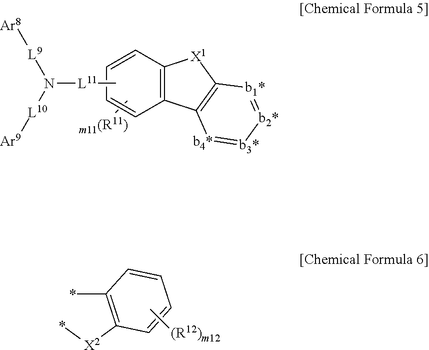

In Chemical Formula 5 and Chemical Formula 6, X¹ and X² may each independently be, e.g., O, S, or CR^{b}R^{c}.

Two adjacent ones of b1* to b4* of Chemical Formula 5 may be linking carbons linked at * of Chemical Formula 6, and the remaining two of b1* to b4* of Chemical Formula 5, not linked at * of Chemical Formula 6, may be C-L^{b}-R^{d}.

L^{b}, and L⁹ to L¹¹ may each independently be or include, e.g., a single bond, a substituted or unsubstituted C6 to C20 arylene group, or a substituted or unsubstituted C2 to C20 heterocyclic group.

Ar⁸ and Ar⁹ may each independently be or include, e.g., a substituted or unsubstituted C6 to C30 aryl group or a substituted or unsubstituted C2 to C30 heterocyclic group.

R^{b} and R^{c} may each independently be or include, e.g., a substituted or unsubstituted C1 to C30 alkyl group or a substituted or unsubstituted C6 to C30 aryl group.

R^{d}, R¹¹, and R¹² may each independently be or include, e.g., hydrogen, deuterium, a cyano group, a halogen, a substituted or unsubstituted C1 to C30 alkyl group, a substituted or unsubstituted C6 to C30 aryl group or a substituted or unsubstituted C2 to C30 heterocyclic group.

m11 may be, e.g., an integer of 1 to 3.

m12 may be, e.g., an integer of 1 to 4.

The third compound may have, e.g., a structure in which a 6-5-6-5-6 fused ring is substituted with an amine group.

By substituting the fused ring with an amine group, a HOMO energy level may be lowered to facilitate hole injection.

In an implementation, degradation and decomposition may be minimized by reducing the deposition temperature due to steric hindrance, and life-span characteristics may be further improved.

When m11 is 2 or 3, each R¹¹ may be the same or different from each other.

When m12 is 2, 3, or 4, each R¹² may be the same or different from each other.

In an implementation, the third compound may be represented by, e.g., one of Chemical Formula 5A to Chemical Formula 5F.

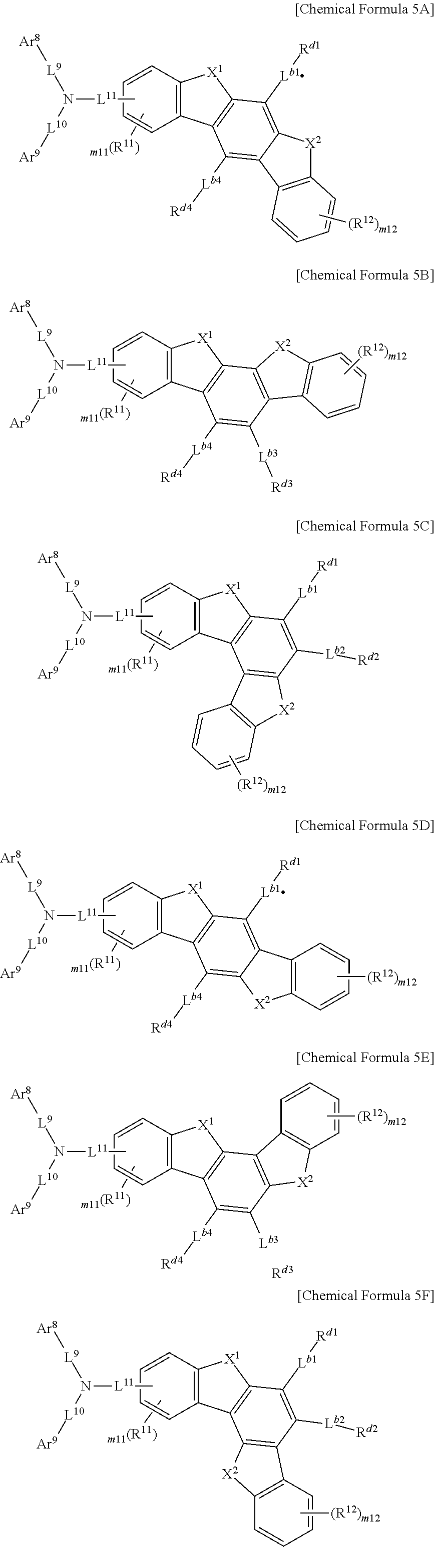

In Chemical Formula 5A to Chemical Formula 5F, X¹, X², R¹¹, R¹², Ar⁸, Ar⁹, L⁹ to L¹¹, m11, and m12 may be defined the same as those described above.

L^{b1} to L^{b4} may each independently be, e.g., a single bond, a substituted or unsubstituted C6 to C20 arylene group, or a substituted or unsubstituted C2 to C20 heterocyclic group.

R^{d1} to R^{d4} may each independently be, hydrogen, deuterium, a cyano group, a halogen, a substituted or unsubstituted C1 to C30 alkyl group, a substituted or unsubstituted C6 to C30 aryl group, or a substituted or unsubstituted C2 to C30 heterocyclic group.

In an implementation, Chemical Formula 5A may be represented by, e.g., one of Chemical Formula 5A-1 to Chemical Formula 5A-4.

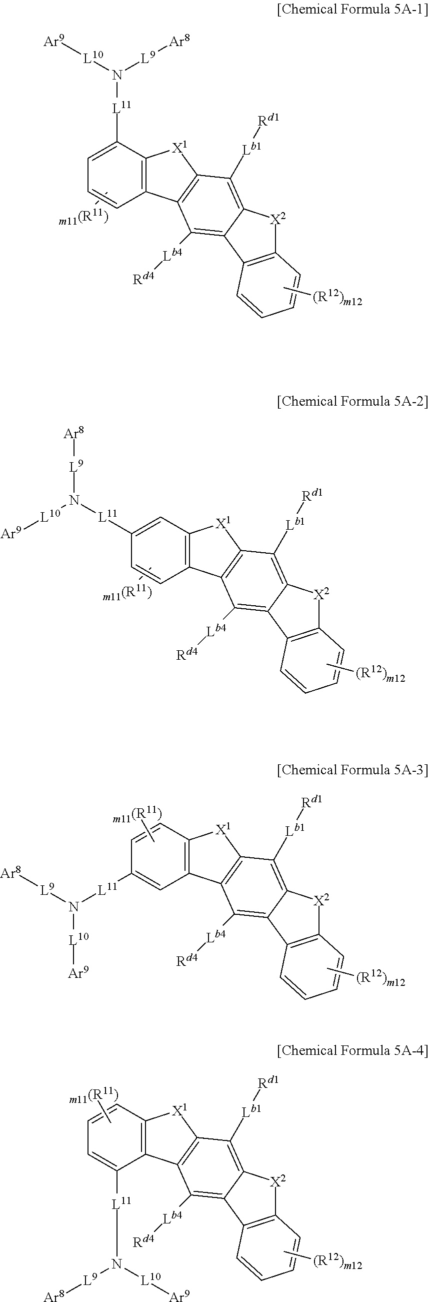

In Chemical Formula 5A-1 to Chemical Formula 5A-4, X¹, X², R¹¹, R¹², Ar^{g}, Ar⁹, L⁹ to L¹¹, m11, m12, L^{b1}, L^{b4}, R^{d1}, and R^{d4} may be defined the same as those described above.

In an implementation, Chemical Formula 5B may be represented by, e.g., one of Chemical Formula 5B-1 to Chemical Formula 5B-4.

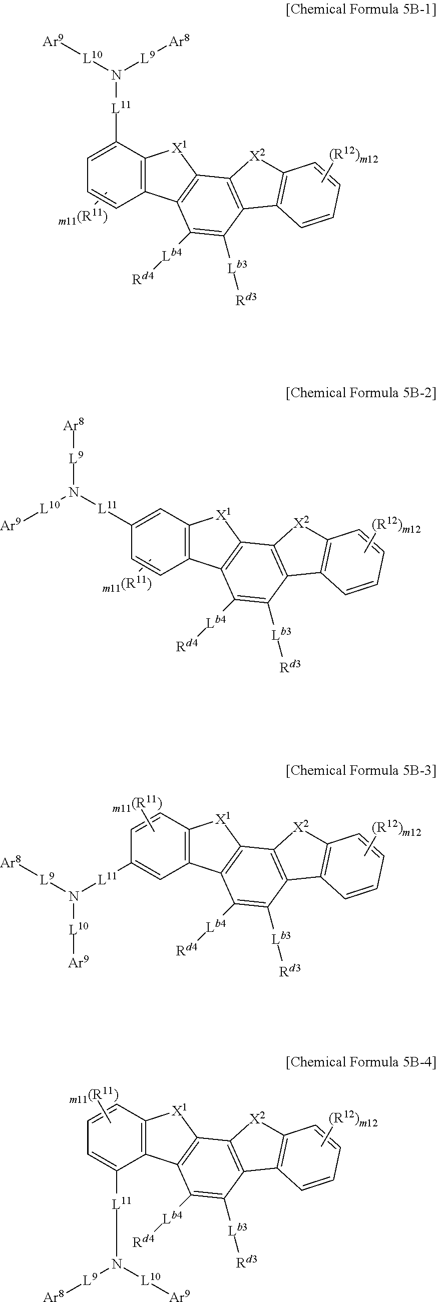

In Chemical Formula 5B-1 to Chemical Formula 5B-4, X¹, X², R, R¹², Ar^{g}, Ar⁹, L⁹ to L¹¹, m11, m12, L^{b3}, L^{b4}, R^{d3}, and R^{d4} may be defined the same as those described above.

In an implementation, Chemical Formula 5C may be represented by, e.g., one of Chemical Formula 5C-1 to Chemical Formula 5C-4.

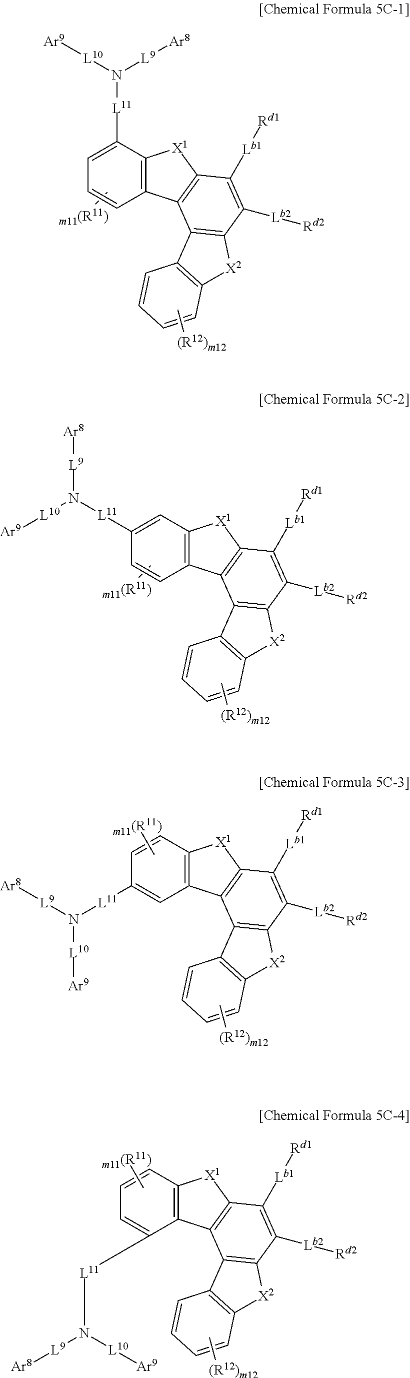

In Chemical Formula 5C-1 to Chemical Formula 5C-4, X¹, X², R¹¹, R¹², Ar^{g}, Ar⁹, L⁹ to L¹¹, m11, m12, L^{b1}, L^{b2}, R^{d1}, and R^{d2} may be defined the same as those described above.

In an implementation, Chemical Formula 5D may be represented by, e.g., one of Chemical Formula 5D-1 to Chemical Formula 5D-4.

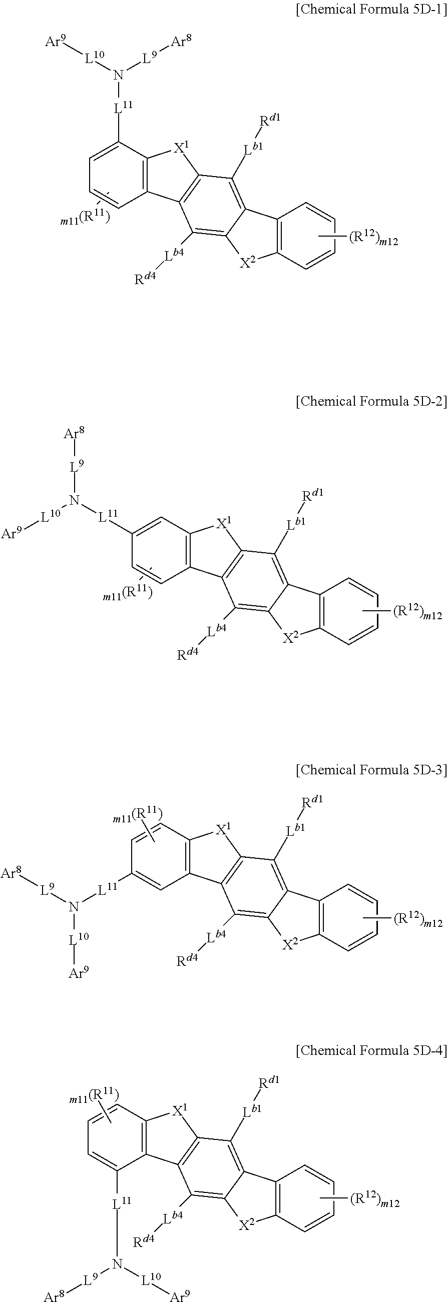

In Chemical Formula 5D-1 to Chemical Formula 5D-4, X¹, X², R¹¹, R¹², Ar⁸, Ar⁹, L⁹ to L¹¹, m11, m12, L^{b1}, L^{b4}, and R^{d4} may be defined the same as those described above.

In an implementation, Chemical Formula 5E may be represented by, e.g., one of Chemical Formula 5E-1 to Chemical Formula 5E-4.

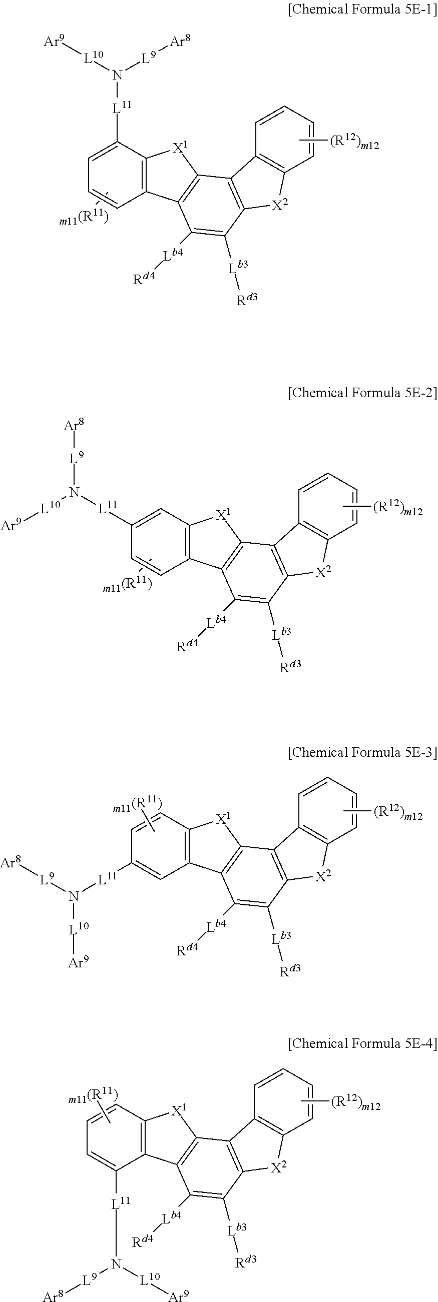

In Chemical Formula 5E-1 to Chemical Formula 5E-4, X¹, X², R¹¹, R¹², Ar^{g}, Ar⁹, L⁹ to L¹¹, m11, m12, L^{b3}, L^{b4}, R^{d3}, and R^{d4} may be defined the same as those described above.

In an implementation, Chemical Formula 5F may be represented by, e.g., one of Chemical Formula 5F-1 to Chemical Formula 5F-4.

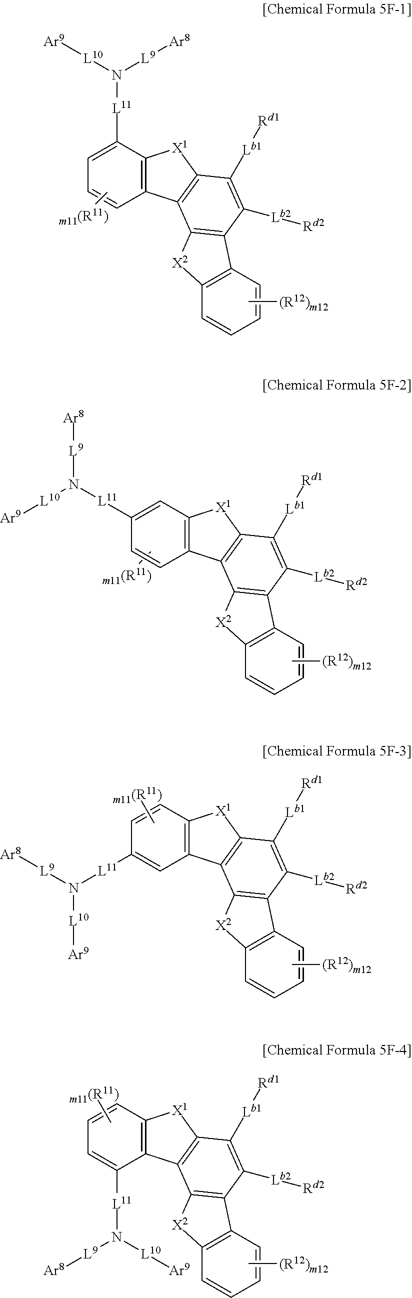

In Chemical Formula 5F-1 to Chemical Formula 5F-4, X¹, X², R¹¹, R¹², Ar^{g}, Ar⁹, L⁹ to L¹¹, m11, m12, L^{b1}, L^{b2}, R^{d1}, and R^{d2} may be defined the same as those described above.

In an implementation, X¹ of Chemical Formula 5 may be, e.g., CR^{b}R^{c}, and X² of Chemical Formula 6 may be, e.g., O or S.

R^{b} and R^{c} may each independently be, e.g., a substituted or unsubstituted C1 to C30 alkyl group or a substituted or unsubstituted C6 to C30 aryl group.

In an implementation, X¹ of Chemical Formula 5 may be, e.g., O or S, X² of Chemical Formula 6 may be, e.g., CR^{b}R^{c}.

R^{b} and R^{c} may each independently be, e.g., a substituted or unsubstituted C1 to C30 alkyl group or a substituted or unsubstituted C6 to C30 aryl group,

Ar⁸ and Ar⁹ may each independently be, e.g., a substituted or unsubstituted C6 to C30 aryl group or a substituted or unsubstituted C2 to C30 heterocyclic group.

In an implementation, at least one of Ar⁸ and Ar⁹ may be, e.g., a substituted or unsubstituted fluorenyl group or a substituted or unsubstituted dibenzosilolyl group.

In an implementation, Ar⁸ and Ar⁹ of Chemical Formula 5 may each independently be, e.g., a substituted or unsubstituted phenyl group, a substituted or unsubstituted biphenyl group, a substituted or unsubstituted fluorenyl group, a substituted or unsubstituted triphenylene group, a substituted or unsubstituted dibenzofuranyl group, or a substituted or unsubstituted dibenzothiophenyl group.

In an implementation, Ar⁸ and Ar⁹ of Chemical Formula 5 may each independently be, e.g., a substituted or unsubstituted biphenyl group, a substituted or unsubstituted fluorenyl group, a substituted or unsubstituted triphenylene group, a substituted or unsubstituted dibenzofuranyl group, or a substituted or unsubstituted dibenzothiophenyl group.

In an implementation, Ar⁸ and Ar⁹ of Chemical Formula 5 may each independently be, e.g., a group of Group III.

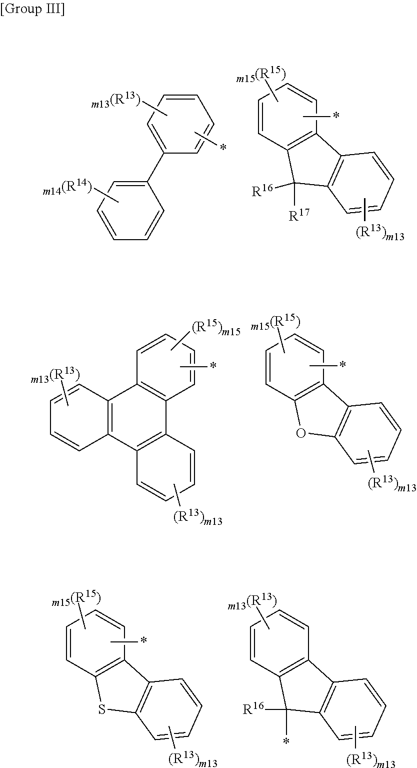

In Group III, R¹⁶ and R¹⁷ may each independently be, e.g., a substituted or unsubstituted C1 to C10 alkyl group or a substituted or unsubstituted C6 to C20 aryl group.

R¹³ to R¹⁵ may each independently be, e.g., hydrogen, deuterium, a C1 to C10 alkyl group, a C6 to C12 aryl group, or a C2 to C20 heterocyclic group.

m13 may be, e.g., an integer of 1 to 4.

m14 may be, e.g., an integer of 1 to 5.

m15 may be, e.g., an integer of 1 to 3.

* is a linking point.

When m13 is 2, 3, or 4, each R¹³ may be the same or different from each other.

When m14 is 2, 3, 4, or 5, each R¹⁴ may be the same or different from each other.

When m15 is 2 or 3, each R¹⁵ may be the same or different from each other.

In an implementation, Ar^{g} and Ar⁹ of Chemical Formula 5 may each independently be, e.g., a substituted or unsubstituted biphenyl group, a substituted or unsubstituted fluorenyl group, a substituted or unsubstituted dibenzosilolyl group, a substituted or unsubstituted dibenzofuranyl group, or a substituted or unsubstituted dibenzothiophenyl group.

In an implementation, the third compound may be, e.g., represented by Chemical Formula 5A, Chemical Formula 5C, or Chemical Formula 5F.

In an implementation, the third compound may be, e.g., represented by Chemical Formula 5A-4, Chemical Formula 5B-4, Chemical Formula 5C-4, Chemical Formula 5D-4, or Chemical Formula 5F-4.

In an implementation, the third compound may be, e.g., a compound of Group 3.

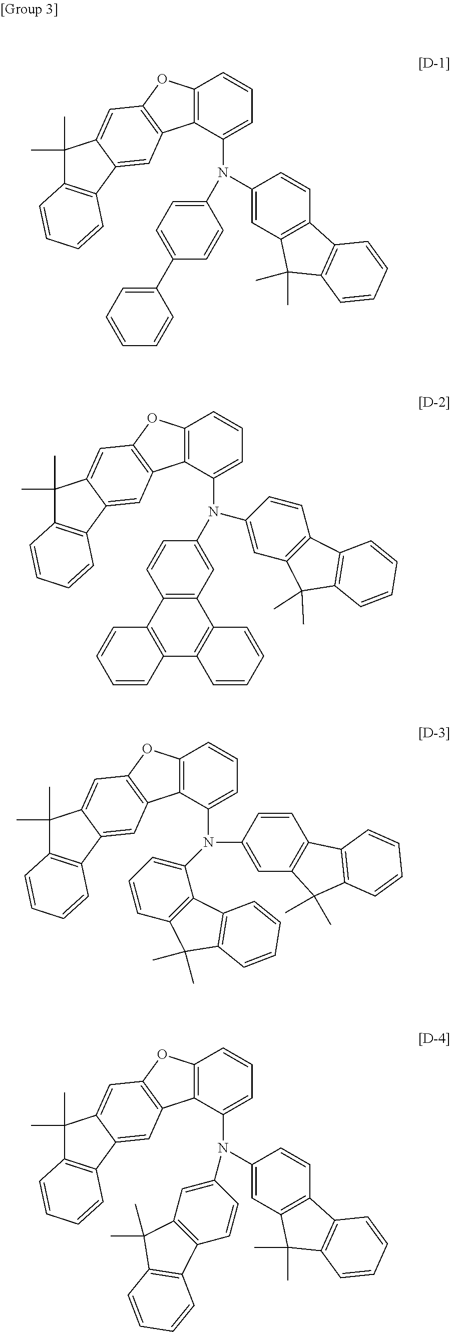 [SMILES: CC1(C)c2ccccc2-c2ccc(N(c3ccc(-c4ccccc4)cc3)c3cccc4oc5cc6c(cc5c34)-c3ccccc3C6(C)C)cc21 ; CC1(C)c2ccccc2-c2ccc(N(c3ccc4c5ccccc5c5ccccc5c4c3)c3cccc4oc5cc6c(cc5c34)-c3ccccc3C6(C)C)cc21 ; CC1(C)c2ccccc2-c2ccc(N(c3cccc4c3-c3ccccc3C4(C)C)c3cccc4oc5cc6c(cc5c34)-c3ccccc3C6(C)C)cc21 ; CC1(C)c2ccccc2-c2ccc(N(c3ccc4c(c3)C(C)(C)c3ccccc3-4)c3cccc4oc5cc6c(cc5c34)-c3ccccc3C6(C)C)cc21]

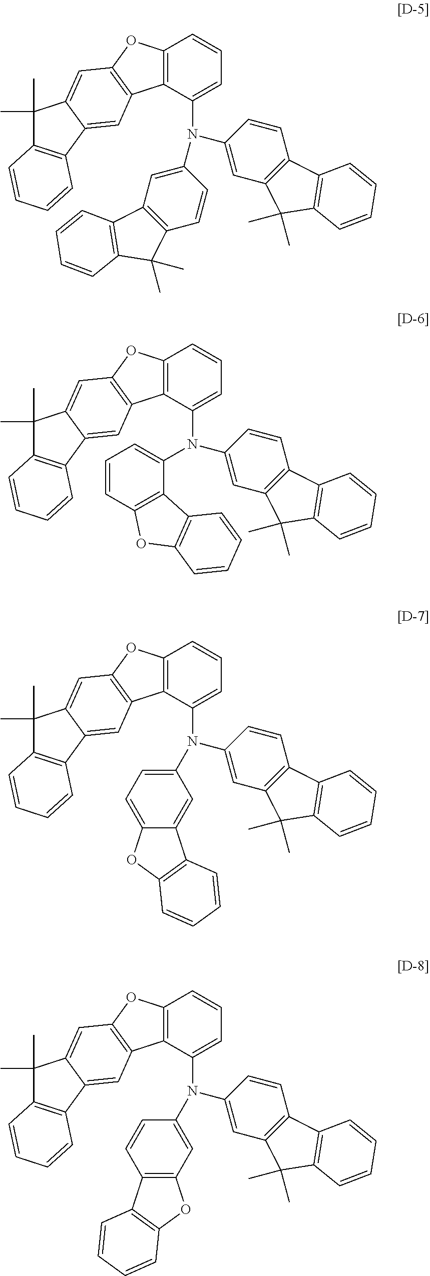 [SMILES: CC1(C)c2ccccc2-c2cc(N(c3ccc4c(c3)C(C)(C)c3ccccc3-4)c3cccc4oc5cc6c(cc5c34)-c3ccccc3C6(C)C)ccc21 ; CC1(C)c2ccccc2-c2ccc(N(c3cccc4oc5ccccc5c34)c3cccc4oc5cc6c(cc5c34)-c3ccccc3C6(C)C)cc21 ; CC1(C)c2ccccc2-c2ccc(N(c3ccc4oc5ccccc5c4c3)c3cccc4oc5cc6c(cc5c34)-c3ccccc3C6(C)C)cc21 ; CC1(C)c2ccccc2-c2ccc(N(c3ccc4c(c3)oc3ccccc34)c3cccc4oc5cc6c(cc5c34)-c3ccccc3C6(C)C)cc21]

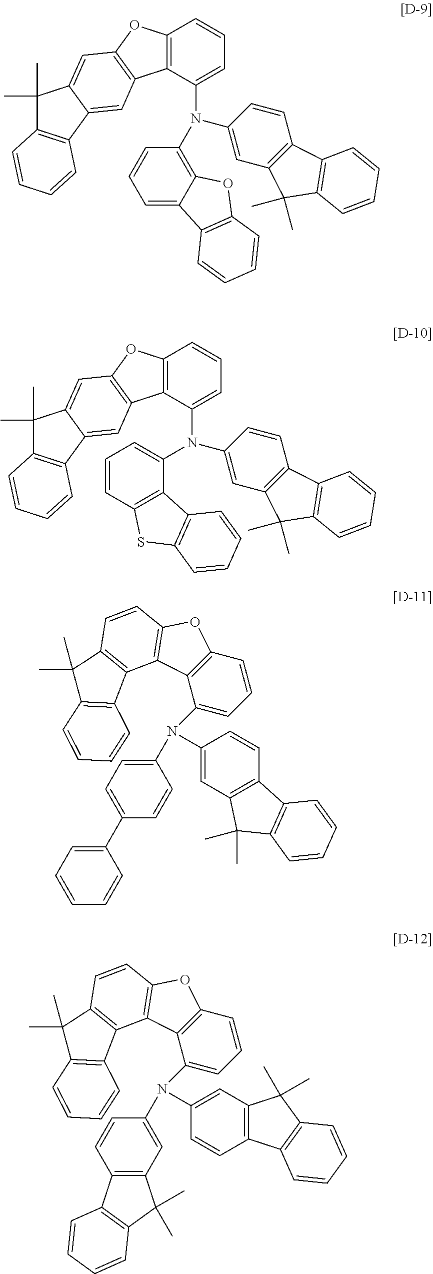 [SMILES: CC1(C)c2ccccc2-c2ccc(N(c3cccc4c3oc3ccccc34)c3cccc4oc5cc6c(cc5c34)-c3ccccc3C6(C)C)cc21 ; CC1(C)c2ccccc2-c2ccc(N(c3cccc4oc5cc6c(cc5c34)-c3ccccc3C6(C)C)c3cccc4sc5ccccc5c34)cc21 ; CC1(C)c2ccccc2-c2ccc(N(c3ccc(-c4ccccc4)cc3)c3cccc4oc5ccc6c(c5c34)-c3ccccc3C6(C)C)cc21 ; CC1(C)c2ccccc2-c2ccc(N(c3ccc4c(c3)C(C)(C)c3ccccc3-4)c3cccc4oc5ccc6c(c5c34)-c3ccccc3C6(C)C)cc21]

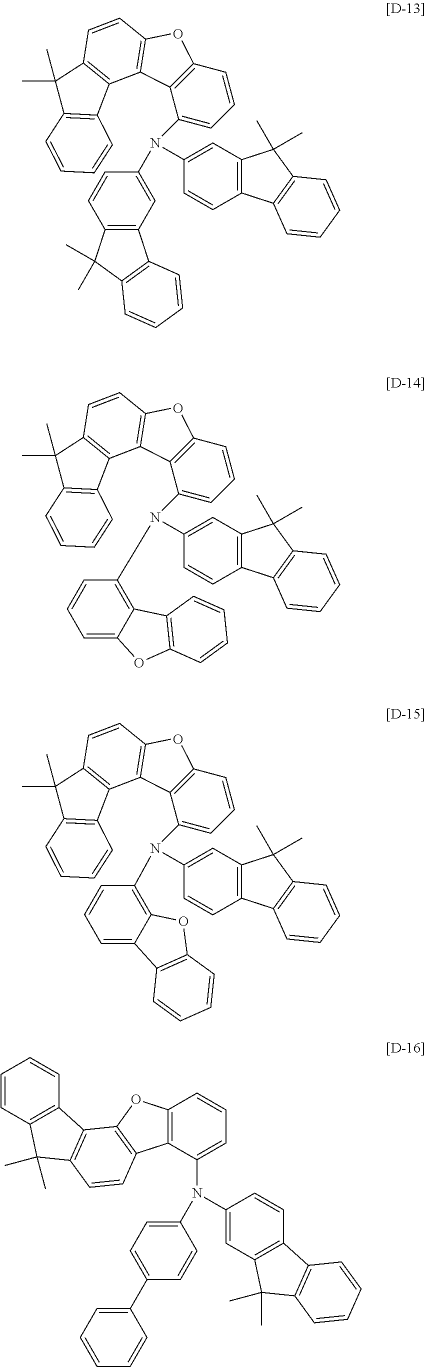 [SMILES: CC1(C)c2ccccc2-c2cc(N(c3ccc4c(c3)C(C)(C)c3ccccc3-4)c3cccc4oc5ccc6c(c5c34)-c3ccccc3C6(C)C)ccc21 ; CC1(C)c2ccccc2-c2ccc(N(c3cccc4oc5ccccc5c34)c3cccc4oc5ccc6c(c5c34)-c3ccccc3C6(C)C)cc21 ; CC1(C)c2ccccc2-c2ccc(N(c3cccc4c3oc3ccccc34)c3cccc4oc5ccc6c(c5c34)-c3ccccc3C6(C)C)cc21 ; CC1(C)c2ccccc2-c2ccc(N(c3ccc(-c4ccccc4)cc3)c3cccc4oc5c6c(ccc5c34)C(C)(C)c3ccccc3-6)cc21]

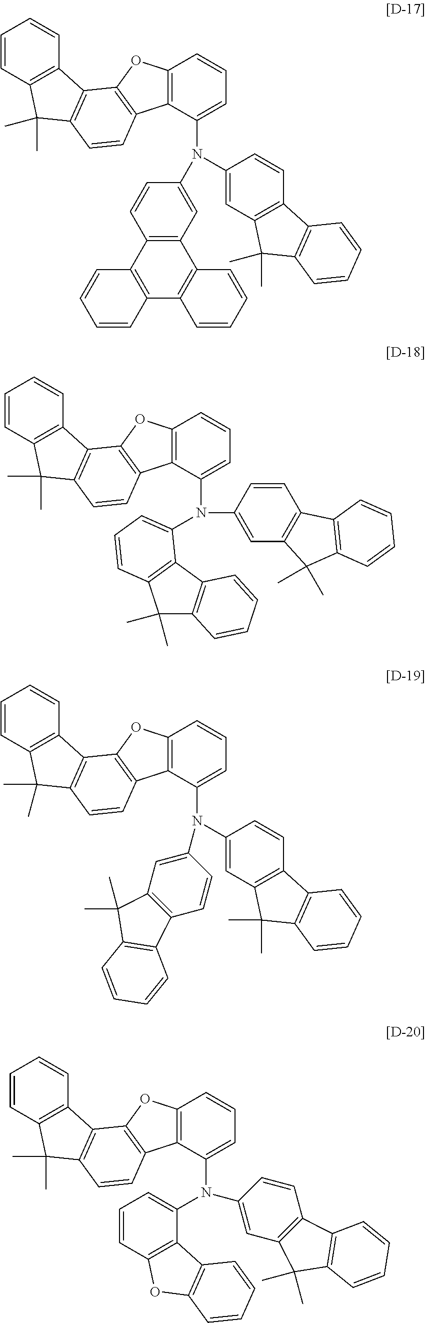 [SMILES: CC1(C)c2ccccc2-c2ccc(N(c3ccc4c5ccccc5c5ccccc5c4c3)c3cccc4oc5c6c(ccc5c34)C(C)(C)c3ccccc3-6)cc21 ; CC1(C)c2ccccc2-c2ccc(N(c3cccc4c3-c3ccccc3C4(C)C)c3cccc4oc5c6c(ccc5c34)C(C)(C)c3ccccc3-6)cc21 ; CC1(C)c2ccccc2-c2ccc(N(c3ccc4c(c3)C(C)(C)c3ccccc3-4)c3cccc4oc5c6c(ccc5c34)C(C)(C)c3ccccc3-6)cc21 ; CC1(C)c2ccccc2-c2ccc(N(c3cccc4oc5ccccc5c34)c3cccc4oc5c6c(ccc5c34)C(C)(C)c3ccccc3-6)cc21]

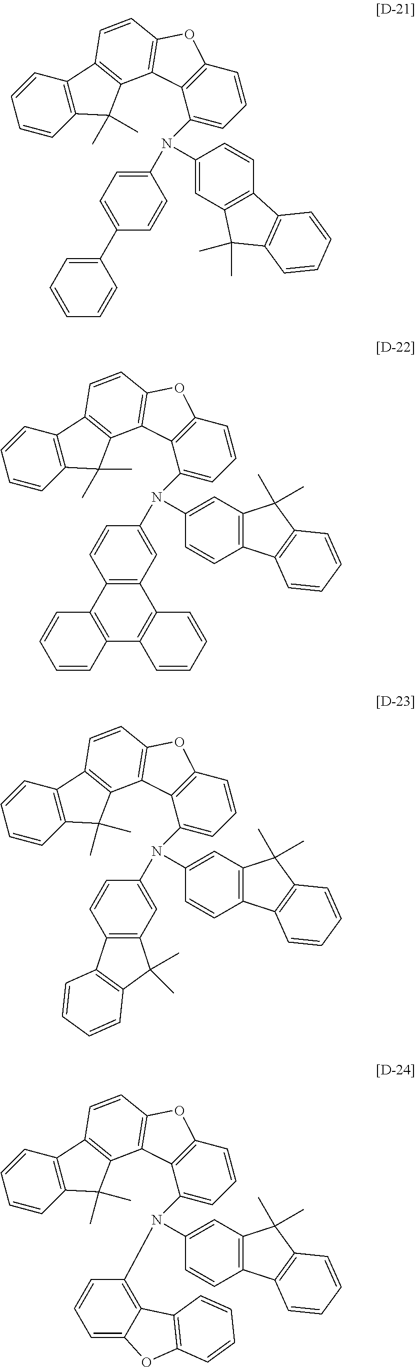 [SMILES: CC1(C)c2ccccc2-c2ccc(N(c3ccc(-c4ccccc4)cc3)c3cccc4oc5ccc6c(c5c34)C(C)(C)c3ccccc3-6)cc21 ; CC1(C)c2ccccc2-c2ccc(N(c3ccc4c5ccccc5c5ccccc5c4c3)c3cccc4oc5ccc6c(c5c34)C(C)(C)c3ccccc3-6)cc21 ; CC1(C)c2ccccc2-c2ccc(N(c3ccc4c(c3)C(C)(C)c3ccccc3-4)c3cccc4oc5ccc6c(c5c34)C(C)(C)c3ccccc3-6)cc21 ; CC1(C)c2ccccc2-c2ccc(N(c3cccc4oc5ccccc5c34)c3cccc4oc5ccc6c(c5c34)C(C)(C)c3ccccc3-6)cc21]

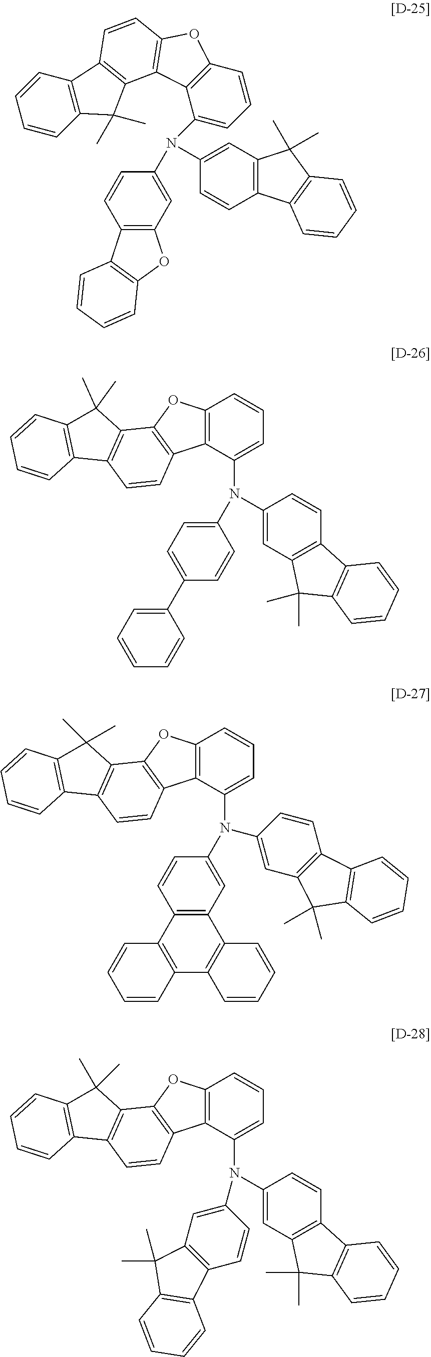 [SMILES: CC1(C)c2ccccc2-c2ccc(N(c3ccc4c(c3)oc3ccccc34)c3cccc4oc5ccc6c(c5c34)C(C)(C)c3ccccc3-6)cc21 ; CC1(C)c2ccccc2-c2ccc(N(c3ccc(-c4ccccc4)cc3)c3cccc4oc5c6c(ccc5c34)-c3ccccc3C6(C)C)cc21 ; CC1(C)c2ccccc2-c2ccc(N(c3ccc4c5ccccc5c5ccccc5c4c3)c3cccc4oc5c6c(ccc5c34)-c3ccccc3C6(C)C)cc21 ; CC1(C)c2ccccc2-c2ccc(N(c3ccc4c(c3)C(C)(C)c3ccccc3-4)c3cccc4oc5c6c(ccc5c34)-c3ccccc3C6(C)C)cc21]

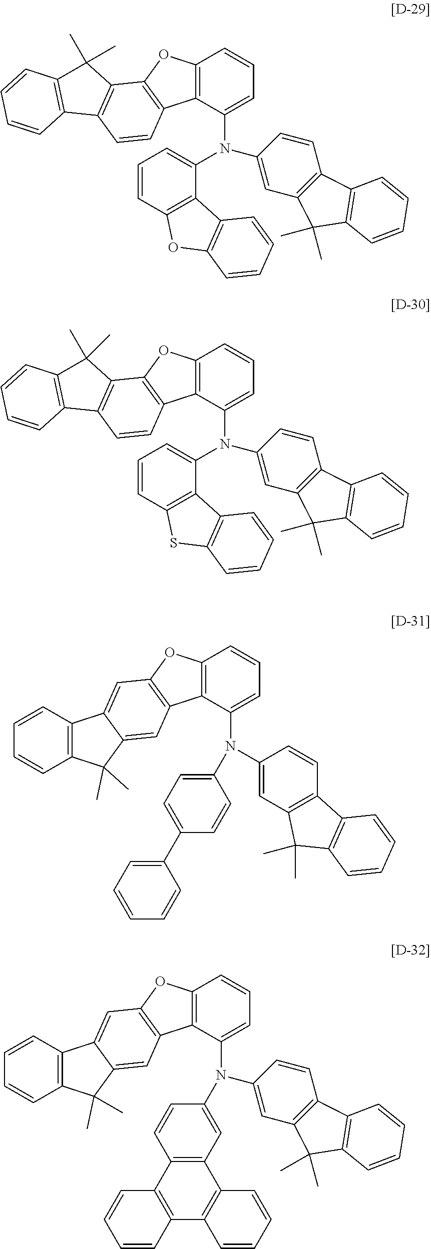 [SMILES: CC1(C)c2ccccc2-c2ccc(N(c3cccc4oc5ccccc5c34)c3cccc4oc5c6c(ccc5c34)-c3ccccc3C6(C)C)cc21 ; CC1(C)c2ccccc2-c2ccc(N(c3cccc4oc5c6c(ccc5c34)-c3ccccc3C6(C)C)c3cccc4sc5ccccc5c34)cc21 ; CC1(C)c2ccccc2-c2ccc(N(c3ccc(-c4ccccc4)cc3)c3cccc4oc5cc6c(cc5c34)C(C)(C)c3ccccc3-6)cc21 ; CC1(C)c2ccccc2-c2ccc(N(c3ccc4c5ccccc5c5ccccc5c4c3)c3cccc4oc5cc6c(cc5c34)C(C)(C)c3ccccc3-6)cc21]

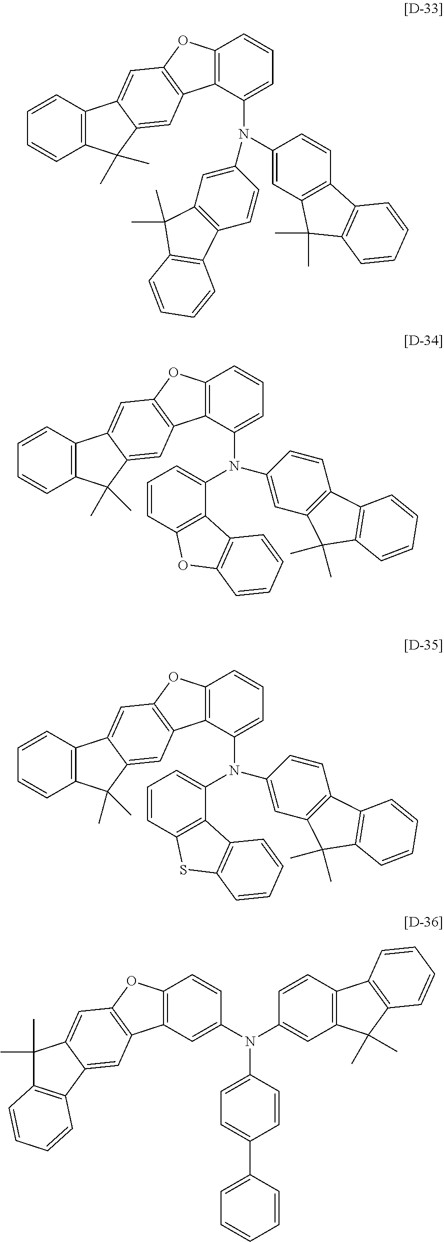 [SMILES: CC1(C)c2ccccc2-c2ccc(N(c3ccc4c(c3)C(C)(C)c3ccccc3-4)c3cccc4oc5cc6c(cc5c34)C(C)(C)c3ccccc3-6)cc21 ; CC1(C)c2ccccc2-c2ccc(N(c3cccc4oc5ccccc5c34)c3cccc4oc5cc6c(cc5c34)C(C)(C)c3ccccc3-6)cc21 ; CC1(C)c2ccccc2-c2ccc(N(c3cccc4oc5cc6c(cc5c34)C(C)(C)c3ccccc3-6)c3cccc4sc5ccccc5c34)cc21 ; CC1(C)c2ccccc2-c2ccc(N(c3ccc(-c4ccccc4)cc3)c3ccc4oc5cc6c(cc5c4c3)-c3ccccc3C6(C)C)cc21]

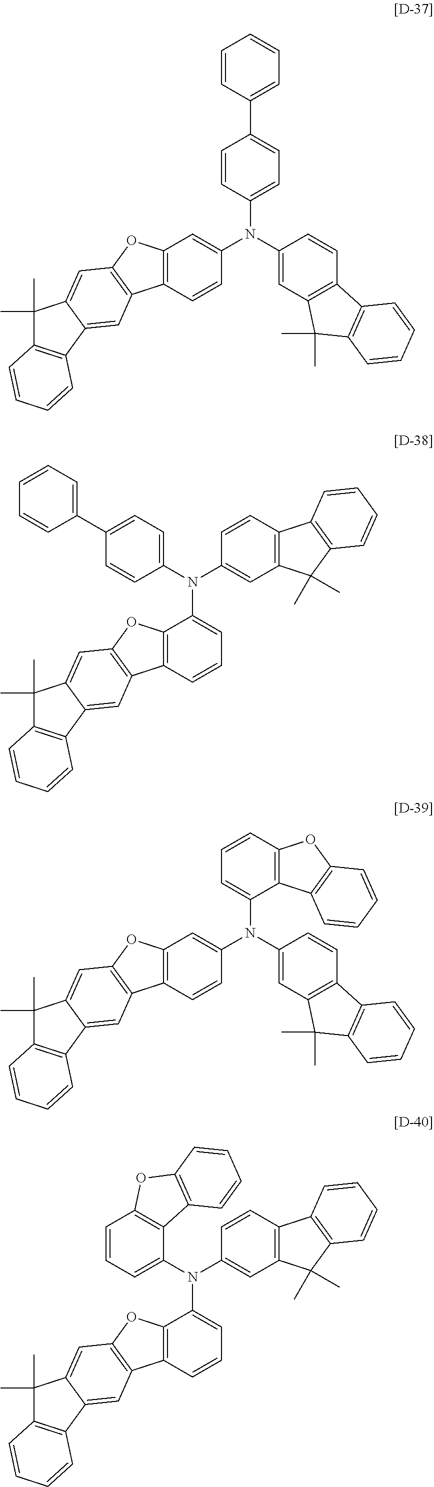 [SMILES: CC1(C)c2ccccc2-c2ccc(N(c3ccc(-c4ccccc4)cc3)c3ccc4c(c3)oc3cc5c(cc34)-c3ccccc3C5(C)C)cc21 ; CC1(C)c2ccccc2-c2ccc(N(c3ccc(-c4ccccc4)cc3)c3cccc4c3oc3cc5c(cc34)-c3ccccc3C5(C)C)cc21 ; CC1(C)c2ccccc2-c2ccc(N(c3ccc4c(c3)oc3cc5c(cc34)-c3ccccc3C5(C)C)c3cccc4oc5ccccc5c34)cc21 ; CC1(C)c2ccccc2-c2ccc(N(c3cccc4c3oc3cc5c(cc34)-c3ccccc3C5(C)C)c3cccc4oc5ccccc5c34)cc21]

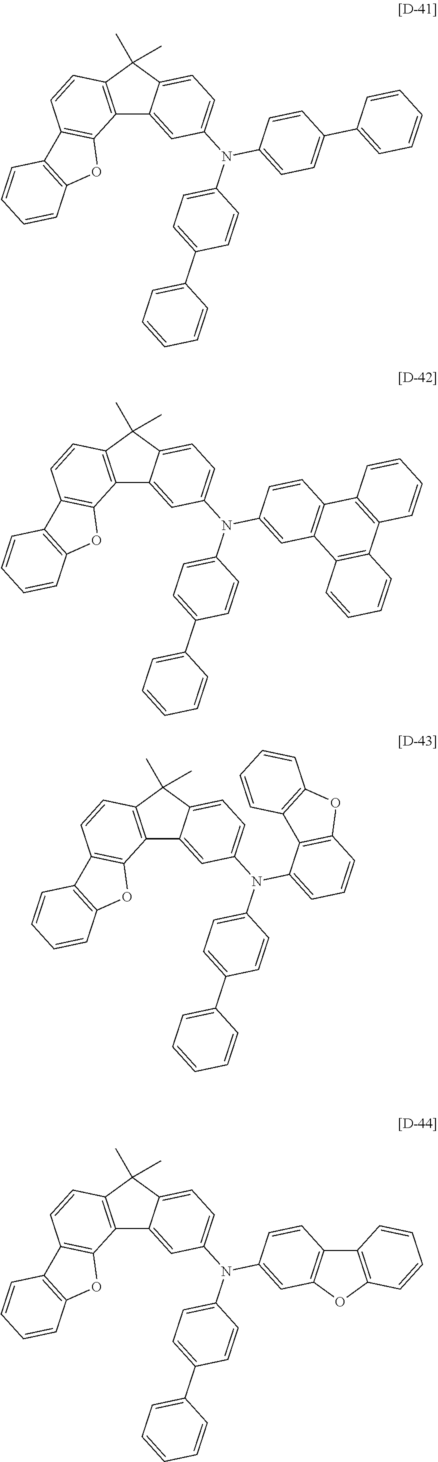 [SMILES: CC1(C)c2ccc(N(c3ccc(-c4ccccc4)cc3)c3ccc(-c4ccccc4)cc3)cc2-c2c1ccc1c2oc2ccccc21 ; CC1(C)c2ccc(N(c3ccc(-c4ccccc4)cc3)c3ccc4c5ccccc5c5ccccc5c4c3)cc2-c2c1ccc1c2oc2ccccc21 ; CC1(C)c2ccc(N(c3ccc(-c4ccccc4)cc3)c3cccc4oc5ccccc5c34)cc2-c2c1ccc1c2oc2ccccc21 ; CC1(C)c2ccc(N(c3ccc(-c4ccccc4)cc3)c3ccc4c(c3)oc3ccccc34)cc2-c2c1ccc1c2oc2ccccc21]

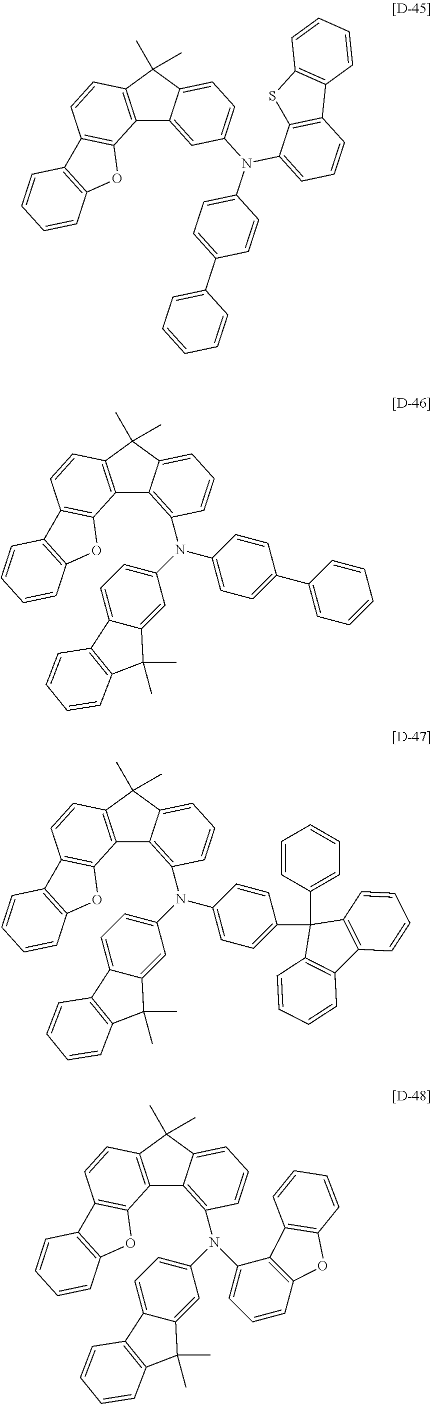 [SMILES: CC1(C)c2ccc(N(c3ccc(-c4ccccc4)cc3)c3cccc4c3sc3ccccc34)cc2-c2c1ccc1c2oc2ccccc21 ; CC1(C)c2ccccc2-c2ccc(N(c3ccc(-c4ccccc4)cc3)c3cccc4c3-c3c(ccc5c3oc3ccccc35)C4(C)C)cc21 ; CC1(C)c2ccccc2-c2ccc(N(c3ccc(C4(c5ccccc5)c5ccccc5-c5ccccc54)cc3)c3cccc4c3-c3c(ccc5c3oc3ccccc35)C4(C)C)cc21 ; CC1(C)c2ccccc2-c2ccc(N(c3cccc4c3-c3c(ccc5c3oc3ccccc35)C4(C)C)c3cccc4oc5ccccc5c34)cc21]

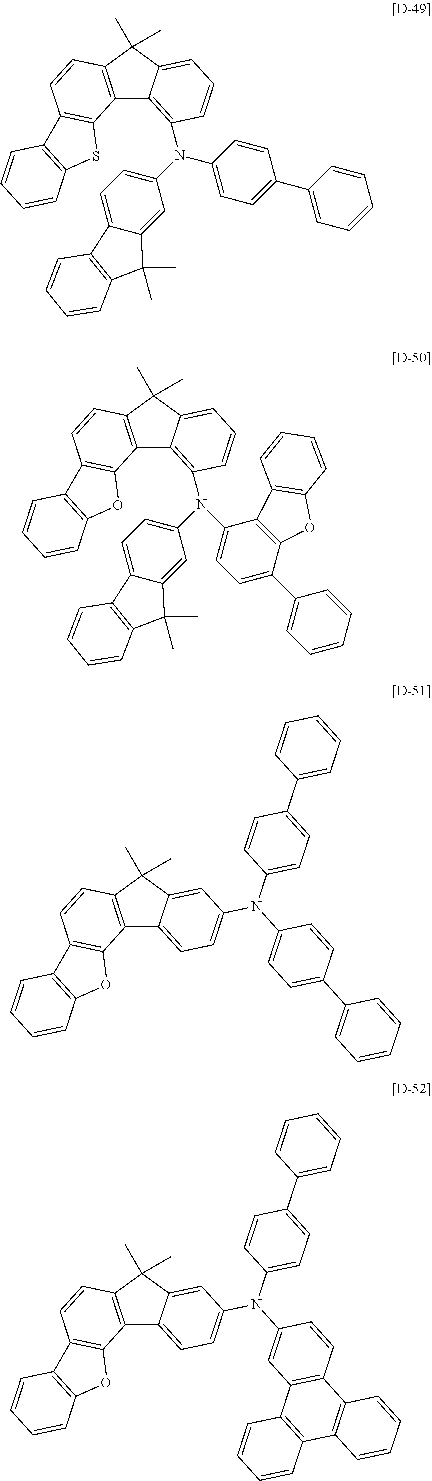 [SMILES: CC1(C)c2ccccc2-c2ccc(N(c3ccc(-c4ccccc4)cc3)c3cccc4c3-c3c(ccc5c3sc3ccccc35)C4(C)C)cc21 ; CC1(C)c2ccccc2-c2ccc(N(c3cccc4c3-c3c(ccc5c3oc3ccccc35)C4(C)C)c3ccc(-c4ccccc4)c4oc5ccccc5c34)cc21 ; CC1(C)c2cc(N(c3ccc(-c4ccccc4)cc3)c3ccc(-c4ccccc4)cc3)ccc2-c2c1ccc1c2oc2ccccc21 ; CC1(C)c2cc(N(c3ccc(-c4ccccc4)cc3)c3ccc4c5ccccc5c5ccccc5c4c3)ccc2-c2c1ccc1c2oc2ccccc21]

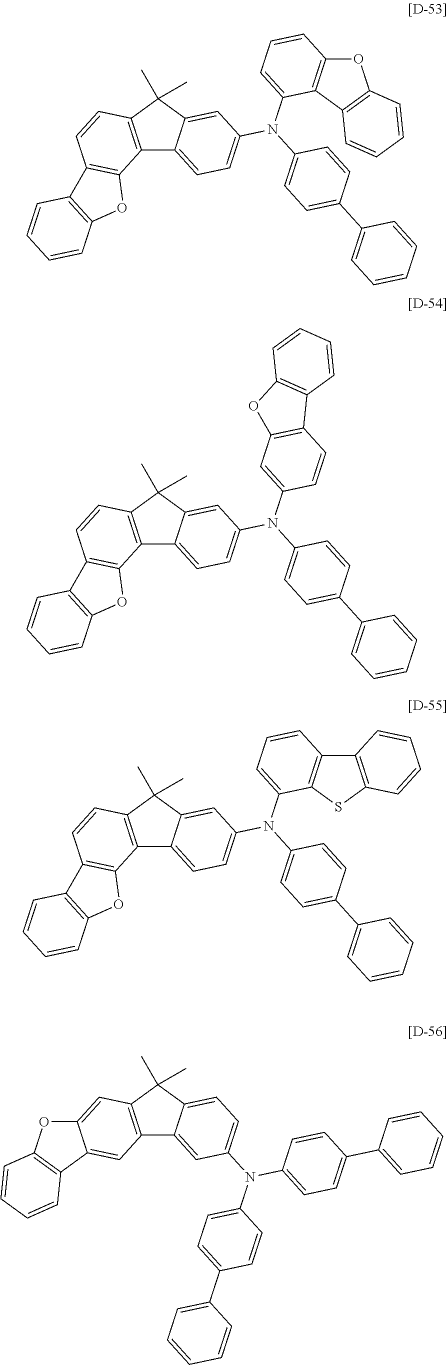 [SMILES: CC1(C)c2cc(N(c3ccc(-c4ccccc4)cc3)c3cccc4oc5ccccc5c34)ccc2-c2c1ccc1c2oc2ccccc21 ; CC1(C)c2cc(N(c3ccc(-c4ccccc4)cc3)c3ccc4c(c3)oc3ccccc34)ccc2-c2c1ccc1c2oc2ccccc21 ; CC1(C)c2cc(N(c3ccc(-c4ccccc4)cc3)c3cccc4c3sc3ccccc34)ccc2-c2c1ccc1c2oc2ccccc21 ; CC1(C)c2ccc(N(c3ccc(-c4ccccc4)cc3)c3ccc(-c4ccccc4)cc3)cc2-c2cc3c(cc21)oc1ccccc13]

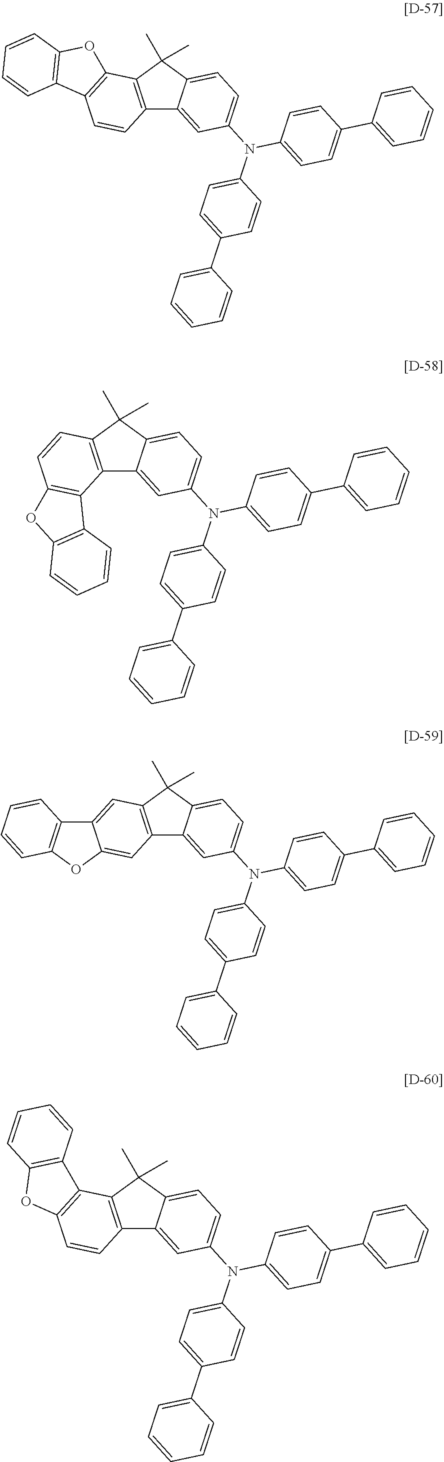 [SMILES: CC1(C)c2ccc(N(c3ccc(-c4ccccc4)cc3)c3ccc(-c4ccccc4)cc3)cc2-c2ccc3c(oc4ccccc43)c21 ; CC1(C)c2ccc(N(c3ccc(-c4ccccc4)cc3)c3ccc(-c4ccccc4)cc3)cc2-c2c1ccc1oc3ccccc3c21 ; CC1(C)c2ccc(N(c3ccc(-c4ccccc4)cc3)c3ccc(-c4ccccc4)cc3)cc2-c2cc3oc4ccccc4c3cc21 ; CC1(C)c2ccc(N(c3ccc(-c4ccccc4)cc3)c3ccc(-c4ccccc4)cc3)cc2-c2ccc3oc4ccccc4c3c21]

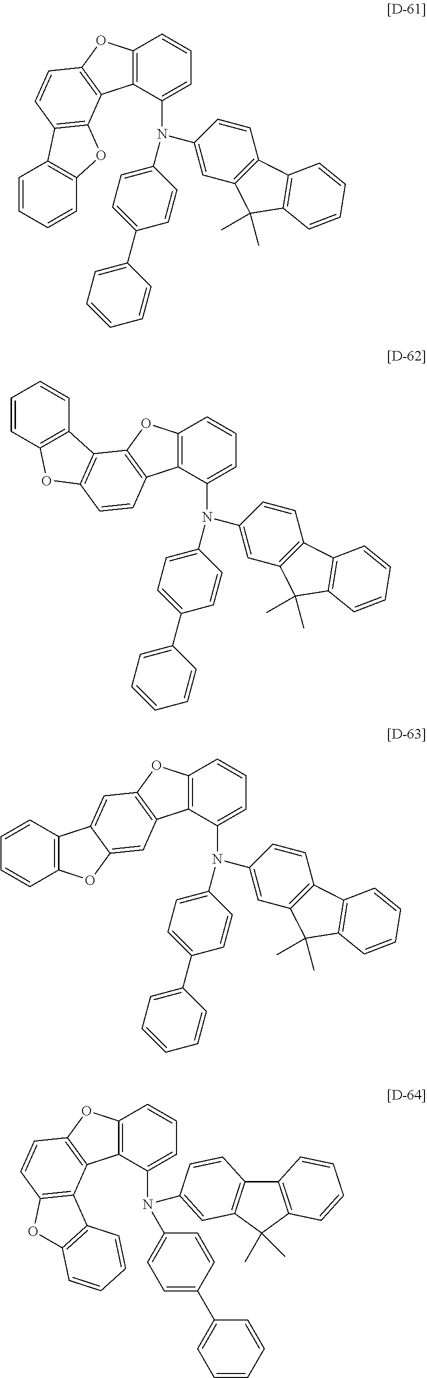 [SMILES: CC1(C)c2ccccc2-c2ccc(N(c3ccc(-c4ccccc4)cc3)c3cccc4oc5ccc6c7ccccc7oc6c5c34)cc21 ; CC1(C)c2ccccc2-c2ccc(N(c3ccc(-c4ccccc4)cc3)c3cccc4oc5c(ccc6oc7ccccc7c65)c34)cc21 ; CC1(C)c2ccccc2-c2ccc(N(c3ccc(-c4ccccc4)cc3)c3cccc4oc5cc6c(cc5c34)oc3ccccc36)cc21 ; CC1(C)c2ccccc2-c2ccc(N(c3ccc(-c4ccccc4)cc3)c3cccc4oc5ccc6oc7ccccc7c6c5c34)cc21]

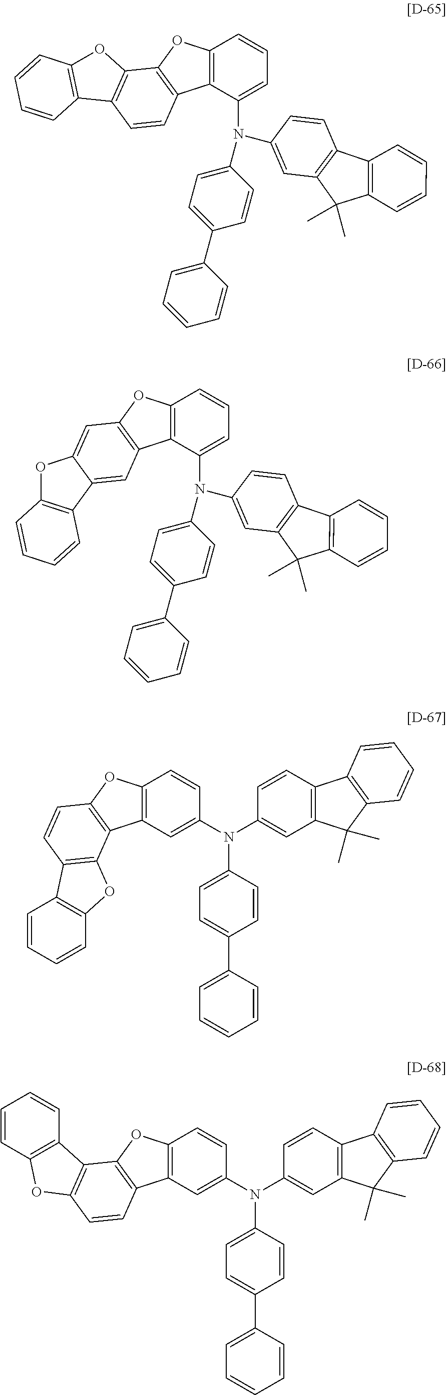 [SMILES: CC1(C)c2ccccc2-c2ccc(N(c3ccc(-c4ccccc4)cc3)c3cccc4oc5c(ccc6c7ccccc7oc65)c34)cc21 ; CC1(C)c2ccccc2-c2ccc(N(c3ccc(-c4ccccc4)cc3)c3cccc4oc5cc6oc7ccccc7c6cc5c34)cc21 ; CC1(C)c2ccccc2-c2ccc(N(c3ccc(-c4ccccc4)cc3)c3ccc4oc5ccc6c7ccccc7oc6c5c4c3)cc21 ; CC1(C)c2ccccc2-c2ccc(N(c3ccc(-c4ccccc4)cc3)c3ccc4oc5c(ccc6oc7ccccc7c65)c4c3)cc21]

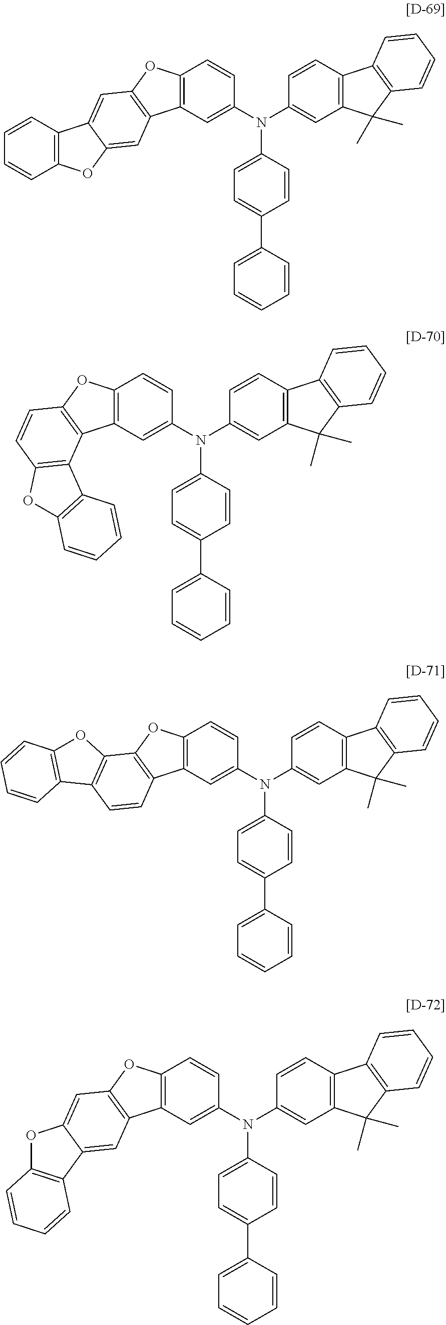 [SMILES: CC1(C)c2ccccc2-c2ccc(N(c3ccc(-c4ccccc4)cc3)c3ccc4oc5cc6c(cc5c4c3)oc3ccccc36)cc21 ; CC1(C)c2ccccc2-c2ccc(N(c3ccc(-c4ccccc4)cc3)c3ccc4oc5ccc6oc7ccccc7c6c5c4c3)cc21 ; CC1(C)c2ccccc2-c2ccc(N(c3ccc(-c4ccccc4)cc3)c3ccc4oc5c(ccc6c7ccccc7oc65)c4c3)cc21 ; CC1(C)c2ccccc2-c2ccc(N(c3ccc(-c4ccccc4)cc3)c3ccc4oc5cc6oc7ccccc7c6cc5c4c3)cc21]

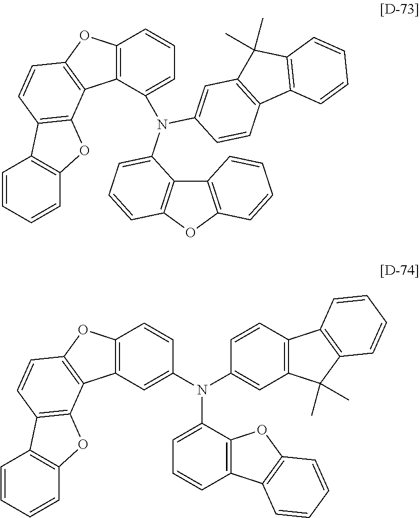 [SMILES: CC1(C)c2ccccc2-c2ccc(N(c3cccc4oc5ccccc5c34)c3cccc4oc5ccc6c7ccccc7oc6c5c34)cc21 ; CC1(C)c2ccccc2-c2ccc(N(c3ccc4oc5ccc6c7ccccc7oc6c5c4c3)c3cccc4c3oc3ccccc34)cc21]

In an implementation, the first compound may be represented by Chemical Formula 1, the second compound may be represented by Chemical Formula 2-8, and the third compound may be represented by Chemical Formula 5A-4.

In an implementation, the organic layer 30 may further include an electron transport region.

The electron transport region may help further increase electron injection and/or electron mobility and block holes between the cathode 20 and the light emitting layer 32.

In an implementation, the electron transport region may include an electron transport layer 34 between the cathode 20 and the light emitting layer 32, and an electron transport auxiliary layer between the light emitting layer 32 and the electron transport layer 34 and a compound of Group B may be included in at least one of the electron transport layer and the electron transport auxiliary layer.

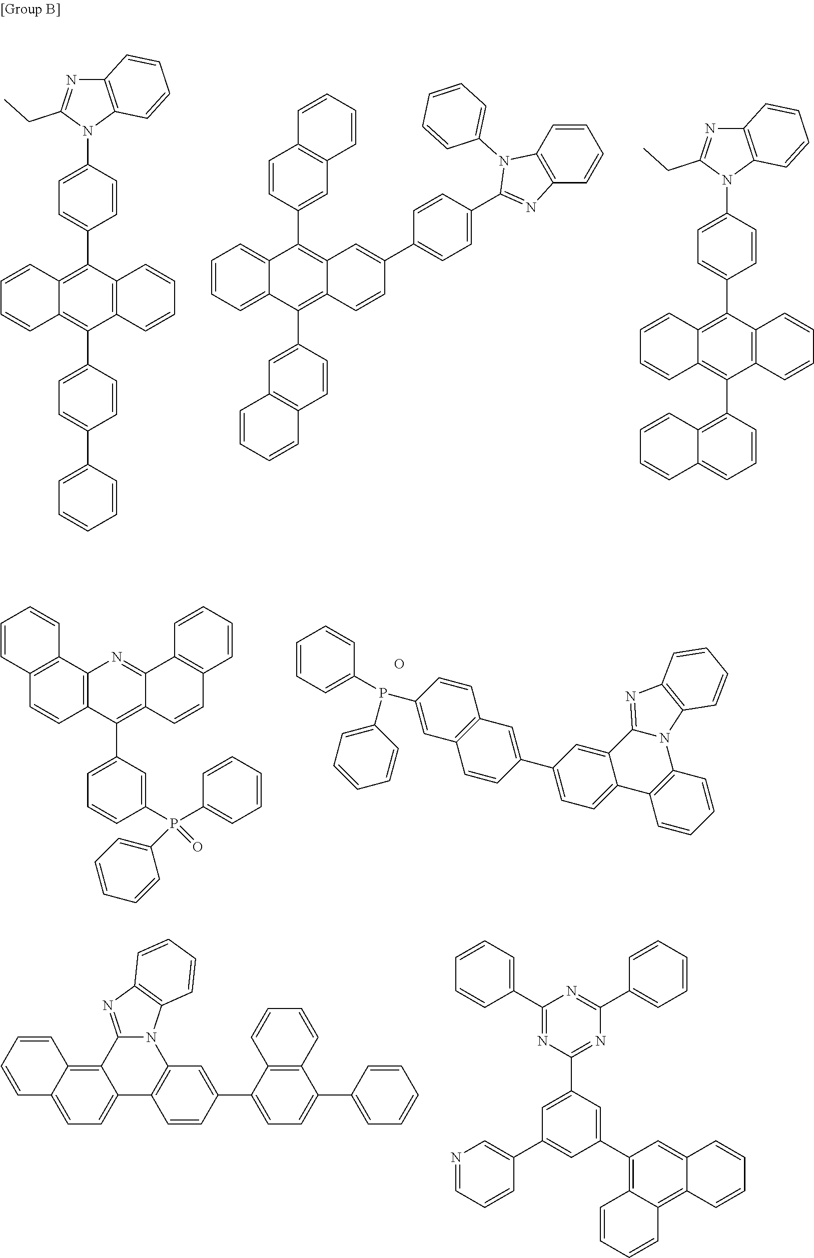

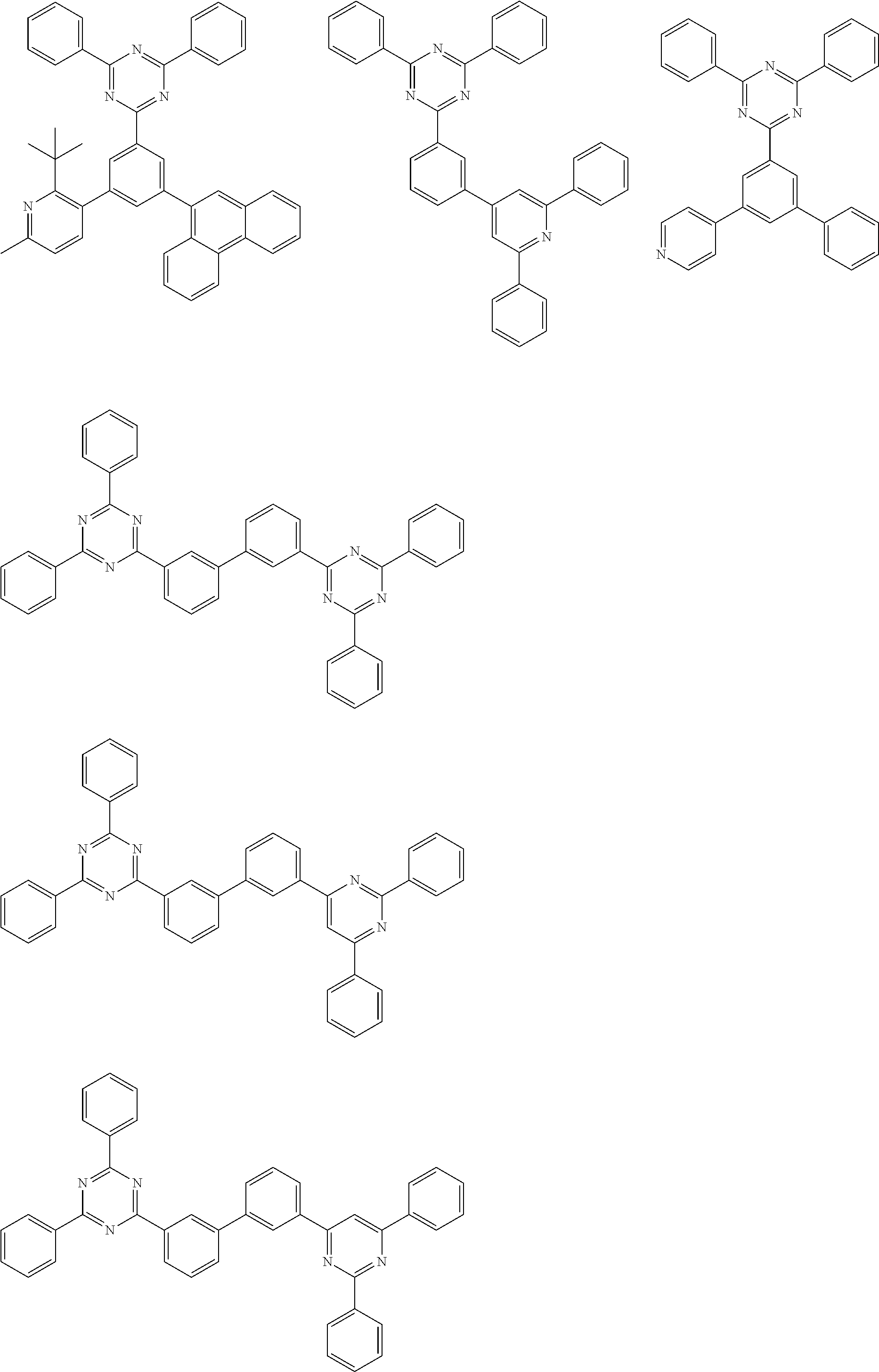 [SMILES: Cc1ccc(-c2cc(-c3nc(-c4ccccc4)nc(-c4ccccc4)n3)cc(-c3cc4ccccc4c4ccccc34)c2)c(C(C)(C)C)n1 ; c1ccc(-c2cc(-c3cccc(-c4nc(-c5ccccc5)nc(-c5ccccc5)n4)c3)cc(-c3ccccc3)n2)cc1 ; c1ccc(-c2cc(-c3ccncc3)cc(-c3nc(-c4ccccc4)nc(-c4ccccc4)n3)c2)cc1 ; c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1]

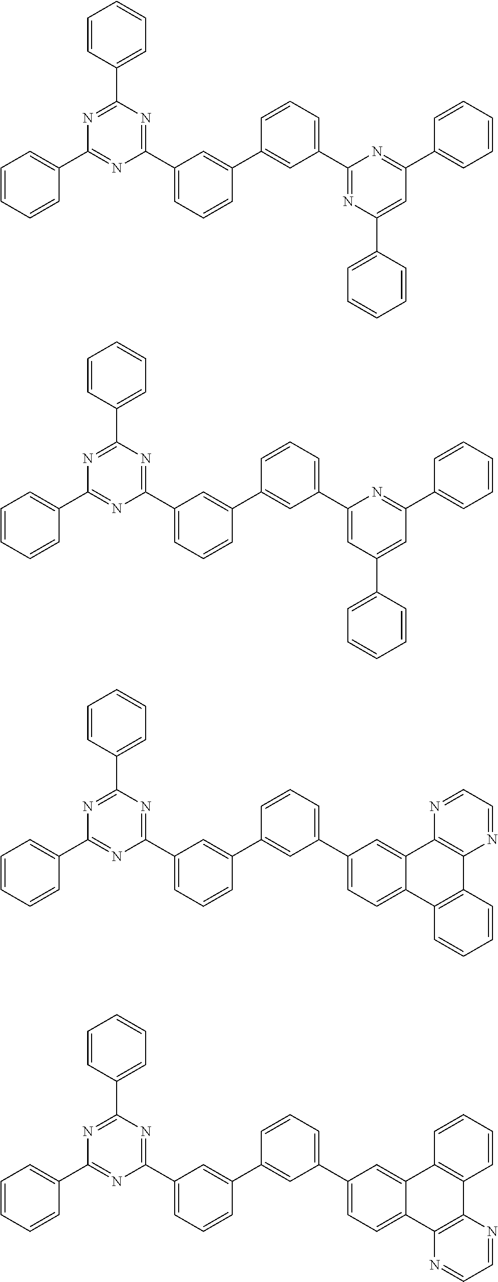 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

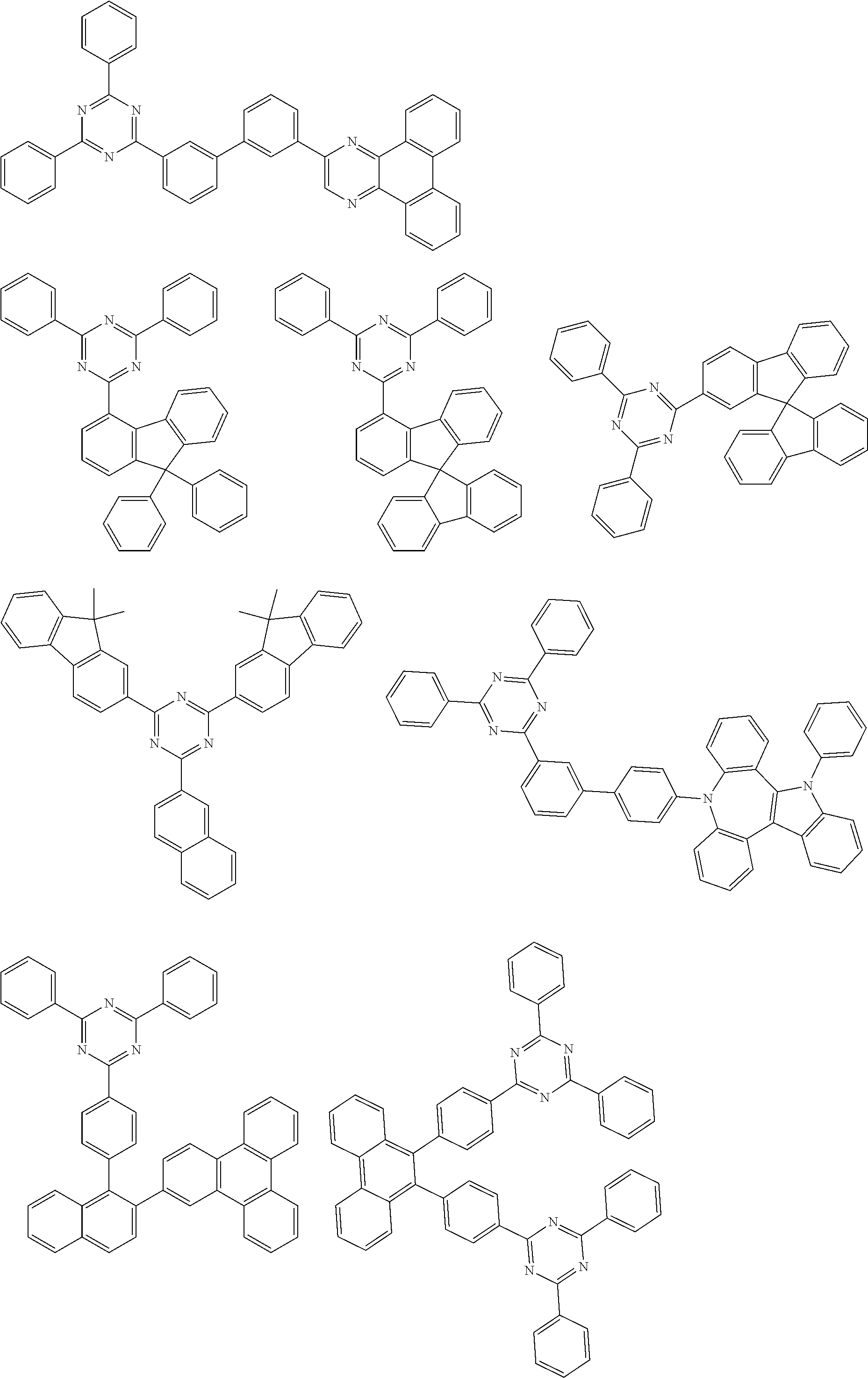

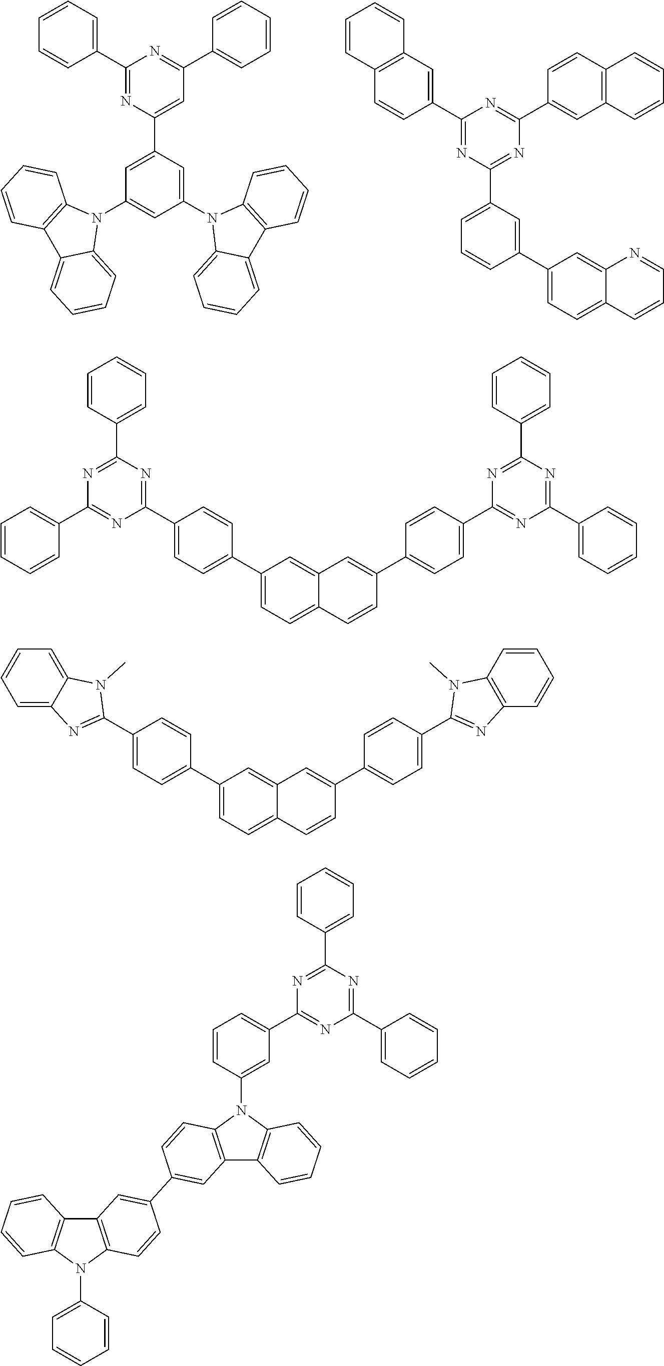

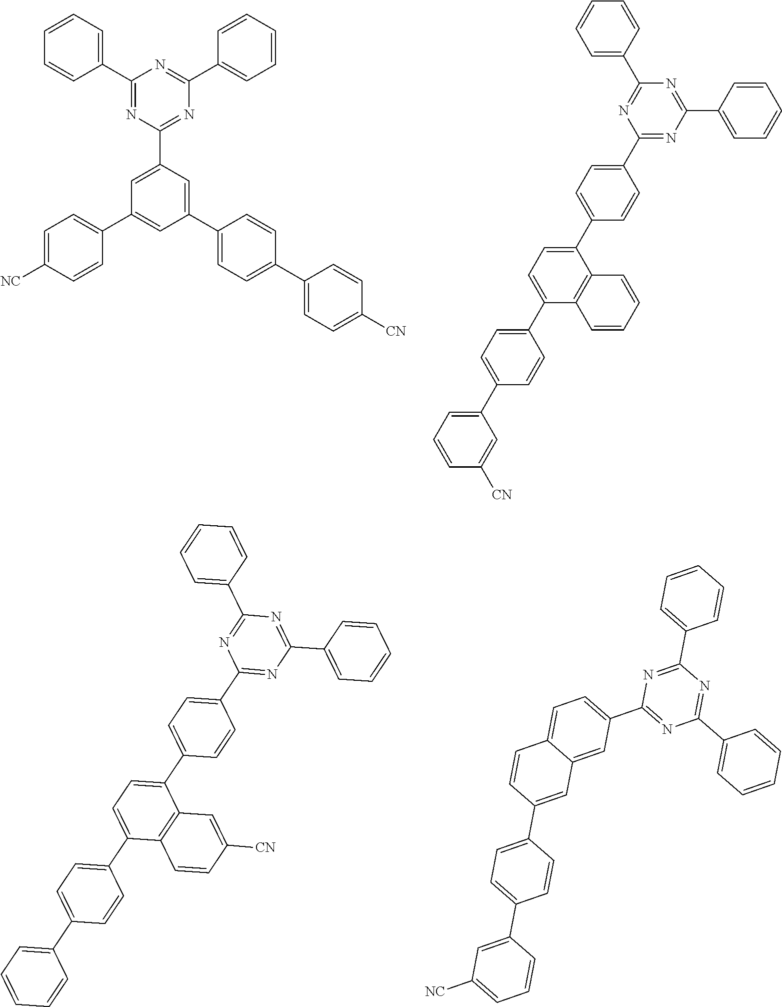 [SMILES: N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1 ; N#Cc1cccc(-c2ccc(-c3ccc4ccc(-c5nc(-c6ccccc6)nc(-c6ccccc6)n5)cc4c3)cc2)c1]

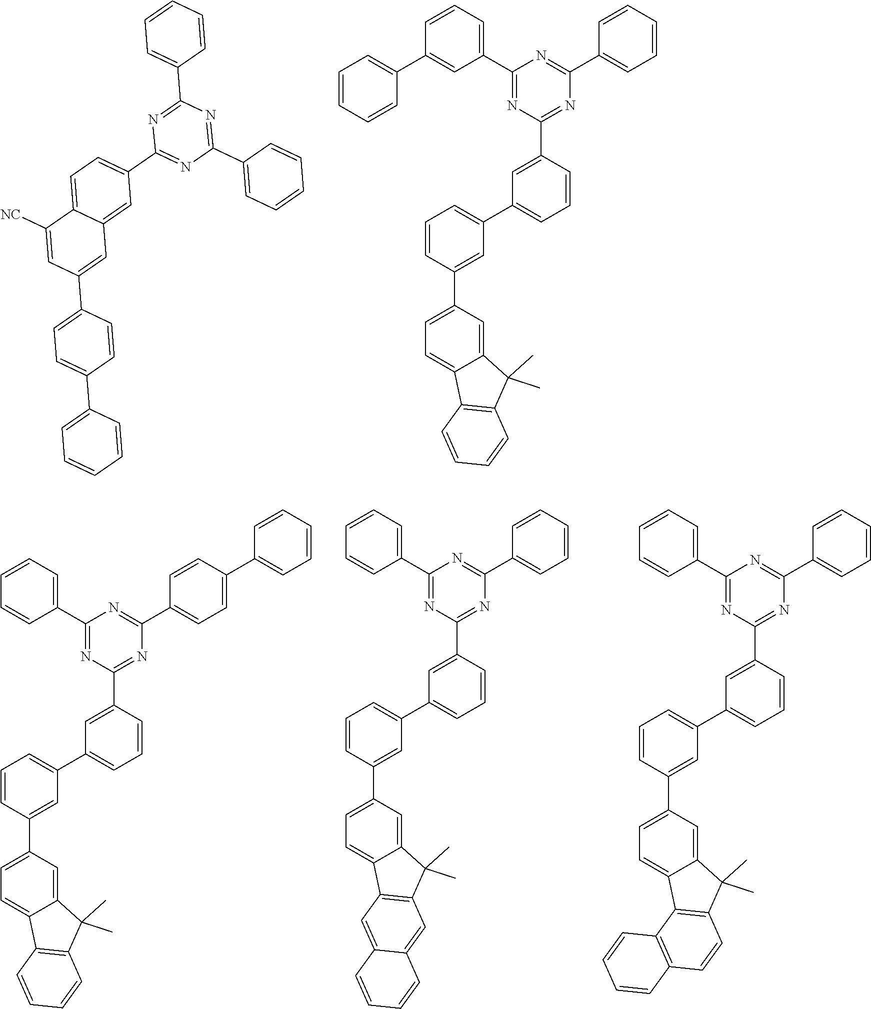

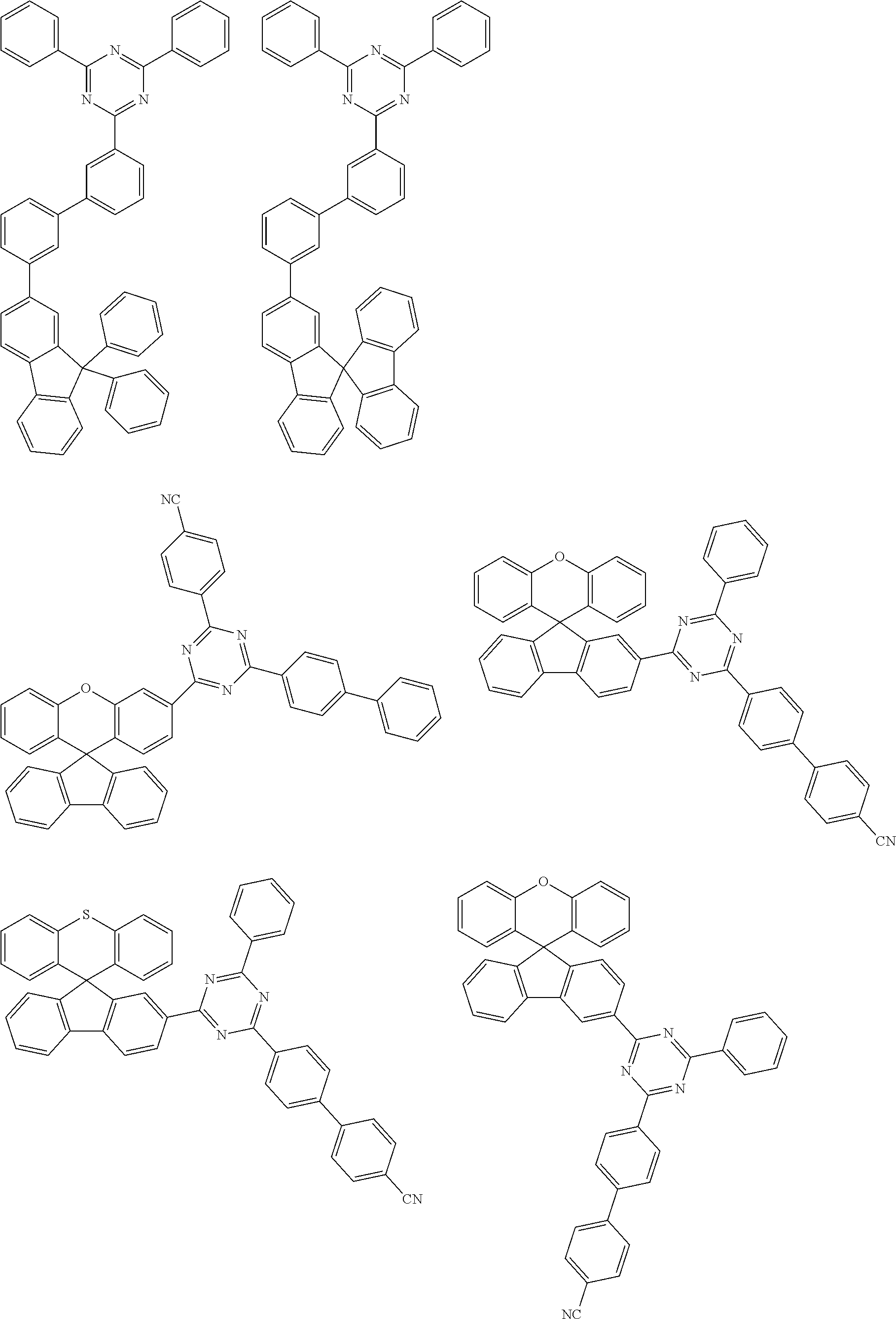

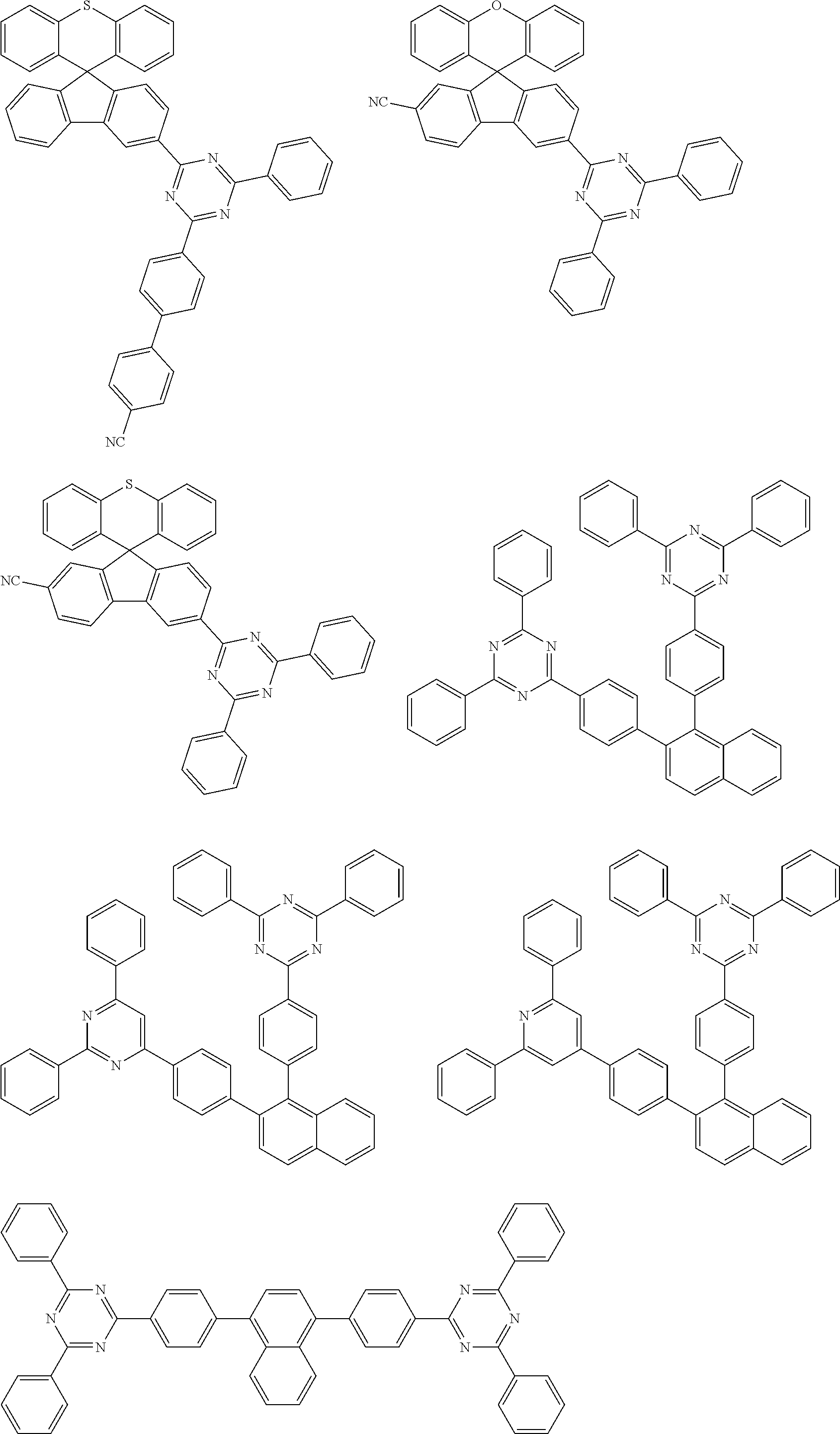

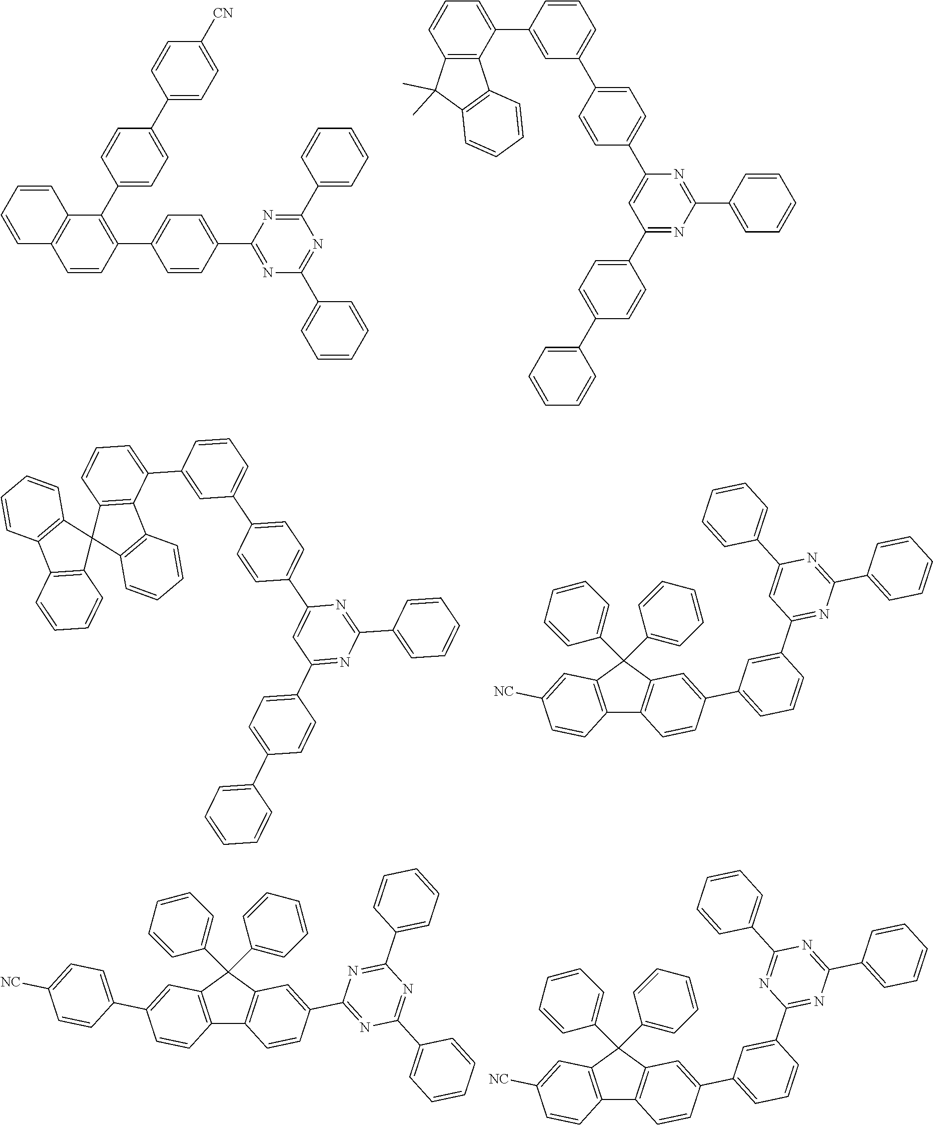

In an implementation, the organic light emitting diode may further include an electron injection layer, a hole injection layer, or the like, in addition to the light emitting layer as the aforementioned organic layer.

The organic light emitting diode may be produced by forming an anode or a cathode on a substrate, forming an organic layer using a dry film formation method such as a vacuum deposition method (evaporation), sputtering, plasma plating, and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in examples and synthesis examples were purchased from Sigma-Aldrich Co. Ltd., TCI Inc., Tokyo chemical industry, or P&H tech as far as there in no particular comment or were synthesized by known methods.

(Synthesis of Compounds)

Compounds were synthesized through the following steps.

### Synthesis of First Compound

### Synthesis Example 1: Synthesis of Compound A-22

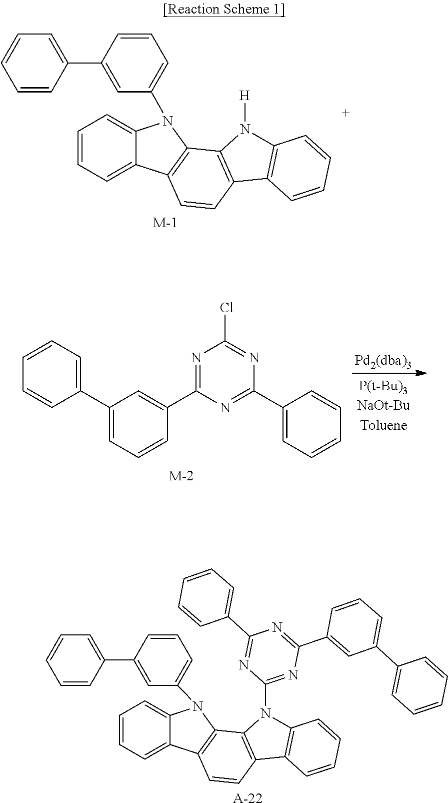

A-22 was synthesized using Intermediate M-1 (CAS No. 1449754-80-6) and Intermediate M-2 (CAS No. 1689576-03-1) with reference to a synthesis method described in Korean Patent Application No. KR 10-2021-0039202.

### Synthesis Example 2: Synthesis of Compound A-29

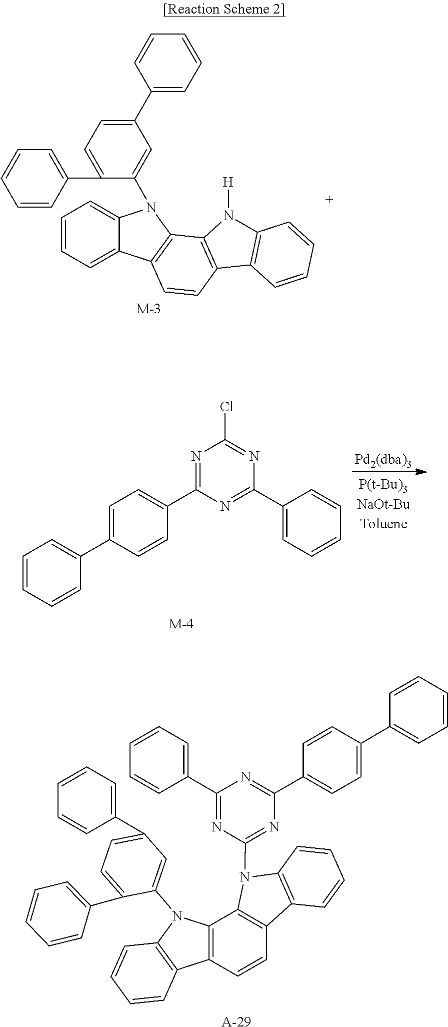

A-29 was synthesized using Intermediate M-3 and Intermediate M-4 (CAS NO. 1472062-94-4) with reference to the synthesis method described in Korean Patent Application No. KR 10-2021-0039202.

### Synthesis Example 3: Synthesis of Compound A-21

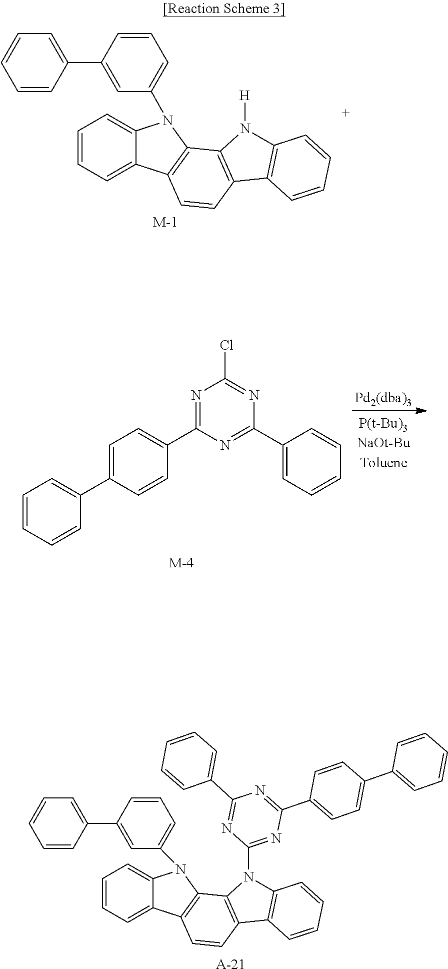

A-21 was synthesized using Intermediate M-1 (CAS NO. 1449754-80-6) and Intermediate M-4 (CAS NO. 1472062-94-4) with reference to the synthesis method described in Korean Patent Application No. KR 10-2021-0039202.

### Synthesis Example 4: Synthesis of Compound A-24

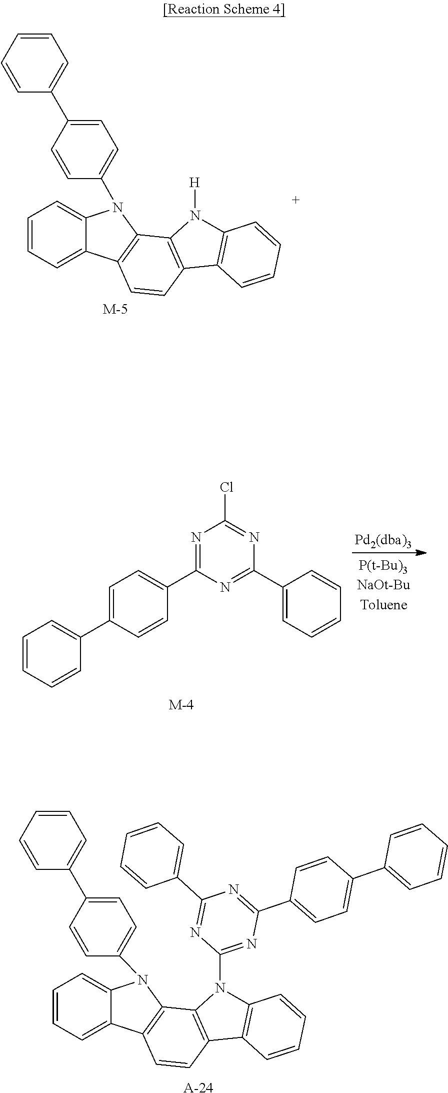

A-24 was synthesized using Intermediate M-5 (CAS NO. 2095887-12-8) and Intermediate M-4 (CAS NO. 1472062-94-4) with reference to the synthesis method described in Korean Patent Application No. KR 10-2021-0039202.

### Synthesis Example 5: Synthesis of Compound A-42

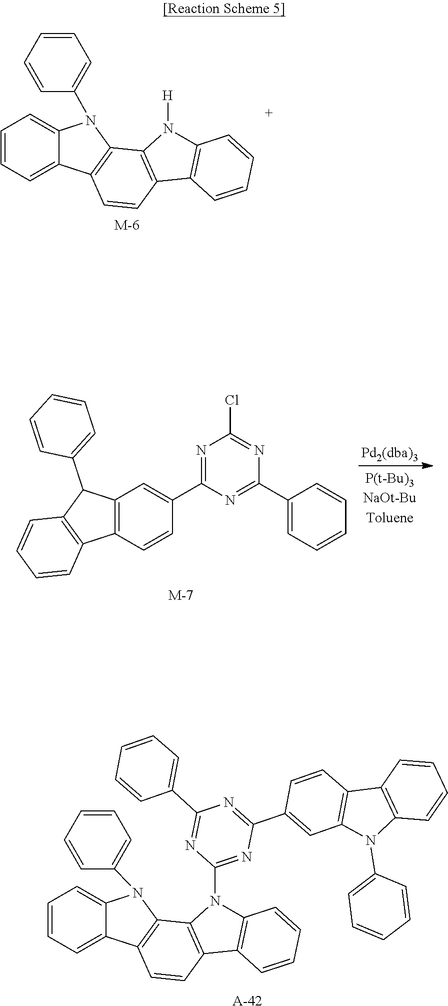

A-42 was synthesized using Intermediate M-6 (CAS NO. 1024598-06-8) and Intermediate M-7 (CAS NO. 2172944-00-0), with reference to the synthesis method described in Korean Patent Application No. KR 10-2021-0039202.

### Comparative Synthesis Example 1: Synthesis of Compound ET-1

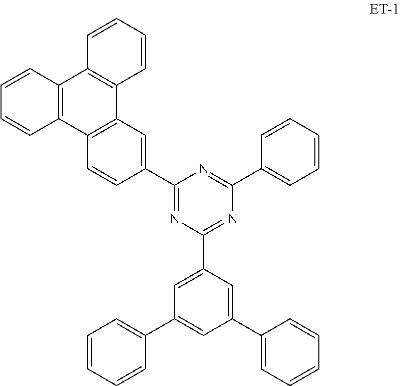 [SMILES: c1ccc(-c2cc(-c3ccccc3)cc(-c3nc(-c4ccccc4)nc(-c4ccc5c6ccccc6c6ccccc6c5c4)n3)c2)cc1]

Compound ET-1 was synthesized with reference to the synthesis method described in Korean Patent No. KR 10-2275343.

### Comparative Synthesis Example 2: Synthesis of Compound ET-2

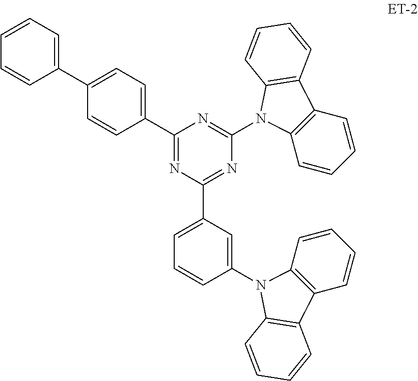 [SMILES: c1ccc(-c2ccc(-c3nc(-c4cccc(-n5c6ccccc6c6ccccc65)c4)nc(-n4c5ccccc5c5ccccc54)n3)cc2)cc1]

Compound ET-2 was synthesized with reference to the synthesis method described in Korean Patent Application No. KR 10-2020-0087020.

### Comparative Synthesis Example 3: Synthesis of Compound ET-3

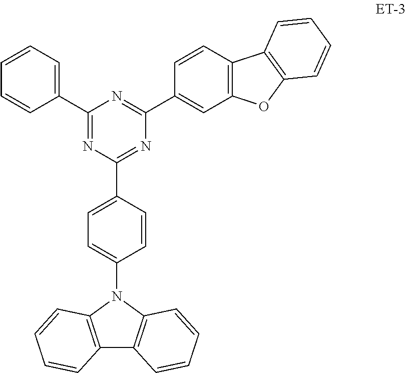 [SMILES: c1ccc(-c2nc(-c3ccc(-n4c5ccccc5c5ccccc54)cc3)nc(-c3ccc4c(c3)oc3ccccc34)n2)cc1]

Compound ET-3 was synthesized with reference to the synthesis method described in Korean Patent No. KR 10-1947747.

### Synthesis of Second Compound

### Synthesis Example 6: Synthesis of Compound B-136

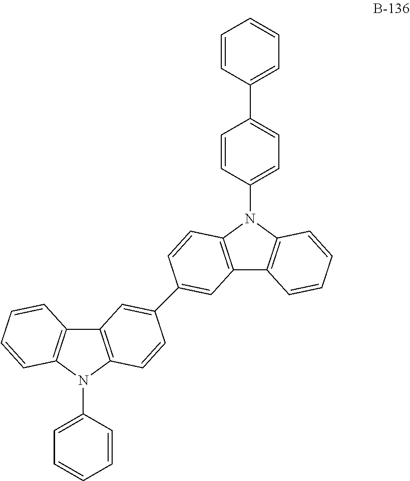 [SMILES: c1ccc(-c2ccc(-n3c4ccccc4c4cc(-c5ccc6c(c5)c5ccccc5n6-c5ccccc5)ccc43)cc2)cc1]

Compound B-136 was synthesized with reference to the synthesis method described in U.S. Pat. No. 10,476,008 B2.

### Synthesis Example 7: Synthesis of Compound B-139

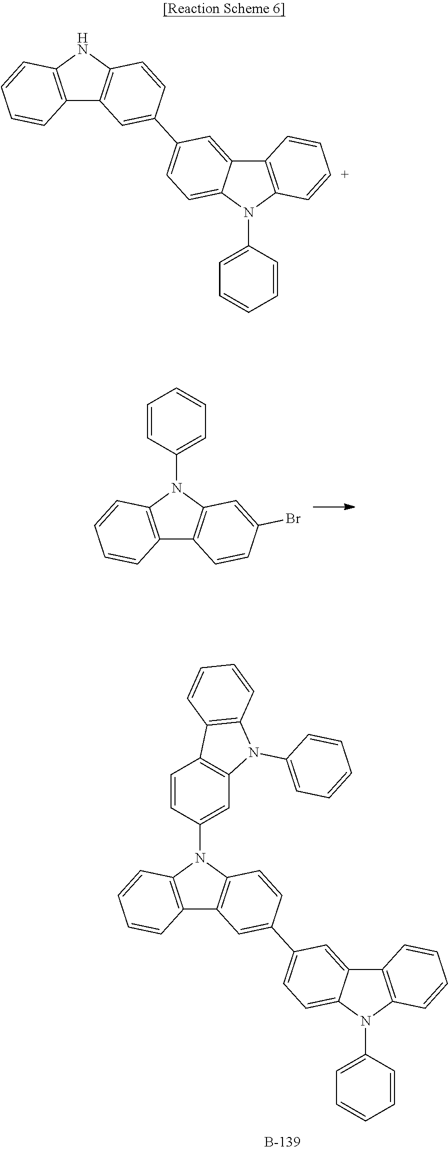 [SMILES: [C+]1=Cc2c(c3cc(-c4ccc5[nH]c6ccccc6c5c4)ccc3n2-c2ccccc2)C=C1 ; Brc1ccc2c3ccccc3n(-c3ccccc3)c2c1 ; c1ccc(-n2c3ccccc3c3cc(-c4ccc5c(c4)c4ccccc4n5-c4ccc5c6ccccc6n(-c6ccccc6)c5c4)ccc32)cc1]

In a 250 mL round flask, 1 equivalent of N-phenyl-3,3-bicarbazole, 1 equivalent of 2-bromo-9-phenylcarbazole, 1.5 equivalents of sodium t-butoxide, 0.03 equivalents of tris(dibenzylideneacetone) dipalladium, and 0.06 equivalents of tri-t-butylphosphine were mixed with xylene (to form a 0.3 M solution) and heated to reflux under a nitrogen stream for 15 hours. The resulting mixture was added to 300 mL of methanol, the crystallized solid was filtered, dissolved in dichlorobenzene, and filtered through silica gel/Celite, an appropriate amount of organic solvent was removed, and then recrystallized from methanol to obtain 67% of Compound B-139 in a yield of 67%.

### Synthesis Example 8: Synthesis of Compound B-141

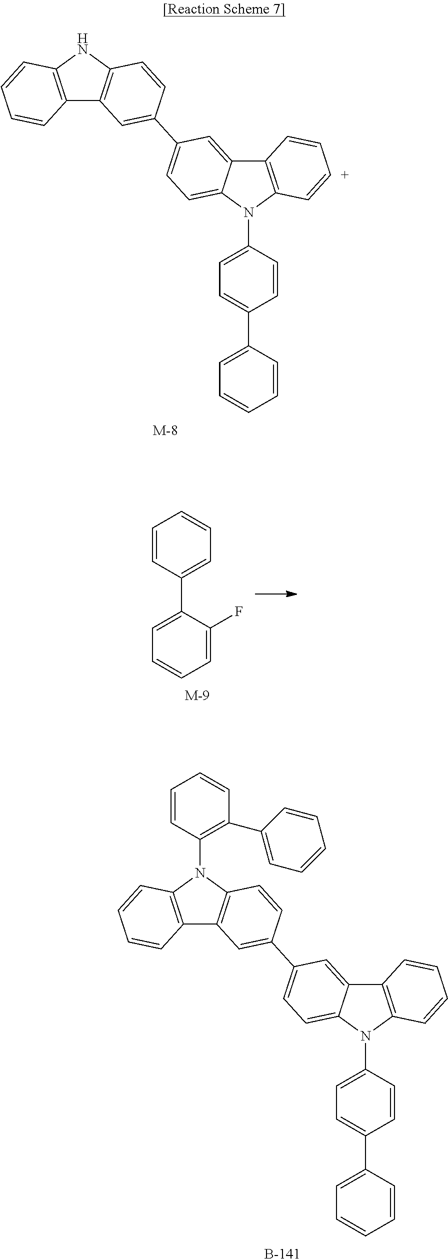

B-141 was synthesized with reference to the synthesis method of Synthesis Example 7 using Intermediate M-8 (CAS NO. 1346669-48-4) and Intermediate M-9 (CAS NO. 321-60-8).

### Synthesis of Third Compound

### Synthesis Example 9: Synthesis of Compound

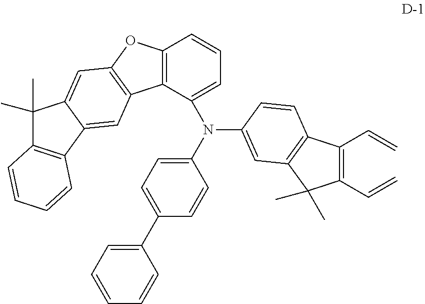 [SMILES: C=CC1=C(C=C)C(C)(C)c2cc(N(c3ccc(-c4ccccc4)cc3)c3cccc4oc5cc6c(cc5c34)-c3ccccc3C6(C)C)ccc21]

Compound D-1 was synthesized with reference to the synthesis method described in Korean Patent Application No. KR 10-2018-0037889.

### Comparative Synthesis Example 4: Synthesis of Compound R-1

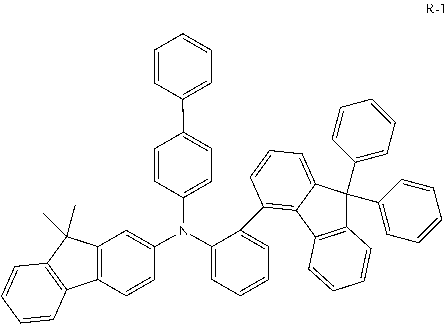 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21]

Compound R-1 was synthesized with reference to the synthesis method described in U.S. Pat. No. 10,355,217 B2.

### Comparative Synthesis Example 5: Synthesis of Compound R-2

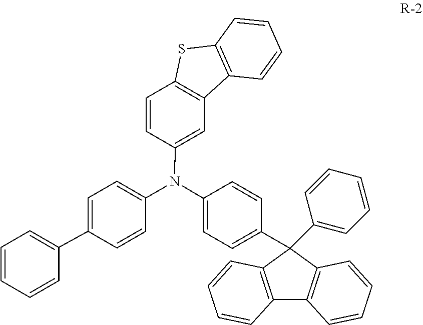 [SMILES: c1ccc(-c2ccc(N(c3ccc(C4(c5ccccc5)c5ccccc5-c5ccccc54)cc3)c3ccc4sc5ccccc5c4c3)cc2)cc1]

Compound R-2 was synthesized with reference to a method described in Korean Patent No. KR 10-1931250.

### Comparative Synthesis Example 6: Synthesis of Compound R-3

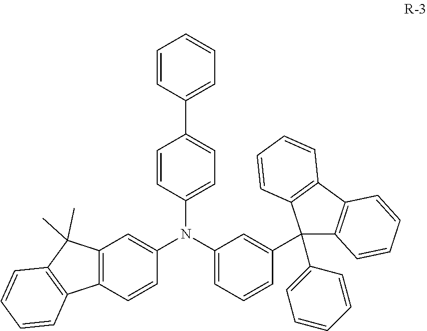 [SMILES: CC1(C)c2ccccc2-c2ccc(N(c3ccc(-c4ccccc4)cc3)c3cccc(C4(c5ccccc5)c5ccccc5-c5ccccc54)c3)cc21]

Compound R-3 was synthesized with reference to the synthesis method described in U.S. Pat. No. 10,505,121 B2.

### Comparative Synthesis Example 7: Synthesis of Compound R-4

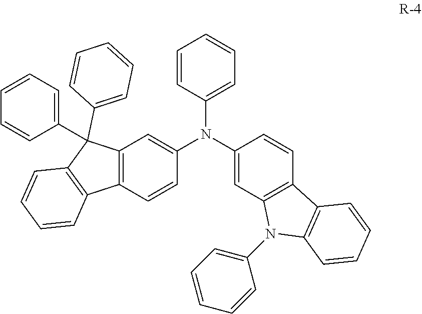 [SMILES: c1ccc(N(c2ccc3c(c2)C(c2ccccc2)(c2ccccc2)c2ccccc2-3)c2ccc3c4ccccc4n(-c4ccccc4)c3c2)cc1]

Compound R-4 was synthesized with reference to the synthesis method described in Korean Patent Application No. KR 10-2019-0108222 published patent.

### Manufacture of Organic Light Emitting Diode

### Example 1

A glass substrate coated with a thin film of indium tin oxide (ITO) was washed with distilled water and ultrasonic waves. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This obtained ITO transparent electrode was used as an anode, Compound A doped with 3% NDP-9 (available from Novaled) was vacuum-deposited on the ITO substrate to form a 100 Å-thick hole injection layer, and Compound A was deposited on the hole transport layer to form a 1,350 Å-thick hole transport layer. On the hole transport layer, Compound D-1 obtained in Synthesis Example 9 was deposited at a thickness of 320 Å to form a hole transport auxiliary layer, and on the hole transport auxiliary layer, Compound A-22 of Synthesis Example 1 and Compound B-136 of Synthesis Example 6 were simultaneously used as a host and 10 wt % of GD as a dopant was used to form a 330 Å-thick light emitting layer by vacuum deposition. Subsequently, on the light emitting layer, Compound B was deposited at a thickness of 50 Å to form an electron transport auxiliary layer and Compound C and LiQ were simultaneously vacuum-deposited in a weight ratio of 1:1 to form a 300 Å-thick electron transport layer. On the electron transport layer, LiQ and Al were sequentially vacuum-deposited to be 15 Å-thick and 1,200 Å-thick, manufacturing an organic light emitting diode.

ITO/Compound A (3% NDP-9 doping, 100 Å)/Compound A (1,350 Å)/Compound D-1 (320 Å)/EML [host (Compound A-22:Compound B-136=35:65): GD=90 wt %:10 wt %] (330 Å)/Compound B (50 Å)/Compound C: LiQ (300 Å)/LiQ (15 Å)/Al (1,200 Å).
Compound A: N-(biphenyl-4-yl)-9,9-dimethyl-N-(4-(9-phenyl-9H-carbazol-3-yl)phenyl)-9H-fluoren-2-amine
Compound B: 2-{3-[3-(9,9-dimethyl-9H-fluoren-2-yl)phenyl]phenyl}-4,6-diphenyl-1,3,5-triazine
Compound C: 4-(4-{4-[4-(diphenyl-1,3,5-triazin-2-yl)phenyl]naphthalene-1-yl}phenyl)benzonitrile

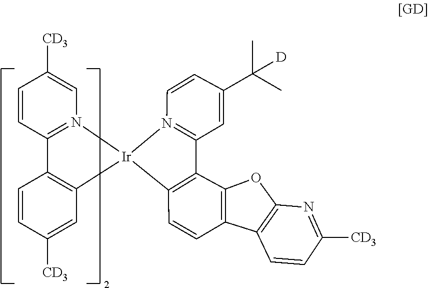

### Examples 2 to 6, and Comparative Examples 1 to 6

Diodes of Examples 2 to 6 and Comparative Examples 1 to 6 were manufactured in the same manner as in Example 1, except that the hole transport auxiliary layers and/or the hosts were changed as shown in Table 1.

Evaluation

The driving voltage characteristics of the organic light emitting diodes according to Examples 1 to 6 and Comparative Examples 1 to 6 were evaluated.

Specific measuring methods are as follows, and the results are shown in Table 1.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000 Å), while the voltage of the organic light emitting diodes was increased from 0 V to 10 V.

(3) Measurement of Driving Voltage

A driving voltage of each diode was measured using a current-voltage meter (Keithley 2400) at 15 mA/cm² to obtain the results.

Relative values based on the driving voltage of Comparative Example 1 are shown in Table 1.

TABLE 1

| | |
|---|---|
| | Hole |
| | transport |
| Host | auxiliary |

| | First | Second | layer | Driving |
|---|---|---|---|---|
| | compound | compound | Third | voltage |
| Nos. | (wt %) | (wt %) | compound | (%) |
| Example 1 | A-22 | B-136 | D-1 | 97 |
| | (35%) | (65%) | | |
| Example 2 | A-29 | B-136 | D-1 | 96 |
| | (30%) | (70%) | | |
| Example 3 | A-21 | B-136 | D-1 | 95 |
| | (30%) | (70%) | | |
| Example 4 | A-24 | B-141 | D-1 | 96 |
| | (30%) | (70%) | | |
| Example 5 | A-42 | B-136 | D-1 | 95 |
| | (30%) | (70%) | | |
| Example 6 | A-22 | B-139 | D-1 | 96 |
| | (30%) | (70%) | | |
| Comparative | A-22 | B-136 | R-1 | 100 |
| Example 1 | (35%) | (65%) | | |
| Comparative | A-22 | B-136 | R-2 | 100 |
| Example 2 | (35%) | (65%) | | |
| Comparative | A-22 | B-136 | R-3 | 100 |
| Example 3 | (35%) | (65%) | | |
| Comparative | A-22 | B-136 | R-4 | 104 |
| Example 4 | (35%) | (65%) | | |
| Comparative | ET-1 | B-136 | D-1 | 99 |
| Example 5 | (30%) | (70%) | | |
| Comparative | ET-2 | B-136 | D-1 | 102 |
| Example 6 | (30%) | (70%) | | |
| Comparative | ET-3 | B-136 | D-1 | 100 |
| Example 7 | (30%) | (70%) | | |

Referring to Table 1, the organic light emitting diodes including the compositions according to the Examples exhibited significantly improved driving voltage characteristics, compared to the organic light emitting diodes according to the Comparative Examples.

One or more embodiments may provide an organic optoelectronic device capable of implementing low driving characteristics.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. An organic optoelectronic device, comprising
an anode and a cathode facing each other;
a light emitting layer between the anode and the cathode;
a hole transport layer between the anode and the light emitting layer; and
a hole transport auxiliary layer between the light emitting layer and the hole transport layer,
wherein:
the light emitting layer includes a first compound represented by Chemical Formula 1 and a second compound represented by Chemical Formula 2 or a combination of Chemical Formula 3 and Chemical Formula 4, and
the hole transport auxiliary layer includes a third compound represented by a combination of Chemical Formula 5 and Chemical Formula 6, 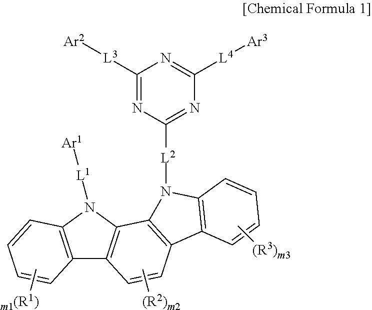
in Chemical Formula 1,
Ar¹ to Ar³ are each independently a substituted or unsubstituted C6 to C30 aryl group or a substituted or unsubstituted C2 to C30 heterocyclic group,
L¹ to L⁴ are each independently a single bond or a substituted or unsubstituted C6 to C20 arylene group,
R¹ to R³ are each independently hydrogen, deuterium, a cyano group, a halogen, a substituted or unsubstituted C1 to C30 alkyl group, or a substituted or unsubstituted C6 to C30 aryl group,
m1 and m3 are each independently an integer of 1 to 4, and
m2 is 1 or 2; 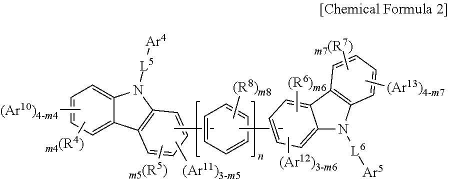
in Chemical Formula 2,
Ar⁴ and Ar⁵ are each independently a substituted or unsubstituted C6 to C20 aryl group or a substituted or unsubstituted C2 to C30 heterocyclic group,
Ar¹⁰ to Ar¹³ are each independently hydrogen or a substituted or unsubstituted C6 to C30 aryl group,
L⁵ and L⁶ are each independently a single bond or a substituted or unsubstituted C6 to C20 arylene group,
R⁴ to R⁸ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
m4, m7, and m8 are each independently an integer of 1 to 4,
m5 and m6 are each independently an integer of 1 to 3, and
n is an integer of 0 to 2; 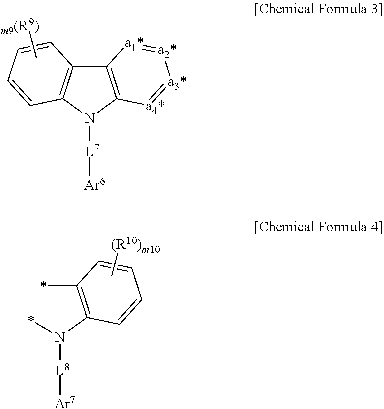
in Chemical Formula 3 and Chemical Formula 4,
Ar⁶ and Ar⁷ are each independently a substituted or unsubstituted C6 to C20 aryl group or a substituted or unsubstituted C2 to C30 heterocyclic group,
two adjacent ones of a1* to a4* of Chemical Formula 3 are linking carbons linked at * of Chemical Formula 4, and the remaining two of a1* to a4* of Chemical Formula 3, not linked at * of Chemical Formula 4, are C-L^{a}-R^{a},
L^{a}, L⁷, and L⁸ are each independently a single bond or a substituted or unsubstituted C6 to C20 arylene group,
R^{a}, R⁹ and R¹⁰ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, and
m9 and m10 are each independently an integer of 1 to 4; 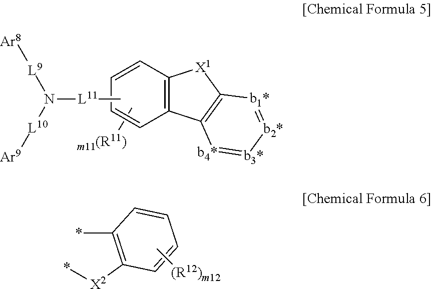
in Chemical Formula 5 and Chemical Formula 6,
X¹ and X² are each independently O, S, or CR^{b}R^{c},
two adjacent ones of b1* to b4* of Chemical Formula 5 are linking carbons linked at * of Chemical Formula 6, and the remaining two of b1* to b4* of Chemical Formula 5, not linked at * of Chemical Formula 6, are C-L^{b}-R^{d},
L^{b} and L⁹ to L¹¹ are each independently a single bond, a substituted or unsubstituted C6 to C20 arylene group, or a substituted or unsubstituted C2 to C20 heterocyclic group,
Ar⁸ and Ar⁹ are each independently a substituted or unsubstituted C6 to C30 aryl group or a substituted or unsubstituted C2 to C30 heterocyclic group,
R^{b} and R^{c} are each independently a substituted or unsubstituted C1 to C30 alkyl group or a substituted or unsubstituted C6 to C30 aryl group,
R^{d}, R¹¹ and R¹² are each independently hydrogen, deuterium, a cyano group, a halogen, a substituted or unsubstituted C1 to C30 alkyl group, a substituted or unsubstituted C6 to C30 aryl group, or a substituted or unsubstituted C2 to C30 heterocyclic group,
m11 is an integer of 1 to 3, and
m12 is an integer of 1 to 4.

2. The organic optoelectronic device as claimed in claim 1, wherein:
Ar¹ to Ar³ are each independently a substituted or unsubstituted phenyl group, a substituted or unsubstituted biphenyl group, a substituted or unsubstituted terphenyl group, a substituted or unsubstituted carbazolyl group, a substituted or unsubstituted dibenzofuranyl group, or a substituted or unsubstituted dibenzothiophenyl group, and
at least one of Ar² and Ar³ is a substituted or unsubstituted biphenyl group, a substituted or unsubstituted carbazolyl group, a substituted or unsubstituted dibenzofuranyl group, or a substituted or unsubstituted dibenzothiophenyl group.

3. The organic optoelectronic device as claimed in claim 1, wherein:
the second compound is represented by Chemical Formula 2-8 or Chemical Formula 3C: 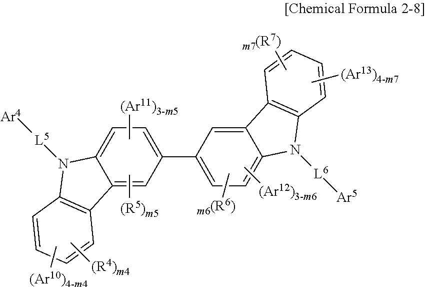
in Chemical Formula 2-8,
R⁴ to R⁷ are each independently hydrogen, deuterium, or a substituted or unsubstituted C6 to C12 aryl group,
Ar¹⁰ to Ar¹³ are each independently hydrogen or a substituted or unsubstituted C6 to C12 aryl group,
m4 and m7 are each independently one of integers from 1 to 4,
m5 and m6 are each independently one of integers from 1 to 3, and
moieties *-L⁵-Ar⁴ and *-L⁶-Ar⁵ are each independently a moiety of Group II, 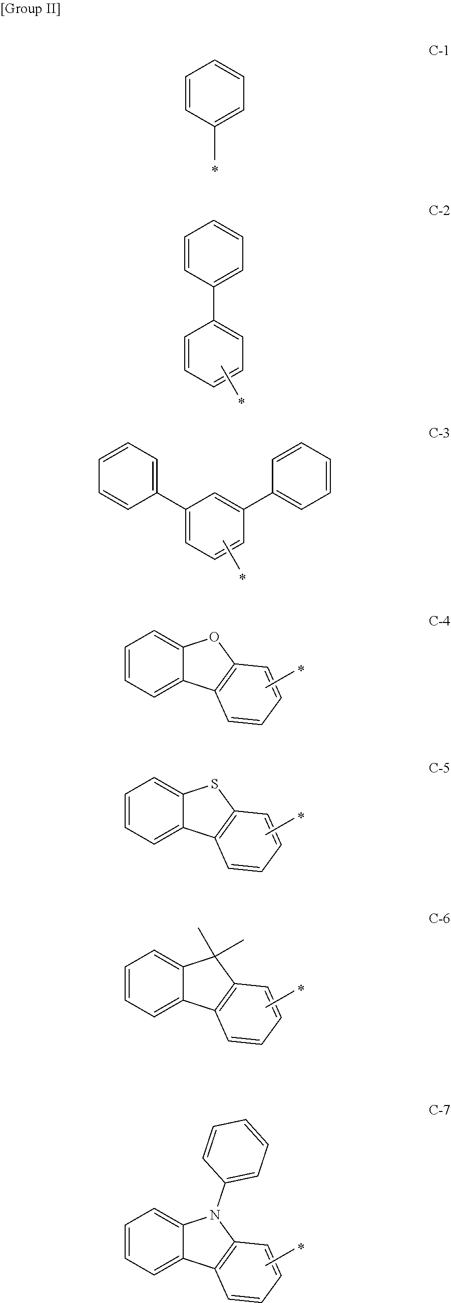 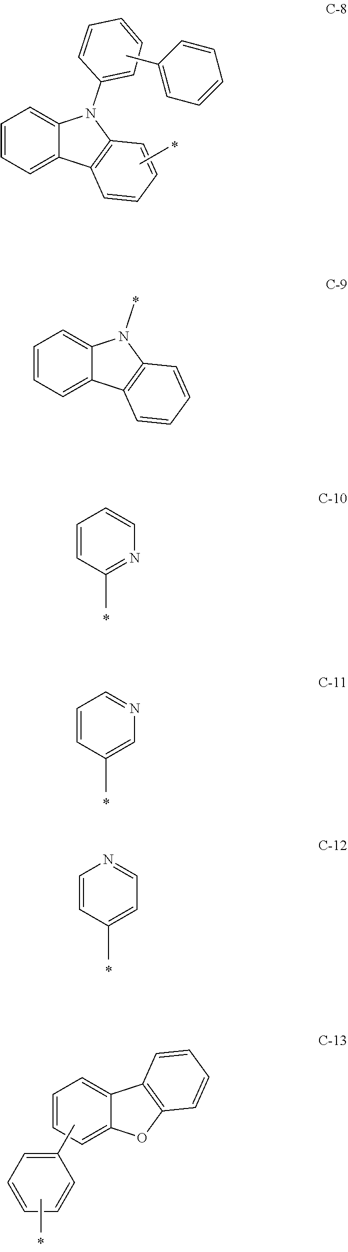 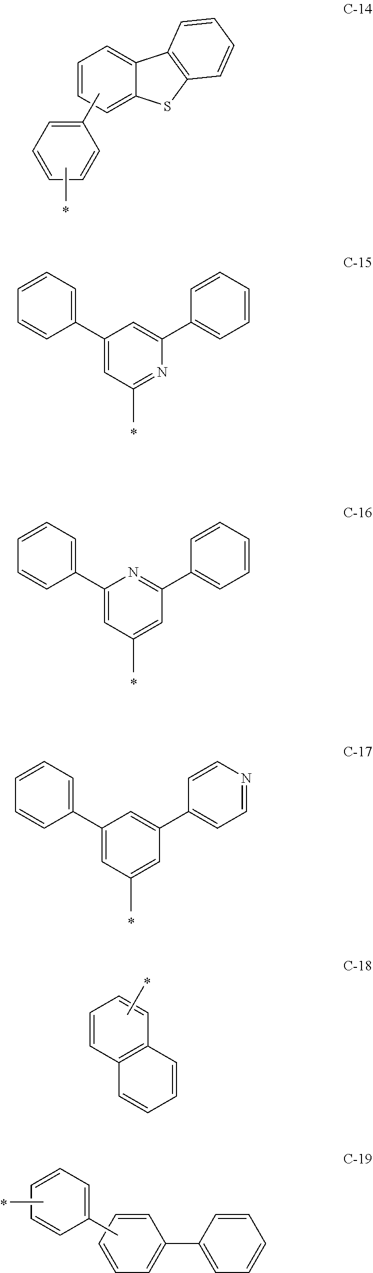 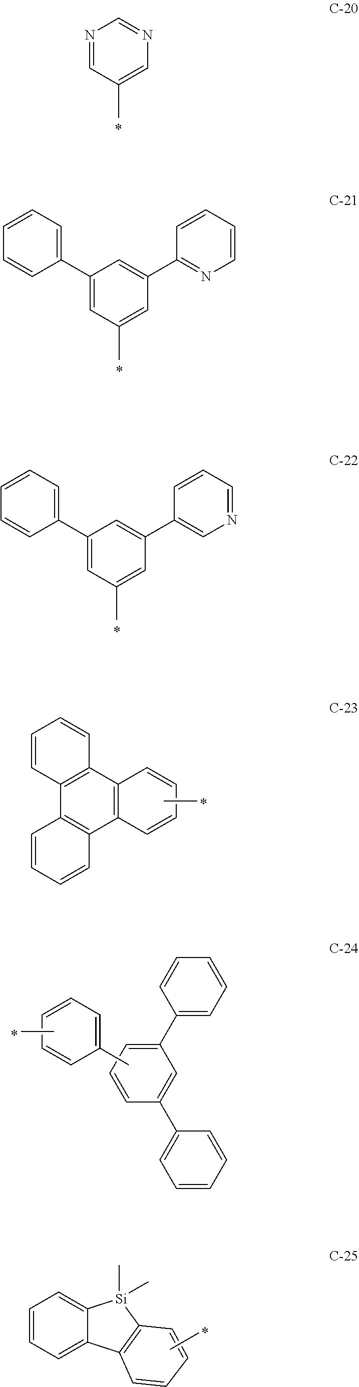 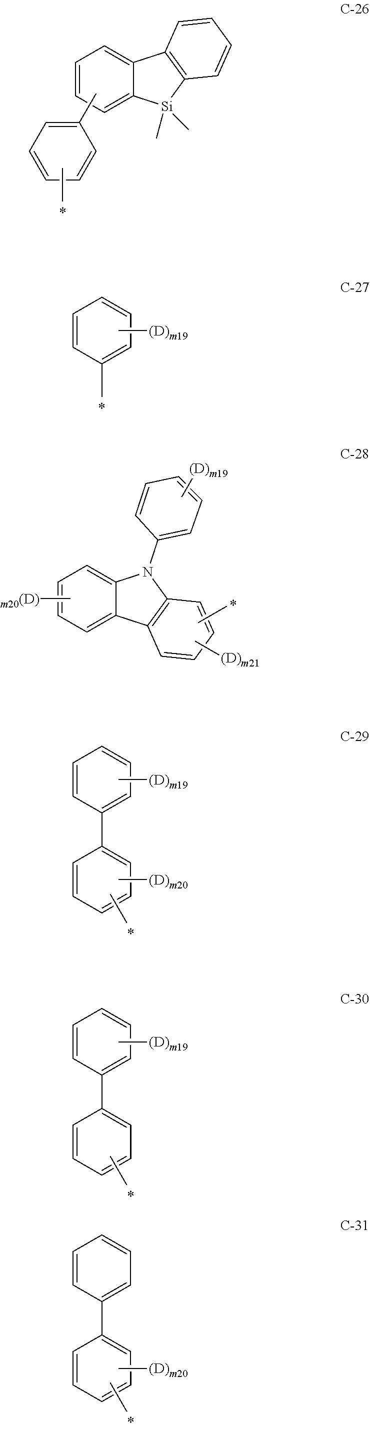 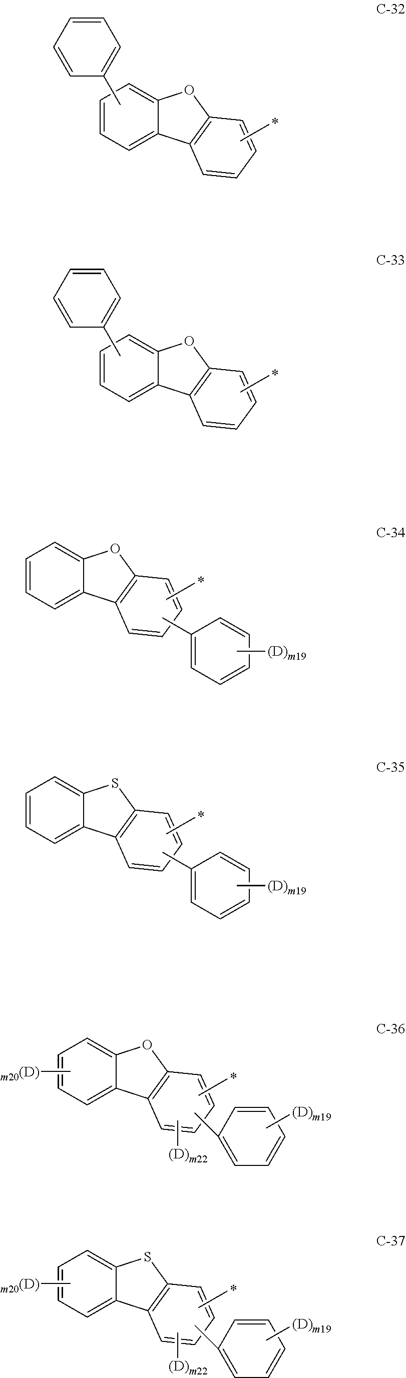 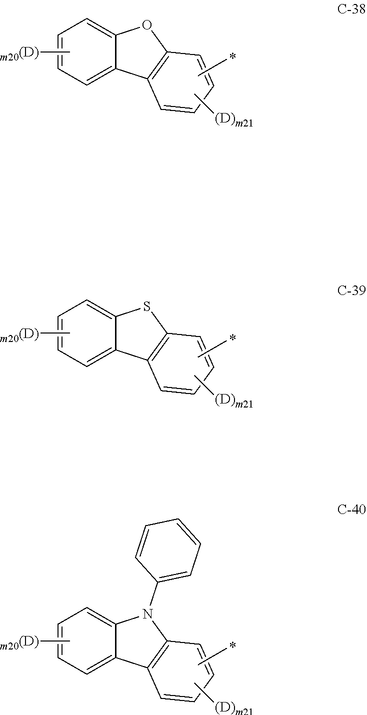
in Group I1,
D is deuterium,
m19 is an integer of 1 to 5,
m20 is an integer of 1 to 4,
m21 is an integer of 1 to 3,
m22 is 1 or 2, and
* is a linking point, 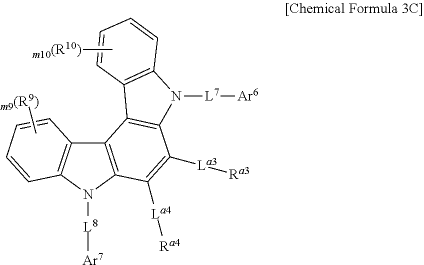
in Chemical Formula 3C,
L^{a3} and L^{a4} are each a single bond,
L⁷ and L⁸ are each independently a single bond or a substituted or unsubstituted C6 to C12 arylene group,
R⁹, R¹⁰, R^{a3}, and R^{a4} are each independently hydrogen, deuterium, or a substituted or unsubstituted C6 to C12 aryl group,
Ar⁶ and Ar⁷ are each independently a substituted or unsubstituted phenyl group, a substituted or unsubstituted naphthyl group, or a substituted or unsubstituted biphenyl group, and
m9 and m10 are each independently an integer of 1 to 4.

4. The organic optoelectronic device as claimed in claim 1, wherein:
the third compound is represented by one of Chemical Formula 5A to Chemical Formula 5F: 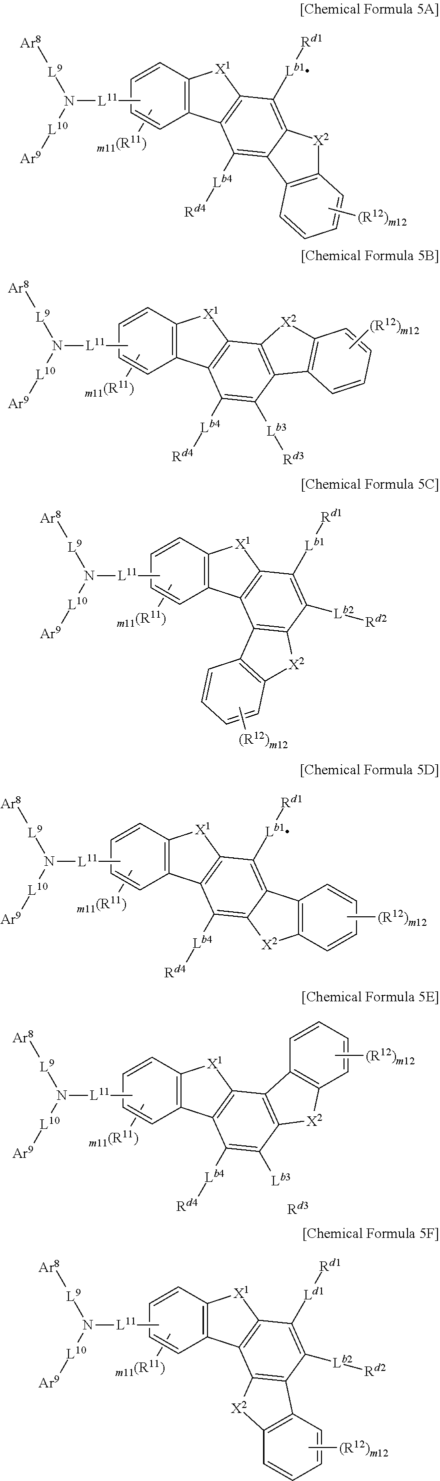
in Chemical Formula 5A to Chemical Formula 5F,
X¹, X², R¹¹, R¹², Ar⁸, Ar⁹, L⁹ to L¹¹, m11, and m12 are defined the same as those of Chemical Formulae 5 and 6,
L^{b1} to L⁴ are each independently a single bond, a substituted or unsubstituted C6 to C20 arylene group, or a substituted or unsubstituted C2 to C20 heterocyclic group, and
R^{d1} to R^{d4} are each independently hydrogen, deuterium, a cyano group, a halogen, a substituted or unsubstituted C1 to C30 alkyl group, a substituted or unsubstituted C6 to C30 aryl group or a substituted or unsubstituted C2 to C30 heterocyclic group.

5. The organic optoelectronic device as claimed in claim 1, wherein, in Chemical Formulae 5 and 6:
X¹ is CR^{b}R^{c},
R^{b} and R^{c} are each independently a substituted or unsubstituted C1 to C30 alkyl group, a substituted or unsubstituted C6 to C30 aryl group, and
X² is O or S.

6. The organic optoelectronic device as claimed in claim 1, wherein, in Chemical Formulae 5 and 6:
X¹ is O or S,
X² is CR^{b}R^{c},
R^{b} and R^{c} are each independently a substituted or unsubstituted C1 to C30 alkyl group or a substituted or unsubstituted C6 to C30 aryl group,
Ar⁸ and Ar⁹ are each independently a substituted or unsubstituted C6 to C30 aryl group or a substituted or unsubstituted C2 to C30 heterocyclic group, and
at least one of Ar⁸ and Ar⁹ is a substituted or unsubstituted fluorenyl group or a substituted or unsubstituted dibenzosilolyl group.

7. The organic optoelectronic device as claimed in claim 1, wherein Ar^{g} and Ar⁹ of Chemical Formula 5 are each independently a substituted or unsubstituted phenyl group, a substituted or unsubstituted biphenyl group, a substituted or unsubstituted fluorenyl group, a substituted or unsubstituted dibenzosilolyl group, a substituted or unsubstituted triphenylene group, a substituted or unsubstituted dibenzofuranyl group, or a substituted or unsubstituted dibenzothiophenyl group.

8. The organic optoelectronic device as claimed in claim 1, wherein:
Ar⁸ and Ar⁹ of Chemical Formula 5 are each independently a group of Group III: 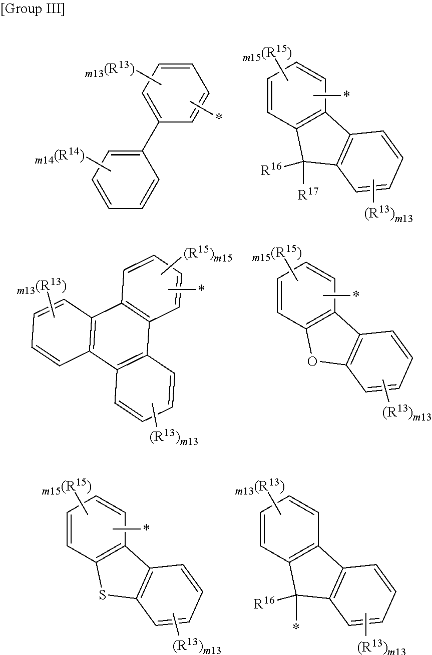
in Group III,
R¹⁶ and R¹¹ are each independently a substituted or unsubstituted C1 to C10 alkyl group or a substituted or unsubstituted C6 to C20 aryl group,
R¹³ to R¹⁵ are each independently hydrogen, deuterium, a substituted or unsubstituted C1 to C10 alkyl group, a substituted or unsubstituted C6 to C12 aryl group, or a substituted or unsubstituted C2 to C20 heterocyclic group,
m13 is an integer of 1 to 4,
m14 is an integer of 1 to 5,
m15 is an integer of 1 to 3, and
* is a linking point.

9. The organic optoelectronic device as claimed in claim 1, wherein:
the third compound is represented by Chemical Formula 5A-4: 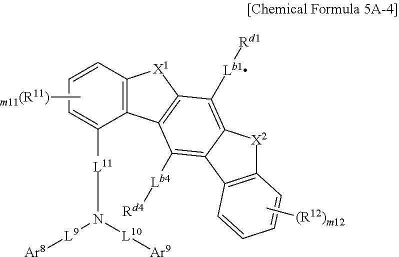
in Chemical Formula 5A-4,
X¹, X², R¹¹, R¹², Ar⁸, Ar⁹, L⁹ to L¹¹, m11, and m12 are defined the same as those of Chemical Formulae 5 and 6,
L^{b1} and L^{b4} are each independently a single bond, a substituted or unsubstituted C6 to C20 arylene group, or a substituted or unsubstituted C2 to C20 heterocyclic group, and
R^{d1} and R^{d4} are each independently hydrogen, deuterium, a cyano group, a halogen, a substituted or unsubstituted C1 to C30 alkyl group, a substituted or unsubstituted C6 to C30 aryl group, or a substituted or unsubstituted C2 to C30 heterocyclic group.

10. The organic optoelectronic device as claimed in claim 1, wherein the third compound is a compound of Group 3:

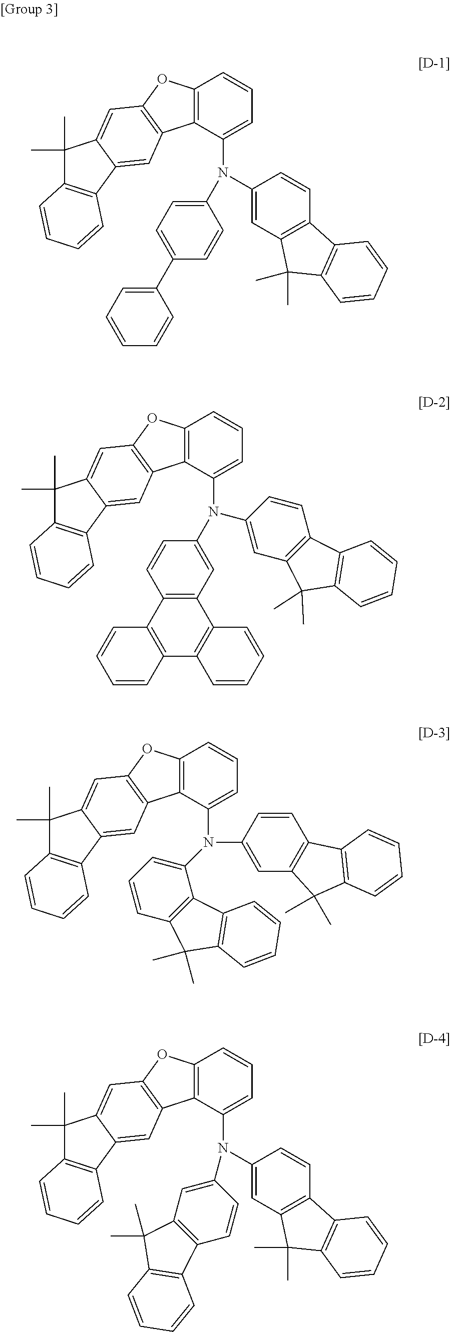 [SMILES: CC1(C)c2ccccc2-c2ccc(N(c3ccc(-c4ccccc4)cc3)c3cccc4oc5cc6c(cc5c34)-c3ccccc3C6(C)C)cc21 ; CC1(C)c2ccccc2-c2ccc(N(c3ccc4c5ccccc5c5ccccc5c4c3)c3cccc4oc5cc6c(cc5c34)-c3ccccc3C6(C)C)cc21 ; CC1(C)c2ccccc2-c2ccc(N(c3cccc4c3-c3ccccc3C4(C)C)c3cccc4oc5cc6c(cc5c34)-c3ccccc3C6(C)C)cc21 ; CC1(C)c2ccccc2-c2ccc(N(c3ccc4c(c3)C(C)(C)c3ccccc3-4)c3cccc4oc5cc6c(cc5c34)-c3ccccc3C6(C)C)cc21]

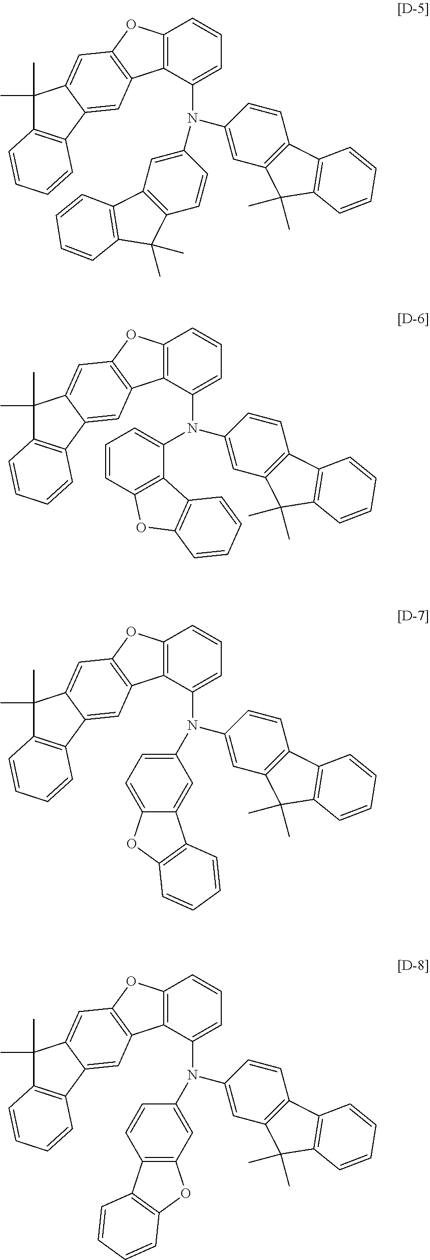 [SMILES: CC1(C)c2ccccc2-c2cc(N(c3ccc4c(c3)C(C)(C)c3ccccc3-4)c3cccc4oc5cc6c(cc5c34)-c3ccccc3C6(C)C)ccc21 ; CC1(C)c2ccccc2-c2ccc(N(c3cccc4oc5ccccc5c34)c3cccc4oc5cc6c(cc5c34)-c3ccccc3C6(C)C)cc21 ; CC1(C)c2ccccc2-c2ccc(N(c3ccc4oc5ccccc5c4c3)c3cccc4oc5cc6c(cc5c34)-c3ccccc3C6(C)C)cc21 ; CC1(C)c2ccccc2-c2ccc(N(c3ccc4c(c3)oc3ccccc34)c3cccc4oc5cc6c(cc5c34)-c3ccccc3C6(C)C)cc21]

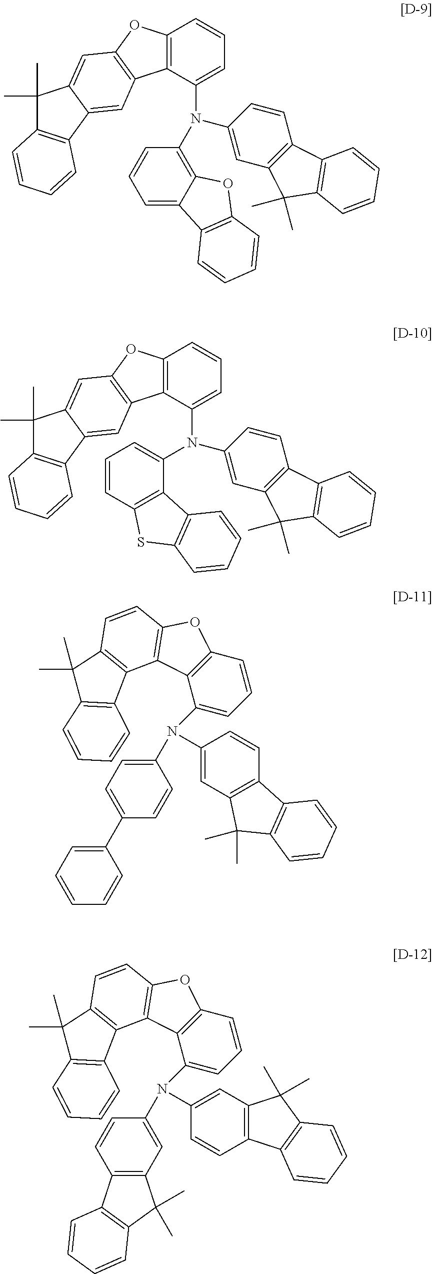 [SMILES: CC1(C)c2ccccc2-c2ccc(N(c3cccc4c3oc3ccccc34)c3cccc4oc5cc6c(cc5c34)-c3ccccc3C6(C)C)cc21 ; CC1(C)c2ccccc2-c2ccc(N(c3cccc4oc5cc6c(cc5c34)-c3ccccc3C6(C)C)c3cccc4sc5ccccc5c34)cc21 ; CC1(C)c2ccccc2-c2ccc(N(c3ccc(-c4ccccc4)cc3)c3cccc4oc5ccc6c(c5c34)-c3ccccc3C6(C)C)cc21 ; CC1(C)c2ccccc2-c2ccc(N(c3ccc4c(c3)C(C)(C)c3ccccc3-4)c3cccc4oc5ccc6c(c5c34)-c3ccccc3C6(C)C)cc21]

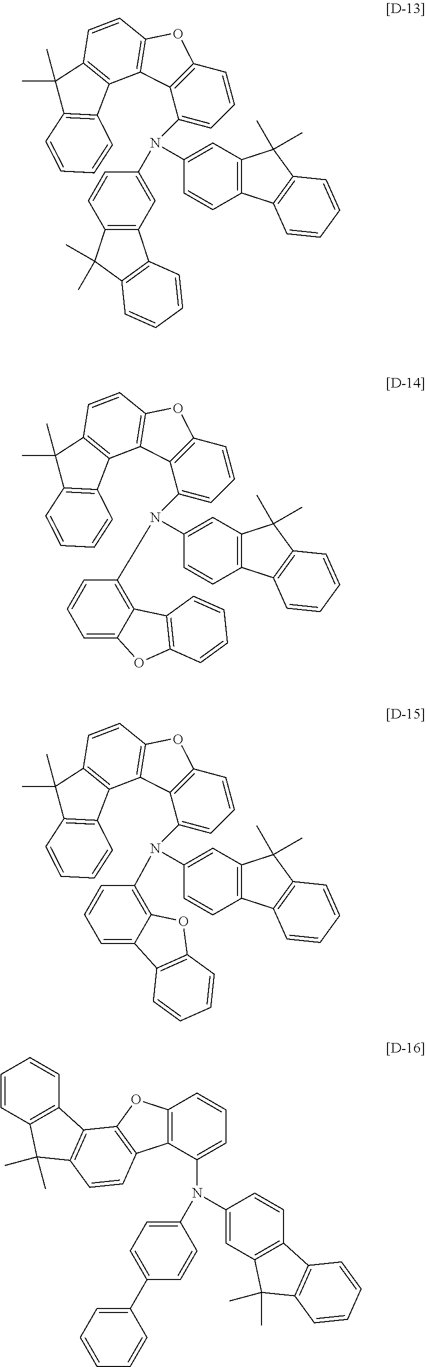 [SMILES: CC1(C)c2ccccc2-c2cc(N(c3ccc4c(c3)C(C)(C)c3ccccc3-4)c3cccc4oc5ccc6c(c5c34)-c3ccccc3C6(C)C)ccc21 ; CC1(C)c2ccccc2-c2ccc(N(c3cccc4oc5ccccc5c34)c3cccc4oc5ccc6c(c5c34)-c3ccccc3C6(C)C)cc21 ; CC1(C)c2ccccc2-c2ccc(N(c3cccc4c3oc3ccccc34)c3cccc4oc5ccc6c(c5c34)-c3ccccc3C6(C)C)cc21 ; CC1(C)c2ccccc2-c2ccc(N(c3ccc(-c4ccccc4)cc3)c3cccc4oc5c6c(ccc5c34)C(C)(C)c3ccccc3-6)cc21]

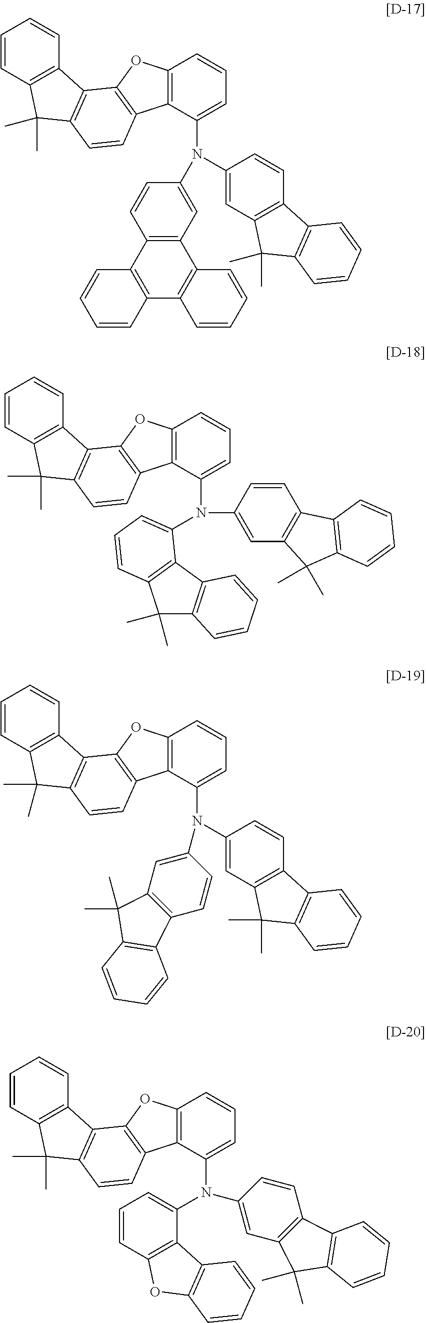 [SMILES: CC1(C)c2ccccc2-c2ccc(N(c3ccc4c5ccccc5c5ccccc5c4c3)c3cccc4oc5c6c(ccc5c34)C(C)(C)c3ccccc3-6)cc21 ; CC1(C)c2ccccc2-c2ccc(N(c3cccc4c3-c3ccccc3C4(C)C)c3cccc4oc5c6c(ccc5c34)C(C)(C)c3ccccc3-6)cc21 ; CC1(C)c2ccccc2-c2ccc(N(c3ccc4c(c3)C(C)(C)c3ccccc3-4)c3cccc4oc5c6c(ccc5c34)C(C)(C)c3ccccc3-6)cc21 ; CC1(C)c2ccccc2-c2ccc(N(c3cccc4oc5ccccc5c34)c3cccc4oc5c6c(ccc5c34)C(C)(C)c3ccccc3-6)cc21]

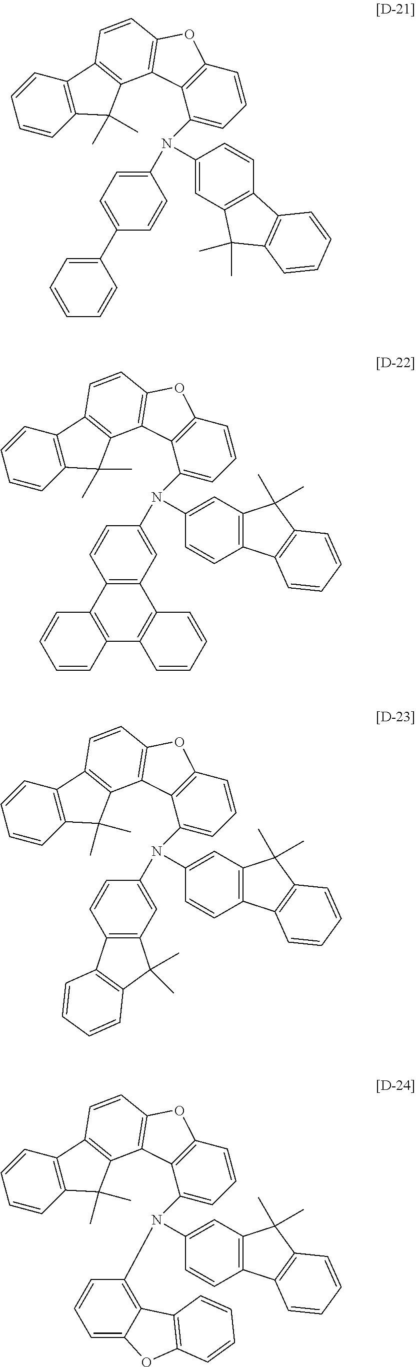 [SMILES: CC1(C)c2ccccc2-c2ccc(N(c3ccc(-c4ccccc4)cc3)c3cccc4oc5ccc6c(c5c34)C(C)(C)c3ccccc3-6)cc21 ; CC1(C)c2ccccc2-c2ccc(N(c3ccc4c5ccccc5c5ccccc5c4c3)c3cccc4oc5ccc6c(c5c34)C(C)(C)c3ccccc3-6)cc21 ; CC1(C)c2ccccc2-c2ccc(N(c3ccc4c(c3)C(C)(C)c3ccccc3-4)c3cccc4oc5ccc6c(c5c34)C(C)(C)c3ccccc3-6)cc21 ; CC1(C)c2ccccc2-c2ccc(N(c3cccc4oc5ccccc5c34)c3cccc4oc5ccc6c(c5c34)C(C)(C)c3ccccc3-6)cc21]

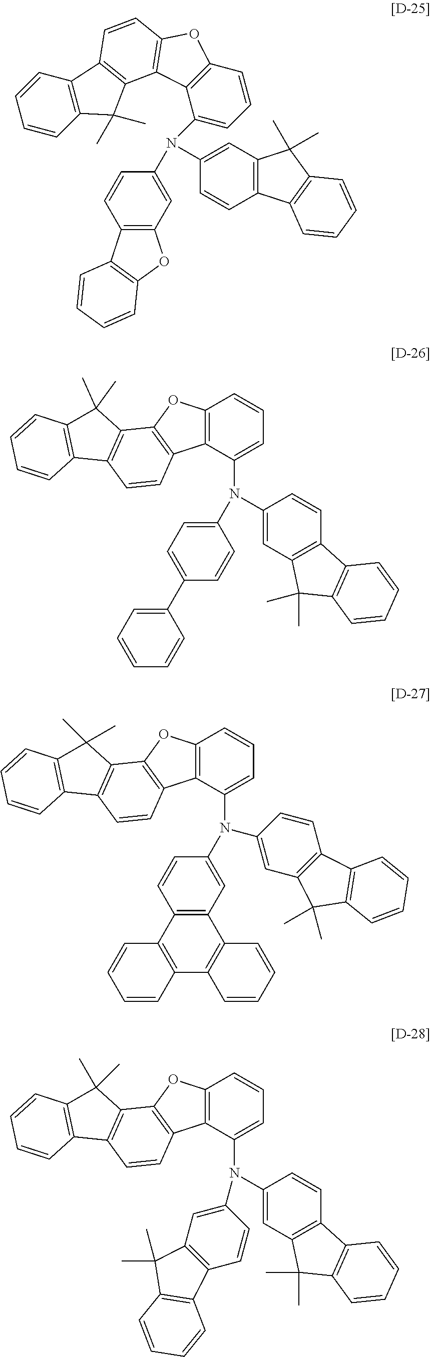 [SMILES: CC1(C)c2ccccc2-c2ccc(N(c3ccc4c(c3)oc3ccccc34)c3cccc4oc5ccc6c(c5c34)C(C)(C)c3ccccc3-6)cc21 ; CC1(C)c2ccccc2-c2ccc(N(c3ccc(-c4ccccc4)cc3)c3cccc4oc5c6c(ccc5c34)-c3ccccc3C6(C)C)cc21 ; CC1(C)c2ccccc2-c2ccc(N(c3ccc4c5ccccc5c5ccccc5c4c3)c3cccc4oc5c6c(ccc5c34)-c3ccccc3C6(C)C)cc21 ; CC1(C)c2ccccc2-c2ccc(N(c3ccc4c(c3)C(C)(C)c3ccccc3-4)c3cccc4oc5c6c(ccc5c34)-c3ccccc3C6(C)C)cc21]

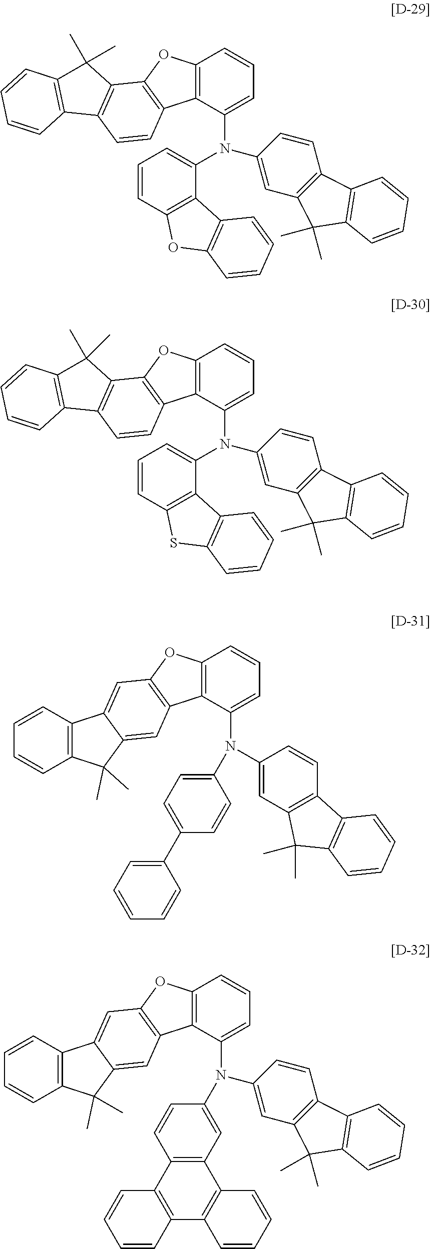 [SMILES: CC1(C)c2ccccc2-c2ccc(N(c3cccc4oc5ccccc5c34)c3cccc4oc5c6c(ccc5c34)-c3ccccc3C6(C)C)cc21 ; CC1(C)c2ccccc2-c2ccc(N(c3cccc4oc5c6c(ccc5c34)-c3ccccc3C6(C)C)c3cccc4sc5ccccc5c34)cc21 ; CC1(C)c2ccccc2-c2ccc(N(c3ccc(-c4ccccc4)cc3)c3cccc4oc5cc6c(cc5c34)C(C)(C)c3ccccc3-6)cc21 ; CC1(C)c2ccccc2-c2ccc(N(c3ccc4c5ccccc5c5ccccc5c4c3)c3cccc4oc5cc6c(cc5c34)C(C)(C)c3ccccc3-6)cc21]

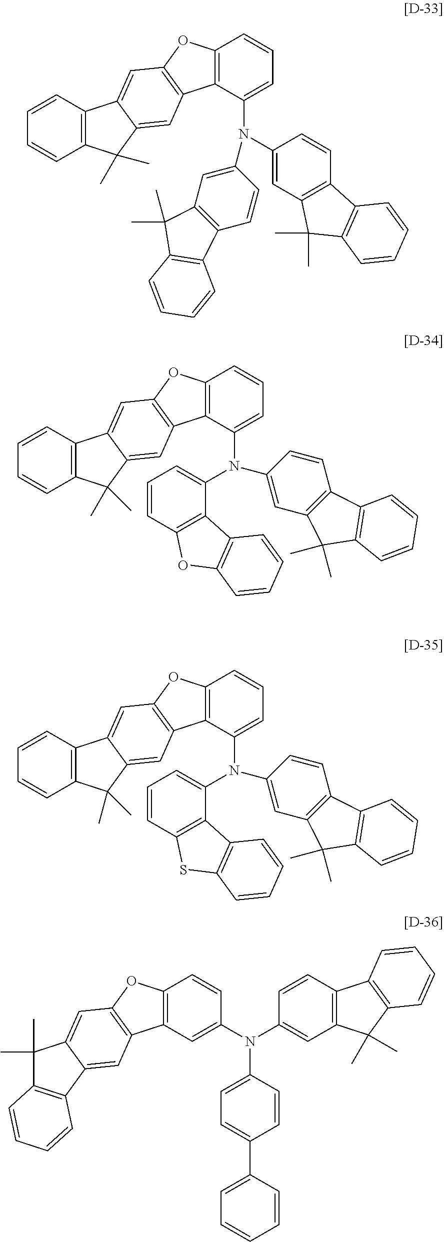 [SMILES: CC1(C)c2ccccc2-c2ccc(N(c3ccc4c(c3)C(C)(C)c3ccccc3-4)c3cccc4oc5cc6c(cc5c34)C(C)(C)c3ccccc3-6)cc21 ; CC1(C)c2ccccc2-c2ccc(N(c3cccc4oc5ccccc5c34)c3cccc4oc5cc6c(cc5c34)C(C)(C)c3ccccc3-6)cc21 ; CC1(C)c2ccccc2-c2ccc(N(c3cccc4oc5cc6c(cc5c34)C(C)(C)c3ccccc3-6)c3cccc4sc5ccccc5c34)cc21 ; CC1(C)c2ccccc2-c2ccc(N(c3ccc(-c4ccccc4)cc3)c3ccc4oc5cc6c(cc5c4c3)-c3ccccc3C6(C)C)cc21]

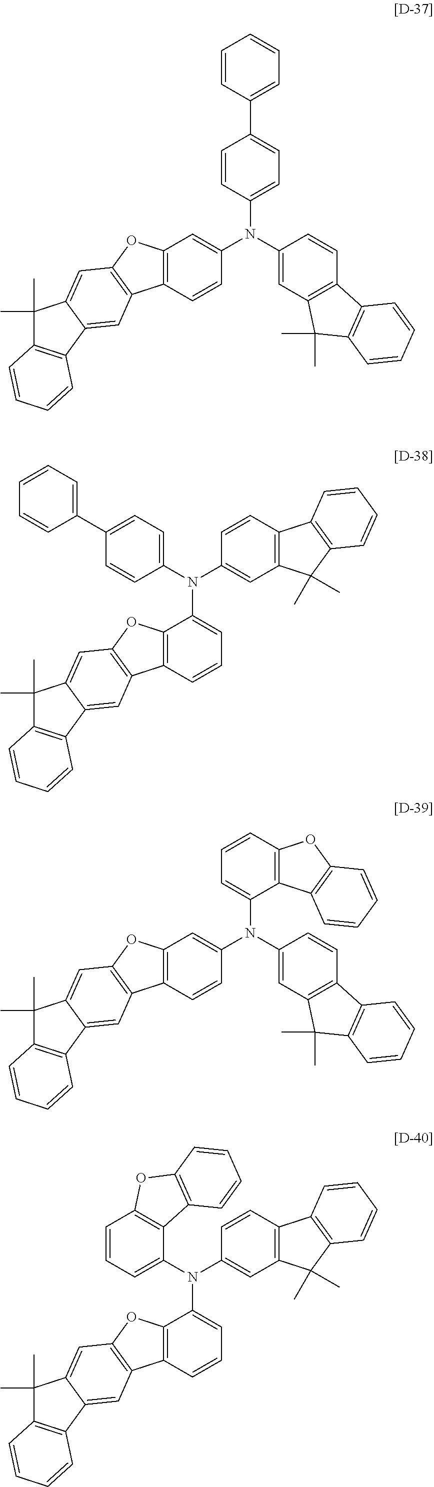 [SMILES: CC1(C)c2ccccc2-c2ccc(N(c3ccc(-c4ccccc4)cc3)c3ccc4c(c3)oc3cc5c(cc34)-c3ccccc3C5(C)C)cc21 ; CC1(C)c2ccccc2-c2ccc(N(c3ccc(-c4ccccc4)cc3)c3cccc4c3oc3cc5c(cc34)-c3ccccc3C5(C)C)cc21 ; CC1(C)c2ccccc2-c2ccc(N(c3ccc4c(c3)oc3cc5c(cc34)-c3ccccc3C5(C)C)c3cccc4oc5ccccc5c34)cc21 ; CC1(C)c2ccccc2-c2ccc(N(c3cccc4c3oc3cc5c(cc34)-c3ccccc3C5(C)C)c3cccc4oc5ccccc5c34)cc21]

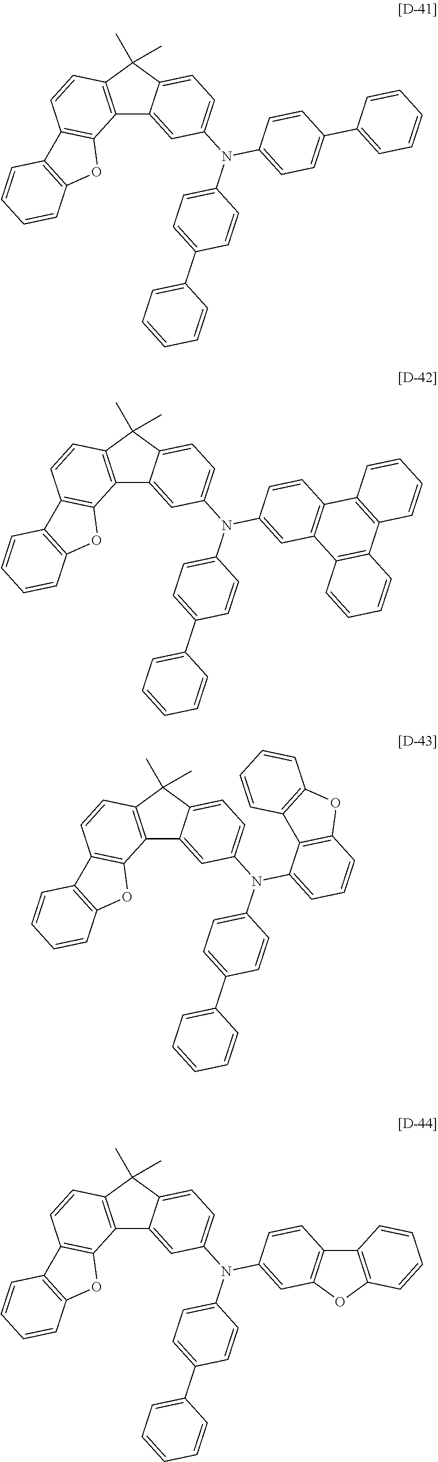 [SMILES: CC1(C)c2ccc(N(c3ccc(-c4ccccc4)cc3)c3ccc(-c4ccccc4)cc3)cc2-c2c1ccc1c2oc2ccccc21 ; CC1(C)c2ccc(N(c3ccc(-c4ccccc4)cc3)c3ccc4c5ccccc5c5ccccc5c4c3)cc2-c2c1ccc1c2oc2ccccc21 ; CC1(C)c2ccc(N(c3ccc(-c4ccccc4)cc3)c3cccc4oc5ccccc5c34)cc2-c2c1ccc1c2oc2ccccc21 ; CC1(C)c2ccc(N(c3ccc(-c4ccccc4)cc3)c3ccc4c(c3)oc3ccccc34)cc2-c2c1ccc1c2oc2ccccc21]

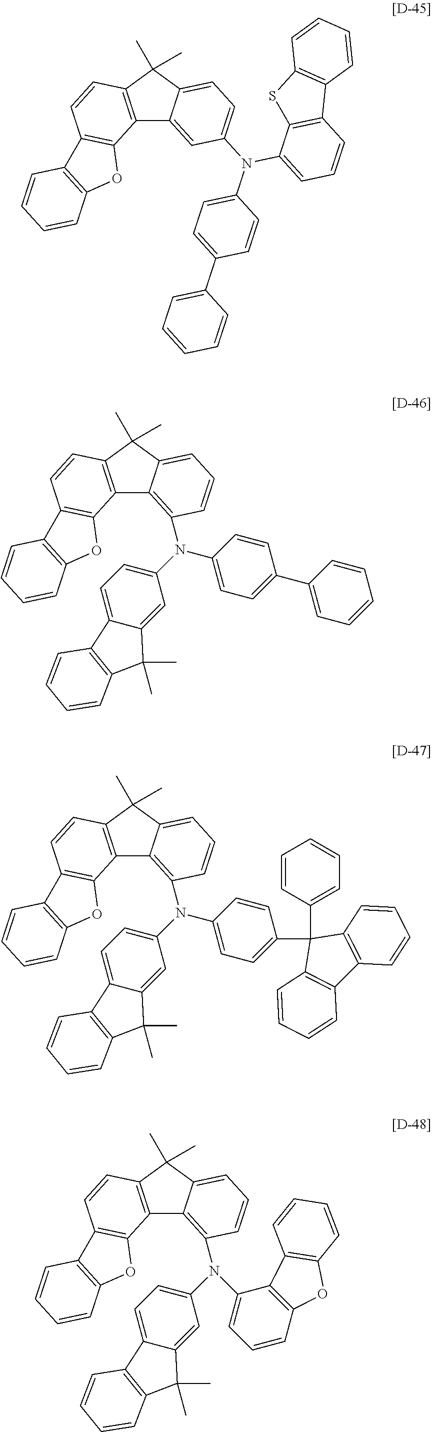 [SMILES: CC1(C)c2ccc(N(c3ccc(-c4ccccc4)cc3)c3cccc4c3sc3ccccc34)cc2-c2c1ccc1c2oc2ccccc21 ; CC1(C)c2ccccc2-c2ccc(N(c3ccc(-c4ccccc4)cc3)c3cccc4c3-c3c(ccc5c3oc3ccccc35)C4(C)C)cc21 ; CC1(C)c2ccccc2-c2ccc(N(c3ccc(C4(c5ccccc5)c5ccccc5-c5ccccc54)cc3)c3cccc4c3-c3c(ccc5c3oc3ccccc35)C4(C)C)cc21 ; CC1(C)c2ccccc2-c2ccc(N(c3cccc4c3-c3c(ccc5c3oc3ccccc35)C4(C)C)c3cccc4oc5ccccc5c34)cc21]

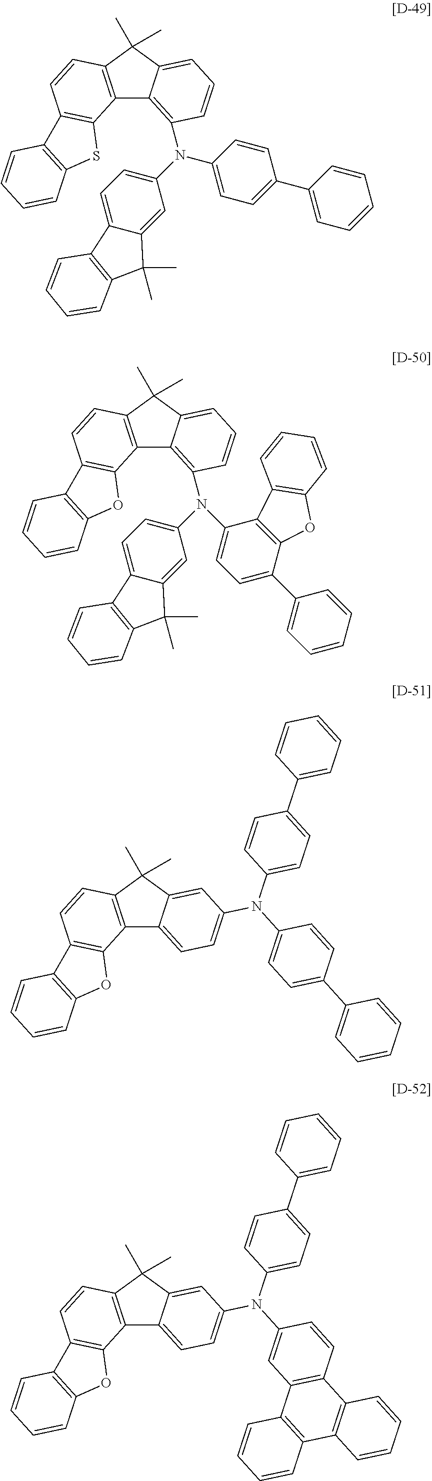 [SMILES: CC1(C)c2ccccc2-c2ccc(N(c3ccc(-c4ccccc4)cc3)c3cccc4c3-c3c(ccc5c3sc3ccccc35)C4(C)C)cc21 ; CC1(C)c2ccccc2-c2ccc(N(c3cccc4c3-c3c(ccc5c3oc3ccccc35)C4(C)C)c3ccc(-c4ccccc4)c4oc5ccccc5c34)cc21 ; CC1(C)c2cc(N(c3ccc(-c4ccccc4)cc3)c3ccc(-c4ccccc4)cc3)ccc2-c2c1ccc1c2oc2ccccc21 ; CC1(C)c2cc(N(c3ccc(-c4ccccc4)cc3)c3ccc4c5ccccc5c5ccccc5c4c3)ccc2-c2c1ccc1c2oc2ccccc21]

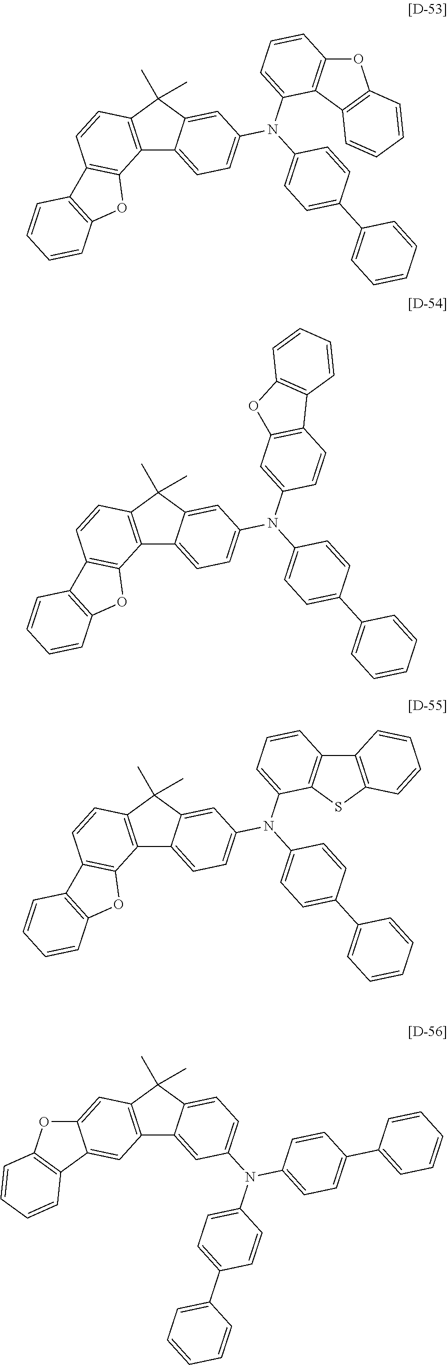 [SMILES: CC1(C)c2cc(N(c3ccc(-c4ccccc4)cc3)c3cccc4oc5ccccc5c34)ccc2-c2c1ccc1c2oc2ccccc21 ; CC1(C)c2cc(N(c3ccc(-c4ccccc4)cc3)c3ccc4c(c3)oc3ccccc34)ccc2-c2c1ccc1c2oc2ccccc21 ; CC1(C)c2cc(N(c3ccc(-c4ccccc4)cc3)c3cccc4c3sc3ccccc34)ccc2-c2c1ccc1c2oc2ccccc21 ; CC1(C)c2ccc(N(c3ccc(-c4ccccc4)cc3)c3ccc(-c4ccccc4)cc3)cc2-c2cc3c(cc21)oc1ccccc13]

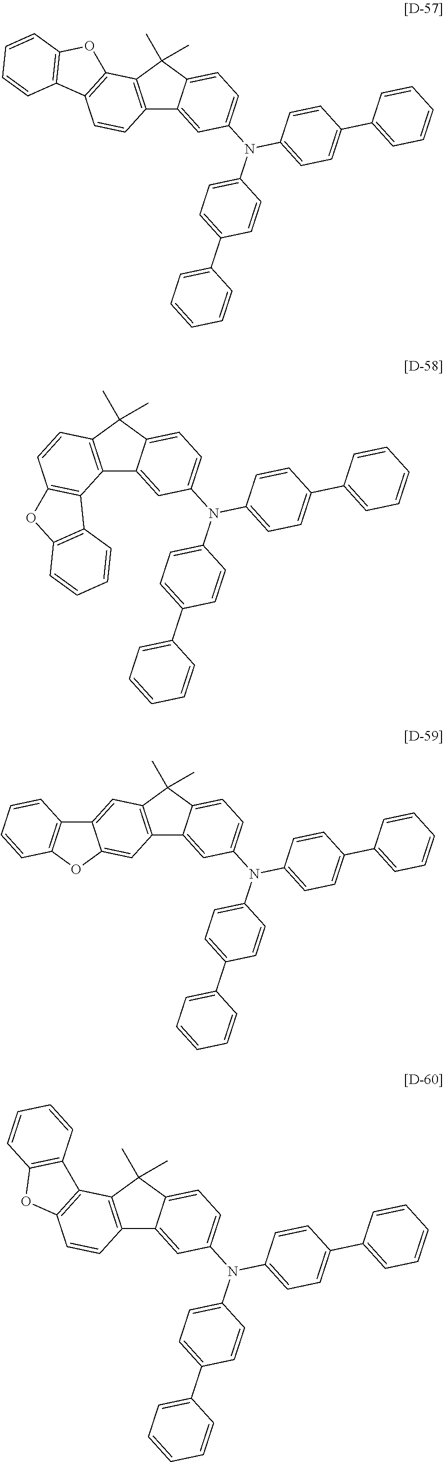 [SMILES: CC1(C)c2ccc(N(c3ccc(-c4ccccc4)cc3)c3ccc(-c4ccccc4)cc3)cc2-c2ccc3c(oc4ccccc43)c21 ; CC1(C)c2ccc(N(c3ccc(-c4ccccc4)cc3)c3ccc(-c4ccccc4)cc3)cc2-c2c1ccc1oc3ccccc3c21 ; CC1(C)c2ccc(N(c3ccc(-c4ccccc4)cc3)c3ccc(-c4ccccc4)cc3)cc2-c2cc3oc4ccccc4c3cc21 ; CC1(C)c2ccc(N(c3ccc(-c4ccccc4)cc3)c3ccc(-c4ccccc4)cc3)cc2-c2ccc3oc4ccccc4c3c21]

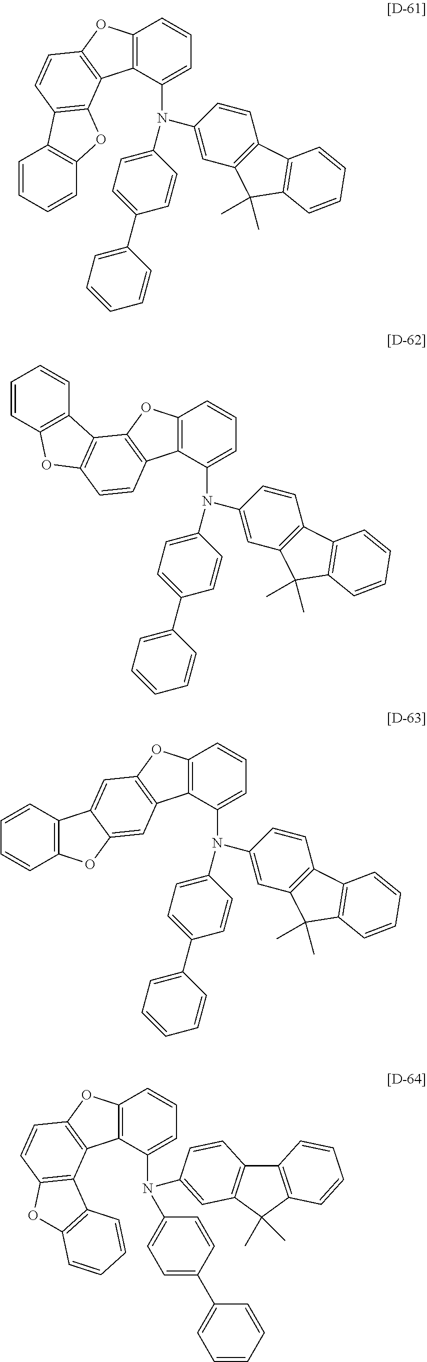 [SMILES: CC1(C)c2ccccc2-c2ccc(N(c3ccc(-c4ccccc4)cc3)c3cccc4oc5ccc6c7ccccc7oc6c5c34)cc21 ; CC1(C)c2ccccc2-c2ccc(N(c3ccc(-c4ccccc4)cc3)c3cccc4oc5c(ccc6oc7ccccc7c65)c34)cc21 ; CC1(C)c2ccccc2-c2ccc(N(c3ccc(-c4ccccc4)cc3)c3cccc4oc5cc6c(cc5c34)oc3ccccc36)cc21 ; CC1(C)c2ccccc2-c2ccc(N(c3ccc(-c4ccccc4)cc3)c3cccc4oc5ccc6oc7ccccc7c6c5c34)cc21]

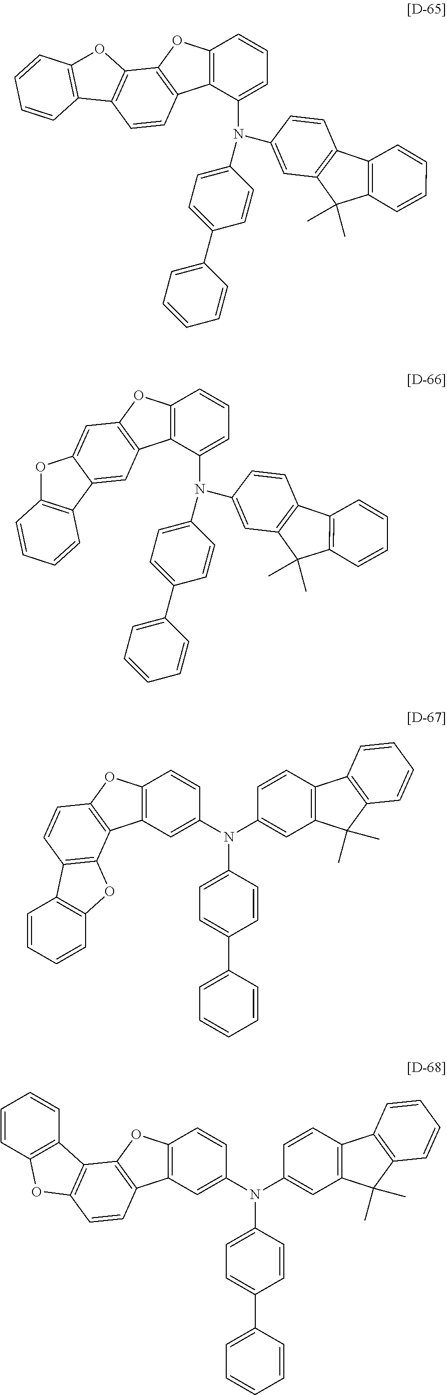 [SMILES: CC1(C)c2ccccc2-c2ccc(N(c3ccc(-c4ccccc4)cc3)c3cccc4oc5c(ccc6c7ccccc7oc65)c34)cc21 ; CC1(C)c2ccccc2-c2ccc(N(c3ccc(-c4ccccc4)cc3)c3cccc4oc5cc6oc7ccccc7c6cc5c34)cc21 ; CC1(C)c2ccccc2-c2ccc(N(c3ccc(-c4ccccc4)cc3)c3ccc4oc5ccc6c7ccccc7oc6c5c4c3)cc21 ; CC1(C)c2ccccc2-c2ccc(N(c3ccc(-c4ccccc4)cc3)c3ccc4oc5c(ccc6oc7ccccc7c65)c4c3)cc21]

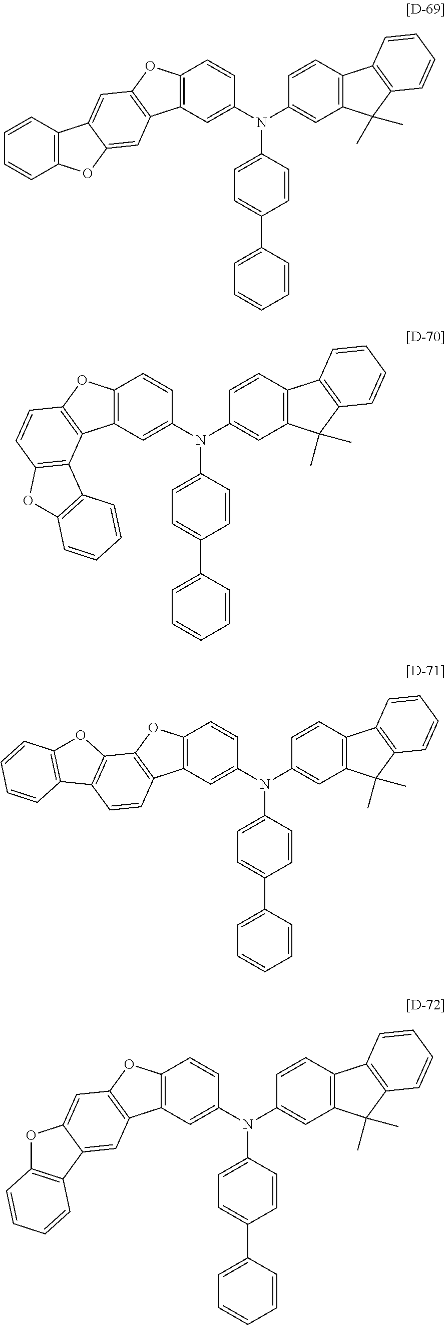 [SMILES: CC1(C)c2ccccc2-c2ccc(N(c3ccc(-c4ccccc4)cc3)c3ccc4oc5cc6c(cc5c4c3)oc3ccccc36)cc21 ; CC1(C)c2ccccc2-c2ccc(N(c3ccc(-c4ccccc4)cc3)c3ccc4oc5ccc6oc7ccccc7c6c5c4c3)cc21 ; CC1(C)c2ccccc2-c2ccc(N(c3ccc(-c4ccccc4)cc3)c3ccc4oc5c(ccc6c7ccccc7oc65)c4c3)cc21 ; CC1(C)c2ccccc2-c2ccc(N(c3ccc(-c4ccccc4)cc3)c3ccc4oc5cc6oc7ccccc7c6cc5c4c3)cc21]

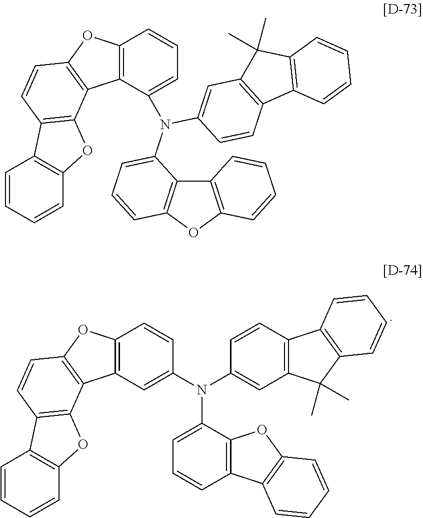 [SMILES: CC1(C)c2ccccc2-c2ccc(N(c3cccc4oc5ccccc5c34)c3cccc4oc5ccc6c7ccccc7oc6c5c34)cc21 ; CC1(C)c2ccccc2-c2ccc(N(c3ccc4oc5ccc6c7ccccc7oc6c5c4c3)c3cccc4c3oc3ccccc34)cc21]

11. The organic optoelectronic device as claimed in claim 1, wherein:
the second compound is represented by Chemical Formula 2-8, and
the third compound is represented by Chemical Formula 5A-4: 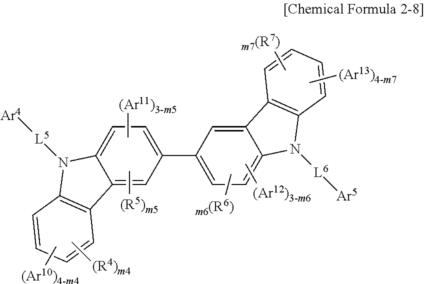
in Chemical Formula 2-8,
R⁴ to R⁷ are each independently hydrogen, deuterium, or a substituted or unsubstituted C6 to C12 aryl group,
Ar¹⁰ to Ar¹³ are each independently hydrogen or a substituted or unsubstituted C6 to C12 aryl group,
m4 and m7 are each independently an integer of 1 to 4,
m5 and m6 are each independently an integer of 1 to 3, and
moieties *L-Ar⁴ and *L⁶Ar⁵ are each independently a moiety of Group II, 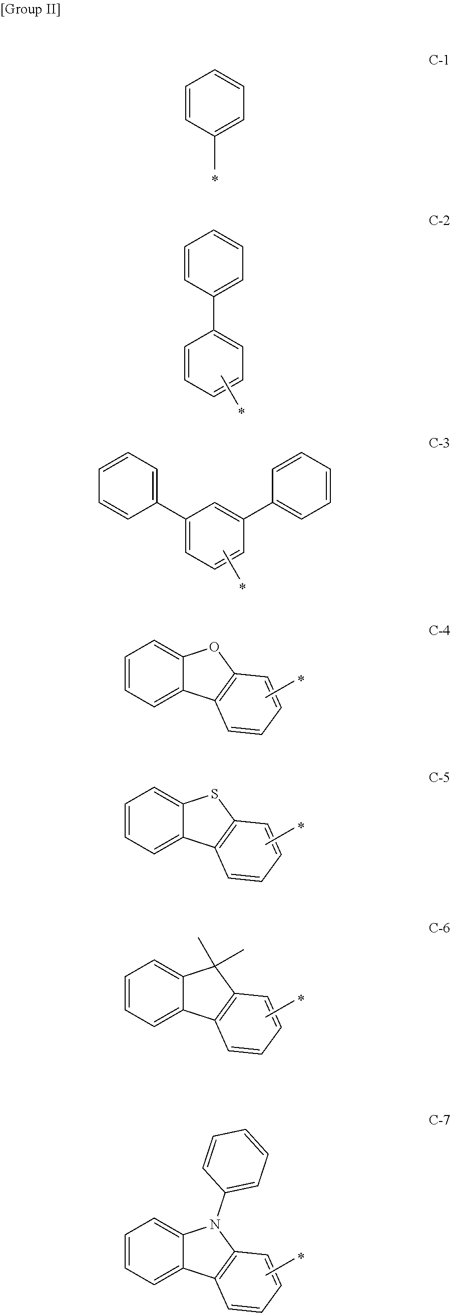 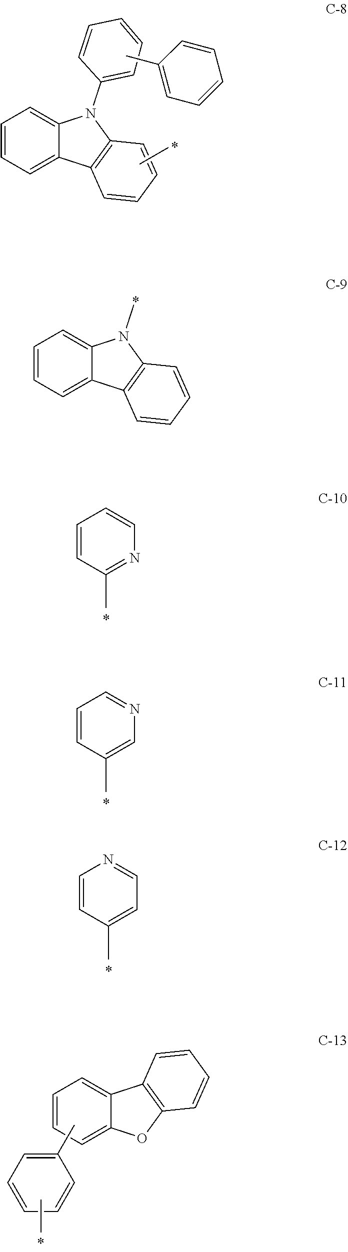 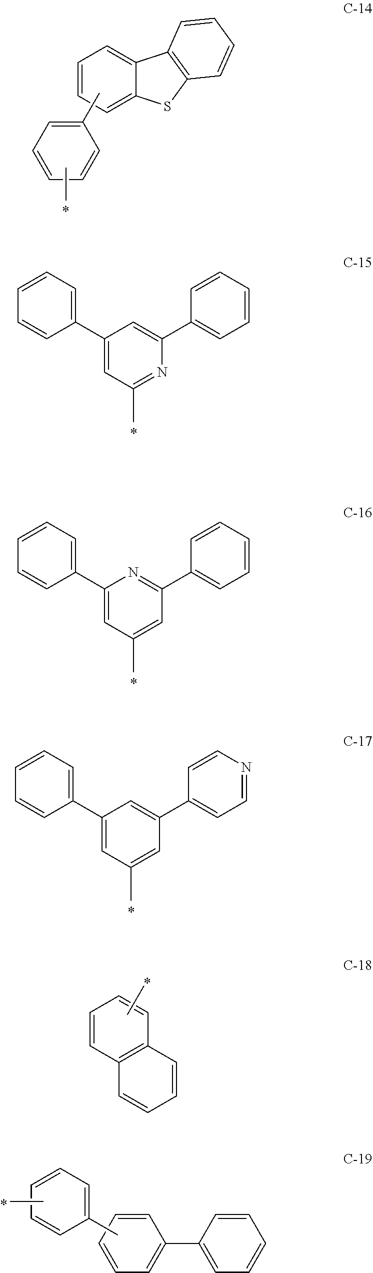 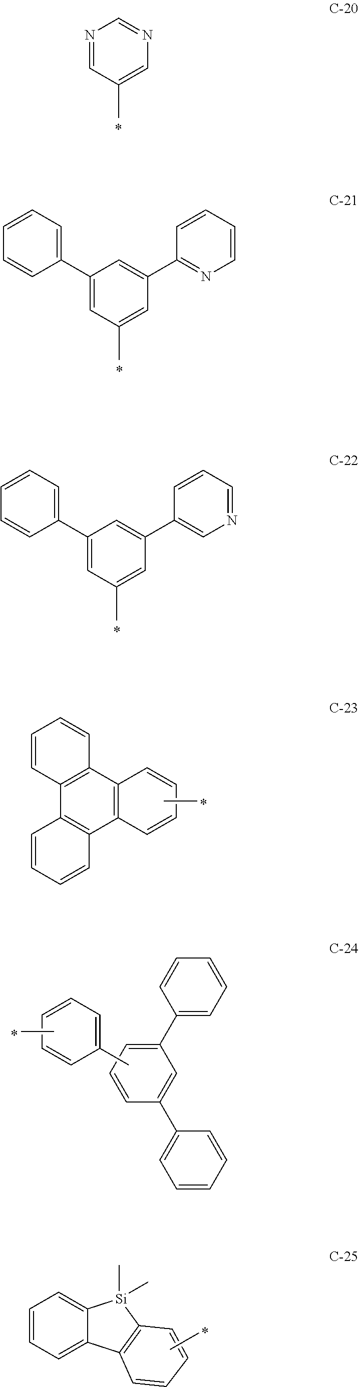 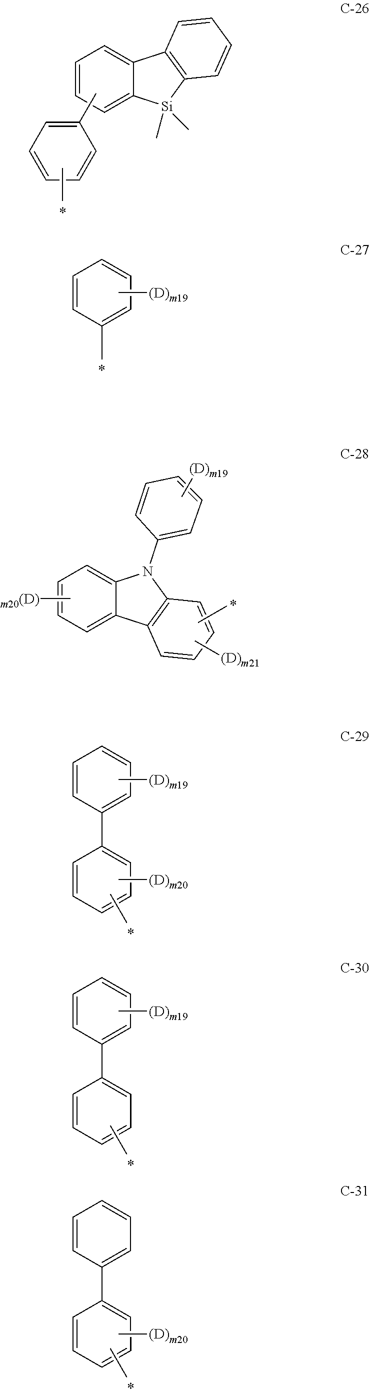 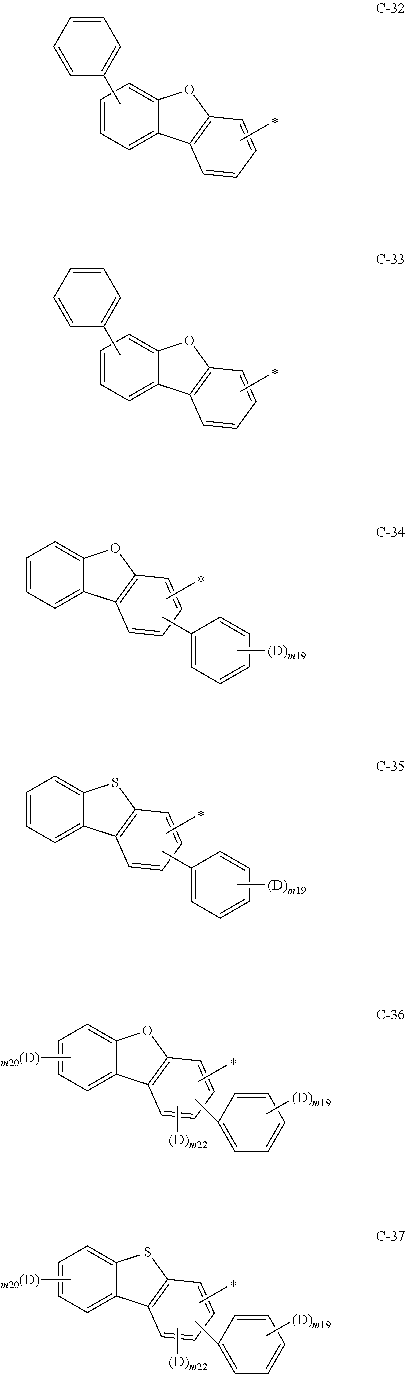 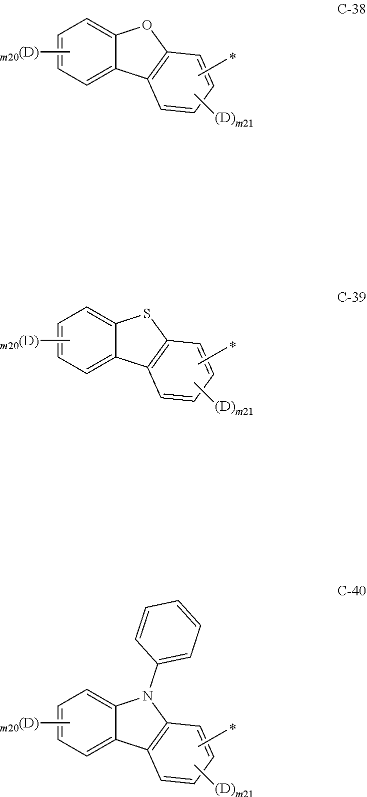
in Group I1,
D is deuterium,
m19 is an integer of 1 to 5,
m20 is an integer of 1 to 4,
m21 is an integer of 1 to 3,
m22 is 1 or 2, and
* is a linking point; 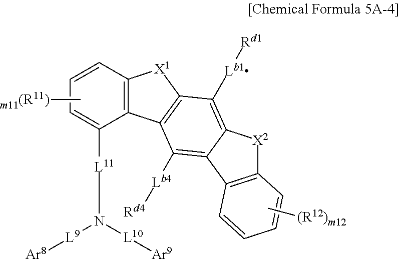
in Chemical Formula 5A-4,
R¹¹, R¹², L^{b1} and L⁴, L⁹ to L¹¹, m11, and m12 are defined the same as those of Chemical Formulae 5 and 6,
R^{d1} and R^{d4} are defined the same as R^{d},
L^{b1} and L^{b4} are defined the same as L^{b},
X¹ is O or S,
X² is CR^{b}R^{c},
R^{b} and R^{c} are each independently a substituted or unsubstituted C1 to C30 alkyl group or a substituted or unsubstituted C6 to C30 aryl group,
Ar⁸ and Ar⁹ are each independently a substituted or unsubstituted biphenyl group, a substituted or unsubstituted fluorenyl group, a substituted or unsubstituted triphenylene group, a substituted or unsubstituted dibenzofuranyl group, or a substituted or unsubstituted dibenzothiophenyl group, and
at least one of Ar⁸ and Ar⁹ is a substituted or unsubstituted fluorenyl group or a substituted or unsubstituted dibenzosilolyl group.

12. A display device comprising the organic optoelectronic device as claimed in claim 1.
